(19) United States
(12) Patent Application Publication
(10) Pub. No.: US 2022/0220186 A1
(43) Pub. Date: Jul. 14, 2022

(54) CHIMERIC CO-STIMULATORY PROTEINS COMPRISING MUTANT INTRACELLULAR DOMAINS WITH INCREASED EXPRESSION

(71) Applicant: Neomics Pharmaceuticals LLC, Gaithersburg, MD (US)

(72) Inventors: William A. COMRIE, Silver Spring, MD (US); Wenshan HAO, Parsippany, NJ (US)

(21) Appl. No.: 17/568,189

(22) Filed: Jan. 4, 2022

Related U.S. Application Data
(60) Provisional application No. 63/133,494, filed on Jan. 4, 2021.
(60) Provisional application No. 63/163,171, filed on Mar. 19, 2021.

(51) Int. Cl. C07K 14/725; C07K 14/705; C07K 14/715; C12N 5/0783; A61K 35/17

(52) U.S. Cl. CPC C07K 14/7051; C07K 14/70578; C07K 14/70521; C07K 14/70507; C07K 14/7155; A61K 2039/5158; A61K 35/17; C07K 2319/03; C07K 2319/02; C12N 2510/00; C12N 5/0636

(57) ABSTRACT

The present application relates to functionally optimized intracellular co-stimulatory domains, optionally in combination with cell-intrinsic immune checkpoint inhibitory receptors or immune-stimulatory receptors or portions thereof, which can be used in adoptive cell therapy to treat human diseases and disorders.

### CROSS-REFERENCE TO RELATED APPLICATIONS

This application claims priority to and the benefit of U.S. Provisional Application No. 63/163,171, filed on Mar. 19, 2021 and U.S. Provisional Application No. 63/133,494, filed on Jan. 4, 2021, the contents of each of which are hereby incorporated by reference in their entirety.

### BACKGROUND

While adoptive cell therapies show efficacy in cancer treatment, the effectiveness of these therapies can be further improved through genetic engineering of T cells for better expansion and persistence. T cells require functionally non-overlapping co-stimulatory signals from CD28 family and tumor necrosis factor receptor (TNFR) family along with antigen triggered TCR signaling to promote full-fledged activation and persistent proliferation. In developing gene-engineered T cell therapeutics, there is a need to introduce chimeric T cell co-stimulatory molecules that can be locally activated upon T cell engaging with pathological antigens to potently augment T cell activation for increased therapeutic efficacy. Currently, the second generation of chimeric co-stimulatory molecules incorporating one co-stimulatory signaling domain from proteins of either CD28 family or TNFR family has been widely adopted in CAR T cell therapy. Chimeric antigen receptors (CARs) integrating one co-stimulatory signaling domain augment T cell function through both activating and co-stimulatory signals, thus resulting in enhanced anti-tumor potency and T cell persistence. Given that the capacity for T cell to expand depends on the structural design, the current second generation co-stimulatory proteins may not be optimal for induction of a durable tumor remissions. Thus, there is a desired effort to develop third-generation chimeric molecules combining two co-stimulatory signaling domains from CD28 family and TNFR family members to further enhance T cell therapeutic potential, capitalizing on non-overlapping functions of the two families of co-stimulatory molecules. In addition, such third-generation chimeric co-stimulatory molecules can be integrated into TCR T therapy where T cell activation remains suboptimal due to insufficient co-stimulatory signals during activation of exogenously expressed TCRs by antigens. However, existing recombinant DNA strategies often suffer from reduced cell surface expression of the chimeric proteins combining two co-stimulatory signaling domains, preventing realization of the functional potential of the chimeric proteins. The present application addresses such needs. The present application discloses third-generation chimeric T cell co-stimulatory molecules that incorporate two signaling domains from CD28 and TNFR families and express at significantly improved levels than what have been conventionally reported for enhanced T cell functions, and methods of making the co-stimulatory molecules.

### SUMMARY

Provided herein are novel chimeric co-stimulatory intracellular domains. The chimeric co-stimulatory intracellular domains provided herein comprise: (a) a first signaling domain that is based on the intracellular signaling domain of a CD28 family protein; and (b) at least a second signaling domain that comprises a mutant intracellular signaling domain of a tumor necrosis factor receptor (TNFR) family protein.

In some embodiments, the first signaling domain that is based on the intracellular signaling domain of a CD28 family proteins is selected from a CD28 protein, ICOS protein or a combination thereof. In some embodiments, the at least second signaling domain is based on a mutant of the intracellular signaling domain of a TNFR family protein selected from CD137 (4-1BB) and CD134 (OX-40).

The chimeric co-stimulatory intracellular domains provided herein comprise: (a) a first signaling domain that is based on the intracellular signaling domain of a CD28 protein, ICOS protein or a combination thereof; and (b) at least a second signaling domain that comprises a mutant CD137 (4-1BB) intracellular domain or a mutant CD134 (OX-40) intracellular domain. In some embodiments, the mutant CD137 (4-1BB) intracellular domain or the mutant CD134 (OX-40) intracellular domain comprises a deletion, an insertion or a substitution of one or more amino acids in the membrane proximal portion of the CD137 or CD134 intracellular domain. Without being bound by theory, in some embodiments, the one or more amino acids in the membrane proximal portion can be ubiquitination sites involved in the ubiquitination and degradation of the CD137 or CD134 protein.

Also disclosed herein is a functionally optimized intracellular co-stimulatory domain for use in novel adoptive cell therapy, optionally in combination with cell-intrinsic immune checkpoint inhibitory receptors or immune-stimulatory receptors or portions thereof, developed to treat human diseases and disorders, including hematological and solid tumors. Also disclosed herein is a functionally optimized intracellular co-stimulatory domain for use in combination with a T cell receptor (TCR), e.g. an endogenous TCR or an affinity enhanced TCR targeting a tumor-associated antigen. Optionally, the intracellular co-stimulatory domain is used in combination with a second component (e.g., a cell surface receptor or portion thereof) that directs migration of an immune cell to bind to a target tissue or cell or induces activation and/or proliferation of an immune cell, such as a PD-1 switch receptor (PD-1 based co-stimulatory molecule), that can increase T cell functionality in tumors, such as a PD-L1/PD-L2-expressing tumor. Also disclosed herein is a therapy that utilizes the PD-1 checkpoint blockade in a cell-intrinsic fashion, which simultaneously minimizes autoimmune side effects and provides increased on-tumor functionality. The present application discloses recombinant T cell co-stimulatory receptors (RTCRs) based on T cell co-receptors or chimeric antigen receptors (CARs) comprising a functionally optimized intracellular co-stimulatory domain of the present application. The present application also discloses T cell co-receptors comprising a functionally optimized intracellular co-stimulatory domain and a PD-1 extracellular domain (i.e., PD-1 switch receptors or PD-1 based co-stimulatory molecules). The present application also discloses CD19 and B cell maturation Ag (BCMA) based CARs comprising a functionally optimized intracellular co-stimulatory domain that promotes CD19 and BCMA binding mediated T cell activation, proliferation, and tumor killing. The RTCRs disclosed in the present application can be used for evaluation of checkpoint targets, safety screening, and for development of pre-clinical animal models to evaluate the effectiveness of the combination of the functionally optimized intracellular co-stimulatory domain of the present application with any TCRs or CARs. Additional cell-intrinsic immune checkpoint inhibitors with the efficacious TCRs are also developed.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant CD137 (4-1BB) intracellular domain or a mutant CD134 (OX-40) intracellular domain.

The present disclosure also provides a nucleic acid encoding the RTCR disclosed herein. The present disclosure also provides a vector comprising the nucleic acid disclosed herein. The present disclosure also provides a cell comprising the nucleic acid or the vector disclosed herein.

The present disclosure also provides a modified T lymphocyte (T cell), comprising: (a) a modification of an endogenous sequence encoding a T cell Receptor (TCR), wherein the modification reduces or eliminates a level of expression or activity of the TCR; and (b) a recombinant T cell co-stimulatory receptor (RTCR) disclosed herein.

The present disclosure also provides a composition comprising the RTCR disclosed herein. The present disclosure also provides a composition comprising the nucleic acid encoding the RTCR disclosed herein. The present disclosure also provides a composition comprising the vector comprising the nucleic acid disclosed herein. The present disclosure also provides a composition comprising the cell disclosed herein. The present disclosure also provides a composition comprising the modified T cell disclosed herein.

The present disclosure also provides a composition comprising a population of cells, wherein the population comprises a plurality of the cell comprising the nucleic acid encoding or a vector comprising the nucleic acid encoding the RTCR disclosed herein. The present disclosure also provides a composition comprising a population of cells, wherein the population comprises a plurality of the modified T cell disclosed herein.

The present disclosure provides a method of producing a plurality of modified T cells, wherein the method comprises: a) providing a plurality of primary T cells disclosed herein; b) providing a composition comprising the RTCR disclosed herein, the nucleic acid encoding the RTCR disclosed herein, or the vector comprising the nucleic acid encoding the RTCR disclosed herein; and c) introducing into the plurality of primary T cells of (a) the composition of (b), to produce a plurality of modified T cells under conditions that stably express the RTCR within the plurality of modified T cells. In some embodiments, the method of producing a plurality of modified T cells disclosed herein, further comprises a step of modifying an endogenous sequence encoding an endogenous T cell Receptor (TCR), wherein the modification reduces or eliminates a level of expression or activity of the endogenous TCR. In some embodiments, the method of producing a plurality of modified T cells disclosed herein, further comprises a step of modifying an endogenous sequence, wherein the modification reduces or eliminates a level of expression or activity of a major histocompatibility complex (MHC) class I (MHC-I). In some embodiments, the method of producing a plurality of modified T cells disclosed herein, further comprises: d) maintaining the plurality of modified T cells in a suitable cell culture media; and e) either: i) cryopreserving the plurality of modified T cells in a suitable cell freezing media; or ii) preparing the plurality of modified T cells for administering to a subject suffering from a disease or disorder.

The present disclosure also provides a method of treating a disease or disorder, comprising administering to a subject in need thereof a therapeutically effective number of the cell comprising the nucleic acid encoding or the vector comprising the nucleic acid encoding the RTCR disclosed herein, a therapeutically effective number of any one of the modified T cell disclosed herein, a therapeutically effective amount of any one of the compositions disclosed herein, or a therapeutically effective number of the plurality of modified T cells produced by the method disclosed herein.

The present disclosure also provides a chimeric co-stimulatory intracellular protein (CIP) comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

The present disclosure also provides a chimeric co-stimulatory intracellular protein (CIP) comprising a first and at least a second signal transduction domains, wherein the first and the at least second signal transduction domains are non-identical; and wherein the at least second signal transduction domain comprises a mutant CD137 (4-1BB) intracellular domain or a mutant CD134 (OX-40) intracellular domain.

Throughout the specification the term “comprising,” or variations such as “comprises” or “comprise,” will be understood to imply the inclusion of a stated element, integer or step, or group of elements, integers or steps, but not the exclusion of any other element, integer or step, or group of elements, integers or steps.

Throughout the specification the term “signal domain”, “signaling domain”, and “signal transduction domain”, are used interchangeably, unless the context dictates otherwise.

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### DESCRIPTION OF THE FIGURES

FIG. 1 depicts alignment of the intracellular tails of TNF receptor superfamily members that are used in the T cell co-stimulatory molecules of the present application. Membrane-proximal poly-basic regions are italicized. Potential PI3K binding sites are bold and underlined. TRAF1/2 binding motifs: major motif Px(Q/E)E and minor motif Px(Q/E)x, are underlined. Potential ubiquitination sites are in bold.

FIGS. 2A-2B depict the modular design of 2^{nd} and 3^{rd} generation co-stimulatory molecules. FIG. 2A depicts modular design of co-stimulatory molecules denoting the signal peptide, extracellular domain, transmembrane domain, and intracellular signaling domain. FIG. 2B depicts structures and sequences of first signal transduction domains: ICOS, CD28 and ICOS intracellular domain with a portion of CD28 domain inserted. Regions and known binding partners of the ICOS and CD28 intracellular domain with specific binding function are indicated. The amino acid/nucleic acid sequences of the co-stimulatory molecules and the intracellular domains are as indicated.

FIG. 3 depicts the combinations of extracellular effector domains and intracellular signaling domains of the present application.

FIGS. 4A-4B depict that deletion of the N-terminal section of the 4-1BB signaling domain, including the polybasic domain and lysine residues, rescues the expression of the co-stimulatory molecules. FIG. 4A depicts expression of human PD1 and huEGFRt on the surface of T-lymphocytes following lentiviral transduction with the indicated construct. FIG. 4B depicts the normalized PD1 surface expression on huEGFRt-expressing cells expressing different co-stimulatory molecules with ICOS or CD28 based chimeric intracellular domains comprising wild type or truncated 4-1BB domains or OX-40 domains, as indicated. The amino acid/nucleic acid sequences of the chimeric intracellular domains are as indicated.

FIGS. 5A-5D depict cytokine production and proliferation of T cells expressing different co-stimulatory molecules with ICOS based chimeric intracellular domains comprising wild type or truncated 4-1BB or OX-40 domains. FIGS. 5A-5C depict that antibody-mediated crosslinking of co-stimulatory molecules increases T cell cytokine production in-vitro. IL-2 (FIG. 5 A), TNF (FIG. 5 B), and IFNγ (FIG. 5 C) were measured by bead-based multiplex assay in culture supernatants following 18 hr stimulation of T cells transduced with the indicated constructs with plate-bound anti-PD1 [2 μg/mL] and the indicated amount of plate-bound anti-CD3. The x-axis indicates the different co-stimulatory molecules and the y-axis indicates the amount of cytokine produced expressed as absorbance unit (A.U.). FIG. 5D depicts proliferation of T cells stimulated with the indicated plate-bound antibodies for 96 hrs. The amino acid/nucleic acid sequences of the chimeric intracellular domains are as indicated.

FIGS. 6A-6C depict that PD-L1 engagement of co-stimulatory molecules increases T cell cytokine production in-vitro. FIG. 6A depicts IL-2 (upper panel), IFNγ (middle panel), and TNF (lower panel) measured by bead-based multiplex assay in culture supernatants following 18 hr stimulation of T cells transduced with the indicated constructs. The x-axis indicates amount of anti-CD3 antibody (μg/ml) and the y-axis indicates cytokine production as percentage of control. FIGS. 6B-6C depict IL-2 (upper panels), IFNγ (middle panel), and TNF (lower panel) production by T cells transduced with the indicated co-stimulatory molecules comprising CD28 intracellular domain (FIG. 6B, upper, middle and lower panels) and ICOS intracellular domain (FIG. 6C, upper, middle and lower panels), respectively, and stimulated with the indicated concentration of soluble anti-CD3 antibody in the presence of K562 cells expressing HLA-A2 (left panels) or HLA-A2 and PD-L1 (right panels). The x-axis indicates amount of anti-CD3 antibody (μg/ml) and the y-axis indicates cytokine production as percentage of control. The amino acid/nucleic acid sequences of the co-stimulatory molecules are as indicated.

FIGS. 7A-7C depict that PD-L1 engagement of co-stimulatory molecules increases T cell cytotoxicity and proliferation in-vitro. T cells expressing PD-1 constructs, as indicated, and K562 cells were mixed and stimulated as in FIG. 6. FIGS. 7A and 7B depict the number of remaining K562 cells (upper panel) and number of T cells (lower panel) evaluated by flow cytometry, after 96 hours of stimulation with the indicated concentration of soluble anti-CD3 antibody in the presence of K562 cells expressing HLA-A2 (left panels) or HLA-A2 and PD-L1 (right panels). FIG. 7C depicts proliferation of T cells expressing the various PD1 constructs co-cultured with K562 cells expressing HLA-A2 (indicated by “X”) or HLA-A2 and PD-L1 (indicated by “*”) and 0.3 μg/ml of anti-CD3, as measured by shift in Cell Trace violet dilution as indicated on x-axis. FIG. 7D is a graph depicting target cell (K562) numbers remaining evaluated by flow cytometry, after 96 hours post stimulation with T cells expressing co-stimulatory molecules, in presence of increasing amounts anti-CD3 antibody (μg/ml), as indicated. FIG. 7E is a graph depicting number of T cells evaluated by flow cytometry, after 96 hours post stimulation with T cells expressing co-stimulatory molecules, in presence of increasing amounts anti-CD3 antibody (μg/ml), as indicated. The x-axis indicates amount of CD3 antibody (μg/ml) and the y-axis indicates number of cells. The amino acid/nucleic acid sequences of the co-stimulatory molecules are as indicated.

FIG. 8 depicts that engagement of co-stimulatory molecules increases T cell proliferation in-vitro. T cells were stimulated for 96 hrs on plate-bound antibodies with 2 μg/mL anti-PD1 and the concentration of anti-CD3 [mg/mL] (indicated by “*”) or only anti-CD3 (indicated by “X”), as indicated on y-axis, and proliferation of T cells expressing the various PD1 constructs as measured by shift in crystal violet tracing as indicated on x-axis. The amino acid/nucleic acid sequences of the co-stimulatory molecules are as indicated.

FIGS. 9A-9C depict the effect of mutation of the polybasic and lysine residues on expression or function of co-stimulatory molecules incorporating ICOS and 4-1BB signaling domains. FIG. 9A is a series of flow cytometry plots depicting proliferation of T-cells expressing either a wild type PD1 receptor (indicated by “*”) or the different PD1 based co-stimulatory molecules (indicated by “X”), as indicated by labeling at top of each plot. T-cells expressing endogenous PD-1 were used as control (line with no indication). FIG. 9B is a graph depicting PD-1 expression (expressed as a fold increase from endogenous levels) from the FACS plots in FIG. 9A. FIG. 9C are graphs depicting cytokine production (IL-2, left panels; IFNy, middle panels; and TNF, right panels) (y-axis) by T cells expressing different co-stimulatory molecules, as indicated, responding to K562 cells (top row) and K562-PDL1 expressing cells (middle row), when stimulated with the indicated concentration of anti-CD3 (x-axis). The difference between the level of cytokine production between T cells responding to K562 cells and K562-PDL1 expressing cells, is depicted in the graphs in the bottom row. FIG. 9D are graphs depicting proliferation of T cells 96 hr post culturing with K562 cells (left graph) or K562 cells expressing PD-L1 (middle graph), when stimulated with the indicated concentration of anti-CD3 (x-axis). The amino acid/nucleic acid sequences of the co-stimulatory molecules are as indicated.

FIGS. 10A-10D depicts the expression and function of co-stimulatory molecules incorporating ICOS and OX-40 signaling domains. FIG. 10A is a series of flow cytometry plots depicting proliferation of T-cells expressing either a wild type PD1 receptor (indicated by “*”) or the different PD1-switch receptors (PD1 based costimulatory molecules) (indicated by “X”), as indicated by labeling at top of each plot. T-cells expressing endogenous PD-1 were used as control (no indication). FIG. 10B is a graph depicting PD-1 expression (fold of endogenous expression) from the FACS plots in FIG. 10A. FIG. 10C are graphs depicting cytokine production (IL-2, left panels; IFNy, middle panels; and TNF, right panels) (y-axis) by T cells expressing different co-stimulatory molecules, as indicated, responding to K562 cells (top row) and K562-PDL1 expressing cells (middle row), when stimulated with the indicated concentration of anti-CD3 (x-axis). The difference between the level of cytokine production between T cells responding to K562 cells and K562-PDL1 expressing cells, is depicted in the graphs in the bottom row. FIG. 10D are graphs depicting T Cell proliferation 96 hr post culturing with K562 cells (left graph) or K562 cells expressing PD-L1 (middle graph), when stimulated with the indicated concentration of anti-CD3 (x-axis). The difference between T cell proliferation in the presence or absence of PD-L1 on the target cells is depicted in the right-most graph. The amino acid/nucleic acid sequences of the co-stimulatory molecules are as indicated.

FIGS. 11A-11B depict that engagement of co-stimulatory molecules increases T cell conjugation with PD-L1 expressing cells. FIG. 11A depicts flow cytometry gating strategy of a 30-minute conjugation of CFSE-labelled T cells with CTV-labelled K562 targets. FIG. 11B depicts quantification of results from two experiments shown in FIG. 11A and normalized to control conjugations. The amino acid/nucleic acid sequences of the co-stimulatory molecules, with and without a signaling peptide, are as indicated.

FIGS. 12A-12C depict increase in T cell proliferation and function upon engagement of co-stimulatory molecules with PD-L1 expressing cells. FIG. 12A are flow cytometry plots depicting surface expression of PD-1 and TCR β chain, in T cells expressing either a wild type HLA-A2/NY-ESO-1 specific TCRs or mutant NY-ESO TCR as indicated, with (lower middle and right plots) and without (upper middle and right plots) a co-stimulatory molecule construct comprising an ICOS_4-1BB (truncated) signaling domain (PD-1_ICOS_BBt), as indicated, when co-cultured with K562 cells), 72 hrs after lentiviral transfection. FIG. 12B are graphs depicting IL-2 (top graph) and IFNγ (bottom graph) production T cells expressing NY-ESO-1/PD1 based co-stimulatory molecule combinations, as indicated, when co-cultured with A375-tumor cells that express HLA-A2 and antigen, at T cell: A375 cell ratio as indicated in x-axis. FIG. 12C is a graph depicting dose dependent killing of A375 cells by T cells expressing the indicated wild type NY-ESO TCR or mutant, high affinity (HA) NY-ESO TCR, as indicated with/without a co-stimulatory molecules construct comprising an ICOS_4-1BB (truncated) signaling domain (PD-1_ICOS_BBt), as indicated. The x-axis depicts the dose (T cell: A375 cell ratio) and percentage of total input A375 cells surviving. The amino acid/nucleic acid sequences of the co-stimulatory molecules, with and without a signaling peptide, are as indicated.

FIGS. 13A-13B depict that mutations of 3^{rd} generation tails increase surface expression of CD-19 CAR receptors on transduced primary T cells. FIG. 13A depicts histograms of CD-19Fc binding to the untransduced T cells (marked by x) or T cells transduced with the indicated constructs (marked by *). FIG. 13B depicts MFI measurements of histograms shown in FIG. 13A, normalized to FMC63scFV_BB_Z. The amino acid/nucleic acid sequences of the co-stimulatory molecules are as indicated.

FIGS. 14A-14C depict that modified 3^{rd} generation tails increase cytokine production and tumor killing. In-vitro killing of CD19-positive cells by CAR-transduced primary T cells is shown. FIG. 14A depicts residual cell number of B cell line (Nalm6 cells), after a 96-hr co-culture with CAR-T cells expressing CD28-based (left panel) and ICOS-based (right panel) 2^{nd} generation and 3^{rd} generation receptors. FIG. 14B depicts residual cell number of B cell line (Raji cells), after a 96-hr co-culture with CAR-T cells expressing CD28-based (left panel) and ICOS-based (right panel) 2^{nd} generation and 3^{rd}-generation receptors. The y-axis depicts number of remaining CD19-positive cells corresponding to the ratio of T cells to CD19-positive cell indicated on x-axis. FIG. 14C depicts 18 hr-IFNγ production, as indicated on the y-axis, by T cells expressing 2^{nd} generation and 3^{rd} generation, CD28-based receptors (left panel) and ICOS-based receptors (right panel), in response to incubation with CD19-positive B cells for 18 hours, at T cell: target cell ratio, as indicated on x-axis. The amino acid/nucleic acid sequences of the co-stimulatory molecules are as indicated.

FIG. 15A-15E depict modified 3^{rd} generation signaling domains increase CD19-CAR function in-vitro, compared to original 3^{rd} generation signaling domains. FIG. 15A is a graph depicting cumulative T cell numbers (indicative of T cell proliferation) (y-axis) of T-cells expressing: a) CD28-based 2^{nd} and 3^{rd} generation receptors (left panel); and b) ICOS-based and 3^{rd}-generation receptors (right panel), as indicated, over repeated stimulations with Nalm6 B cells, as indicated on x-axis. FIG. 15B is a graph depicting cumulative T cell numbers (indicative of T cell proliferation) (y-axis) of T-cells expressing: a) CD28-based 2^{nd} and 3^{rd} generation receptors (left panel); and b) ICOS-based 2^{nd} and 3^{rd} generation receptors (right panel), as indicated, over repeated stimulations with RAJI B cells, as indicated on x-axis. FIG. 15C is a graph depicting cumulative target cell (Nalm6) numbers (indicative of target cell killing) (y-axis) of T-cells expressing: a) CD28-based 2^{nd} and 3^{rd} generation receptors (left panel); and b) ICOS-based 2^{nd} and 3^{rd} generation receptors (right panel), as indicated, over repeated stimulations with Nalm6 B cells, as indicated on x-axis. FIG. 15D is a graph depicting cumulative target cell (Raji) numbers (indicative of target cell killing) (y-axis) of T-cells expressing: a) CD28-based 2^{nd} and 3^{rd} generation receptors (left panel); and b) ICOS-based 2^{nd} and 3^{rd} generation receptors (right panel), as indicated, over repeated stimulations with Raji B cells, as indicated on x-axis. FIG. 15E is a series of flow cytometry plots depicting Tim3 and PD-1 expression on CAR-T cells, as indicated, at time zero or after 5 consecutive stimulations, with RAJI B cell targets. The amino acid/nucleic acid sequences of the co-stimulatory molecules are as indicated.

FIGS. 16A-16E depicts modified 3^{rd}-generation signaling domains increase BCMA-CAR function in-vitro, compared to original 3^{rd}-generation sequences. FIG. 16A depicts flow cytometry histograms of BCMA-Fc binding to untransduced T cells (indicated by “X”) or T cells transduced with the indicated BCMA CAR-T receptor comprising CD28-based and ICOS based-2^{nd} generation and 3^{rd} generation co-stimulatory molecules (indicated by “*”), as indicated. FIG. 16 B is a graph depicting BCMA-Fc binding (MFI) (x-axis) by from the transduced T cells of the FACS plots in FIG. 16A. FIG. 16C is a set of graphs depicting cumulative T cell numbers (indicative of T cell proliferation) (y-axis) of T-cells expressing: a) CD28-based 2^{nd} and 3^{rd} generation receptors (left panel); and b) ICOS-based 2^{nd} and 3^{rd} generation receptors (right panel), as indicated, over repeated stimulations with RPMI-8226 multiple myeloma target cells. FIG. 16D is a graph depicting cumulative target cell (RPMI-8226 cells) numbers (indicative of target cell killing) (y-axis) of T-cells expressing: a) CD28-based 2^{nd} and 3^{rd} generation receptors (left panel); and b) ICOS-based 2^{nd} and 3^{rd} generation receptors (right panel), as indicated, over repeated stimulations with RPMI-8226 multiple myeloma target cells, as indicated on x-axis. FIG. 16E is a set of graphs depicting cytokine production (IL-2, left panel, TNF, middle panel and IFNγ, right panel) (y-axis) with CAR-T cells transduced with the indicated BCMA CAR constructs comprising CD28-based or ICOS-based co-stimulatory molecules, as indicated, incubated at the indicated effector to target ratio indicated on x-axis, for 18 hrs. The amino acid/nucleic acid sequences of the co-stimulatory molecules are as indicated.

### DETAILED DESCRIPTION

Provided herein are novel chimeric co-stimulatory intracellular domains. The chimeric co-stimulatory intracellular domains provided herein comprise: (a) a first signaling domain that is based on the intracellular signaling domain of a CD28 family protein; and (b) at least a second signaling domain that comprises a mutant intracellular signaling domain of a TNFR family protein.

The CD28 family proteins have a single extracellular immunoglobulin variable-like (IgV) domain followed by a short cytoplasmic tail. Members of the CD28 family proteins include CD28, CD28H, inducible costimulator (ICOS), cytotoxic T lymphocyte-associated antigen 4 (CTLA-4, CD152), program death-1 (PD-1), and B- and T-lymphocyte attenuator (BTLA). CD28, CD28H and ICOS are co-stimulatory proteins that are expressed on T cells that promote activation, high levels of cytokine/chemokine expression, resistance to apoptosis, and proliferation of T cells.

The Tumor Necrosis Factor Receptor (TNFR) family proteins includes TNFR1 (tumor necrosis factor receptor 1/TNFRSF1A), TNFR2 (tumor necrosis factor receptor 2/TNFRSF1B), lymphotoxin β receptor/TNFRSF3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRSF10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TACI/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17, GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27 and ectodysplasin 1-anhidrotic receptor. Interactions between tumor necrosis factor superfamily (TNFSF) ligands and TNF receptor superfamily (TNFRSF) receptors provide the co-stimulatory signals that control the survival, proliferation, differentiation, and effector function of immune cells. Depending upon the specific intracellular signal induced by TNFRSF members, they can be categorized into three groups—death domain (DD)-containing receptors, decoy receptors, and TNF receptor-associated factor (TRAF)-binding receptors. Some TNFRSFs such as TNFR-1, Fas, DR3, DR4, DR5, and DR6, contain their own DDs and/or interact with other cytoplasmic DD-containing adaptor molecules. Some other TNFRSFs, such as TNFR-2, CD27, CD30, CD40, glucocorticoid-induced TNFR family-related gene (GITR), Fn1, lymphotoxin beta-receptor (LTβR), OX40, receptor activator of NF-κB (RANK), and XEDAR, lack a DD and contain motifs with four to six amino acids called TRAF-interacting motifs (TIMs) which recruits TRAF proteins. TRAF proteins are adaptor molecules that activate multiple downstream signaling pathways such as NF-κB, Janus kinase (JNK), ERK, p38MAPK, and PI3K that help in cell survival, proliferation, and cytokine production. In some embodiments, the first signaling domain that is based on the intracellular signaling domain of a CD28 family protein is selected from a CD28 protein, ICOS protein or a combination thereof. In some embodiments, the at least second signaling domain is based on a mutant of the intracellular signaling domain of a TNFR family protein is selected from CD137 (4-1BB) and CD134 (OX-40).

Provided herein are novel chimeric co-stimulatory intracellular domains based on the third-generation co-stimulatory domains of the present application. Reduced surface expression is a major hindrance in the development of chimeric co-stimulatory proteins for therapeutic purposes. The present disclosure provides novel chimeric co-stimulatory intracellular domains generated through mutations in the third-generation co-stimulatory domains of the present application that are both highly expressed and highly functional compared to the current second-generation and third-generation chimeric receptors that are effective in inducing costimulation. The chimeric co-stimulatory intracellular domains provided herein comprise: (a) a first signaling domain that is based on the intracellular signaling domain of a CD28 protein, ICOS protein or a combination thereof; and (b) at least a second signaling domain that is a mutant CD137 (4-1BB) intracellular domain or a mutant CD134 (OX-40) intracellular domain. In some embodiments, the mutant CD137 (4-1BB) intracellular domain or the mutant CD134 (OX-40) intracellular domain comprises a deletion, an insertion or a substitution of one or more amino acids in the membrane proximal portion of the CD137 or CD134 intracellular domain. In some embodiments, the one or more amino acids in the membrane proximal portion are ubiquitination sites involved in the ubiquitination and degradation of the CD137 or CD134 protein. In some embodiments, the mutant CD137 (4-1BB) intracellular domain or a mutant CD134 (OX-40) intracellular domain comprises substitution or deletion of one or more lysine residues in the membrane proximal portion of the CD137 or CD134 intracellular domain. In some embodiments, the lysine residues are ubiquitination sites involved in the ubiquitination and degradation of the CD137 or CD134 protein. In some embodiments, the chimeric co-stimulatory intracellular domains provided herein further comprise a third signaling domain. In some embodiments, the third signaling domain can be based on a CD3 signaling domain.

In some embodiments, the novel co-stimulatory intracellular domain of the present application can be combined or fused in frame with the extracellular domain of any known co-stimulatory protein, a cell intrinsic immune checkpoint inhibitor, a chimeric antigen receptor, an antibody or a portion thereof, a ligand or a receptor thereof, a cytokine or a receptor thereof, a chemokine or a receptor thereof or a complement receptor, to form a functional recombinant T cell co-stimulatory receptor (RTCR). In some embodiments, the RTCR can be expressed in a cell in combination with another T cell receptor (TCR), chimeric antigen receptor or co-stimulatory protein. A RTCR comprising the novel co-stimulatory intracellular domain disclosed herein, when co-expressed with a TCR in a T cell, significantly increases the cell surface expression of the RTCR, and/or cell proliferation, activation, persistence, cytokine production and/or effector function of the T cell, as compared to a second-generation co-stimulatory receptor.

A highly efficacious adoptive cell therapeutic targeting a shared and safe tumor associated antigen and comprising a cell-intrinsic inhibitor of T cell exhaustion able to withstand the suppressive tumor microenvironment is described in the present application. An exemplary chimeric molecule expressing the extracellular domain of PD-1 and a functionally optimized chimeric intracellular co-stimulatory domain are disclosed herein. Modified T cells expressing the chimeric molecule of the present disclosure are generated to show the efficacy of the chimeric molecule in enhancing T cell stimulation, activation and proliferation. Both molecules are expressed on the same T cell, creating a TCR-T product that responds robustly to tumor cells expressing both the cognate MHC/peptide complex and high levels of PD-L1//PD-L2.

For exemplification, a cell-intrinsic inhibitor of T cell exhaustion is developed by co-expression of third generation chimeric PD-1 receptors combined with T cell receptors targeting tumor associated or specific antigens to enhance the efficacy of T cell mediated killing of tumor cells. The 3^{rd} generation chimeric receptors disclosed herein can be used in combination with any endogenous or modified T cell receptors as well as with chimeric artificial receptors (CARs). The results herein show that the 3^{rd}-generation co-stimulatory molecules disclosed herein produces T cells with high physiological avidity and persistent proliferative potential, while negating negative signaling by PD-1, delivering instead co-stimulatory signals in a PD-L1 rich environment. The novel co-stimulatory molecule can be co-expressed with a tumor associated antigen (TAA) specific TCR and used to target PD-L1/PD-L2 and the TAA expressing tumors. This demonstrates that the synergistic effect between the TCR activation and co-stimulatory molecule significantly increases the therapeutic window and generate a potentially more effective candidate for clinical investigation. In the disclosure described below, the design of the third-generation chimeric proteins is systematically optimized, to further validate in vitro the improved anti-cancer effectiveness, and to investigate the in vivo anti-tumor efficacy. Co-stimulatory molecules incorporating the extracellular domains of PD-1 with the intracellular domains of CD28, ICOS, CD134, and CD137 alone and in various combinations are generated. These sequences are optimized for surface expression and functionality by incorporating key mutations/deletions within the signaling domain of the chimeric receptors, focusing on the junction between CD28 and TNF-receptor family signaling domains. The functionality of these receptors is tested based on surface expression, in-vitro signaling, in-vitro T cell conjugation, cytokine production, proliferation, and cytotoxicity using a combination of soluble and plate-bound antibody stimulations and K562 target cells expressing PD-L1 or A375 tumor cells.

The disclosure herein provides an approach in which the TCR-T product co-expresses a chimeric co-stimulatory molecule alongside a recombinant TAA-specific TCR or an endogenous TCR. This approach allows for the targeting of the tumor associated antigen with simultaneous antagonization of checkpoint inhibition and delivery of co-stimulatory signals to the transfused T cell product. This approach not only results in a much-improved product, but also help to develop a universal function-boosting platform for additional TCR-T products.

The key technical challenge hindering the clinical adoption of 3^{rd}-generation CARs combining the two domains has been the abnormally low expression of chimeric proteins at the cell surface and associated diminished functionality (Zhao, 2015, Guedan, 2018). This has held true in co-stimulatory molecules where the combination of the CD28 and CD137 signaling domains resulted in a poorly expressed and non-functional receptor (Ankri, 2013). Disclosed herein is a switch receptor/co-stimulatory molecule based on the ICOS/CD137 signaling domain and optimized for surface expression that is a significant improvement over past trials using the CD28 signaling domain alone, mediating both increased effector function and persistence of adoptively transferred cells. Results described herein show that the 3^{rd}-generation co-stimulatory molecule disclosed herein produces T cells with high physiological avidity and persistent proliferation potential, while negating negative signaling by PD-1, delivering instead a co-stimulatory signal in a PD-L1 rich environment. The novel switch receptor/co-stimulatory molecule disclosed herein can be co-expressed with an endogenous TCR or a TAA specific TCR and used to target PD-L1/PD-L2 expressing tumors. This demonstrates that the synergistic effect between the TCR activation and co-stimulatory molecule significantly increases the therapeutic window for a potentially more effective candidate for clinical investigation.

Tumor associated antigens and tumor specific antigens allow for the immunological targeting of the tumor with relatively minimal risk of off-tumor, on-target side effects. Tumor cells can upregulate these antigens which can then be targeted by the human immune response or ACT. The disclosure herein combines a co-stimulatory molecule based on 3^{rd}-generation CARs that exhibits superior functionality to CD28-based receptors with a new affinity enhanced TCR targeting TAAs to generate a TCR-T product that resists the suppressive function of the TME.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

In some embodiments, the mutant intracellular signaling domain of a TNFR family protein is any one of a mutant CD137 (4-1BB) intracellular domain or a mutant CD134 (OX-40) intracellular domain.

The present disclosure provides a recombinant T cell co-stimulatory receptor (RTCR), comprising: (a) an extracellular domain; (b) a transmembrane domain; and (c) a chimeric intracellular domain comprising a first and at least a second signal transduction domains, wherein the first and the at least second signal transduction domains are non-identical; and wherein the at least second signal transduction domain comprises a mutant CD137 (4-1BB) intracellular domain or a mutant CD134 (OX-40) intracellular domain.

Unless indicated otherwise, the terms “co-stimulatory molecule”, “costimulatory molecule”, “co stimulatory molecule”, “co-stimulatory protein”, “costimulatory protein”, “co stimulatory protein”, “co-stimulatory receptor”, “costimulatory receptor” “co stimulatory receptor” and “switch receptor” are used interchangeably, to refer to the recombinant T cell co-stimulatory receptors (RTCRs) comprising the novel chimeric co-stimulatory intracellular domains of the present application. These terms may be used in combination with terms such as “recombinant T cell”, “recombinant”, “chimeric T cell”, and “chimeric”, to refer to the RTCRs of the present application.

As described herein, “a recombinant T cell co-stimulatory receptor” or “switch receptor” of the present disclosure is a “costimulatory molecule” “co-stimulatory receptor” or “co-stimulatory protein” generated by operably linking an extracellular domain to an intracellular chimeric intracellular protein of the present disclosure.

“CD137” as described herein is a member of the tumor necrosis factor (TNF) receptor family, and also referred to as 4-1BB, CD137, tumor necrosis factor receptor superfamily member 9 (TNFRSF9) and induced by lymphocyte activation (ILA). As described herein, the terms “CD137”, “4-1BB”, “4-1BB wt”, “4-1BB wild type”, “BB”, “BB wt” and “BB wild type” are used interchangeably throughout, for example, when describing constructs or co-stimulatory molecules of the present application, unless otherwise indicated.

In some embodiments, the CD137 intracellular domain can be from a mammalian CD137. In some embodiments, the mammalian CD137 can be a human CD137, a mouse CD137, a rat CD137 or a monkey CD137. In some embodiments, the CD137 intracellular domain can be from a human CD137, or an isoform or a variant thereof, comprising an amino acid sequence identical to any one of the human CD137 amino acid sequence according to GenBank Accession Nos: U03397, AAA62478, NP_001552, Q07011, AAH06196 and XP_006710681. In some embodiments, the CD137 intracellular domain can be from a mouse CD137, or an isoform or a variant thereof, comprising an amino acid sequence identical to any one of the mouse CD137 amino acid sequence according to GenBank Accession Nos: NP_001070977.1, NP_001070976.1, NP_035742.1, NP_033430.1, P20334.1, XP_011248530.1, XP_011248530.1, ABI30213.1, BAE32724.1 and AAH28507.1. In some embodiments, the CD137 intracellular domain can be from a rat CD137, or an isoform or a variant thereof, comprising an amino acid sequence identical to any one of the rat CD137 amino acid sequence according to GenBank Accession Nos: NP_852049.1, NP_001020944.1, BAD99404.1, XP_008762504.1, XP_006239534.1, EDL81196.1, AAH97483.1, EHB16663.1, EHB16663.1, KF038282.1, XP_010618177.1, XP_029414155.1, XP_029414154.1, XP_021099219.1 and XP_012888584.1. In some embodiments, the CD137 intracellular domain can be from a monkey CD137, or an isoform or a variant thereof, comprising an amino acid sequence identical to any one of the monkey CD137 amino acid sequence according to GenBank Accession Nos: ABY47575.1, ABI30212.1, ABY47577.1, ABY47576.1 and ABY47578.1.

In some embodiments, the CD137 intracellular domain, as described herein, comprises an amino acid sequence starting from the amino acid position 214 to the last amino acid at the C-terminal end of the amino acid sequence of the human CD137 protein, described herein. In some embodiments, the CD137 intracellular domain, as described herein, comprises an amino acid sequence starting from the amino acid position 215 to the last amino acid at the C-terminal end of the amino acid sequence of the mouse CD137 protein, described herein.

In some embodiments, the mutant CD137 intracellular domain described herein is from any one of the CD137 proteins as described herein, comprising one or more mutation(s), wherein the mutation can be addition/insertion, deletion/truncation or substitution/replacement of one or more amino acids within the amino acid sequence of the CD137 protein. In some embodiments, the mutant CD137 intracellular domain described herein is any one of the CD137 intracellular domain sequences, as described herein, comprising one or more mutation(s), wherein the mutation can be addition/insertion, deletion/truncation or substitution/replacement of one or more amino acids within the amino acid sequence of the CD137 intracellular domain. In some embodiments, the mutant CD137 intracellular domain described herein is a CD137 intracellular domain as described herein, comprising a deletion or substitution of one or more amino acids within the amino acid sequence of the CD137 intracellular domain that can be targets for ubiquitination. In some embodiments, the mutant CD137 intracellular domain described herein is a CD137 protein as described herein, comprising a deletion or substitution, of one or more lysine residues within the amino acid sequence of the CD137 intracellular domain that can be targets for ubiquitination. In some embodiments, the mutant CD137 intracellular domain described herein is a CD137 protein as described herein, comprising a deletion or substitution, of one, two, three or four lysine residues within the amino acid sequence of the CD137 intracellular domain that can be targets for ubiquitination. In some embodiments, the lysine residues within the amino acid sequence of the CD137 intracellular domain described herein, that can be deleted or substituted are at amino acid positions 214, 218, 219 and/or 225 of the CD137 intracellular domain.

In some embodiments, the mutant CD137 intracellular domain can be a truncated CD137 intracellular domain. A truncated CD137 intracellular domain as described herein can be any one of the CD137 proteins described herein, in which a continuous stretch of more than one, two, three, four, five, six, seven, eight, nine, ten, eleven, twelve, thirteen, fourteen, fifteen, twenty, twenty-five, fifty, hundred, two hundred or more amino acids are deleted from the N-terminus the CD137 protein as described herein. A truncated CD137 intracellular domain as described herein can be any one of the CD137 intracellular domain sequences described herein, in which a continuous stretch of more than one, two, three, four, five, six, seven, eight, nine, ten or more amino acids are deleted from the N-terminus the CD137 intracellular domain as described herein. In some embodiments, the amino acids deleted from the N-terminus the CD137 intracellular domain includes one or more proximal polybasic amino acids of the CD137 intracellular domain.

In some embodiments, the mutant CD137 intracellular domain can be a truncated CD137 intracellular domain. In some embodiments, the truncated CD137 intracellular domain comprises an amino acid sequence according to amino acid position 13 to amino acid position 42 of the CD137 intracellular domain, of the present disclosure. In some embodiments, the truncated CD137 intracellular domain comprises a deletion of a continuous stretch of one, two, three, four, five, six, seven, eight, nine, ten or more amino acids from the N-terminus of the CD137 intracellular domain, of the present disclosure. In some embodiments, the truncated CD137 intracellular domain comprises a deletion of one, two, three, four, five, six, seven, eight, nine, ten or more amino acids from amino acid position 1 to amino acid position 12 of the N-terminus of the CD137 intracellular domain, of the present disclosure. In some embodiments, the truncated CD137 intracellular domain comprises a deletion of amino acid position 1 to amino acid position 12 of the N-terminus of the CD137 intracellular domain, of the present disclosure. In some embodiments, the CD137 intracellular domain comprises an amino acid sequence according to SEQ ID NO: 1.

In some embodiments, the truncated CD137 intracellular domain comprises an amino acid sequence according to SEQ ID NO: 3. In some embodiments, the truncated CD137 intracellular domain comprises an amino acid sequence having at least 50%, 55%, 60%, 65%, 70%, 75%, 80%, 85%, 90%, 95%, 97% or 99% identity to SEQ ID NO: 3.

A truncated CD137 intracellular domain as described herein, is referred to as “truncated CD137”, “CD137t”, “truncated 4-1BB”, “4-1BBt”, “truncated BB” or “BBt” interchangeably throughout, for example, when describing constructs or co-stimulatory molecules of the present application, unless otherwise indicated. In some embodiments, the mutant CD137 intracellular domain comprises a deletion of one, two, three or four lysine residue(s) from amino acid position 1 to amino acid position 12 of the N-terminus of the CD137 intracellular domain, of the present disclosure. In some embodiments, the mutant CD137 intracellular domain comprises one or more lysine mutation(s) from amino acid position 1 to amino acid position 12 of the N-terminus of the CD137 intracellular domain, of the present disclosure. In some embodiments, the mutant CD137 intracellular domain comprises one or more lysine mutation(s) at amino acid positions selected from amino acid positions 1, 5, 6 and 12 of the N-terminus of the CD137 intracellular domain, of the present disclosure. In some embodiments, the one or more lysine mutation(s) are lysine to alanine mutations. In some embodiments, the CD137 intracellular domain comprises an amino acid sequence according to SEQ ID NO: 1.

In some embodiments, the mutant CD137 intracellular domain comprises a deletion of one or more proximal basic amino acids from amino acid position 1 to amino acid position 12 of the N-terminus of the CD137 intracellular domain, of the present disclosure. In some embodiments, the mutant CD137 intracellular domain comprises one or more proximal basic amino acid mutation(s) from amino acid position 1 to amino acid position 12 of the N-terminus of the CD137 intracellular domain, of the present disclosure. In some embodiments, the mutant CD137 intracellular domain comprises one or more proximal basic amino acid mutation(s) at amino acid positions selected from amino acid positions 1, 2, 3, 4, 5 and 6 of the N-terminus of the CD137 intracellular domain, of the present disclosure. In some embodiments, the mutant CD137 intracellular domain comprising one or more proximal basic amino acid mutation(s), of the present disclosure, further comprises a lysine mutation at amino acid position 12 of the N-terminus of the CD137 intracellular domain, of the present disclosure. In some embodiments, the lysine mutation is a lysine to alanine mutation. In some embodiments, the CD137 intracellular domain comprises an amino acid sequence according to SEQ ID NO: 1.

“CD134” as described herein is a member of the tumor necrosis factor (TNF) receptor family, and also referred to as OX-40, ACT35, IMD16, TXGP1L and tumor necrosis factor receptor superfamily member 4 (TNFRSF4). As described herein, the terms “CD134”, “OX-40”, “OX40”, “OX-40 wild type”, “OX-40 wt”, “OX40 wild type”, “OX40 wt”, “40”, “40 wild type” and “40 wt” are used interchangeably throughout, for example, when describing constructs or co-stimulatory molecules of the present application, unless otherwise indicated.

In some embodiments, the CD134 intracellular domain can be from a mammalian CD134. In some embodiments, the mammalian CD134 can be a human CD134, a mouse CD134, a rat CD134 or a monkey CD134. In some embodiments, the CD134 intracellular domain can be from a human CD134, or an isoform or a variant thereof, comprising an amino acid sequence identical to any one of the human CD134 amino acid sequence according to GenBank Accession Nos: NP_003318, AA105071, AA105073, XP_016857721.1, XP_016857720.1, XP_011540377.1, XP_011540379.1, XP_011540378.1, XP_011540376.1, P43489.1, NP_001284491.1, NP_003317.1, EAW56278.1 and CAB96543.1. In some embodiments, the CD134 intracellular domain can be from a mouse CD134, or an isoform or a variant thereof, comprising an amino acid sequence identical to any one of the mouse CD134 amino acid sequence according to GenBank Accession Nos: NP_035789.1, AAI39267.1, AAI39240.1, NP_033478.1, XP_006538787.3, P47741.1, EDL15067.1, CAA79772.1, CAA59476.1, XP_021017102.2, and XP_021056714.1. In some embodiments, the CD134 intracellular domain can be from a rat CD134, or an isoform or a variant thereof, comprising an amino acid sequence identical to any one of the rat CD134 amino acid sequence according to GenBank Accession Nos: NP_035789.1, NP_037181.1, P15725.1, EDL81353.1, CAB96543.1, and CAA34897.1. In some embodiments, the CD134 intracellular domain can be from a monkey CD134, or an isoform or a variant thereof, comprising an amino acid sequence identical to any one of the monkey CD134 amino acid sequence according to GenBank Accession Nos: XP_010375483.1, XP_001090870.1, XP_021523144.1, XP_017750744.1, XP_003939714.1, XP_026313229.1, XP_026313228.1, XP_003890998.2, XP_025242473.1, XP_011768627.1, XP_005545179.1, XP_011886513.1, XP_011886512.1, XP_011857387.1 and XP_011811769.1.

In some embodiments, the CD134 intracellular domain, as described herein, comprises an amino acid sequence starting from amino acid position 241 to the last amino acid at the C-terminal end of the amino acid sequence of any one of the human CD134 protein, described herein. In some embodiments, the CD134 intracellular domain, as described herein, comprises an amino acid sequence starting from the amino acid position 236 to the last amino acid at the C-terminal of the amino acid sequence of the mouse CD134 protein, described herein.

In some embodiments, the mutant CD134 intracellular domain described herein is from any one of the CD134 proteins as described herein, comprising one or more mutation(s), wherein the mutation can be addition/insertion, deletion/truncation or substitution/replacement of one or more amino acids within the amino acid sequence of the CD134 protein. In some embodiments, the mutant CD134 intracellular domain described herein, is any one of the CD134 intracellular domain sequences as described herein, comprising one or more mutation(s), wherein the mutation can be addition/insertion, deletion/truncation or substitution/replacement of one or more amino acids within the amino acid sequence of the CD134 intracellular domain. In some embodiments, the mutant CD134 intracellular domain described herein is a CD134 intracellular domain as described herein, comprising a deletion or substitution of one or more amino acids within the amino acid sequence of the CD134 intracellular domain that can be targets for ubiquitination. In some embodiments, the mutant CD134 intracellular domain described herein is a CD134 protein as described herein, comprising a deletion or substitution, of one or more lysine residues within the amino acid sequence of the CD134 intracellular domain that can be targets for ubiquitination. In some embodiments, the mutant CD134 intracellular domain described herein is a CD134 protein as described herein, comprising a deletion or substitution, of one or two lysine residues within the amino acid sequence of the CD134 intracellular domain that can be targets for ubiquitination. In some embodiments, the lysine residues within the amino acid sequence of the CD134 intracellular domain described herein, that can be deleted or substituted are at amino acid positions 252 and/or 276 of the CD134 intracellular domain.

In some embodiments, the mutant CD134 intracellular domain can be a truncated CD134 intracellular domain. A truncated CD134 intracellular domain as described herein can be any one of the CD134 proteins described herein, in which a continuous stretch of more than one, two, three, four, five, six, seven, eight, nine, ten, eleven, twelve, thirteen, fourteen, fifteen, twenty, twenty-five, fifty, hundred, two hundred or more amino acids are deleted from the N-terminus the CD137 protein as described herein. A truncated CD134 intracellular domain as described herein can be any one of the CD134 intracellular domain sequences described herein, in which a continuous stretch of more than one, two, three, four, five, six, seven, eight, nine, ten, eleven, twelve, thirteen or more amino acids are deleted from the N-terminus the CD134 intracellular domain as described herein. In some embodiments, the amino acids deleted from the N-terminus the CD134 intracellular domain includes one or more proximal polybasic amino acids of the CD134 intracellular domain.

In some embodiments, the truncated CD134 intracellular domain comprises an amino acid sequence according to amino acid position 15 to amino acid position 37 of a CD134 intracellular domain, of the present disclosure. In some embodiments, the truncated CD134 intracellular domain comprises a deletion of a continuous stretch of one, two, three, four, five, six, seven, eight, nine, ten, eleven, twelve, thirteen or more amino acids from the N-terminus of the CD134 intracellular domain, of the present disclosure. In some embodiments, the truncated CD134 intracellular domain comprises a deletion of one, two, three, four, five, six, seven, eight, nine, ten, eleven, twelve, thirteen or more amino acids from amino acid position 1 to amino acid position 14 of the N-terminus of the CD134 intracellular domain, of the present disclosure. In some embodiments, the truncated CD137 intracellular domain comprises a deletion of amino acid position 1 to amino acid position 14 of the N-terminus of the CD134 intracellular domain, of the present disclosure. In some embodiments, the CD134 intracellular domain comprises an amino acid sequence according to SEQ ID NO: 4.

In some embodiments, the mutant CD134 intracellular domain comprises an amino acid sequence according to SEQ ID NO: 6. In some embodiments, the mutant CD134 intracellular domain comprises an amino acid sequence having at least 50%, 55%, 60%, 65%, 70%, 75%, 80%, 85%, 90%, 95%, 97% or 99% identity to SEQ ID NO: 6.

A truncated CD134 intracellular domain as described herein, is referred to as “truncated CD134”, “CD134t”, “truncated OX-40”, “truncated OX40”, “OX-40t”, “OX40t” and “40t” are used interchangeably throughout, for example, when describing constructs or co-stimulatory molecules of the present application, unless otherwise indicated.

In some embodiments, the mutant CD134 intracellular domain comprises a deletion of a lysine residue from amino acid position 1 to amino acid position 14 of the N-terminus of the CD134 intracellular domain, of the present disclosure. In some embodiments, the mutant CD134 intracellular domain comprises a lysine mutation at amino acid position 12 of the N-terminus of the CD134 intracellular domain, of the present disclosure. In some embodiments, the lysine mutation is a lysine to alanine mutation. In some embodiments, the CD134 intracellular domain comprises an amino acid sequence according to SEQ ID NO: 4.

In some embodiments, the mutant CD134 intracellular domain comprises a deletion of one or more proximal basic amino acids from amino acid position 1 to amino acid position 14 of the N-terminus of the CD134 intracellular domain, of the present disclosure. In some embodiments, the mutant CD134 intracellular domain comprises one or more proximal basic amino acid mutation(s) from amino acid position 1 to amino acid position 14 of the N-terminus of the CD134 intracellular domain, of the present disclosure. In some embodiments, the mutant CD134 intracellular domain comprises one or more proximal basic amino acid mutation(s) at amino acid positions selected from amino acid positions 1, 2, and 5 of the N-terminus of the CD134 intracellular domain, of the present disclosure. In some embodiments, the mutant CD137 intracellular domain further comprises a lysine mutation at amino acid position 12 of the N-terminus of the CD137 intracellular domain, of the present disclosure. In some embodiments, the CD134 intracellular domain comprises an amino acid sequence according to SEQ ID NO: 4.

TABLE 1
Amino acid sequences of second signal
transduction domains of RTCR (CD137/4-1BB and
CD134/OX-40 intracellular signaling domain).

| | |
|---|---|
| Human CD137/4-1BB | KRGRKKLLYIFKQPFMRPVQTTQEED |
| (SEQ ID NO: 1) | GCSCRFPEEEEGGCEL |
| Mouse CD137/4-1BB | RKKFPHIFKQPFKKTTGAAQEEDACS |
| (SEQ ID NO: 2) | CRCPQEEEGGGGGYEL |
| truncated/mutated | QPFMRPVQTTQEEDGCSCRFPEEEEG |
| CD137/4-1BB | GCEL |
| (SEQ ID NO: 3) | |
| Human CD134/OX-40 | RRDQRLPPDAHKPPGGGSFRTPIQEE |
| (SEQ ID NO: 4) | QADAHSTLAKI |
| Mouse CD134 | RKAWRLPNTPKPCWGNSFRTPIQEEH |
| (SEQ ID NO: 5) | TDAHFTLAKI |
| truncated/mutated | GGGSFRTPIQEEQADAHSTLA |
| CD134/OX-40 | |
| (SEQ ID NO: 6) | |
| Human CD27 | QRRKYRSNKGESPVEPAEPCHYSCPR |
| (SEQ ID NO: 7) | EEEGSTIPIQEDYRKPEPACSP |
| Human GITR | QLGLHIWQLRSQCMWPRETQLLLEVP |
| (SEQ ID NO: 8) | PSTEDARSCQFPEEERGERSAEEKGR |
| | LGDLWV |

Membrane-proximal poly-basic regions are italicized. Potential PI3K binding sites are bold and underlined. TRAF1/2 binding motifs, major motif Px(Q/E)E and minor motifs Px(Q/E)x, are highlighted in underlined. Potential ubiquitination sites are in bold.

In some embodiments, the chimeric intracellular domain comprises a first signal transduction domain derived from a protein of the CD28 family. In some embodiments, the first signal transduction domain derived from any one of CD28, CD28H, ICOS or a combination thereof.

In some embodiments, the chimeric intracellular domain comprises a first signal transduction domain derived from ICOS protein.

The “ICOS protein” as described herein is an inducible T cell co-stimulatory protein, also referred to as AILIM, CD278, CCLP, CRP-1, H4, Ly115 and CVID1. In some embodiments, the ICOS intracellular domain can be from a mammalian ICOS. In some embodiments, the mammalian ICOS can be a human ICOS, a mouse ICOS, a rat ICOS or a monkey ICOS. In some embodiments, the ICOS intracellular domain can be from a human ICOS, or an isoform or a variant thereof, comprising an amino acid sequence identical to any one of the human ICOS amino acid sequence according to GenBank Accession Nos: AAH28006.1, NP_036224.1, AIC51287.1, AIC60036.1, NP_036224.1, Q9Y6W8.1, EAW70357.1, EAW70356.1, EAW70355.1, AAL40934.1, AAL40933.1, CAC06612.1, AAX93073.1, AAM00909.1, AAH28210.1 and CAD59742.1. In some embodiments, the ICOS intracellular domain can be from a mouse ICOS, or an isoform or a variant thereof, comprising an amino acid sequence identical to any one of the mouse ICOS amino acid sequence according to GenBank Accession Nos: NP_059508.2, Q9WVS0.2, EDL00161.1, CAM13242.1, CAM13241.1, CAB71153.1, AAG48732.1, AAH34852.1, XP_006496203.1, XP_006496202.1, XP_006496201.1, ACX50464.1, ACX50463.1, AAH28006.1, XP_021052880.1, XP_029334968.1 and XP_021030282.1. In some embodiments, the ICOS intracellular domain can be from a rat ICOS, or an isoform or a variant thereof, comprising an amino acid sequence identical to any one of the rat ICOS amino acid sequence according to GenBank Accession Nos: NP_072132.1, Q9R1T7.1, XP_008765358.1, XP_006245100.1, XP_006245099.1, EDL98922.1, EDL98921.1, XP_038940099.1, XP_032755449.1, XP_017457364.1, XP_006256324.1, XP_006256323.1, XP_006256322.1, XP_029425757.1, XP_029425757.1, XP_021119236.1, XP_012929934.1, XP_012867370.1 and XP_012867363.1. In some embodiments, the ICOS intracellular domain can be from a monkey ICOS, or an isoform or a variant thereof, comprising an amino acid sequence identical to any one of the monkey ICOS amino acid sequence according to GenBank Accession Nos: XP_007964137.1, NP_001253918.1, XP_010350939.1, XP_012301785.1, XP_012301784.1, XP_017739861.1, XP_010334714.1, XP_003925677.1, AFH29328.1, XP_008997520.1, XP_023075107.1, XP_023075099.1, XP_021779593.1, XP_003907887.1, XP_025260988.1, XP_025260987.1, XP_025260986.1, XP_011716287.1, XP_011716285.1, XP_005574075.1, XP_011903009.1, XP_011805288.1, XP_011805287.1, XP_011847867.1, XP_011847866.1, XP_017392362.1, XP_033086489.1, XP_032134414.1, XP_032134413.1, and XP_017802331.1.

In some embodiments, the human ICOS intracellular domain as described herein, comprises an amino acid sequence from amino acid position 133 to the last amino acid at the C-terminus of the amino acid sequence of the human ICOS protein, described herein. In some embodiments, the human ICOS intracellular domain as described herein, comprises an amino acid sequence from an amino acid position at one, two, three, four, five, six, seven, eight, nine, ten or more amino acids N-terminus to the amino acid position 133, to the last amino acid at the C-terminus of the amino acid sequence of the human ICOS protein, described herein. In some embodiments, the human ICOS intracellular domain as described herein, comprises an amino acid sequence from amino acid position 133 to an amino acid position at one, two, three, four, five, six, seven, eight, nine, ten or more amino acids N-terminus to the last amino acid at the C-terminus of the amino acid sequence of the human ICOS protein, described herein. In some embodiments, the human ICOS intracellular domain as described herein, comprises an amino acid sequence from an amino acid position at one, two, three, four, five, six, seven, eight, nine, ten or more amino acids N-terminus to the amino acid position 133, to an amino acid position at one, two, three, four, five, six, seven, eight, nine, ten or more amino acids N-terminus to the last amino acid at the C-terminus of the amino acid sequence of the human ICOS protein, described herein.

In some embodiments, the human ICOS(28) intracellular domain as described herein, comprises a portion of the ICOS domain amino acid sequence from amino acid position 133 to amino acid position 183, and a portion of the ICOS domain amino acid sequence from amino acid position 184 to the last amino acid at the C-terminus of the amino acid sequence of the human ICOS protein, described herein. In some embodiments, the human ICOS(28) intracellular domain as described herein, comprises a portion of the ICOS domain amino acid sequence from an amino acid position at one, two, three, four, five, six, seven, eight, nine, ten or more amino acids N-terminus to the amino acid position 133, to amino acid position 183 of the human ICOS protein, described herein. In some embodiments, the human ICOS(28) intracellular domain as described herein, comprises a portion of the ICOS domain amino acid sequence from amino acid position 133, to an amino acid position at one, two, three, four, five, six, seven, eight, nine, ten or more amino acids C-terminus to the amino acid position 183 of the human ICOS protein, described herein. In some embodiments, the human ICOS(28) intracellular domain as described herein, comprises a portion of the ICOS domain amino acid sequence from an amino acid position at one, two, three, four, five, six, seven, eight, nine, ten or more amino acids N-terminus to the amino acid position 133, to an amino acid position at one, two, three, four, five, six, seven, eight, nine, ten or more amino acids C-terminus to the amino acid position 183 of the human ICOS protein, described herein.

The “CD28 protein”, also referred to as Tp44, is a constitutively expressed receptor for CD80 (B7.1) and CD86 (B7.2) proteins on naïve T cells and is important for T cell activation. In some embodiments, the CD28 intracellular domain can be from a mammalian CD28. In some embodiments, the mammalian CD28 can be a human CD28, a mouse CD28, a rat CD28 or a monkey CD28. In some embodiments, the CD28 intracellular domain can be from a human CD28, or an isoform or a variant thereof, comprising an amino acid sequence identical to any one of the human CD28 amino acid sequence according to GenBank Accession Nos: P10747.1, NP_001230007.1, NP_001230006.1, NP_006130.1, EAW70350.1, EAW70349.1, EAW70348.1, EAW70347.1, AIC48451.1, CAC29237.1, AAA51945.1, AAA51944.1, AAL40931.1, AAF33794.1, AAF33793.1, AAF33792.1, XP_011510499.1, XP_011510497.1, XP_011510496.1, AAI12086.1, AAH93698.1, ABK41938.1, AAY24123.1, CAD57003.1 and AAA60581. In some embodiments, the CD28 intracellular domain can be from a mouse CD28, or an isoform or a variant thereof, comprising an amino acid sequence identical to any one of the mouse CD28 amino acid sequence according to GenBank Accession Nos: AAA37396.1, NP_031668.3, P31041.2, AAH64058.1, EDL00156.1, CAM13249.1, XP_036012281.1, XP_021054806.1, XP_021027481.1, XP_036015651.1, and XP_030104805. In some embodiments, the CD28 intracellular domain can be from a rat CD28, or an isoform or a variant thereof, comprising an amino acid sequence identical to any one of the rat CD28 amino acid sequence according to GenBank Accession Nos: CAA39003.1, NP_037253.2, P31042.1, XP_008765300.1, EDL98926.1, XP_032755445.1, XP_034354910.1, XP_019061859.2, XP_008844474.1, XP_004851403.1 and XP_012865504.1. In some embodiments, the CD28 intracellular domain can be from a monkey CD28, or an isoform or a variant thereof, comprising an amino acid sequence identical to any one of the monkey CD28 amino acid sequence according to GenBank Accession Nos: ABH06891.1, ABH08508.1, ABH06892.1, ABH08509.1, ABQ09493.1, NP_001274262.1, NP_001036106.2, ABG77998.1, ABG77997.1 and XP_0149662071.

The “CD28H protein”, also referred to CD28 homolog, transmembrane and immunoglobulin domain-containing protein 2, has co-stimulatory activity in T cells by binding to B7H7. CD28H was initially described as a molecule involved in cell-cell interaction, cell migration, and angiogenesis of epithelial and endothelial cells (7, 8). CD28H has a single extracellular immunoglobulin domain followed by a transmembrane domain and a 110 amino acid-long cytoplasmic region. In some embodiments, the CD28 intracellular domain can be from a mammalian CD28H. In some embodiments, the mammalian CD28 can be a human CD28H, a mouse CD28H, a rat CD28H or a monkey CD28H. In some embodiments, the CD28H intracellular domain can be from a human CD28H, or an isoform or a variant thereof, comprising an amino acid sequence identical to any one of the human CD28H amino acid sequence according to GenBank Accession Nos: NP_001295161.1, NP_001162597.1, Q96BF3.2, XP_024307127.1 and XP_0168817731.

In some embodiments, the human CD28 intracellular domain as described herein, comprises an amino acid sequence from amino acid position 145 to the last amino acid at the C-terminus of the amino acid sequence of the human CD28 protein, described herein. In some embodiments, a portion of the human CD28 intracellular domain as described herein, can comprise an amino acid sequence from about amino acid position 195 to about amino acid position 212 of the amino acid sequence of the human CD28 protein, described herein. In some embodiments, a portion of the human CD28 intracellular domain as described herein, can comprises an amino acid sequence from one, two, three, four, five, six, seven, eight, nine or 10 or more amino acid amino acid position N-terminus to amino acid position 195 to one, two, three, four, five, six, seven, eight, nine or 10 or more amino acid amino acid position C-terminus amino acid position 220 of the amino acid sequence of the human CD28 protein, described herein.

In some embodiments, the first signal transduction domain derived from ICOS comprises an amino acid sequence according to SEQ ID NO: 9. In some embodiments, the first signal transduction domain derived from ICOS comprises an amino acid sequence having at least 50%, 55%, 60%, 65%, 70%, 75%, 80%, 85%, 90%, 95%, 97% or 99% identity to SEQ ID NO: 9.

In some embodiments, the chimeric intracellular domain comprises a first signal transduction domain comprising a portion of a CD28 intracellular domain combined with an ICOS protein (ICOS (28) domain) according to SEQ ID NO: 9. In some embodiments, the ICOS (28) domain comprises the portion of CD28 intracellular domain inserted N-terminal to the PI-3K binding site of the ICOS protein according to SEQ ID NO: 9. In some embodiments, the ICOS (28) domain comprises the portion of CD28 inserted at 1, 2, 3, 4 or 5 amino acid position N-terminal to the PI-3K binding site of the ICOS protein according to SEQ ID NO: 9. In some embodiments, the ICOS(28) domain comprises the portion of CD28 inserted C-terminal to the PI-3K binding site of the ICOS protein according to SEQ ID NO: 9. In some embodiments, the ICOS(28) domain comprises the portion of CD28 inserted at 1, 2, 3, 4 or 5 amino acid position C-terminal to the PI-3K binding site of the ICOS protein according to SEQ ID NO: 9.

In some embodiments, the portion of CD28 is inserted at any amino acid position before amino acid position 48 within an ICOS protein of amino acid sequence according to SEQ ID NO: 9. In some embodiments, the portion of CD28 is inserted at any amino acid position between amino acid position 1 and amino acid position 48, within an ICOS protein of amino acid sequence according to SEQ ID NO: 9. In some embodiments, the portion of CD28 is inserted between the amino acid position 47 and amino acid position 48 of an ICOS protein with the amino acid sequence according to SEQ ID NO: 9. In some embodiments, the portion of CD28 is inserted between the amino acid position 46 and amino acid position 47 of an ICOS protein with the amino acid sequence according to SEQ ID NO: 9. In some embodiments, the portion of CD28 is inserted between the amino acid position 45 and amino acid position 46 of an ICOS protein with the amino acid sequence according to SEQ ID NO: 9. In some embodiments, the portion of CD28 is inserted between the amino acid position 44 and amino acid position 45 of an ICOS protein with the amino acid sequence according to SEQ ID NO: 9. In some embodiments, the portion of CD28 is inserted between the amino acid position 43 and amino acid position 44 of an ICOS protein with the amino acid sequence according to SEQ ID NO: 9.

In some embodiments, the portion of CD28 is inserted at any position after amino acid position 51 within an ICOS protein of amino acid sequence according to SEQ ID NO: 9. In some embodiments, the portion of CD28 is inserted at any amino acid position between amino acid position 51 and amino acid position 67, within an ICOS protein of amino acid sequence according to SEQ ID NO: 9. In some embodiments, the portion of CD28 is inserted between the amino acid position 51 and amino acid position 52 of an ICOS protein with the amino acid sequence according to SEQ ID NO: 9. In some embodiments, the portion of CD28 is inserted between the amino acid position 53 and amino acid position 54 of an ICOS protein with the amino acid sequence according to SEQ ID NO: 9. In some embodiments, the portion of CD28 is inserted between the amino acid position 54 and amino acid position 55 of an ICOS protein with the amino acid sequence according to SEQ ID NO: 9. In some embodiments, the portion of CD28 is inserted between the amino acid position 56 and amino acid position 57 of an ICOS protein with the amino acid sequence according to SEQ ID NO: 9. In some embodiments, the portion of CD28 is inserted between the amino acid position 57 and amino acid position 58 of an ICOS protein with the amino acid sequence according to SEQ ID NO: 9.

In some embodiments, the portion of CD28 of the ICOS(28) domain disclosed herein comprises an amino acid sequence according to amino acid position 51 to amino acid position 68 of a CD28 signaling domain according to SEQ ID NO: 10. In some embodiments, the portion of CD28 of the ICOS(28) domain disclosed herein comprises an amino acid sequence according to amino acid position 51 to amino acid position 76 of a full length CD28 signaling domain according to SEQ ID NO: 10. In some embodiments, the portion of CD28 of the ICOS(28) domain disclosed herein comprises an amino acid sequence according to amino acid position 45 to amino acid position 68 of a CD28 signaling domain according to SEQ ID NO: 10. In some embodiments, the portion of CD28 inserted within the ICOS(28) domain comprises a PRRP motif. In some embodiments, the portion of CD28 inserted within the ICOS(28) domain comprises an amino acid sequence according to SEQ ID NO: 11. In some embodiments, the portion of CD28 inserted within the ICOS(28) domain comprises an amino acid sequence having at least 50%, 55%, 60%, 65%, 70%, 75%, 80%, 85%, 90%, 95%, 97% or 99% identity to SEQ ID NO: 11.

In some embodiments, the ICOS(28) domain comprises an amino acid sequence according to SEQ ID NO: 12. In some embodiments, the ICOS(28) comprises an amino acid sequence having at least 50%, 55%, 60%, 65%, 70%, 75%, 80%, 85%, 90%, 95%, 97% or 99% identity to SEQ ID NO: 12.

In some embodiments, the chimeric intracellular domain comprises a first signal transduction domain derived from CD28. In some embodiments, the first signal transduction domain derived from CD28 comprises an amino acid sequence according to SEQ ID NO: 10. In some embodiments, the first signal transduction domain derived from CD28 comprises an amino acid sequence having at least 50%, 55%, 60%, 65%, 70%, 75%, 80%, 85%, 90%, 95%, 97% or 99% identity to SEQ ID NO: 10.

A signal transduction domain derived from CD28 as described herein, is referred to as “CD28” or “28”, interchangeably throughout.

TABLE 2
Amino acid sequences of first intracellular signaling domains.

ICOS signaling domain (SEQ ID NO: 9) (Other name: ICOS): Stalk (underlined), TM (regular
font), intracellular domain (IC) (bold) and PI-3K binding site (bold and underlined)
SQLCCQLKFWLPIGCAAFVVVCILGCILICWLTKKKYSSSVHDPNGEYMFMRAVNT
AKKSRLTDVTL
CD28 Transmembrane_CD28 Signaling Domain (Other names: CD28 or 28) (SEQ ID NO:
10): CD28 Stalk (underlined), TM (regular font), intracellular domain (IC) (bold), PI3K
regulatory subunit binding, GRB2, GADS association domain (bold and dotted-underlined),
ITK interaction site (bold and double-underlined, GRB2, GADS, LCK interaction site (bold
and dash-underlined)

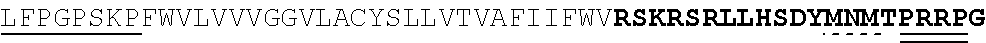

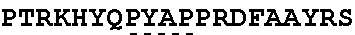

CD28 fragment (Other names: mini-CD28 or (28)) (SEQ ID NO: 11): PRRP motif in bold
TPRRPGPTRKHYQPYAPP
ICOS (28) domain (Other name: ICOS (mini-CD28)) (SEQ ID NO: 12): intracellular domain
(italicized), TBK1 binding site (TRAF-like motif) (italicized and dotted-underlined), PI-3K
regulatory subunit binding site (italicized and underlined), CD28 portion (bold), distal domain
(italicized and dash-underlined)

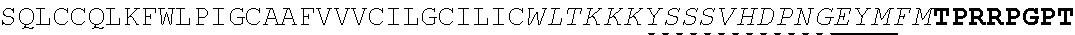

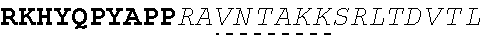

In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 13. In some embodiments, the chimeric intracellular domain comprises an amino acid sequence according to any one of SEQ ID NOs: 14-17. In some embodiments, the chimeric intracellular domain comprises an amino acid sequence having at least 50%, 55%, 60%, 65%, 70%, 75%, 80%, 85%, 90%, 95%, 97% or 99% identity to any one of SEQ ID NOs: 14-17. In some embodiments, the chimeric intracellular domain comprises an amino acid sequence according to SEQ ID NO: 14. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 14. In some embodiments, the chimeric intracellular domain comprises an amino acid sequence according to SEQ ID NO: 15. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 15. In some embodiments, the chimeric intracellular domain comprises an amino acid sequence according to SEQ ID NO: 16. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 16. In some embodiments, the chimeric intracellular domain comprises an amino acid sequence according to SEQ ID NO: 17. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 17.

In some embodiments, the chimeric intracellular domain comprises an amino acid sequence according to any one of SEQ ID NOs: 120-129. In some embodiments, the chimeric intracellular domain comprises an amino acid sequence having at least 50%, 55%, 60%, 65%, 70%, 75%, 80%, 85%, 90%, 95%, 97% or 99% identity to any one of SEQ ID NOs: 120-129. In some embodiments, the chimeric intracellular domain comprises an amino acid sequence according to SEQ ID NO: 120. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 120. In some embodiments, the chimeric intracellular domain comprises an amino acid sequence according to SEQ ID NO: 121. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 121. In some embodiments, the chimeric intracellular domain comprises an amino acid sequence according to SEQ ID NO: 122. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 122.

In some embodiments, the chimeric intracellular domain comprises an amino acid sequence according to SEQ ID NO: 123. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 123. In some embodiments, the chimeric intracellular domain comprises an amino acid sequence according to SEQ ID NO: 124. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 124. In some embodiments, the chimeric intracellular domain comprises an amino acid sequence according to SEQ ID NO: 125. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 125. In some embodiments, the chimeric intracellular domain comprises an amino acid sequence according to SEQ ID NO: 126. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 126. In some embodiments, the chimeric intracellular domain comprises an amino acid sequence according to SEQ ID NO: 127. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 127. In some embodiments, the chimeric intracellular domain comprises an amino acid sequence according to SEQ ID NO: 128.

In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 128. In some embodiments, the chimeric intracellular domain comprises an amino acid sequence according to SEQ ID NO: 129. In some embodiments, the chimeric intracellular domain comprises an amino acid sequence having at least 50%, 55%, 60%, 65%, 70%, 75%, 80%, 85%, 90%, 95%, 97% or 99% identity to SEQ ID NO: 129.

TABLE 3
Amino acid sequences of
chimeric intracellular domains of RTCR.

ICOS Transmembrane_ICOS Signaling Domain_4-1BB Signaling Domain (other names:
ICOS-4-1BB (CD137) intracellular domain, ICOS-137; ICOS_137; ICOS137; ICOS_BB;
ICOS-BB; ICOSBB; ICOSBBwt; ICOS_BBwt or ICOS_BB wild type)(SEQ ID NO: 13):
ICOS sequence underlined and 4-1BB (BB) domain in normal font
SQLCCQLKFWLPIGCAAFVVVCILGCILICWLTKKKYSSSVHDPNGEYMFMRAVNTA
KKSRLTDVTLKRGRKKLLYIFKQPFMRPVQTTQEEDGCSCRFPEEEEGGCEL
ICOS Transmembrane_ICOS Signaling Domain_Truncated 4-1BB Signaling Domain (other
names: ICOS-truncated 4-1BB (CD137) intracellular domain; ICOS_137t; ICOS137t; ICOS-
137t; ICOS_BBt; ICOSBBt or ICOS-BBt)(SEQ ID NO: 14): ICOS sequence underlined and
mutated/truncated 4-1BB (BBt) domain in normal font
SQLCCQLKFWLPIGCAAFVVVCILGCILICWLTKKKYSSSVHDPNGEYMFMRAVNTA
KKSRLTDVTLQPFMRPVQTTQEEDGCSCRFPEEEEGGCEL
ICOS Transmembrane_ICOS Signaling Domain_Truncated OX-40 Signaling Domain (other
names: ICOS-truncated OX-40 (CD134) intracellular domain, ICOS_OX40t; ICOS-OX40t;
ICOS_40t; ICOS40t or ICOS-40t (SEQ ID NO: 15): ICOS sequence underlined and
mutated/truncated OX-40 (OX40t, 40t) domain in normal font
SQLCCQLKFWLPIGCAAFVVVCILGCILICWLTKKKYSSSVHDPNGEYMFMRAVNTA
KKSRLTDVTLGGGSFRTPIQEEQADAHSTLA
ICOS Transmembrane_ICOS Signaling Domain (mini-CD28)_Truncated 4-1BB Signaling
Domain (other names: ICOS(28)-truncated 4-1BB (BBt) intracellular domain, ICOS(28)_BBt;
ICOS(28)BBt; ICOS(28)-BBt; ICOS(28)_4-1BBt; ICOS(28)4-1BBt or ICOS(28)-4-1BBt)
(SEQ ID NO: 16): ICOS sequence underlined, CD28 portion in bold and BBt domain in
normal font
SQLCCQLKFWLPIGCAAFVVVCILGCILICWLTKKKYSSSVHDPNGEYMFMTPRRPGP
TRKHYQPYAPPRAVNTAKKSRLTDVTLQPFMRPVQTTQEEDGCSCRFPEEEEGGCEL
ICOS Transmembrane_ICOS Signaling Domain (mini-CD28)_Truncated OX-40 Signaling
Domain (other names: ICOS(28)-truncated OX-40; ICOS(28)-OX40t; ICOS(28)_OX40t;
ICOS(28)OX40t; ICOS (28)-40t; ICOS (28)_40t or ICOS(28)40t)(CD134) intracellular
domain (SEQ ID NO: 17): ICOS sequence underlined, CD28 portion in bold and OX40 (OX-
40t, 40t) domain in normal font
SQLCCQLKFWLPIGCAAFVVVCILGCILICWLTKKKYSSSVHDPNGEYMFMTPRRPGP
TRKHYQPYAPPRAVNTAKKSRLTDVTLGGGSFRTPIQEEQADAHSTLA
ICOS(28) (shortened ICOS(28)) (SEQ ID NO: 109): ICOS Transmembrane_ICOS Signaling
Domain (mini-CD28)
CWLTKKKYSSSVHDPNGEYMFMTPRRPGPTRKHYQPYAPPRAVNTAKKSRLTDVTL
CD28 Transmembrane_CD28 Signaling Domain_4-1BB Signaling Domain (other names:
CD28_BBwt signaling domain; CD28_BB; CD28BB; CD28-BB; 28_BBwt; 28BB; 28_BB or
28-BB) (SEQ NO: 120): CD28 Transmembrane domain_CD28 Signaling Domain_4-1BB
Signaling Domain
LFPGPSKPFWVLVVVGGVLACYSLLVTVAFIIFWVRSKRSRLLHSDYMNMTPRRPGPT
RKHYQPYAPPRDFAAYRSKRGRKKLLYIFKQPFMRPVQTTQEEDGCSCRFPEEEEGGC
EL
CD28 Transmembrane_CD28 Signaling Domain_truncated 4-1BB Signaling Domain (other
names: CD28_BBt signaling domain; CD28BBt; CD28-BBt; 28_BBt; 28BB; or 28-BBt) (SEQ
ID NO: 121):
LFPGPSKPFWVLVVVGGVLACYSLLVTVAFIIFWVRSKRSRLLHSDYMNMTPRRPGPT
RKHYQPYAPPRDFAAYRSQPFMRPVQTTQEEDGCSCRFPEEEEGGCEL
CD28 Transmembrane_CD28 Signaling Domain_truncated OX-40 Signaling Domain (other
names: CD28_OX40t signaling domain; 28-OX40t; 28-40t; 28_OX40t; 28_40t; 28OX40t or
2840t) (SEQ ID NO: 122):
LFPGPSKPFWVLVVVGGVLACYSLLVTVAFIIFWVRSKRSRLLHSDYMNMTPRRPGPT
RKHYQPYAPPRDFAAYRSGGGSFRTPIQEEQADAHSTLA
Transmembrane_ICOS Signaling Domain_4-1BB Signaling Domain with mutated polybasic
region (other names: ICOS_BB(xPB); ICOSBB(xPB) or ICOS-BB(xPB)) (SEQ ID NO: 123):
ICOS
SQLCCQLKFWLPIGCAAFVVVCILGCILICWLTKKKYSSSVHDPNGEYMFMRAVNTA
KKSRLTDVTLAAGAAALLYIFKQPFMRPVQTTQEEDGCSCRFPEEEEGGCEL
ICOS Transmembrane_ICOS Signaling Domain_4-1BB Signaling Domain with mutated
lysines (Other names: ICOS_BB(xUB); ICOS-BB(xUB) or ICOSBB(xUB))((SEQ ID NO:
124):
SQLCCQLKFWLPIGCAAFVVVCILGCILICWLTKKKYSSSVHDPNGEYMFMRAVNTA
KKSRLTDVTLKRGRKKLLYIFAQPFMRPVQTTQEEDGCSCRFPEEEEGGCEL
ICOS Transmembrane_ICOS Signaling Domain_4-1BB Signaling Domain with mutated
lysines and polybasic regions (Other names: ICOS_BB(xPB xUB); ICOS-BB(xPB xUB) or
ICOSBB(xPB xUB))(SEQ ID NO: 125):
SQLCCQLKFWLPIGCAAFVVVCILGCILICWLTKKKYSSSVHDPNGEYMFMRAVNTA
KKSRLTDVTLAAGAAALLYIFAQPFMRPVQTTQEEDGCSCRFPEEEEGGCEL
ICOS Transmembrane_ICOS Signaling Domain_OX-40 Signaling Domain (Other names:
ICOS_OX40wt; ICOS-40; ICOS_40 wild type; ICOS_40wt; ICOS_40wt; ICOS-40wt; ICOS-
OX40wt; ICOSOX40wt; ICOS40 or ICOS40wt) (SEQ ID NO: 126):
SQLCCQLKFWLPIGCAAFVVVCILGCILICWLTKKKYSSSVHDPNGEYMFMRAVNTA
KKSRLTDVTLRRDQRLPPDAHKPPGGGSFRTPIQEEQADAHSTLAKI
ICOS Transmembrane_ICOS Signaling Domain_OX-40 Signaling Domain with mutated
polybasic region (other names: ICOS_OX40(xPB); ICOS_40(xPB); ICOS-40(xPB);
ICOS40(xPB); ICOS-OX40(xPB); or ICOSOX40(xPB))(SEQ ID NO: 127):
SQLCCQLKFWLPIGCAAFVVVCILGCILICWLTKKKYSSSVHDPNGEYMFMRAVNTA
KKSRLTDVTLAADQALPPDAHKPPGGGSFRTPIQEEQADAHSTLAKI
ICOS Transmembrane_ICOS Signaling Domain_OX-40 Signaling Domain with mutated
lysine residues (other names: ICOS_OX40(xUB) ICOS_40(xUB); ICOS-40(xUB);
ICOS40(xUB); ICOS-OX40(xUB); or ICOSOX40(xUB))(SEQ ID NO: 128):
SQLCCQLKFWLPIGCAAFVVVCILGCILICWLTKKKYSSSVHDPNGEYMFMRAVNTA
KKSRLTDVTLRRDQRLPPDAHAPPGGGSFRTPIQEEQADAHSTLAAI
ICOS Transmembrane_ICOS Signaling Domain_OX-40 Signaling Domain with mutated
lysine residues and polybasic regions (other names: ICOS_OX40(xPBxUB)
ICOS_40(xPBxUB); ICOS-40(xPBxUB); ICOS40(xPBxUB); ICOS-OX40(xPBxUB); or
ICOSOX40(xPBxUB))(SEQ ID NO: 129):
SQLCCQLKFWLPIGCAAFVVVCILGCILICWLTKKKYSSSVHDPNGEYMFMRAVNTA
KKSRLTDVTLAADQALPPDAHAPPGGGSFRTPIQEEQADAHSTLAAI
ICOS intracellular domain (SEQ ID NO: 147)
CWLTKKKYSSSVHDPNGEYMFMRAVNTAKKSRLTDVTL
CD28 intracellular domain (SEQ ID NO: 193)
RSKRSRLLHSDYMNMTPRRPGPTRKHYQPYAPPRDFAAYRS

In some embodiments, the chimeric intracellular domain further comprises a third signal transduction domain. In some embodiments, the third signal transduction domain is derived from any one of a CD3 signaling domain, a CD2 signaling domain, or an interleukin 2 receptor binding (IL-2RB) protein signaling domain. In some embodiments, the CD3 signaling domain is derived form a CD3ζ or a CD3ε domain or a combination thereof.

In some embodiments, the chimeric intracellular domain further comprises a third signal transduction domain derived from a CD3ζ protein. In some embodiments, the third signal transduction domain of the chimeric intracellular domain is a CD3ζ protein of amino acid sequence according to SEQ ID NO: 18. In some embodiments, the third signal transduction domain of the chimeric intracellular domain is a CD3ζ comprising an amino acid sequence having at least 50%, 55%, 60%, 65%, 70%, 75%, 80%, 85%, 90%, 95%, 97% or 99% identity to according to SEQ ID NOs: 18.

Human CD3 ζ full length (CD3Z full length)
(SEQ ID NO: 18)
MKWKALFTAAILQAQLPITEAQSFGLLDPKLCYLLDGILFIYG
VILTALFLRVKFSRSADAPAYQQGQNQLYNELNLGRREEYDVL
DKRRGRDPEMGGKPQRRKNPQEGLYNELQKDKMAEAYSEIGMK
GERRRGKGHDGLYQGLSTATKDTYDALHMQALPPR

In some embodiments, the third signal transduction domain of the chimeric intracellular domain is a CD3 signaling domain comprising an amino acid sequence according to any one of SEQ ID NOs: 45, 46, 47 and 48. In some embodiments, the third signal transduction domain of the chimeric intracellular domain is a CD3 comprising an amino acid sequence according to SEQ ID NO: 45. In some embodiments, the third signal transduction domain of the chimeric intracellular domain is a CD3 comprising an amino acid sequence having at least 50%, 55%, 60%, 65%, 70%, 75%, 80%, 85%, 90%, 95%, 97% or 99% identity to SEQ ID NO: 45. In some embodiments, the third signal transduction domain of the chimeric intracellular domain is a truncated CD3ζ comprising an amino acid sequence according to SEQ ID NO: 46. In some embodiments, the third signal transduction domain of the chimeric intracellular domain is a truncated CD3ζ comprising an amino acid sequence having at least 50%, 55%, 60%, 65%, 70%, 75%, 80%, 85%, 90%, 95%, 97% or 99% identity to SEQ ID NO: 46. In some embodiments, the third signal transduction domain of the chimeric intracellular domain is a truncated CD3ε comprising an amino acid sequence according to SEQ ID NO: 47. In some embodiments, the third signal transduction domain of the chimeric intracellular domain is a truncated CD3ε comprising an amino acid sequence having at least 50%, 55%, 60%, 65%, 70%, 75%, 80%, 85%, 90%, 95%, 97% or 99% identity to SEQ ID NO: 47. In some embodiments, the third signal transduction domain of the chimeric intracellular domain is a combination of a CD3ε and a truncated CD3ζ domains (CD3ζε domain). In some embodiments, the third signal transduction domain of the chimeric intracellular domain is a CD3ζε comprising an amino acid sequence according to SEQ ID NO: 48. In some embodiments, the third signal transduction domain of the chimeric intracellular domain is a CD3ζε comprising an amino acid sequence having at least 50%, 55%, 60%, 65%, 70%, 75%, 80%, 85%, 90%, 95%, 97% or 99% identity to SEQ ID NO: 48.

In some embodiments, the third signal transduction domain of the chimeric intracellular domain is a mutant CD2 signaling domain. In some embodiments, the third signal transduction domain of the chimeric intracellular domain is a truncated CD2 signaling domain. In some embodiments, the third signal transduction domain of the chimeric intracellular domain is a truncated CD2 signaling domain comprising an amino acid sequence according to SEQ ID NO: 49. In some embodiments, the third signal transduction domain of the chimeric intracellular domain is a truncated CD2 signaling domain comprising an amino acid sequence having at least 50%, 55%, 60%, 65%, 70%, 75%, 80%, 85%, 90%, 95%, 97% or 99% identity to SEQ ID NOs: 49.

In some embodiments, the third signal transduction domain of the chimeric intracellular domain is an IL-2RB protein signaling domain comprising an amino acid sequence according to SEQ ID NO: 50. In some embodiments, the third signal transduction domain of the chimeric intracellular domain is an IL-2RB protein signaling domain comprising an amino acid sequence having at least 50%, 55%, 60%, 65%, 70%, 75%, 80%, 85%, 90%, 95%, 97% or 99% identity to SEQ ID NOs: 50.

In some embodiments, the chimeric intracellular domain further comprises a fourth signal transduction domain. In some embodiments, the fourth signal transduction domain is derived from any one of a CD3 signaling domain, a CD2 signaling domain or an interleukin 2 receptor binding (IL-2RB) protein signaling domain or a combination thereof, wherein the third and the fourth signal transduction domain are not identical.

In some embodiments, the fourth signal transduction domain of the chimeric intracellular domain is a CD3 signaling domain comprising an amino acid sequence according to any one of SEQ ID NOs: 45, 46, 47 and 48. In some embodiments, the fourth signal transduction domain of the chimeric intracellular domain is a CD3ζ comprising an amino acid sequence according to SEQ ID NO: 45. In some embodiments, the fourth signal transduction domain of the chimeric intracellular domain is a CD3ζ comprising an amino acid sequence having at least 50%, 55%, 60%, 65%, 70%, 75%, 80%, 85%, 90%, 95%, 97% or 99% identity to SEQ ID NO: 45. In some embodiments, the fourth signal transduction domain of the chimeric intracellular domain is a truncated CD3ζ comprising an amino acid sequence according to SEQ ID NO: 46. In some embodiments, the fourth signal transduction domain of the chimeric intracellular domain is a truncated CD3ζ comprising an amino acid sequence having at least 50%, 55%, 60%, 65%, 70%, 75%, 80%, 85%, 90%, 95%, 97% or 99% identity to SEQ ID NO: 46. In some embodiments, the fourth signal transduction domain of the chimeric intracellular domain is a truncated CD3ε comprising an amino acid sequence according to SEQ ID NO: 47. In some embodiments, the fourth signal transduction domain of the chimeric intracellular domain is a truncated CD3ε comprising an amino acid sequence having at least 50%, 55%, 60%, 65%, 70%, 75%, 80%, 85%, 90%, 95%, 97% or 99% identity to SEQ ID NO: 47. In some embodiments, the fourth signal transduction domain of the chimeric intracellular domain is a combination of a CD3ε and a truncated CD3ζ domains (CD3ζε domain). In some embodiments, the fourth signal transduction domain of the chimeric intracellular domain is a CD3ε comprising an amino acid sequence according to SEQ ID NO: 48. In some embodiments, the fourth signal transduction domain of the chimeric intracellular domain is a CD3ζε comprising an amino acid sequence having at least 50%, 55%, 60%, 65%, 70%, 75%, 80%, 85%, 90%, 95%, 97% or 99% identity to SEQ ID NO: 48.

In some embodiments, the fourth signal transduction domain of the chimeric intracellular domain is a mutant CD2 signaling domain. In some embodiments, the fourth signal transduction domain of the chimeric intracellular domain is a truncated CD2 signaling domain. In some embodiments, the fourth signal transduction domain of the chimeric intracellular domain is a truncated CD2 signaling domain comprising an amino acid sequence according to SEQ ID NO: 49. In some embodiments, the fourth signal transduction domain of the chimeric intracellular domain is a truncated CD2 signaling domain comprising an amino acid sequence having at least 50%, 55%, 60%, 65%, 70%, 75%, 80%, 85%, 90%, 95%, 97% or 99% identity to SEQ ID NOs: 49.

In some embodiments, the fourth signal transduction domain of the chimeric intracellular domain is an IL-2RB protein signaling domain comprising an amino acid sequence according to SEQ ID NO: 50. In some embodiments, the fourth signal transduction domain of the chimeric intracellular domain is an IL-2RB protein signaling domain comprising an amino acid sequence having at least 50%, 55%, 60%, 65%, 70%, 75%, 80%, 85%, 90%, 95%, 97% or 99% identity to SEQ ID NOs: 50.

The terms T cell, T-cell, t cell, t-cell, and T lymphocyte can be used interchangeably in the present disclosure.

In some embodiments, the extracellular domain comprises a protein or a portion thereof that binds to a target to induce activation and/or proliferation of an immune cell. In some embodiments, the extracellular domain comprises any one of: a) a component of a T cell Receptor (TCR) complex; b) a component of a chimeric antigen receptor (CAR); c) a component of a T cell co-receptor, wherein the T cell co-receptor is a T cell co-stimulatory protein or T cell inhibitory protein; d) a ligand that binds to a cell surface receptor or a component thereof; e) a component of a cytokine receptor; e) a component of a chemokine receptor; g) a component of an integrin receptor; h) a component of an endothelial cell surface protein receptor or a fragment thereof; i) a component of a neuronal guidance protein receptor; and f) a component of a complement receptor. In some embodiments, the component of the T cell co-receptor or the CAR is a component of PD1, CD28, CD2, OX-40, ICOS, CTLA-4, CD28, CD3, CD4, CD8, CD40L, Lag-3, Tim-3, or TIGIT, or a combination thereof. In some embodiments, the ligand or component of the T cell co-receptor or CAR binds to CD19, B cell maturation Ag (BCMA), PD-L1, PD-L2, IL-10, a proliferation-inducing ligand (APRIL), BAFF, OX-40L, ICOS-L, B7-1, B7-2, CD40, CD58, CD59, nectin, CD155, or CD112, or a combination thereof. In some embodiments, the cytokine receptor binds to IL-10, IL-27, TGF-β, IL-12, IL-1, IL-2, IL-4, IL-5, IFN-γ, or IFN-α/β, or a combination thereof. In some embodiments, the component of the complement receptor is a component of a single C3aR, C5aR, CD46/MCP, CD55, CD97, or DAF, or a combination thereof.

In some embodiments, the extracellular domain comprises an amino acid sequence of a component of any one of: a) a chemokine receptor; b) a cytokine receptor; c) a ligand for a cell surface receptor; d) an integrin receptor; e) a cell adhesion molecule or a receptor thereof; f) an endothelial cell surface protein receptor or a fragment thereof; g) a complement receptor; and h) a neuronal guidance protein receptor. In some embodiments, the extracellular domain comprises an amino acid sequence of a component of any one of epithelial growth factor receptor (EGFR), vascular-endothelial growth factor (VEGFR), chemokine receptor (CCR) 4, CCR5, CCR7, CCR10, Lymphocyte function-associated antigen-1 (LFA-1), leukocyte-specific β2 integrins (αLβ2, αMβ2, αXβ2, αDβ2), β7 integrins (α4β7 and αEβ7), extracellular matrix (ECM)-binding β1 integrins (α1-α6β1), L-selectin, or sialyl Lewis^{x}.

In some embodiments, the extracellular domain is a protein, a peptide, a glycoprotein, an antibody or a fragment thereof. In some embodiments, the antibody or fragment thereof is a Fab fragment, a F(ab)₂ fragment, a diabody, a nanobody, a sdAb, Fv, a VHH fragment, or a single chain Fv fragment.

In some embodiments, the extracellular domains comprises two or more binding sites for targeting two or more non-identical target antigens. In some embodiments, the extracellular domains comprises two or more binding sites for targeting two or more non-identical sites on a target antigen. In some embodiments, the extracellular domain comprises two antigen binding domains or fragments of a bispecific antibody. In some embodiments, the extracellular domain comprises a F(ab)₂ fragment of a bispecific antibody. In some embodiments, the extracellular domain comprises two or more antigen binding domains or fragments of a multi-specific antibody.

In some embodiments, the extracellular domain binds to a target that is a tumor antigen, a pathogen associated protein, or an antigen associated with the disease or disorder that is a cancer, an autoimmune disease or disorder, an infectious disease, an inflammatory disease, a renal disease or disorder, a lung disease or disorder, a liver disease or disorder, a cardiovascular disease or disorder, a neurodegenerative disorder or disorder, or a metabolic disorder or disorder.

In some embodiments, the tumor antigen is any one of a tumor associated antigen (TAA), a tumor secreted antigen (TSA) or an unconventional antigen (UCA). In some embodiments, the TAA is any one of a cancer germline antigen (CGA), a Human endogenous retroviruses (HERVs), tissue differentiation antigen (TDA) and overexpressed tumor antigen. In some embodiments, the TSA is derived from any one of a mosaic single nucleotide variations (mSNVs), a insertion-deletion mutations (INDELs), gene fusions and viral oncoproteins. In some embodiments, the UCA is derived from non-coding regions of the genome or from coding regions of the genome. In some embodiments, the UCA is derived from aberrant transcription, translation, or post-translational modifications.

In some embodiments, the TAA is associated with a solid tumor or cancer or a hematologic cancer. In some embodiments, the TAA is associated with a solid tumor or cancer is selected from a sarcoma, a carcinoma or a lymphoma that manifests as, leads to, or is associated with a solid tumor.

In some embodiments, the TAA is associated with a sarcoma that is a soft tissue sarcoma or a bone sarcoma (osteosarcoma). In some embodiments, the TAA is associated with a sarcoma selected from vesicular rhabdomyosarcoma, vesicular soft tissue sarcoma, ameloblastoma, angiosarcoma, chondrosarcoma, chordoma, bright tissue sarcoma, dedifferentiated liposarcoma, Hyperplastic small round cell tumor of connective tissue, embryonic rhabdomyosarcoma, epithelioid fibrosarcoma, epithelioid hemangioendothelioma, epithelioid sarcoma; sensitive neuroblastoma (esthesioneuroblastoma), Ewing sarcoma, extrarenal rhabdomyosarcoma, extraosseous myxoid chondrosarcoma, extraosseous osteosarcoma, fibrosarcoma, giant cell tumor, hemangiopericytoma, infantile fibrosarcoma, inflammatory myofibroblastoma, Kaposi sarcoma, bone smooth muscle sarcoma, liposarcoma, osteosarcoma, malignant fibrous histiocytoma (WE), malignant fibrous histiocytoma (WE), malignant mesenchymal tumor, malignant peripheral nerve sheath tumor, mesenchymal chondrosarcoma, myxoid liposarcoma, myxoid inflammatory fibroblastic sarcoma, multiple tumors with perivascular epithelioid cell differentiation, osteosarcoma, extraperiosteal osteosarcoma, tumors with perivascular epithelial cell differentiation, periosteum osteosarcoma, polymorphic liposarcoma, polymorphic rhabdomyosarcoma, PNET/extraosseous Ewing's tumor, rhabdomyosarcoma, small cell osteosarcoma, single fibroids, synovial sarcoma or capillary dilated osteosarcoma.

In some embodiments, the TAA is associated with a carcinoma selected from basal cell carcinoma, squamous cell carcinoma, renal cell carcinoma, ductal carcinoma in situ (DCIS), invasive ductal carcinoma or adenocarcinoma. In some embodiments, the TAA is associated with a carcinoma selected from adenosquamous carcinoma, anaplastic carcinoma, large cell carcinoma, colorectal carcinoma, pancreatic carcinoma, nasopharyngeal carcinoma or small cell carcinoma.

In some embodiments, the TAA is associated with a solid tumor or cancer selected from anal cancer, appendix cancer; cholangiocarcinoma (i.e., biliary tract cancer), breast cancer, bladder cancer, brain tumor, breast cancer, cervical cancer, colon cancer, colorectal cancer, colon polyp, unidentified primary cancer (cup), esophagus cancer, eye cancer, tubal cancer, kidney cancer, liver cancer, lung cancer, medulloblastoma, melanoma, oral cancer, ovarian cancer, prostate cancer, pancreatic cancer, gastric cancer, testicular cancer, laryngeal cancer, thyroid cancer, uterine cancer, vaginal cancer, or vulvar cancer.

In some embodiments, the breast cancer is an invasive breast duct cancer, carcinoma in situ of the duct, invasive lobular carcinoma or lobular carcinoma in situ. In some embodiments, the pancreatic cancer is adenocarcinoma or islet cell carcinoma. In some embodiments, the colorectal cancer is adenocarcinoma. In some embodiments, colonic polyps are associated with familial adenomatous polyposis. In some embodiments, the bladder cancer is transitional cell bladder cancer, squamous cell bladder cancer, or adenocarcinoma. In some embodiments, the lung cancer is non-small cell lung cancer. In some embodiments, the non-small cell lung cancer is adenocarcinoma, squamous cell lung cancer, or large cell lung cancer. In some embodiments, the non-small cell lung cancer is large cell lung cancer. In some embodiments, the lung cancer is small cell lung cancer. In some embodiments, the prostate cancer is adenocarcinoma or small cell carcinoma. In some embodiments, the ovarian cancer is epithelial ovarian cancer. In some embodiments, the cholangiocarcinoma is proximal cholangiocarcinoma or distal cholangiocarcinoma.

In some embodiments, the TAA is associated with any one of the hematological cancer selected from a leukemia, a myeloma or a lymphoma. In some embodiments, the TAA is associated with a leukemia selected from acute leukemia, acute lymphoblastic leukemia (ALL), acute lymphocytic leukemia, a B cell, T cell or FAB ALL, acute myeloid leukemia (AML), acute myelogenous leukemia, chronic myelocytic leukemia (CML), chronic lymphocytic leukemia (CLL), hairy cell leukemia, acute promyelocytic leukemia (APL), mixed-lineage leukemia (MLL) or myelodysplastic syndrome (MDS).

In some embodiments, the TAA is associated with a myeloma that is a multiple myeloma. In some embodiments, the TAA is associated with a multiple myeloma selected from the hyperdiploid (HMM) or the non-hyperdiploid or hypodiploid subtypes of multiple myeloma. In some embodiments, the TAA is associated with a multiple myeloma selected from light chain myeloma, non-secretory myeloma, solitary plasmacytoma, extramedullary plasmacytoma, monoclonal gammopathy of undetermined significance (MGUS), smoldering multiple myeloma (SMM), immunoglobulin D (IgD) myeloma or, immunoglobulin E (IgE) myeloma.

In some embodiments, the TAA is associated with a lymphoma that is a Hodgkin's lymphoma or a non-Hodgkin's lymphoma. In some embodiments, the TAA is associated with a non-Hodgkin's lymphoma. In some embodiments, the TAA is associated with a non-Hodgkin's lymphoma selected from a Small lymphocytic lymphoma (SLL), Lymphoplasmacytic lymphoma, Diffuse large cell lymphoma, Follicle center cell lymphoma, Burkitt's lymphoma, Burkitt-like lymphoma, Mantle cell lymphoma or Marginal zone B-cell lymphoma. In some embodiments, the TAA is associated with a lymphoma that is a Hodgkin's lymphoma. In some embodiments, the TAA is associated with a Hodgkin's lymphoma selected from nodular sclerosis classical Hodgkin lymphoma, lymphocyte-rich classical Hodgkin lymphoma or lymphocyte-depleted classical Hodgkin lymphoma.

In some embodiments, the TAA is associated with a cancer that is any one of acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CIVIL), chronic lymphocytic leukemia (CLL), hairy cell leukemia, myelodysplastic syndrome (MDS), Hodgkin's lymphoma, Hodgkin's disease, non-Hodgkin's lymphoma, multiple myeloma, colorectal carcinoma, pancreatic carcinoma, nasopharyngeal carcinoma, malignant histiocytosis, paraneoplastic syndrome/hypercalcemia of malignancy, bladder cancer, breast cancer, colorectal cancer, endometrial cancer, head cancer, neck cancer, hereditary nonpolyposis cancer, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the extracellular domain binds to a TAA selected from kallikrein 4, papillomavirus binding factor (PBF), preferentially expressed antigen of melanoma (PRAME), Wilms' tumor-I (WTI), Hydroxysteroid Dehydrogenase Like I (HSDLI), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CAI25), folate receptor a, sperm protein 17, tumor-associated differentially expressed gene-12 (TADG-12), mucin-16 (MUC-16), LI cell adhesion molecule (LICAM), mannan-MUC-1, Human endogenous retrovirus K (HERV-K-MEL), Kita-kyushu lung cancer antigen-I (KK-LC-1), human cancer/testis antigen (KM-HN-1), cancer testis antigen (LAGE-I), melanoma antigen-A1 (MAGE-A1), Sperm surface zona pellucida binding protein (Spl 7), Synovial Sarcoma, X Breakpoint 4 (SSX-4), Transient axonal glycoprotein-1 (TAG-I), Transient axonal glycoprotein-2 (TAG-2), Enabled Homolog (ENAH), mammoglobin-A, NY-BR-I, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin I (MUC I), p62, survivin, lymphocyte common antigen (CD45), DickkopfWNT Signaling Pathway Inhibitor I (DKKI), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor I Homolog (COA-1), 0-linked β-N-acetylglucosamine transferase (OGT), Osteosarcoma Amplified 9, Endoplasmic Reticulum Lectin (OS-9), Transforming Growth Factor Beta Receptor 2 (TGF-betaRll), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (MEI), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAP1), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTC1), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FN1), Gruwih Anest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDX5), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OAl), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gplOO), N-acetylglucosaminyltransferase V gene (GnTVf), Lymphocyte Antigen 6 Complex Locus K (LY6K), melanoma antigen-AlO (MAGE-AlO), melanoma antigen-Al2 (MAGE-Al2), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-Al, Fibronectin Type III Domain Containing 3B (FDNC3B) promyelocytic leukemia/retinoic acid receptor alpha fusion protein (pml-RARalpha), melanoma antigen-Cl (MAGE-Cl), membrane protein alternative spliced isoform (D393-CD20), melanoma antigen-A4 (MAGE-A4), or melanoma antigen-A3 (MAGE-A3).

In some embodiments, the autoimmune condition or disorder is any one of Type 1 Diabetes, rheumatoid arthritis (RA), systemic lupus erythematosis (SLE), multiple sclerosis (MS), celiac disease, sjÖgren syndrome, polymyalgia rheumatica, ankylosing spondylitis, alopecia areata, vasculitis and temporal arteritis. In some embodiments, the tumor associated antigen (TAA) associated with the autoimmune condition or disorder is derived from any one of Carboxypeptidase H, Chromogranin A, Glutamate decarboxylase, Imogen-38, Insulin, Insulinoma antigen-2 and 2β, Islet-specific glucose-6-phosphatase catalytic subunit related protein (IGRP), Proinsulin, α-enolase, Aquaporin-4, β-arrestin, Myelin basic protein, Myelin oligodendrocytic glycoprotein, Proteolipid protein, S100-β, Citrullinated protein, Collagen II, Heat shock proteins, Human cartilage glycoprotein, Double-stranded DNA, La antigen, Nucleosomal histones and ribonucleoproteins (snRNP), Phospholipid-β-2 glycoprotein I complex, Poly(ADP-ribose) polymerase, and Sm antigens of U-1 small ribonucleoprotein complex.

In some embodiments, the pathogen associated antigen is an antigen from a bacterial, a fungal or a parasitic protein or fragment thereof. In some embodiments, the pathogen associated antigen is associated with HIV infection, human Cytomegalovirus infection, Hepatitis B infection, Hepatitis C infection, Ebola virus infection, Dengue, Yellow fever, Listeriosis, Tuberculosis, Cholera, Malaria, Leishmaniasis, or Trypanosoma infection, or a combination thereof.

In some embodiments, the neurodegenerative disorder or condition is any one of Alzheimer's disease (AD) and other dementias, Parkinson's disease (PD) and PD-related disorders, Prion disease, Motor neurone diseases (MND), Huntington's disease (HD), Spinocerebellar ataxia (SCA) or Spinal muscular atrophy (SMA). In some embodiments, the antigen associated with the neurodegenerative disorder or condition is any one of Amyloid (Ab), tau, alpha-synuclein (α-syn), mHTT or prion PrP^{sc} or a combination thereof.

In some embodiments, the extracellular domain binds to a target with a binding affinity of 1 fM to 100 μM. In some embodiments, the extracellular domain binds to a target with a binding affinity of 1 pM to 100 μM. In some embodiments, the extracellular domain binds to a target with a binding affinity of 1 pM to 10 pM. In some embodiments, the extracellular domain binds to a target with a binding affinity of 10 pM to 50 pM. In some embodiments, the extracellular domain binds to a target with a binding affinity of 10 pM to 100 pM. In some embodiments, the extracellular domain binds to a target with a binding affinity of 100 pM to 500 pM. In some embodiments, the extracellular domain binds to a target with a binding affinity of 500 pM to 1 nM. In some embodiments, the extracellular domain binds to a target with a binding affinity of 1 nM to 10 nM. In some embodiments, the extracellular domain binds to a target with a binding affinity of 10 nM to 100 nM. In some embodiments, the extracellular domain binds to a target with a binding affinity of 100 nM to 500 nM. In some embodiments, the extracellular domain binds to a target with a binding affinity of 500 nM to 1 μM. In some embodiments, the extracellular domain binds to a target with a binding affinity of 1 μM to 10 μM. In some embodiments, the extracellular domain binds to a target with a binding affinity of 1 μM to 5 μM. In some embodiments, the extracellular domain binds to a target with a binding affinity of 5 μM to 7.5 μM. In some embodiments, the extracellular domain binds to a target with a binding affinity of 7.5 μM to 10 μM.

In some embodiments, the extracellular domain comprises a signal peptide at the N-terminus. In some embodiments, the signal peptide can be derived from a surface expressing protein or a secretory protein. In some embodiments, the signal peptide can be derived from Preprolactin, HIV pre-Env, HCV polyprotein, CB virus polyprotein, Pestivirus polyprotein, Precalreticulin, pre-VSV-G, HLA class I histocompatibility antigen or PD-1 signal peptide (PD-1 SP), interleukin 12 (IL12), GM-CSF or CD8 alpha chain (CD8a). In some embodiments, the signal peptide is PD-1 signal peptide (PD-1 SP). In some embodiments, the signal peptide is a HLA class I histocompatibility antigen or a portion thereof. In some embodiments, the extracellular domain is derived from PD1. In some embodiments, the extracellular domain comprises the amino acid sequence from position 1 to 163 of the amino acid sequence according to any one of SEQ ID NOs: 19-21. In some embodiments, the extracellular domain comprises the amino acid sequence from position 1 to 163 of the amino acid sequence according to SEQ ID NOs: 19. In some embodiments, the extracellular domain comprises the amino acid sequence from position 1 to 163 of the amino acid sequence according to SEQ ID NOs: 20. In some embodiments, the extracellular domain comprises the amino acid sequence from position 1 to 163 of the amino acid sequence according to SEQ ID NOs: 21.

In some embodiments, the extracellular domain comprises the amino acid sequence according to any one of SEQ ID NOs: 22-23. In some embodiments, the extracellular domain comprises the amino acid sequence according to SEQ ID NOs: 22. In some embodiments, the extracellular domain comprises the amino acid sequence having at least 50%, 55%, 60%, 65%, 70%, 75%, 80%, 85%, 90%, 95%, 97% or 99% identity to SEQ ID NOs: 22. In some embodiments, the extracellular domain comprises the amino acid sequence according to SEQ ID NOs: 23. In some embodiments, the extracellular domain comprises the amino acid sequence having at least 50%, 55%, 60%, 65%, 70%, 75%, 80%, 85%, 90%, 95%, 97% or 99% identity to SEQ ID NOs: 23.

TABLE 4
Amino acid sequences of PD1 extracellular
domains of RTCR PD1

(PD1 Signal Peptide_PD1 Extracellular_PD1
Transmembrane_PD1 Intracellular)
(other names: PD1 wt (human-wild type);
PD1:WT; PD-1; PD-1 wt; PD-1 wild type; PD1;
PD1wt or PD1 wild type) (SEQ ID NO: 19):
Signal Peptide (italicized), Extracellular
domain (IG-like V domain in bold and stalk
in bold and underlined), Trans Membrane
(underlined) and Intracellular domain
in double underline
MQIPQAPWPVFWAVLQLGWRPGWFLDSPDRPWNP
PTFSPALLVVTEGDNATFTCSFSNTSESFVLNWY
RMSPSNQTDKLAAFPEDRSQPGQDCRFRVTQLPN
GRDFHMSVVRARRNDSGTYLCGAISLAPKAQIKE
SLRAELRVT [symbol] [symbol]
VGVVGGLLGSLVLLVWVLAVI
CSRAARGTIGARRTGQPLKEDPSAVPVFSVDY
GELDFQWREKTPEPPVPCVPEQTEYATIV
FPSGMGTSSPARRGSADGPRSAQPLRPED
GHCSWPL
PD1 Signal Peptide_PD1 Extracellular PD1
Transmembrane (Other name: PD-1 truncated)
(SEQ ID NO: 20): PD1 Signal Peptide
(italicized), Extracellular domain
(IG-like V domain in bold and stalk
in bold and underlined), Trans Membrane
(underlined) and Intracellular tail
in double underline
MQIPQAPWPVFWAVLQLGWRPGWFLDSPDRPWNP
PTFSPALLVVTEGDNATFTCSFSNTSESFVLNWYR
MSPSNQTDKLAAFPEDRSQPGQDCRFRVTQLPNGR
DFHMSVVRARRNDSGTYLCGAISLAPKAQIKESLR
AELRVT [symbol] [symbol]
VGVVGGLLGSLVLLVWVLAVICSR
HLA-A2 Signal Peptide_PD1 Extracellular PD1
Transmembrane (PD1-TLs; HLASP-Truncated,
PD1-TLs, PD1:TLs) (SEQ ID NO: 21):
HLA-A2 Signal Peptide (italicized),
Extracellular domain (IG-like V domain
in bold and stalk in bold and underlined),
Trans Membrane (underlined) and Intracellular
tail in double underline
MAVMAPRTLVLLLSGALALTQTWAFLDSPDRPWNP
PTFSPALLVVTEGDNATFTCSFSNTSESFVLNWYR
MSPSNQTDKLAAFPEDRSQPGQDCRFRVTQLPNGR
DFHMSVVRARRNDSGTYLCGAISLAPKAQIKESLR
AELRVT [symbol] [symbol]
VVGVVGGLLGSLVLLVWVLAVICSR
PD-1 (extracellular domain) (SEQ ID NO: 22):
Signal Peptide (italicized), Extracellular
domain (IG-like V domain in bold and stalk
in bold and underlined)
MQIPQAPWPVFWAVLQLGWRPGWFLDSPDRPWNP
PTFSPALLVVTEGDNATFTCSFSNTSESFVLNWY
RMSPSNQTDKLAAFPEDRSQPGQDCRFRVTQLPN
GRDFHMSVVRARRNDSGTYLCGAISLAPKAQIKE
SLRAELRVT [symbol]
PD-1 (HLA A2-Signal Peptide extracellular
domain) (other name: PD-1 (HLA A2-SP
extracellular domain) (SEQ ID NO: 23):
Signal Peptide (italicized), Extracellular
domain (IG-like V domain in bold and
stalk in bold and underlined)
MAVMAPRTLVLLLSGALALTQTWAFLDSPDRPWNP
PTFSPALLVVTEGDNATFTCSFSNTSESFVLNWYR
MSPSNQTDKLAAFPEDRSQPGQDCRFRVTQLPNG
RDFHMSVVRARRNDSGTYLCGAISLAPKAQIKES
LRAELRVT

[symbol]

In some embodiments, the RTCR disclosed herein comprises the amino acid sequence according to any one of SEQ ID NOs: 24-44 and 130-132.

In some embodiments, the RTCR disclosed herein comprises the amino acid sequence according to SEQ ID NO: 24. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 24.

In some embodiments, the RTCR disclosed herein comprises the amino acid sequence according to SEQ ID NO: 25. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 25.

In some embodiments, the RTCR disclosed herein comprises the amino acid sequence according to SEQ ID NO: 26. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 26.

In some embodiments, the RTCR disclosed herein comprises the amino acid sequence according to SEQ ID NO: 27. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 27.

In some embodiments, the RTCR disclosed herein comprises the amino acid sequence according to SEQ ID NO: 28. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 28.

In some embodiments, the RTCR disclosed herein comprises the amino acid sequence according to SEQ ID NO: 29. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 29.

In some embodiments, the RTCR disclosed herein comprises the amino acid sequence according to SEQ ID NO: 30. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 30.

In some embodiments, the extracellular domain of the RTCR disclosed herein comprises the amino acid sequence according to SEQ ID NO: 31. In some embodiments, the extracellular domain of the RTCR disclosed herein comprises the amino acid sequence having at least 50%, 55%, 60%, 65%, 70%, 75%, 80%, 85%, 90%, 95%, 97% or 99% identity to SEQ ID NO: 31.

In some embodiments, the RTCR disclosed herein comprises the amino acid sequence according to SEQ ID NO: 32. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 32.

In some embodiments, the RTCR disclosed herein comprises the amino acid sequence according to SEQ ID NO: 33. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 33.

In some embodiments, the RTCR disclosed herein comprises the amino acid sequence according to SEQ ID NO: 34. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 34.

In some embodiments, the RTCR disclosed herein comprises the amino acid sequence according to SEQ ID NO: 35. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 35.

In some embodiments, the RTCR disclosed herein comprises the amino acid sequence according to SEQ ID NO: 36. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 36.

In some embodiments, the RTCR disclosed herein comprises the amino acid sequence according to SEQ ID NO: 37. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 37.

In some embodiments, the RTCR disclosed herein comprises the amino acid sequence according to SEQ ID NO: 38. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 38.

In some embodiments, the RTCR disclosed herein comprises the amino acid sequence according to SEQ ID NO: 39. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 39.

In some embodiments, the RTCR disclosed herein comprises the amino acid sequence according to SEQ ID NO: 40. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 40.

In some embodiments, the RTCR disclosed herein comprises the amino acid sequence according to SEQ ID NO: 41. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 41.

In some embodiments, the RTCR disclosed herein comprises the amino acid sequence according to SEQ ID NO: 42. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 42.

In some embodiments, the RTCR disclosed herein comprises the amino acid sequence according to SEQ ID NO: 43. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 43.

In some embodiments, the RTCR disclosed herein comprises the amino acid sequence according to SEQ ID NO: 44. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 44.

In some embodiments, the RTCR disclosed herein comprises the amino acid sequence according to SEQ ID NO: 130. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 130.

In some embodiments, the RTCR disclosed herein comprises the amino acid sequence according to SEQ ID NO: 131. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 131.

In some embodiments, the RTCR disclosed herein comprises the amino acid sequence according to SEQ ID NO: 132. In some embodiments, the RTCR disclosed herein comprises the amino acid sequence having at least 50%, 55%, 60%, 65%, 70%, 75%, 80%, 85%, 90%, 95%, 97% or 99% identity to SEQ ID NO: 132.

TABLE 5
Amino acid sequences of PD1 based recombinant T cell co-stimulatory receptor
(RTCR).

HLA-A2 Signal Peptide_PD1 Extracellular_CD28 Transmembrane_CD28 Signaling Domain
(Other names: PD-1-CD28 Domain Swap; HLA A2-SP-PD-1_28; HLA A2-SP-PD-1_CD28
DS; HLA A2-SP-PD-1_CD28; PD1_CD28 or PD1:CD28 or PD_28) (SEQ ID NO: 24):
HLA-A2 Signal Peptide (italicized), PD 1 extracellular domain (IG-like V domain in bold and
stalk in bold and underlined), CD28 Transmembrane (underlined) and Intracellular domain in
double underline; and CD28 signal domain: Stalk (underlined and italicized), transmembrane
domain (double underlined), intracellular domain (IC) (dashed underlined) (SEQ ID NO: 10)
MAVMAPRTLVLLLSGALALTQTWAFLDSPDRPWNPPTFSPALLVVTEGDNATFTCSFS
NTSESFVLNWYRMSPSNQTDKLAAFPEDRSQPGQDCRFRVTQLPNGRDFHMSVV

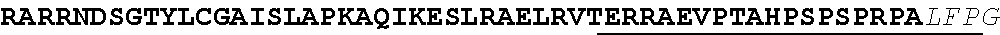

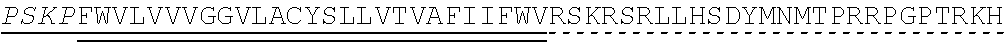

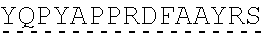

HLA-A2 Signal Peptide_PD1 Extracellular_ICOS Transmembrane_ICOS Signaling Domain
(Other names: PD-1-ICOS Domain Swap; HLASP-PD-1-ICOS DS; HLA A2-SP-PD-1_ICOS;
PD-1-ICOS; PD-1 :ICOS; PD1_ICOS) (SEQ ID NO: 25): HLA-A2 Signal Peptide (italicized),
PD 1 extracellular domain (IG-like V domain in bold and stalk in bold and underlined), ICOS
Transmembrane (underlined) and Intracellular domain in double underline; and ICOS domain:
Stalk (underlined and italicized), transmembrane domain (double underlined), intracellular
domain (IC) (dashed underlined)
MAVMAPRTLVLLLSGALALTQTWAFLDSPDRPWNPPTFSPALLVVTEGDNATFTCSFS
NTSESFVLNWYRMSPSNQTDKLAAFPEDRSQPGQDCRFRVTQLPNGRDFHMSVV

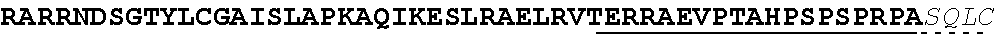

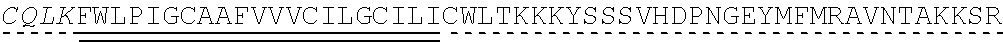

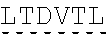

HLA-A2 Signal Peptide_PD1 Extracellular_ICOS Transmembrane_ICOS Signaling
Domain_4-1BB Signaling Domain (other names: PD1 HLASP-ICOS-4-1BB; HLA A2-SP-
PD-1_ICOS BBwt; HLA A2-SP-PD-1_ICOS_BB; HLA A2-SP-PD-1_ICOS CD137; HLA
A2-SP-PD-1_ICOS CD137 wt; PD1_ICOS BBwt; PD1:ICOSBBwt; PD1:ICOSBB) (SEQ ID
NO: 26): PD 1 extracellular domain in regular font; ICOS domain underlined; and 4-1BB
domain (CD137 signaling domain, wild type) in bold
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPASQLCCQLKF
WLPIGCAAFVVVCILGCILICWLTKKKYSSSVHDPNGEYMFMRAVNTAKKSRLTDVT
LKRGRKKLLYIFKQPFMRPVQTTQEEDGCSCRFPEEEEGGCEL
HLA-A2 Signal Peptide_PD1 Extracellular_ICOS Transmembrane_ICOS Signaling
Domain Truncated 4-1BB Signaling Domain
(Other names: PD1 HLASP-ICOS-truncated 4-1BB; HLA A2-SP-PD-1-ICOS BBt; HLA A2-
SP-PD-1_ICOS BBt. HLA A2-SP-PD-1_ICOS truncated CD137; HLA A2-SP-PD-
1_ICOS truncated CD137 wt; PD1_ICOS BBt; PD1;ICOSBBt) (SEQ ID NO: 27): PD 1
extracellular domain in regular font; ICOS domain underlined; and truncated 4-1BB (truncated
CD137 signaling domain in bold
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPASQLCCQLKF
WLPIGCAAFVVVCILGCILICWLTKKKYSSSVHDPNGEYMFMRAVNTAKKSRLTDVT
LQPFMRPVQTTQEEDGCSCRFPEEEEGGCEL
HLA-A2 Signal Peptide_PD1 Extracellular_ICOS Transmembrane_ICOS Signaling
Domain Truncated OX-40 Signaling Domain
(Other names: PD1 HLASP-ICOS-truncated OX40; HLA A2-SP-PD-1_ICOS OX40t, HLA
A2-SP-PD-1_ICOS_40t; HLA A2-SP-PD-1_ICOS truncated CD134; PD_ICOS_OX40t;
PD1:ICOS40t) (SEQ ID NO: 28): PD 1 extracellular domain in regular font; ICOS domain
underlined; and truncated OX40 (truncated CD134 signaling domain) in bold
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPASQLCCQLKF
WLPIGCAAFVVVCILGCILICWLTKKKYSSSVHDPNGEYMFMRAVNTAKKSRLTDVT
LGGGSFRTPIQEEQADAHSTLA
HLA-A2 Signal Peptide_PD1 Extracellular_ICOS Transmembrane_ICOS Signaling Domain
(mini-CD28)_Truncated 4-1BB Signaling Domain (Other names: PD1_ICOS(28) BBt;
PD1_ICOS(28)_truncated CD137; PD1:ICOS(28)-BBt or PD1:ICOS(28)BBt) (SEQ ID NO:
29): PD 1 extracellular domain in regular font; ICOS (28) domain: ICOS portions are
underlined and inserted CD28 portion bold and underline; and truncated CD137 domain in
bold
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPASQLCCQLKF
WLPIGCAAFVVVCILGCILICWLTKKKYSSSVHDPNGEYMFMTPRRPGPTRKHYQPY

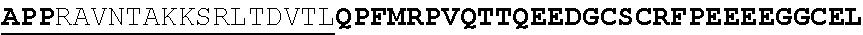

HLA-A2 Signal Peptide_PD1 Extracellular_ICOS Transmembrane_ICOS Signaling Domain
(mini-CD28)_Truncated OX-40 Signaling Domain (Other names: HLASP-ICOS DS-
CD28(PRRP)-mutated CD134; PD_ICOS(28) OX40t; PD_ICOS(28)_40t;
PD1_ICOS(28) truncated CD134; PD1:ICOS(28)-OX40t; PD1:ICOS(28)OX40t;
PD1:ICOS(28)-40t or PD1:ICOS(28)40t) (SEQ ID NO: 30): PD 1 extracellular domain in
regular font; ICOS (28) domain: ICOS portions are underlined and inserted CD28 portion bold
and underline; and truncated CD134 domain in bold
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPASQLCCQLKF

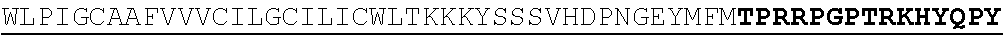

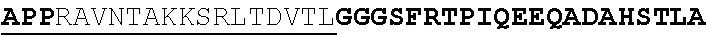

PD1 Extracellular (without HLA A2 Signal Peptide) (SEQ ID NO: 31)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPA
PD1 extracellular domain without signal peptide-CD28 domain swap (DS) (Other name:
PD_28) (SEQ ID NO: 32)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPALFPGPSKPFWVLVVVGGVLACYSLLVTVAFIIFWV
RSKRSRLLHSDYMNMTPRRPGPTRKHYQPYAPPRDFAAYRS
PD1 extracellular domain without signal peptide-CD28 DS-CD137 domain (Other name:
PD1_28_BBwt) (SEQ ID NO: 33)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPALFPGPSKPFWVLVVVGGVLACYSLLVTVAFIIFWV
RSKRSRLLHSDYMNMTPRRPGPTRKHYQPYAPPRDFAAYRSKRGRKKLLYIFKQPFM
RPVQTTQEEDGCSCRFPEEEEGGCEL
PD1 extracellular domain without signal peptide-CD28 DS-truncated CD137 domain (Other
name: PD1_28_BBt) (SEQ ID NO: 34)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPALFPGPSKPFWVLVVVGGVLACYSLLVTVAFIIFWV
RSKRSRLLHSDYMNMTPRRPGPTRKHYQPYAPPRDFAAYRSQPFMRPVQTTQEEDGC
SCRFPEEEEGGCEL
PD1 extracellular domain without signal peptide-CD28 DS-truncated CD134 domain (Other
name: PD1_28_OX40t) (SEQ ID NO: 35)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPALFPGPSKPFWVLVVVGGVLACYSLLVTVAFIIFWV
RSKRSRLLHSDYMNMTPRRPGPTRKHYQPYAPPRDFAAYRSGGGSFRTPIQEEQADA
HSTLA
PD1 extracellular domain without signal peptide-ICOS DS (Other name: PD1_ICOS) (SEQ ID
NO: 36)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTL
PD1 extracellular domain without signal peptide-ICOS DS-CD137 (Other name:
PD1_ICOS_BBwt) (SEQ ID NO: 37)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTAHPSPSPRPASQLCCQLKFWLPIGCAAFVVVCILGCILICWLTKK
KYSSSVHDPNGEYMFMRAVNTAKKSRLTDVTLKRGRKKLLYIFKQPFMRPVQTTQEE
DGCSCRFPEEEEGGCEL
PD1 extracellular domain without signal peptide-ICOS DS-truncated CD137 (Other name:
PD1_ICOS_BBt) (SEQ ID NO: 38)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTLQPFMRPVQTTQEEDGCSCRFPEEEE
GGCEL
PD1 extracellular domain without signal peptide-ICOS DS-truncated CD134 (Other name:
PD1_ICOS OX40t (SEQ ID NO: 39)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTLGGGSFRTPIQEEQADAHSTLA
PD1 extracellular domain without signal peptide-ICOS DS-CD28(PRRP)-truncated CD137)
(Other name: PD1_ICOS(28)_BBt (SEQ ID NO: 40)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMTPRRPGPTRKHYQPYAPPRAVNTAKKSRLTDVTLQPFMRPV
QTTQEEDGCSCRFPEEEEGGCEL
PD1 extracellular domain without signal peptide-ICOS DS-CD28(PRRP)-truncated CD134
(Other name: PD1_ICOS(28)_OX40t) (SEQ ID NO: 41)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMTPRRPGPTRKHYQPYAPPRAVNTAKKSRLTDVTLGGGSFRT
PIQEEQADAHSTLA
PD1 extracellular domain without signal peptide-ICOS DS-truncated CD137 domain-truncated
CD2 signaling domain (Other name: PD1_ICOS_BBt:CD2t) (SEQ ID NO: 42)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTLQPFMRPVQTTQEEDGCSCRFPEEEE
GGCELQNPATSQHPPPPPGHRSQAPSHRPPPPGHRVQHQPQKRPPAPSGTQVHQQKGP
PLPRPRVQPKPPHGAAENSLSPSSN
PD1 extracellular domain without signal peptide-ICOS DS-truncated CD137 domain-truncated
CD2 signaling domain (Other name: PD1_ICOS_BBt:IL2RB(YLRQ)) (SEQ ID NO: 43)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTLQPFMRPVQTTQEEDGCSCRFPEEEE
GGCELNCRNTGPWLKKVLKCNTPDPSKFFSQLSSEHGGDVQKWLSSPFPSSSFSPGGL
APEISPLEVLERDKVTQLLPLNTDAYLSLQELQGQDPTHLVSYLRQWVVIPPPLSSPGP
QAS
PD1 extracellular domain without signal peptide-ICOS DS-truncated CD137 domain-truncated
CD2 signaling domain-IL-2 receptor binding (IL2RB)(YLRQ) protein (Other name:
PD1_ICOS_BBt_CD2t_IL2RB(YLRQ) (SEQ ID NO: 44)
FLDSPDRPWNPPTFSPALLVVTEGDNATFTCSFSNTSESFVLNWYRMSPSNQTDKLAA
FPEDRSQPGQDCRFRVTQLPNGRDFHMSVVRARRNDSGTYLCGAISLAPKAQIKESLR
AELRVTERRAEVPTARPSPSPRPASQLCCQLKFWLPIGCAAFVVVCILGCILICWLTKK
KYSSSVHDPNGEYMFMRAVNTAKKSRLTDVTLQPFMRPVQTTQEEDGCSCRFPEEEE
GGCELQNPATSQHPPPPPGHRSQAPSHRPPPPGHRVQHQPQKRPPAPSGTQVHQQKGP
PLPRPRVQPKPPHGAAENSLSPSSNNCRNTGPWLKKVLKCNTPDPSKFFSQLSSEHGG
DVQKWLSSPFPSSSFSPGGLAPEISPLEVLERDKVTQLLPLNTDAYLSLQELQGQDPTH
LVSYLRQWVVIPPPLSSPGPQAS
HLA-A2 Signal Peptide_PD1 Extracellular_CD28 Transmembrane_CD28 Signaling
Domain 4-1BB Signaling Domain (Other names: PD1_28_BBwt; PD1_28_BB;
PD1_CD28_BB; PD1_CD28_BB wt; PD1_CD28_CD137; PD1:28BB; PD1:28BB wt;
PD1:28-BB or PD1:28-BB wt) (SEQ ID NO: 130)
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPALFPGPSKPF
WVLVVVGGVLACYSLLVTVAFIIFWVRSKRSRLLHSDYMNMTPRRPGPTRKHYQPY
APPRDFAAYRSKRGRKKLLYIFKQPFMRPVQTTQEEDGCSCRFPEEEEGGCEL
HLA-A2 Signal Peptide_PD1 Extracellular_CD28 Transmembrane_CD28 Signaling
Domain_Truncated 4-1BB Signaling Domain (Other names: PD1_28_BBt; PD1_CD28_BBt;
PD1_CD28_truncated CD137; PD1:28BBt; or PD1:28-BBt) (SEQ ID NO: 131)
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPALFPGPSKPF
WVLVVVGGVLACYSLLVTVAFIIFWVRSKRSRLLHSDYMNMTPRRPGPTRKHYQPY
APPRDFAAYRSQPFMRPVQTTQEEDGCSCRFPEEEEGGCEL
HLA-A2 Signal Peptide_PD1 Extracellular_CD28 Transmembrane_CD28 Signaling
Domain_Truncated OX-40 Signaling Domain (Other names: PD1_28_OX40t; PD1_28_40t;
PD_CD28_OX40t; PD_CD28_40t; PD-1_CD28_truncated CD134; PD1:2840t;
PD1:28OX40t; PD1:20-OX40t or PD1:28-40t) (SEQ ID NO: 132): PD1 Extracellular (with
HLA-A2 Signal Peptide)_CD28 Transmembrane_CD28 Signaling Domain_Truncated OX-40
Signaling Domain
MAVMAPRTLVLLLSGALALTQTWAFLDSPDRPWNPPTFSPALLVVTEGDNATFTCSF
SNTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPALFPGPSKPF
WVLVVVGGVLACYSLLVTVAFIIFWVRSKRSRLLHSDYMNMTPRRPGPTRKHYQPY
APPRDFAAYRSGGGSFRTPIQEEQADAHSTLA

TABLE 6
Amino acid sequences of third and fourth
signaling domains of RTCR

Human CD3 ζ, intracellular signaling
domain (Other names: CD3Z (intracellular
Signaling Domain);
CD3Z) (SEQ ID NO: 45)
RVKFSRSADAPAYQQGQNQLYNELNLGRREEYDVLDKRR
GRDPEMGGKPQRRKNPQEGLYNELQKDKMAEAYSEIGMK
GERRRGKGHDGLYQGLSTATKDTYDALHMQALPPR
Human CD3 Z signaling domain truncated
(CD3 Z truncated domain) (Other names:
Human CD3 ζ signaling domain
truncated Z; CD3 ζ truncated
domain; CD3Zt or Zt)
(SEQ ID NO: 46)
RVKFSRSADAPAYQQGQNQLYNELNLGRREEYDVLDKRR
GRDPEMGGK
Human CD3 E signaling domain truncated
(CD3 E truncated domain) (Other name: Human
CD3 ϵ signaling domain truncated;
CD3 ϵ truncated domain; CD3Et or
Et (SEQ ID NO: 47)
PVTRGAGAGGRQRGQNKERPPPVPNPDYEPIRKGQRD
LYSGLNQRRI
Human CD3 ZE signaling domain (CD3 ZE
domain) (Other name: Human CD3 ζϵ signaling
domain; CD3 ζϵ domain; CD3ZE or ZE)
(SEQ ID NO: 48)
RVKFSRSADAPAYQQGQNQLYNELNLGRREEYDVLDKRR
GRDPEMGGKPVTRGAGAGGRQRGQNKERPPPVPNPDYEP
IRKGQRDLYSGLNQRRI
CD2 truncated Signaling Domain (Other name:
CD2 or 2) (SEQ ID NO: 49)
QNPATSQHPPPPPGHRSQAPSHRPPPPGHRVQHQPQKRPPA
PSGTQVHQQKGPPLPRPRVQPKPPHGAAENSLSPSSN
IL-2 receptor binding (IL2RB) protein
Signaling Domain (YLRQ shown in bold)
(Other name: IL2RB(YLRQ)
(SEQ ID NO: 50)
NCRNTGPWLKKVLKCNTPDPSKFFSQLSSEHGGDVQK
WLSSPFPSSSFSPGGLAPEISPLEVLERDKVTQLLPL
NTDAYLSLQELQGQDPTHLVSYLRQWVVIPPPLSSPG
PQAS

In some embodiments, the extracellular domain is derived from CD19 binding protein. In some embodiments, the CD19 binding protein is a CD19 binding chimeric antigen receptor (CAR). In some embodiments, the extracellular domain comprises the amino acid sequence according to SEQ ID NO: 51. In some embodiments, the extracellular domain comprises the amino acid sequence having at least 50%, 55%, 60%, 65%, 70%, 75%, 80%, 85%, 90%, 95%, 97% or 99% identity to SEQ ID NO: 51.

In some embodiments, the CD19 binding chimeric antigen receptor comprises the amino acid sequence according to any one of SEQ ID NOs: 52-69. In some embodiments, the CD19 binding chimeric antigen receptor comprises the amino acid sequence according to SEQ ID NO: 52. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 52.

In some embodiments, the CD19 binding chimeric antigen receptor comprises the amino acid sequence according to SEQ ID NO: 53. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 53.

In some embodiments, the CD19 binding chimeric antigen receptor comprises the amino acid sequence according to SEQ ID NO: 54. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 54.

In some embodiments, the CD19 binding chimeric antigen receptor comprises the amino acid sequence according to SEQ ID NO: 55. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 55.

In some embodiments, the CD19 binding chimeric antigen receptor comprises the amino acid sequence according to SEQ ID NO: 56. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 56.

In some embodiments, the CD19 binding chimeric antigen receptor comprises the amino acid sequence according to SEQ ID NO: 57. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 57.

In some embodiments, the CD19 binding chimeric antigen receptor comprises the amino acid sequence according to SEQ ID NO: 58. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 58.

In some embodiments, the CD19 binding chimeric antigen receptor comprises the amino acid sequence according to SEQ ID NO: 59. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 59.

In some embodiments, the CD19 binding chimeric antigen receptor comprises the amino acid sequence according to SEQ ID NO: 60. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 60.

In some embodiments, the CD19 binding chimeric antigen receptor comprises the amino acid sequence according to SEQ ID NO: 61. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 61.

In some embodiments, the CD19 binding chimeric antigen receptor comprises the amino acid sequence according to any one of SEQ ID NO: 62. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 62.

In some embodiments, the CD19 binding chimeric antigen receptor comprises the amino acid sequence according to SEQ ID NO: 63. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 63.

In some embodiments, the CD19 binding chimeric antigen receptor comprises the amino acid sequence according to SEQ ID NO: 64. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 64.

In some embodiments, the CD19 binding chimeric antigen receptor comprises the amino acid sequence according to SEQ ID NO: 65. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 65.

In some embodiments, the CD19 binding chimeric antigen receptor comprises the amino acid sequence according to SEQ ID NO: 66. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 66.

In some embodiments, the CD19 binding chimeric antigen receptor comprises the amino acid sequence according to SEQ ID NO: 67. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 67.

In some embodiments, the CD19 binding chimeric antigen receptor comprises the amino acid sequence according to SEQ ID NO: 68. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 68.

In some embodiments, the CD19 binding chimeric antigen receptor comprises the amino acid sequence according to SEQ ID NO: 69. In some embodiments, the CD19 binding chimeric antigen receptor comprises the amino acid sequence having at least 50%, 55%, 60%, 65%, 70%, 75%, 80%, 85%, 90%, 95%, 97% or 99% identity to SEQ ID NO: 69.

TABLE 7
Amino acid sequences related to
CD19 CAR based RTCR.

CD19 binding extracellular domain, FMC63scFV
(Other name: CD19) (SEQ ID NO: 51):
CD8a leader/signal peptide (bold,
SEQ ID NO: 117) and CD8a Hinge
(underlined, SEQ ID NO: 118)
[FMC63 scFV (CD8a Leader_Light Chain_
Linker_Heavy Chain_CD8a Hinge)]
MALPVTALLLPLALLLHAARPDIQMTQTTSSLSAS
LGDRVTISCRASQDISKYLNAVYQQKPDGTVKLLI
YHTSRLHSGVPSRFSGSGSGTDYSLTISNLEQEDI
ATYFCQQGNTLPYTFGGGTKLEITGGGGSGGGGSG
GGGSEVKLQESGPGLVAPSQSLSVTCTVSGVSLPD
YGVSWIRQPPRKGLEWLGVIWGSETTYYNSALKSR
LTIIKDNSKSQVFLKMNSLQTDDTAIYYCAKHYYY
GGSYAMDYWGQGTSVTVSSTTTPAPRPPTPAPTIA
SQPLSLRPEACRPAAGGAVHTRGLDFAC
CD19 (FMC63 scFV) CD8a Transmembrane 4-1BB
Signaling_CD3Z chimeric antigen receptor
(CAR) (Other names: FMC63scFV_BB_Z;
CD19_BB_Z; CD19_BBwt_Z; CD19_CD137_Z;
CD19-BBZ; or CD19:BBZ) (SEQ ID NO: 52):
CD19 binding extracellular
domain (underlined)-CD137 intracellular
domain-CD3ζ signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACDIYIWAPL
AGTCGVLLLSLVITLYCKRGRKKLLYIFKQPFMRP
VQTTQEEDGCSCRFPEEEEGGCELRVKFSRSADAP
AYQQGQNQLYNELNLGRREEYDVLDKRRGRDPEMG
GKPQRRKNPQEGLYNELQKDKMAEAYSEIGMKGER
RRGKGHDGLYQGLSTATKDTYDALHMQALPPR
CD19 (FMC63 scFV)_CD28 Transmembrane
CD28 Signaling_CD3Z CAR (Other names:
FMC63scFV_28_Z; CD19_28_Z; CD19_CD28_Z;
CD19 28Z or CD19-28Z or CD19:28Z)
(SEQ ID NO: 53):
CD19 binding extracellular domain
(underlined)-CD28 DS-CD3ζ signaling
domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACLFPGPSKP
FWVLVVVGGVLACYSLLVTVAFIIFWVRSKRSRLL
HSDYMNMTPRRPGPTRKHYQPYAPPRDFAAYRSRV
KFSRSADAPAYQQGQNQLYNELNLGRREEYDVLDK
RRGRDPEMGGKPQRRKNPQEGLYNELQKDKMAEAY
SEIGMKGERRRGKGHDGLYQGLSTATKDTYDALHM
QALPPR
CD19 (FMC63 scFV)_CD28 Transmembrane_
CD28 Signaling 4-1BB Signaling_CD3Z CAR
(Other names: FMC63scFV_28_BBwt_Z;
CD19_CD28_BB_Z; CD19_28_BBwt_Z; CD19-
28BBwt_Z; CD19-28BBZ; or CD19:28BBZ)
(SEQ ID NO: 54):
[CD19 binding extracellular
domain (underlined)-CD28 DS-CD137
intracellular domain-CD3t signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACLFPGPSKP
FWVLVVVGGVLACYSLLVTVAFIIFWVRSKRSRLL
HSDYMNMTPRRPGPTRKHYQPYAPPRDFAAYRSKR
GRKKLLYIFKQPFMRPVQTTQEEDGCSCRFPEEEE
GGCELRVKFSRSADAPAYQQGQNQLYNELNLGRRE
EYDVLDKRRGRDPEMGGKPQRRKNPQEGLYNELQK
DKMAEAYSEIGMKGERRRGKGHDGLYQGLSTATKD
TYDALHMQALPPR
CD19 (FMC63 scFV)_CD28 Transmembrane
CD28 Signaling 4-1BB truncated
Signaling_CD3Z CAR (Other names:
FMC63scFV_28_BBt_Z; CD19_CD28_BBt_Z;
CD19_28_BBt_Z; CD19-28BBt_Z; CD19-28BBtZ
or CD19:28BBtZ) (SEQ ID NO: 55): []
CD19 binding extracellular domain
(underlined)-CD28 DS-truncated CD137
intracellular domain-CD3
signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACLFPGPSKP
FWVLVVVGGVLACYSLLVTVAFIIFWVRSKRSRLL
HSDYMNMTPRRPGPTRKHYQPYAPPRDFAAYRSQP
FMRPVQTTQEEDGCSCRFPEEEEGGCELRVKFSRS
ADAPAYQQGQNQLYNELNLGRREEYDVLDKRRGRD
PEMGGKPQRRKNPQEGLYNELQKDKMAEAYSEIGM
KGERRRGKGHDGLYQGLSTATKDTYDALHMQALPP
R
CD19 (FMC63 scFV)_CD28 Transmembrane CD28
Signaling OX-40 Truncated Signaling_CD3Z
CAR (Other names: FMC63scFV_28_OX40t_Z;
CD19_CD28_OX40t_Z; CD19_28_OX40t_Z;
CD19_28_OX40tZ; CD19_28_40t_Z;
CD19_28_40tZ; CD19-2840tZ or
CD19:2840tZ) (SEQ ID NO: 56):
CD19 binding extracellular domain
(underlined)-CD28 DS-
truncated CD134 intracellular
domain-CD3ζ signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACLFPGPSKP
FWVLVVVGGVLACYSLLVTVAFIIFWVRSKRSRLL
HSDYMNMTPRRPGPTRKHYQPYAPPRDFAAYRSGG
GSFRTPIQEEQADAHSTLARVKFSRSADAPAYQQG
QNQLYNELNLGRREEYDVLDKRRGRDPEMGGKPQR
RKNPQEGLYNELQKDKMAEAYSEIGMKGERRRGKG
HDGLYQGLSTATKDTYDALHMQALPPR
CD19 (FMC63 scFV) ICOS Transmembrane
ICOS Signaling_CD3Z CAR (Other names:
FMC63scFV_ICOS_Z; CD19_ICOS_Z;
CD19_ICOSZ; CD19-ICOSZ or CD19:ICOSZ)
(SEQ ID NO: 57): [(CD19 binding
extracellular domain (underlined)-
ICOS DS-CD3ζ signaling domain)
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLRVKFSRSADAP
AYQQGQNQLYNELNLGRREEYDVLDKRRGRDPEMG
GKPQRRKNPQEGLYNELQKDKMAEAYSEIGMKGER
RRGKGHDGLYQGLSTATKDTYDALHMQALPPR
CD19 (FMC63 scFV) ICOS Transmembrane
ICOS Signaling 4-1BB Signaling_CD3Z
CAR (Other names:
FMC63scFV_ICOS_BBwt_Z;
CD19_ICOS_BB_Z; CD19_ICOS_BBwt_Z;
CD19-ICOSBBwt_Z; CD19-ICOSBBZ or
CD19:ICOSBBZ) (SEQ ID NO: 58): []
CD19 binding extracellular domain
(underlined)-ICOS DS-CD137
intracellular domain-
CD3ζ signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLKRGRKKLLYIF
KQPFMRPVQTTQEEDGCSCRFPEEEEGGCELRVKF
SRSADAPAYQQGQNQLYNELNLGRREEYDVLDKRR
GRDPEMGGKPQRRKNPQEGLYNELQKDKMAEAYSE
IGMKGERRRGKGHDGLYQGLSTATKDTYDALHMQA
LPPR
CD19 (FMC 63 scFV)_ICOS Transmembrane
ICOS Signaling 4-1BB Truncated
Signaling_CD3Z CAR (Other names:
FMC63scFV_ICOS_BBt_Z; CD19_ICOS_BBt_Z;
CD19-ICOSBBtZ or CD19:ICOSBBtZ)
(SEQ ID NO: 59): []
CD19 binding extracellular
domain (underlined)-ICOS DS-truncated
CD137 intracellular domain-CD3ζ
signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELRVKFSRSADAPAYQQG
QNQLYNELNLGRREEYDVLDKRRGRDPEMGGKPQR
RKNPQEGLYNELQKDKMAEAYSEIGMKGERRRGKG
HDGLYQGLSTATKDTYDALHMQALPPR
CD19 (FMC 63 scFV) ICOS Transmembrane
ICOS Signaling OX-40 Truncated
Signaling_CD3Z CAR (Other names:
FMC63scFV_ICOS_OX40t_Z;
CD19_ICOS_OX40t_Z;
CD19_ICOS_40t_Z; CD19_ICOS_OX40tZ;
CD19_ICOS_40tZ; CD19-ICOS4OtZ or
CD19:ICOS4OtZ) (SEQ ID NO: 60):
CD19 binding extracellular domain
(underlined)-ICOS
DS-truncated CD134 intracellular
domain-CD3ζ signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLGGGSFRTPIQE
EQADAHSTLARVKFSRSADAPAYQQGQNQLYNELN
LGRREEYDVLDKRRGRDPEMGGKPQRRKNPQEGLY
NELQKDKMAEAYSEIGMKGERRRGKGHDGLYQGLS
TATKDTYDALHMQALPPR
CD19 (FMC63 scFV)_ICOS_Transmembrane_
ICOS Signaling (mini CD28 Signaling)_CD3Z
CAR (Other names: FMC63scFV_ICOS(28)_Z;
CD19_ICOS(28)_Z; CD19-ICOS(28)_Z;
CD19-ICOS(28)Z or CD19:ICOS(28)Z)
(SEQ ID NO: 61): []
CD19 binding extracellular
domain (underlined)-ICOS DS-CD28(PRRP)
-CD3ζ signaling domain)
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMTPRRPGPTRKHYQPYAPPRAVNTAKKS
RLTDVTLRVKFSRSADAPAYQQGQNQLYNELNLGR
REEYDVLDKRRGRDPEMGGKPQRRKNPQEGLYNEL
QKDKMAEAYSEIGMKGERRRGKGHDGLYQGLSTAT
KDTYDALHMQALPPR
CD19 (FMC63 scFV) ICOS Transmembrane
ICOS Signaling (mini CD28 Signaling)
4-1BB Truncated Signaling_CD3Z CAR
(Other names: FMC63scFV_ICOS(28)_BBt_Z;
CD19_ICOS(28)_BBt_Z; CD19_ICOS(28)_BBtZ;
CD19-ICOS(28)BBtZ or
CD19:ICOS(28)BBtZ) (SEQ ID NO: 62):
(CD19 binding extracellular domain
(underlined)-ICOS DS-CD28(PRRP)-
truncated CD137 intracellular
domain-CD3ζ signaling domain)
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMTPRRPGPTRKHYQPYAPPRAVNTAKKS
RLTDVTLQPFMRPVQTTQEEDGCSCRFPEEEEGGC
ELRVKFSRSADAPAYQQGQNQLYNELNLGRREEYD
VLDKRRGRDPEMGGKPQRRKNPQEGLYNELQKDKM
AEAYSEIGMKGERRRGKGHDGLYQGLSTATKDTYD
ALHMQALPPR
CD19 (FMC 63 scFV)_ICOS_Transmembrane_
ICOS Signaling (mini CD28 Signaling)_
OX-40 Truncated Signaling_CD3Z CAR
(Other names: FMC63scFV_ICOS(28)_OX40t_Z
CD19_ICOS(28)_OX40t_Z; CD19_ICOS(28)_OX40tZ;
CD19_IC05(28)_40t_Z; CD19_ICOS(28)_40tZ;
CD19-ICOS(28)40tZ or CD19:ICOS(28)40tZ)
(SEQ ID NO: 63):
CD19 binding extracellular domain
(underlined)-ICOS DS-CD28(PRRP)-truncated
CD134 intracellular domain-CD3ζ
signaling domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMTPRRPGPTRKHYQPYAPPRAVNTAKKS
RLTDVTLGGGSFRTPIQEEQADAHSTLARVKFSRS
ADAPAYQQGQNQLYNELNLGRREEYDVLDKRRGRD
PEMGGKPQRRKNPQEGLYNELQKDKMAEAYSEIGM
KGERRRGKGHDGLYQGLSTATKDTYDALHMQALPP
R
CD19_ICOSBBt_Zt CAR (CD19 binding
extracellular domain-ICOS DS-
truncated CD137 intracellular
domain-CD3Ztruncated domain)
CAR (Other names:
FMC63scFV_ICOS_BBt_Zt;
CD19_ICOS_BBt_Zt; CD19-ICOSBBtZt
or CD19:ICOSBBtZt) (SEQ ID NO: 64):
CD19 binding extracellular domain
(underlined)-ICOS DS-truncated
CD137 intracellular domain-CD3ζ
truncated domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELRVKFSRSADAPAYQQG
QNQLYNELNLGRREEYDVLDKRRGRDPEMGGK
CD19_ICOSBBt_ZE_CAR (CD19 binding
extracellular domain-ICOS DS-
truncated CD137 intracellular
domain-CD3ZE domain) (Other
names: FMC63scFV_ICOS_BBt_ZE; CD19
CD19-ICOSBBtZE ICOS_BBt_ZE; or
CD19:ICOSBBtZE) (SEQ ID NO: 65):
CD19 binding extracellular domain
(underlined)-ICOS DS-truncated
CD137 intracellular domain-
CD3ζ domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELRVKFSRSADAPAYQQG
QNQLYNELNLGRREEYDVLDKRRGRDPEMGGKPVT
RGAGAGGRQRGQNKERPPPVPNPDYEPIRKGQRDL
YSGLNQRRI
CD19_ICOSBBt:CD2t_Z CAR (CD19 binding
extracellular domain-ICOS DS-truncated
CD137 intracellular domain-CD2 truncated
Signaling Domain-CD3 domain) CAR (Other
names: FMC63scFV_ICOS_BBt_CD2tZ; CD19_
ICOS_BBt_CD2t_Z; CD19_ICOS_BBt CD2tZ;
CD19_ICOS_BBtCD2tZ; CD19-ICOSBBtCD2tZ or
CD19:ICOSBBtCD2tZ) (SEQ ID NO: 66):
CD19 binding extracellular domain
(underlined)-ICOS DS-truncated CD137
intracellular domain-CD2 truncated
Signaling Domain-CD3ζ domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELQNPATSQUPPPPPGHR
SQAPSHRPPPPGHRVQHQPQKRPPAPSGTQVHQQK
GPPLPRPRVQPKPPHGAAENSLSPSSNRVKFSRSA
DAPAYQQGQNQLYNELNLGRREEYDVLDKRRGRDP
EMGGKPQRRKNPQEGLYNELQKDKMAEAYSEIGMK
GERRRGKGHDGLYQGLSTATKDTYDALHMQALPPR
CD19_ICOSBBt:CD2t ZE CAR (CD19 binding
extracellular domain-ICOS DS-truncated
CD137 intracellular domain-CD2 truncated
Signaling Domain-CD3ZE domain) (Other
names: FMC63scFV_ICOS_BBt_CD2tZE CAR CD19_
ICOS_BBt_CD2tZE; CD19_ICOS_BBt_CD2t_ZE;
CD19_ICOS_BBt_2tZE; CD19_ICOS_BBt_2t_ZE;
CD19-ICOSBBtCD2tZE; CD19-ICOSBBt2tZE;
CD19:ICOSBBtCD2tZE or CD19:ICOSBBt2tZE)
(SEQ ID NO: 67):
CD19 binding extracellular domain
(underlined)-ICOS DS-truncated
CD137 intracellular domain-CD2
truncated Signaling Domain-CD3ζϵ domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELQNPATSQUPPPPPGHR
SQAPSHRPPPPGHRVQHQPQKRPPAPSGTQVHQQK
GPPLPRPRVQPKPPHGAAENSLSPSSNRVKFSRSA
DAPAYQQGQNQLYNELNLGRREEYDVLDKRRGRDP
EMGGKPVTRGAGAGGRQRGQNKERPPPVPNPDYEP
IRKGQRDLYSGLNYRHQRRI
CD19_ICOSBBt:IL2RB(YLRQ)_Z CAR (CD19
binding extracellular domain-ICOS DS-
truncated CD137 intracellular domain-
IL2RB(YLRQ) domain-CD3Z domain)
(Other names: FMC63scFV_ICOS_BBt_IL2RB
(YLRQ)_Z_CAR_CD19_ICOS_BBt_IL2RB(YLRQ)Z;
CD19_ICOS_BBt_IL2RB(YLRQ)_Z; CD19_ICOS_
BBtIL2RB(YLRQ)Z; CD19-ICOSBBtIL2RB(YLRQ)Z
or CD191COSBBtIL2RB(YLRQ)Z)
(SEQ ID NO: 68): CD19
binding extracellular domain
underlined)-ICOS DS-truncated CD137
intracellular domain-IL2RB(YLRQ)
domain-CD3ζ domain)
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELNCRNTGPWLKKVLKCN
TPDPSKFFSQLSSEHGGDVQKWLSSPFPSSSFSPG
GLAPEISPLEVLERDKVTQLLPLNTDAYLSLQELQ
GQDPTHLVRVKFSRSADAPAYQQGQNQLYNELNLG
RREEYDVLDKRRGRDPEMGGKPQRRKNPQEGLYNE
LQKDKMAEAYSEIGMKGERRRGKGHDGLYQGLSTA
TKDTYDAYRHQALPPR
CD19_ICOSBBt:IL2RB(YLRQ)_ZE CAR (CD19
binding extracellular domain-ICOS DS-
truncated CD137 intracellular domain-
IL2RB(YLRQ) domain-CD3ZE domain) CAR
(Other names: FMC63scFV_ICOS_BBt
IL2RB(YLRQ)_ZE_CD19_COS_BBt_IL2RB
(YLRQ)_ZE; CD19_ICOS_BBtIL2RB(YLRQ)ZE;
CD19-ICOSBBtIL2RB(YLRQ)ZE or
CD191COSBBtIL2RB(YLRQ)ZE)
(SEQ ID NO: 69): CD19 binding
extracellular domain
(underlined)-ICOS DS-truncated CD137
intracellular domain-IL2RB(YLRQ)
domain-CD3ζϵ domain
MALPVTALLLPLALLLHAARPDIQMTQTTSSLSAS
LGDRVTISCRASQDISKYLNWYQQKPDGTVKLLIY
HTSRLHSGVPSRFSGSGSGTDYSLTISNLEQEDIA
TYFCQQGNTLPYTFGGGTKLEITGGGGSGGGGSGG
GGSEVKLQESGPGLVAPSQSLSVTCTVSGVSLPDY
GVSWIRQPPRKGLEWLGVIWGSETTYYNSALKSRL
TIIKDNSKSQVFLKMNSLQTDDTAIYYCAKHYYYG
GSYAMDYWGQGTSVTVSSTTTPAPRPPTPAPTIAS
QPLSLRPEACRPAAGGAVHTRGLDFACSQLCCQLK
FWLPIGCAAFVVVCILGCILICWLTKKKYSSSVHD
PNGEYMFMRAVNTAKKSRLTDVTLQPFMRPVQTTQ
EEDGCSCRFPEEEEGGCELNCRNTGPWLKKVLKCN
TPDPSKFFSQLSSEHGGDVQKWLSSPFPSSSFSPG
GLAPEISPLEVLERDKVTQLLPLNTDAYLSLQELQ
GQDPTHLVRVKFSRSADAPAYQQGQNQLYNELNLG
RREEYDVLDKRRGRDPEMGGKPVTRGAGAGGRQRG
QNKERPPPVPNPDYEPIRKGQRDLYSGLNYRHQRR
I

In some embodiments, the extracellular domain comprises a hinge region. In some embodiments, the hinge region is derived from CD8, PD-1, CD28, ICOS, or IgG. In some embodiments, the transmembrane domain of the RTCR disclosed herein, is derived from CD8, PD1, CD28, ICOS, or IgG.

The present disclosure also provides a nucleic acid encoding the RTCR disclosed herein. In some embodiments, the nucleic acid encoding the RTCR disclosed herein is according to SEQ ID NO: 75-86 and 92-110. In some embodiments, the nucleic acid disclosed herein comprises a nucleic acid sequence encoding a chimeric intracellular domain. In some embodiments, the RTCR disclosed herein is for expression in a T cell, wherein the T cell co-expresses at least one of the endogenous co-stimulatory molecules CD28, CD2, OX-40, ICOS, CD28, CD3, CD4, CD8 and CD40L or a combination thereof.

The present disclosure also provides a vector comprising the nucleic acid disclosed herein. In some embodiments, the vector disclosed herein is any one of a viral vector, a plasmid, a cosmid, a yeast artificial chromosome, a bacterial artificial chromosome or a transposon/transposase system. In some embodiments, the viral vector is an adeno-viral vector or a lentiviral vector. In some embodiments, the vector is a lentiviral vector.

The present disclosure also provides a cell comprising the nucleic acid or the vector disclosed herein. In some embodiments, the cell disclosed herein is a modified T cell. In some embodiments, the modified T cell is an allogenic T cell. In some embodiments, the modified T cell is an autologous T cell. In some embodiments, the modified T cell is any one of a naïve T cell, an early memory T cell, a stem cell-like T cell, a stem memory T cell (T_{SCM}), a central memory T cell (T_{CM}) and a regulatory T cell (T_{reg}).

In some embodiments, the extracellular domain is a B cell maturation Ag (BCMA) binding protein. In some embodiments, the BCMA binding protein is a BCMA specific T cell receptor (TCR). In some embodiments, the BCMA binding protein is a BCMA specific chimeric antigen receptor (CAR). In some embodiments, the BCMA binding chimeric antigen receptor comprises the amino acid sequence according to any one of: SEQ ID NOs: 137-146.

In some embodiments, the BCMA binding chimeric antigen receptor comprises the amino acid sequence according to SEQ ID NO: 137. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 137.

In some embodiments, the BCMA binding chimeric antigen receptor comprises the amino acid sequence according to SEQ ID NO: 138. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 138.

In some embodiments, the BCMA binding chimeric antigen receptor comprises the amino acid sequence according to SEQ ID NO: 139. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 139.

In some embodiments, the BCMA binding chimeric antigen receptor comprises the amino acid sequence according to SEQ ID NO: 140. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 140.

In some embodiments, the BCMA binding chimeric antigen receptor comprises the amino acid sequence according to SEQ ID NO: 141. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 141.

In some embodiments, the BCMA binding chimeric antigen receptor comprises the amino acid sequence according to SEQ ID NO: 142. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 142.

In some embodiments, the BCMA binding chimeric antigen receptor comprises the amino acid sequence according to SEQ ID NO: 143. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 143.

In some embodiments, the BCMA binding chimeric antigen receptor comprises the amino acid sequence according to SEQ ID NO: 144. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 144.

In some embodiments, the BCMA binding chimeric antigen receptor comprises the amino acid sequence according to SEQ ID NO: 145. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 145.

In some embodiments, the BCMA binding chimeric antigen receptor comprises the amino acid sequence according to SEQ ID NO: 146. In some embodiments, the BCMA binding chimeric antigen receptor comprises the amino acid sequence having at least 50%, 55%, 60%, 65%, 70%, 75%, 80%, 85%, 90%, 95%, 97% or 99% identity to SEQ ID NO: 146.

In some embodiments, the extracellular domain is a B cell maturation Ag (BCMA) binding protein. In some embodiments, the BCMA binding protein is a BCMA specific T cell receptor (TCR). In some embodiments, the BCMA binding protein is a BCMA specific chimeric antigen receptor (CAR). In some embodiments, the BCMA binding chimeric antigen receptor comprises the amino acid sequence according to any one of: SEQ ID NOs: 141, 142, 145 and 146.

TABLE 8
Amino acid sequences of BCMA specific chimeric antigen
receptors (CAR) and BCMA specific CAR-based RTCR.

11-D5-3 scFv (CD8a Signal Peptide_11-D5-3 scFv, mouse_CD8a_
Hinge_BB_Z) (SEQ ID NO: 133):
CD8a Signal Peptide (bold, SEQ ID NO: 117)
11-D5-3 scFv, mouse (italics)_CD8a Hinge (bold,
SEQ ID NO: 118)_BB_Z)
MALPVTALLLPLALLLHAARPDIVLTQSPPSLAMSLGKRATISCRASESVTILGSH
LIHWYQQKPGQPPTLLIQLASNVQTGVPARFSGSGSRTDFTLTIDPVEEDDVAVYYC
LQSRTIPRTFGGGTKLEIKGSTSGSGKPGSGEGSTKGQIQLVQSGPELKKPGETVKI
SCKASGYTFTDYSINWVKRAPGKGLKWMGWINTETREPAYAYDFRGRFAFSLETSAS
TAYLQINNLKYEDTATYFCALDYSYAMDYWGQGTSVTVSSTTTPAPRPPTPAPTIAS
QPLSLRPEACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAYQQGQNQL
YNELNLGRREEYDVLDKRRGRDPEMGGKPQRRKNPQEGLYNELQKDKMAEAYSEIGM
KGERRRGKGHDGLYQGLSTATKDTYDALHMQALPPR
FHVH33 HVV (CD8a Signal Peptide_FHVH33 HVV_CD8a Hinge_BB_Z)
(SEQ ID NO: 134):
(CD8a Signal Peptide (bold, SEQ ID NO: 117)_FHVH33 HVV
(italicized)_CD8a Hinge
(bold, SEQ ID NO: 118)_BB_Z)
MALPVTALLLPLALLLHAARPEVQLLESGGGLVQPGGSLRLSCAASGFTFSSYAMSWVR
QAPGKGLEWVSSISGSGDYIYYADSVKGRFTISRDISKNTLYLQMNSLRAEDTAVYYCA
KEGTGANSSLADYRGQGTLVTVSSFVPVFLPAKPTTTPAPRPPTPAPTIASQPLSLRPE
ACRPAAGGAVHTRGLDFACDIYIWAPLAGTCGVLLLSLVITLYCNHRNKRGRKKLLYIF
KQPFMRPVQTTQEEDGCSCRFPEEEEGGCELRVKFSRSADAPAYQQGQNQLYNELNLGR
REEYDVLDKRRGRDPEMGGKPQRRKNPQEGLYNELQKDKMAEAYSEIGMKGERRRG
KGHDGLYQGLSTATKDTYDALHMQALPPR
BCAR003 HVV (CD8a Signal Peptide BCAR003 HVV_CD8a Hinge_BB_Z)
(SEQ ID NO: 135): CD8a Signal Peptide (Bold)_BCAR003 HVV
(italicized)_CD8a Hinge (Bold)_BB_Z
MALPVTALLLPLALLLHAARPQVKLEESGGGLVQAGRSLRLSCAASEHTFSSHVMGWF
RQAPGKERESVAVIGWRDISTSYADSVKGRFTISRDNAKKTLYLQMNSLKPEDTAVYY
CAARRIDAADFDSWGQGTQVTVSSGGGGSGGGGSGGGGSGGGGSGGGGSAVQLVESGG
GLVQAGDSLRLTCTASGRAFSTYFMAWFRQAPGKEREFVAGIAWSGGSTAYADSVKGR
FTISRDNAKNTVYLQMNSLKSEDTAVYYCASRGIEVEEFGAWGQGTQVTVSSTSTTTP
APRPPTPAPTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELRVKFSRSAD
APAYQQGQNQLYNELNLGRREEYDVLDKRRGRDPEMGGKPQRRKNPQEGLYNELQKDK
MAEAYSEIGMKGERRRGKGHDGLYQGLSTATKDTYDALHMQALPPR
GSI5022 HVV (CD8a Signal Peptide_G515022 HVV_CD8a Hinge_BB_Z)
(SEQ ID NO: 136): CD8a Signal Peptide (bold, SEQ ID NO: 117)
G5I5022 HVV (italicized)_CD8a Hinge
(bold, SEQ ID NO: 118)_BB_Z
MALPVTALLLPLALLLHAARPQVKLEESGGGLVQAGRSLRLSCAASEHTFSSHVMGWF
RQAPGKERESVAVIGWRDISTSYADSVKGRFTISRDNAKKTLYLQMNSLKPEDTAVYY
CAARRIDAADFDSWGQGTQVTVSSGGGGSGGGGSGGGGSEVQLVESGGGLVQAGGSLR
LSCAASGRTFTMGWFRQAPGKEREFVAAISLSPTLAYYAESVKGRFTISRDNAKNTVV
LQMNSLKPEDTALYYCAADRKSVMSIRPDYWGQGTQVTVSSTSTTTPAPRPPTPAPTI
ASQPLSLRPEACRPAAGGAVHTRGLDFACDIYIWAPLAGTCGVLLLSLVITLYCKRGR
KKLLYIFKQPFMRPVQTTQEEDGCSCRFPEEEEGGCELRVKFSRSADAPAYQQGQNQL
YNELNLGRREEYDVLDKRRGRDPEMGGKPQRRKNPQEGLYNELQKDKMAEAYSEIGMK
GERRRGKGHDGLYQGLSTATKDTYDALHMQALPPR
BCMAsdAb1 HVV (CD8a Signal Peptide anti-BCMAsdAB1 HHV_CD8a
Hinge) (SEQ ID NO: 137): CD8a Signal Peptide (bold,
SEQ ID NO: 117)_anti-BCMAsdAB1 HHV (italicized)_
CD8a Hinge (bold, SEQ ID NO: 118)
MALPVTALLLPLALLLHAARPEVQLQASGGGLAQPGGSLRLSCAASGRTFSTYFMAWF
RQPPGKGLEYVGGIRWSDGVPHYADSVKGRFTISRDNAKNTVYLQMNSLRAEDTAVYF
CASRGIADGSDFGSYGQGTQVTVSSTTTPAPRPPTPAPTIASQPLSLRPEACRPAAGG
AVHTRGLDFAC
BCMAsdAb1 HVV CD8a Transmembrane 4-1BB Signaling_CD3Z
(Other names: BCMAsdAb1_BB_Z or BCMAsdAb1-BBZ)
(SEQ ID NO: 138): BCMAsdAb1 HVV (bold, SEQ ID NO: 137)_
CD8a_Transmembrane_4-1BB Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPAYQQGQN
QLYNELNLGRREEYDVLDKRRGRDPEMGGKPQRRKNPQEGLYNELQKDKMAEAY
SEIGMKGERRRGKGHDGLYQGLSTATKDTYDALHMQALPPR
BCMAsdAb1 HVV_CD28 Transmembrane_CD28 Signaling_CD3Z
(Other name: BCMAsdAb1_28_Z; BCMAsdAb1-28Z)
(SEQ ID NO: 139): BCMAsdAb1 HVV (bold, SEQ ID NO: 137)
CD28 Transmembrane_CD28 Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACLFPGPSKPFWVLVVVGGVLACYSLLVTVAFI
IFWVRSKRSRLLHSDYMNMTPRRPGPTRKHYQPYAPPRDFAAYRSRVKFSRSAD
GAPAYQQQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPR
BCMAsdAb1 HVV_CD28 Transmembrane CD28 Signaling 4-1BB
Signaling_CD3Z (Other 28 BBZ.
names: BCMAsdAb1_28_BBwt_Z; BCMAsdAb1, 28_BB_Z_BCMAsdAb1,
BCMAsdAb1-28_BBz; or BCMAsdAb1-28BBZ)
(SEQ ID NO: 140): BCMAsdAb1 HVV (bold, SEQ ID NO: 137)_
CD28 Transmembrane_CD28 Signaling 4-1BB Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACLFPGPSKPFWVLVVVGGVLACYSLLVTVAFI
IFWLVRSKRSRLLHSDYMNMTPRRPGPTRKHYQPYAPPRDFAAYRSKRGRKKLL
YIFKQPFMRPVQTTQEEDGCSCRFPEEEEGGCELRVKFSRSADAPAYQQGQNQL
YNELNLGRREEYDVLDKRRGRDPEMGGKPQRRKNPQEGLYNELQKDKMAEAYSE
IGMKGERRRGKGHDGLYQGLSTATKDTYDALHMQALPPR
BCMAsdAb1 HVV_CD28 Transmembrane CD28 Signaling
4-1BB truncated Signaling_CD3Z (Other names:
BCMAsdAb1_28_BBt_Z; BCMAsdAb1_28_BBtZ
BCMAsdAb1-28 BBtZ; or BCMAsdAb1-28BBtZ)
(SEQ ID NO: 141): BCMAsdAb1 HVV (bold, SEQ ID NO: 137)_
CD28 Transmembrane_CD28 Signaling_4-1BB truncated
Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACLFPGPSKPFWVLVVVGGVLACYSLLVTVAFI
IFWVRSKRSRLLHSDYMNMTPRRPGPTRKHYQPYAPPRDFAAYRSQPFMRPVQT
TQEEDGCSCRFPEEEEGGCELRVKFSRSADAPAYQQGQNQLYNELNLGRREEYD
VLDKRRGRDPEMGGKPQRRKNPQEGLYNELQKDKMAEAYSEIGMKGERRRGKGH
DGLYQGLSTATKDTYDALHMQALPPR
BCMAsdAb1 HVV_CD28 Transmembrane CD28 Signaling
OX-40 Truncated Signaling_CD3Z (Other names: BCMAsdAb1
28_OX40t_Z; BCMAsdAb1_28_OX40tZ;
BCMAsdAb1_28_40t_Z; BCMAsdAb1_28_40tZ BCMAsdAb1-28
40tZ; or BCMAsdAb1-2840tZ) (SEQ ID NO: 142):
BCMAsdAb1 HVV (bold, SEQ ID NO: 137)_CD28
Transmembrane_CD28 Signaling_OX-40 Truncated
Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACLFPGPSKPFWVLVVVGGVLACYSLLVTVAFI
IFWVRSKRSRLLHSDYMNMTPRRPGPTRKHYQPYAPPRDFAAYRSGGGSFRTPI
QEEQADAHSTLARVKFSRSADAPAYQQGQNQLYNELNLGRREEYDVLDKRRGRD
PEMGGKPQRRKNPQEGLYNELQKDKMAEAYSEIGMKGERRRGKGHDGLYQGLST
ATKDTYDALHMQALPPR
BCMAsdAb1 HVV_ICOS Transmembrane_ICOS Signaling_
CD3Z (Other names: BCMAsdAb1_ICOS_Z; BCMAsdAb1-ICOSZ;
or BCMAsdAb1-ICOSZ) (SEQ ID NO: 143): BCMAsdAb1 HVV (bold,
SEQ ID NO: 137)_ICOS Transmembrane_ICOS Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACSQLCCQLKFWLPIGCAAFVVVCILGCILICW
LTKKKYSSSVHDPNGEYMFMRAVNTAKKSRLTDVTLRVKFSRSADAPAYQQGQN
QLYNELNLGRREEYDVLDKRRGRDPEMGGKPQRRKNPQEGLYNELQKDKMAEAY
SEIGMKGERRRGKGHDGLYQGLSTATKDTYDALHMQALPPR
BCMAsdAb1 HVV_ICOS Transmembrane_ICOS Signaling 4-1BB
Signaling_CD3Z (Other names: BCMAsdAb1 ICOS_BBwt_Z;
BCMAsdAb1_ICOS_BB_Z; BCMAsdAb1_ICOS_BBZ; BCMAsdAb1-28_BBZ;
or BCMAsdAb1-ICOSBBZ) (SEQ ID NO: 144): BCMAsdAb 1 HVV
(bold, SEQ ID NO: 137)_ICOS Transmembrane_ICOS Signaling_
4-1BB Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACSQLCCQLKFWLPIGCAAFVVVCILGCILICW
LTKKKYSSSVHDPNGEYMFMRAVNTAKKSRLTDVTLKRGRKKLLYIFKQPFMRP
VQTTQEEDGCSCRFPEEEEGGCELRVKFSRSADAPAYQQGQNQLYNELNLGRRE
EYDVLDKRRGRDPEMGGKPQRRKNPQEGLYNELQKDKMAEAYSEIGMKGERRRG
KGHDGLYQGLSTATKDTYDALHMQALPPR
BCMAsdAb1 HVV_ICOS Transmembrane_ICOS Signaling 4-1BB
Truncated Signaling_CD3Z (Other names: BCMAsdAb1 ICOS_BBt_Z;
BCMAsdAb1 ICOS_BBtZ; BCMAsdAb 1-ICOS_BBtZ; or BCMAsdAb
1-ICOSBBtz) (SEQ ID NO: 145): BCMAsdAb1 HVV (bold,
SEQ ID NO: 137)_ICOS Transmembrane_ICOS Signaling
4-1BB Truncated Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACSQLCCQLKFWLPIGCAAFVVVCILGCILICW
LTKKKYSSSVHDPNGEYMFMRAVNTAKKSRLTDVTLQPFMRPVQTTQEEDGCSC
RFPEEEEGGCELRVKFSRSADAPAYQQGQNQLYNELNLGRREEYDVLDKRRGRD
PEMGGKPQRRKNPQEGLYNELQKDKMAEAYSEIGMKGERRRGKGHDGLYQGLST
ATKDTYDALHMQALPPR
BCMAsdAb1 HVV_ICOS Transmembrane_ICOS Signaling_OX-40
Truncated Signaling_CD3Z
(Other names: BCMAsdAb1_ICOS_OX40t_Z;
BCMAsdAb1_ICOS_OX40tZ; BCMAsdAb1_ICOS_40t_Z;
BCMAsdAb1_ICOS_40tZ; BCMAsdAb1-ICOS_40tZ; or
BCMAsdAb1_ICOS4OtZ) (SEQ ID NO: 146): BCMAsdAb1
HVV (bold, SEQ ID NO: 137)_Transmembrane_ICOS
Signaling_OX-40 Truncated Signaling_CD3Z
MALPVTALLLPLALLLHAARPEVQLQASGGGLAQPGGSLRLSCAASGRTFSTYF
MAWFRQPPGKGLEYVGGIRWSDGVPHYADSVKGRFTISRDNAKNTVYLQMNSLR
AEDTAVYFCASRGIADGSDFGSYGQGTQVTVSSTTTPAPRPPTPAPTIASQPLS
LRPEACRPAAGGAVHTRGLDFACSQLCCQLKFWLPIGCAAFVVVCILGCILICW
LTKKKYSSSVHDPNGEYMFMRAVNTAKKSRLTDVTLGGGSFRTPIQEEQADAHS
TLARVKFSRSADAPAYQQGQNQLYNELNLGRREEYDVLDKRRGRDPEMGGKPQR
RKNPQEGLYNELQKDKMAEAYSEIGMKGERRRGKGHDGLYQGLSTATKDTYDAL
HMQALPPR

TABLE 9
Nucleic acid sequences of intracellular signaling
domains, extracellular domains of RTCRs and RTCRs.

CD2 truncated Signaling Domain (SEQ ID NO: 70)
CAGAATCCTGCCACCTCTCAGCACCCTCCACCTCCACCTGGACAC
AGATCTCAGGCCCCATCTCACAGACCTCCACCACCTGGTCATCGG
GTGCAGCATCAGCCCCAGAAAAGACCTCCTGCTCCTAGCGGAACA
CAGGTGCACCAGCAAAAGGGACCTCCACTGCCTAGACCTAGAGTG
CAGCCTAAGCCTCCTCATGGCGCTGCCGAGAATAGCCTGTCTCCT
AGCAGCAAC
IL2RB(YLRQ) Signaling Domain (SEQ ID NO: 71)
AATTGCAGAAACACAGGCCCCTGGCTGAAGAAAGTGCTGAAGTGC
AACACCCCTGATCCGAGCAAGTTCTTTAGCCAGCTGAGCAGCGAG
CATGGCGGCGACGTTCAGAAATGGCTGTCTAGCCCATTTCCTAGC
AGCAGCTTCAGCCCTGGTGGACTGGCCCCTGAGATTAGCCCTCTG
GAAGTGCTGGAACGGGACAAAGTGACCCAGCTGCTGCCCCTGAAT
ACCGACGCTTACCTGAGCCTGCAAGAGCTGCAAGGACAGGACCCT
ACACACCTGGTGTCCTACCTGAGACAGTGGGTCGTGATCCCTCCA
CCTCTCTCTAGTCCTGGACCTCAGGCCTCT
PD1 (PD1 Signal Peptide_PD1 Extracellular PD1
Transmembrane_PD1 Intracellular) PD-1
(Other name: PD1-wt or PD1 (SEQ ID NO: 72)
ATGCAGATTCCTCAAGCTCCTTGGCCTGTCGTGTGGGCCGTTCTG
CAACTTGGATGGCGGCCTGGCTGGTTCCTGGACTCTCCTGACAGA
CCCTGGAATCCTCCAACATTCAGCCCCGCTCTGCTGGTGGTTACC
GAGGGCGATAATGCCACCTTCACCTGTAGCTTCAGCAACACCAGC
GAGAGCTTCGTGCTGAACTGGTACAGAATGAGCCCCAGCAACCAG
ACCGACAAGCTGGCCGCCTTTCCTGAGGATAGATCTCAGCCCGGC
CAGGACTGCCGGTTCAGAGTTACACAGCTGCCCAACGGCCGGGAC
TTCCACATGTCTGTCGTCCGGGCCAGAAGAAACGACAGCGGCACA
TATCTGTGCGGCGCCATTTCTCTGGCCCCTAAGGCTCAGATCAAA
GAGAGCCTGAGAGCCGAGCTGAGAGTGACAGAAAGACGGGCCGAA
GTGCCCACAGCTCACCCTTCACCTTCTCCAAGACCTGCCGGCCAG
TTCCAGACACTGGTCGTGGGAGTTGTTGGCGGACTGCTGGGATCT
CTGGTGCTGCTTGTTTGGGTGCTCGCCGTGATCTGTAGCAGAGCC
GCCAGAGGAACAATCGGCGCCAGAAGGACAGGCCAGCCTCTGAAA
GAGGATCCCTCTGCTGTCCCCGTGTTCAGCGTGGACTATGGCGAG
CTGGATTTCCAGTGGCGGGAAAAGACACCCGAGCCTCCAGTGCCT
TGTGTGCCTGAGCAGACAGAGTACGCCACCATCGTGTTCCCTAGC
GGCATGGGCACATCTAGCCCTGCCAGAAGAGGATCTGCCGACGGA
CCTAGATCTGCCCAGCCTCTCAGACCTGAGGATGGCCACTGTTCT
TGGCCTCTT
PD1 Extracellular (Other name: PD1)
(SEQ ID NO: 73)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCT
PD-1 (extracellular domain without signal
peptide)-CD28 domain swap (DS)
(SEQ ID NO: 74)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCTCTGTTCCCCGGACCTAGCAAGCCCTTTTGG
GTGCTCGTTGTTGTTGGCGGCGTGCTGGCCTGTTATAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCT
PD-1 (extracellular domain without signal
peptide)-CD28 DS-CD137 domain
(SEQ ID NO: 75)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCTCTGTTCCCCGGACCTAGCAAGCCCTTTTGG
GTGCTCGTTGTTGTTGGCGGCGTGCTGGCCTGTTATAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCCAAGCGGGGCAGAAAGAAGCTGCTGTAC
ATCTTCAAGCAGCCCTTCATGCGGCCCGTGCAGACCACACAAGAG
GAAGATGGCTGCTCCTGTCGGTTCCCCGAGGAAGAAGAAGGCGGT
TGCGAACTG
PD-1 (extracellular domain without signal
peptide)-CD28 DS-truncated CD137 domain
(SEQ ID NO: 76)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCTCTGTTCCCCGGACCTAGCAAGCCCTTTTGG
GTGCTCGTTGTTGTTGGCGGCGTGCTGGCCTGTTATAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCCCAGCCTTTCATGAGGCCTGTGCAGACC
ACACAAGAAGAGGACGGCTGCTCCTGTCGGTTCCCCGAGGAAGAG
GAAGGCGGTTGCGAACTT
PD-1 (extracellular domain without signal
peptide)-CD28 DS-truncated CD134 domain
(SEQ ID NO: 77)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCTCTGTTCCCCGGACCTAGCAAGCCCTTTTGG
GTGCTCGTTGTTGTTGGCGGCGTGCTGGCCTGTTATAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCTGGCGGCGGAAGCTTCAGAACCCCTATC
CAAGAGGAACAGGCCGACGCTCACTCTACACTGGCT
PD-1 (extracellular domain without signal
peptide)-ICOS DS (SEQ ID NO: 78)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACACTT
PD-1 (extracellular domain without signal
peptide)-ICOS DS-truncated CD137
(SEQ ID NO: 79)
TTTCTGGACAGCCCCGACAGACCCTGGAATCCTCCTACATTCAGCC
CCGCTCTGCTGGTGGTTACCGAGGGCGATAATGCCACCTTCACCTG
TAGCTTCAGCAACACCAGCGAGAGCTTCGTGCTGAACTGGTACAG
AATGAGCCCCAGCAACCAGACCGACAAGCTGGCCGCCTTTCCTGA
GGATAGATCTCAGCCCGGCCAGGACTGCCGGTTCAGAGTTACACA
GCTGCCCAACGGCCGGGACTTCCACATGTCTGTCGTTCGGGCCAG
AAGAAACGACAGCGGCACATATCTGTGCGGCGCCATTTCTCTGGC
CCCTAAGGCTCAGATCAAAGAGAGCCTGAGAGCCGAGCTGAGAGT
GACAGAAAGACGGGCCGAAGTGCCCACAGCTCACCCTTCACCTTC
TCCAAGACCTGCCAGCCAGCTGTGCTGCCAGCTGAAGTTTTGGCT
GCCTATCGGCTGTGCCGCCTTCGTGGTTGTGTGTATCCTGGGCTG
CATCCTGATCTGCTGGCTGACCAAGAAAAAGTACAGCAGCTCCGT
GCACGACCCCAACGGCGAGTACATGTTCATGAGAGCCGTGAACAC
CGCCAAGAAGTCCAGACTGACCGACGTGACCCTGAAGCGGGGCAG
AAAGAAACTGCTGTACATCTTCAAGCAGCCCTTCATGCGGCCCGT
GCAGACCACACAAGAGGAAGATGGCTGCTCCTGCAGATTCCCCGA
GGAAGAAGAAGGCGGCTGCGAACTT
PD-1 (extracellular domain without signal
peptide)-ICOS DS-truncated CD137
(SEQ ID NO: 80)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACACTTCAGCCTTTC
ATGAGGCCCGTGCAGACCACACAAGAAGAGGACGGCTGCTCCTGC
AGATTCCCCGAGGAAGAGGAAGGCGGTTGCGAACTT
PD-1 (extracellular domain without signal
peptide)-ICOS DS-truncated CD134
(SEQ ID NO: 81)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACACTTGGCGGCGGA
AGCTTTAGAACCCCTATCCAAGAGGAACAGGCCGACGCTCACTCT
ACACTGGCT
PD1 (extracellular domain without signal
peptide)-ICOS DS-CD28(PRRP)-truncated CD137)
(SEQ ID NO: 82)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGACCCCTAGAAGG
CCTGGACCTACCAGAAAGCACTACCAGCCTTACGCTCCTCCTAGA
GCCGTGAACACCGCCAAGAAGTCCAGACTGACCGACGTGACACTT
CAGCCTTTCATGAGGCCCGTGCAGACCACACAAGAAGAGGACGGC
TGCTCCTGCAGATTCCCCGAGGAAGAGGAAGGCGGTTGCGAACTT
PD1 (extracellular domain without signal
peptide)-ICOS DS-CD28(PRRP)-truncated CD134)
(SEQ ID NO: 83)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGACCCCTAGAAGG
CCTGGACCTACCAGAAAGCACTACCAGCCTTACGCTCCTCCTAGA
GCCGTGAACACCGCCAAGAAGTCCAGACTGACCGACGTGACACTT
GGCGGCGGAAGCTTTAGAACCCCTATCCAAGAGGAACAGGCCGAC
GCTCACTCTACACTGGCT
PD1:ICOSBBt:CD2t (PD1 extracellular domain
without signal peptide)-ICOS DS-truncated
CD137 domain-truncated CD2 signaling domain)
(SEQ ID NO: 84)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACCCTCCAGCCTTTC
ATGAGGCCTGTGCAGACCACACAAGAAGAGGACGGCTGCTCCTGT
CGGTTCCCCGAGGAAGAGGAAGGCGGTTGCGAACTCCAGAATCCT
GCCACCTCTCAGCACCCTCCACCTCCACCTGGACACAGATCTCAG
GCCCCATCTCACAGACCTCCACCACCTGGTCATCGGGTGCAGCAT
CAGCCCCAGAAAAGACCTCCTGCTCCTAGCGGAACACAGGTGCAC
CAGCAAAAGGGACCTCCACTGCCTAGACCTAGAGTGCAGCCTAAG
CCTCCTCATGGCGCTGCCGAGAATAGCCTGTCTCCTAGCAGCAAC
PD1 extracellular domain without signal
peptide-ICOS DS-truncated CD134 domain-
truncated CD2 signaling domain
(SEQ ID NO: 85)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACCCTCCAGCCTTTC
ATGAGGCCTGTGCAGACCACACAAGAAGAGGACGGCTGCTCCTGT
CGGTTCCCCGAGGAAGAGGAAGGCGGCTGCGAACTGAATTGCAGA
AACACAGGCCCCTGGCTGAAGAAAGTGCTGAAGTGCAACACCCCT
GATCCGAGCAAGTTCTTTAGCCAGCTGAGCAGCGAGCATGGCGGC
GACGTTCAGAAATGGCTGTCTAGCCCATTTCCTAGCAGCAGCTTC
AGCCCTGGTGGACTGGCCCCTGAGATTAGCCCTCTGGAAGTGCTG
GAACGGGACAAAGTGACCCAGCTGCTGCCCCTGAATACCGACGCT
TACCTGAGCCTGCAAGAGCTGCAAGGACAGGACCCTACACACCTG
GTGTCCTACCTGAGACAGTGGGTCGTGATCCCTCCACCTCTCTCT
AGTCCTGGACCTCAGGCCTCT
PD1 extracellular domain without signal
peptide)-ICOS DS-truncated CD137 domain-
truncated CD2 signaling domain-IL-2 receptor
binding (IL2RB)(YLRQ) (SEQ ID NO: 86)
TTTCTGGACAGCCCCGACAGACCCTGGAATCCTCCTACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTTCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCCAGCCAGCTGTGCTGCCAGCTGAAGTTTTGG
CTGCCTATCGGCTGTGCCGCCTTCGTGGTTGTGTGTATCCTGGGC
TGCATCCTGATCTGCTGGCTGACCAAGAAAAAGTACAGCAGCTCC
GTGCACGACCCCAACGGCGAGTACATGTTCATGAGAGCCGTGAAC
ACCGCCAAGAAGTCCAGACTGACCGACGTGACCCTCCAGCCTTTC
ATGAGGCCTGTGCAGACCACACAAGAAGAGGACGGCTGCTCCTGT
CGGTTCCCCGAGGAAGAGGAAGGCGGTTGCGAACTCCAGAATCCT
GCCACCTCTCAGCACCCTCCACCTCCACCTGGACACAGATCTCAG
GCCCCATCTCACAGACCTCCACCACCTGGTCATCGGGTGCAGCAT
CAGCCCCAGAAAAGACCTCCTGCTCCTAGCGGAACACAGGTGCAC
CAGCAAAAGGGACCTCCACTGCCTAGACCTAGAGTGCAGCCTAAG
CCTCCTCATGGCGCTGCCGAGAATAGCCTGTCTCCTAGCAGCAAC
AACTGCCGCAACACAGGCCCCTGGCTGAAGAAAGTGCTGAAGTGC
AACACCCCTGATCCGAGCAAGTTCTTTAGCCAGCTGAGCAGCGAG
CATGGCGGCGACGTTCAGAAATGGCTGTCTAGCCCATTTCCAAGC
AGCAGCTTCAGCCCTGGTGGACTGGCCCCTGAGATTAGCCCTCTG
GAAGTGCTGGAACGGGACAAAGTGACCCAGCTGCTGCCCCTGAAT
ACCGACGCTTACCTGAGCCTGCAAGAGCTGCAAGGACAGGACCCT
ACACACCTGGTGTCCTACCTGAGACAGTGGGTCGTGATCCCACCT
CCTTTGAGCAGTCCAGGACCTCAGGCCTCT
CD3Z (intracellular Signaling Domain (Human
CD3ζ signaling domain) (SEQ ID NO: 87)
AGAGTGAAGTTCAGCAGATCCGCCGACGCTCCTGCCTATCAGCAG
GGCCAAAACCAGCTGTACAACGAGCTGAACCTGGGGAGAAGAGAA
GAGTACGACGTGCTGGACAAGCGGAGAGGCAGAGATCCTGAAATG
GGCGGCAAGCCCCAGCGGAGAAAGAATCCTCAAGAGGGCCTGTAT
AATGAGCTGCAAAAGGACAAGATGGCCGAGGCCTACAGCGAGATC
GGAATGAAGGGCGAGCGCAGAAGAGGCAAGGGACACGATGGACTG
TACCAGGGCCTGAGCACCGCCACCAAGGATACCTATGATGCCCTG
CACATGCAGGCCCTGCCTCCAAGA
Human CD3 Z signaling domain truncated
(CD3 ζ truncated domain)
(Other name: Human CD3ζ signaling domain
truncated; CD3 truncated domain)
(SEQ ID NO: 88)
AGAGTGAAGTTCAGCAGATCCGCCGACGCTCCTGCCTATCAGCAG
GGCCAAAACCAGCTGTACAACGAGCTGAACCTGGGGAGAAGAGAA
GAGTACGACGTGCTGGACAAGCGGAGAGGCAGAGATCCTGAAATG
GGCGGCAAA
Human CD3 E signaling domain truncated
(CD3 E truncated domain (Human
CD3 ϵ signaling domain truncated
(CD3 ϵ truncated domain) (SEQ ID NO: 89)
CCTGTGACTAGAGGTGCTGGTGCTGGCGGCAGACAGAGAGGCCAG
AACAAAGAAAGACCTCCTCCTGTGCCTAATCCTGACTACGAGCCC
ATCCGGAAGGGCCAGAGAGATCTGTACAGCGGCCTGAACCAGCGG
AGAATC
Human CD3 ZE signaling domain (CD3 ζϵ domain)
(Human CD3ζϵ signaling domain (CD3 domain)
(SEQ ID NO: 90)
AGAGTGAAGTTCAGCAGATCCGCCGACGCTCCTGCCTATCAGCAG
GGCCAAAACCAGCTGTACAACGAGCTGAACCTGGGGAGAAGAGAA
GAGTACGACGTGCTGGACAAGCGGAGAGGCAGAGATCCTGAAATG
GGCGGCAAGCCTGTGACTAGAGGTGCTGGTGCTGGCGGCAGACAG
AGAGGCCAGAACAAAGAAAGACCTCCTCCTGTGCCTAATCCTGAC
TACGAGCCCATCCGGAAGGGCCAGAGAGATCTGTACAGCGGCCTG
AACCAGCGGAGAATC
FMC63 scFV (CD8a Leader_Light_Chain_Linker_
Heavy_Chain_CD8a Hinge) (CD19 binding
extracellular domain) (SEQ ID NO: 91)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGC
CD19 (FMC63 scFV)_CD8a Transmembrane_4-1BB
Signaling_CD3Z (Other names: FMC63scFV_BB_Z
chimeric antigen receptor (CAR), CD19_BB_Z)
(SEQ ID NO: 92)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCGACATCTACATCTGGGCCCCTCTG
GCTGGAACATGTGGCGTGCTGCTGCTGAGCCTGGTCATCACCCTG
TATTGCAAGCGGGGCAGAAAGAAACTGCTCTACATCTTCAAGCAG
CCCTTCATGCGGCCCGTGCAGACCACACAAGAGGAAGATGGCTGC
TCCTGTCGGTTCCCCGAGGAAGAAGAAGGCGGCTGCGAGCTGAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAGCCCCAGCGGAGAAAGAATCCTCAAGAGGGCCTGTATAAT
GAGCTGCAAAAGGACAAGATGGCCGAGGCCTACAGCGAGATCGGA
ATGAAGGGCGAGCGCAGAAGAGGCAAGGGACACGATGGACTGTAC
CAGGGCCTGAGCACCGCCACCAAGGATACCTATGATGCCCTGCAC
ATGCAGGCCCTGCCTCCAAGATGA
CD19 (FMC63 scFV) CD28 Transmembrane CD28
Signaling_CD3Z (Other names: FMC63scFV_28_Z CAR,
CD19_28_Z) (SEQ ID NO: 93)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCCTGTTTCCCGGACCTAGCAAGCCT
TTCTGGGTGCTCGTTGTTGTTGGCGGCGTGCTGGCCTGTTACAGC
CTGCTGGTTACCGTGGCCTTCATCATCTTTTGGGTCCGAAGCAAG
CGGAGCCGGCTGCTGCACAGCGACTACATGAACATGACCCCTAGA
CGGCCCGGACCAACCAGAAAGCACTACCAGCCTTACGCTCCTCCT
AGAGACTTCGCCGCCTACAGATCTAGAGTGAAGTTCAGCAGATCC
GCCGACGCTCCTGCCTATCAGCAGGGCCAAAACCAGCTGTACAAC
GAGCTGAACCTGGGGAGAAGAGAAGAGTACGACGTGCTGGACAAG
CGGAGAGGCAGAGATCCTGAAATGGGCGGCAAGCCCCAGCGGAGA
AAGAATCCTCAAGAGGGCCTGTATAATGAGCTGCAAAAGGACAAG
ATGGCCGAGGCCTACAGCGAGATCGGAATGAAGGGCGAGCGCAGA
AGAGGCAAGGGACACGATGGACTGTACCAGGGCCTGAGCACCGCC
ACCAAGGATACCTATGATGCCCTGCACATGCAGGCCCTGCCTCCA
AGATGA
CD19 (FMC63 scFV)_CD28 Transmembrane_CD28
Signaling_4-1BB Signaling_CD3Z
(Other names: FMC63scFV_28_BBwt_Z CAR,
CD19_28_BBwt_Z) (SEQ ID NO: 94)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCCTGTTTCCCGGACCTAGCAAGCCT
TTCTGGGTGCTCGTTGTTGTTGGCGGCGTGCTGGCCTGTTACAGC
CTGCTGGTTACCGTGGCCTTCATCATCTTTTGGGTCCGAAGCAAG
CGGAGCCGGCTGCTGCACAGCGACTACATGAACATGACCCCTAGA
CGGCCCGGACCAACCAGAAAGCACTACCAGCCTTACGCTCCTCCT
AGAGACTTCGCCGCCTACAGATCCAAGCGGGGCAGAAAGAAGCTG
CTGTACATCTTCAAGCAGCCCTTCATGCGGCCCGTGCAGACCACA
CAAGAGGAAGATGGCTGCTCCTGTCGGTTCCCCGAGGAAGAAGAA
GGCGGTTGCGAACTGAGAGTGAAGTTCAGCAGATCCGCCGACGCT
CCTGCCTATCAGCAGGGCCAAAACCAGCTGTACAACGAGCTGAAC
CTGGGGAGAAGAGAAGAGTACGACGTGCTGGACAAGCGGAGAGGC
AGAGATCCTGAAATGGGCGGCAAGCCCCAGCGGAGAAAGAATCCT
CAAGAGGGCCTGTATAATGAGCTGCAAAAGGACAAGATGGCCGAG
GCCTACAGCGAGATCGGAATGAAGGGCGAGCGCAGAAGAGGCAAG
GGACACGATGGACTGTACCAGGGCCTGAGCACCGCCACCAAGGAT
ACCTATGATGCCCTGCACATGCAGGCCCTGCCTCCAAGATGA
CD19 (FMC63 scFV) CD28 Transmembrane CD28
Signaling_4-1BB truncated Signaling_CD3Z
(Other names: FMC63scFV_28_BBt_Z CAR,
CD19_28_BBt_Z (SEQ ID NO: 95)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCCTGTTTCCCGGACCTAGCAAGCCT
TTCTGGGTGCTCGTTGTTGTTGGCGGCGTGCTGGCCTGTTACAGC
CTGCTGGTTACCGTGGCCTTCATCATCTTTTGGGTCCGAAGCAAG
CGGAGCCGGCTGCTGCACAGCGACTACATGAACATGACCCCTAGA
CGGCCCGGACCAACCAGAAAGCACTACCAGCCTTACGCTCCTCCT
AGAGACTTCGCCGCCTACAGATCCCAGCCTTTCATGAGGCCTGTG
CAGACCACACAAGAAGAGGACGGCTGCTCCTGTCGGTTCCCCGAG
GAAGAGGAAGGCGGTTGCGAACTTAGAGTGAAGTTCAGCAGATCC
GCCGACGCTCCTGCCTATCAGCAGGGCCAAAACCAGCTGTACAAC
GAGCTGAACCTGGGGAGAAGAGAAGAGTACGACGTGCTGGACAAG
CGGAGAGGCAGAGATCCTGAAATGGGCGGCAAGCCCCAGCGGAGA
AAGAATCCTCAAGAGGGCCTGTATAATGAGCTGCAAAAGGACAAG
ATGGCCGAGGCCTACAGCGAGATCGGAATGAAGGGCGAGCGCAGA
AGAGGCAAGGGACACGATGGACTGTACCAGGGCCTGAGCACCGCC
ACCAAGGATACCTATGATGCCCTGCACATGCAGGCCCTGCCTCCA
AGATGA
CD19 (FMC_63_scFV)_CD28_Transmembrane CD28
Signaling_OX-40 Truncated Signaling_CD3Z
(Other names: FMC63scFV_28_OX40t_Z CAR,
CD19_28_OX40t_Z) (SEQ ID NO: 96)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCCTGTTTCCCGGACCTAGCAAGCCT
TTCTGGGTGCTCGTTGTTGTTGGCGGCGTGCTGGCCTGTTACAGC
CTGCTGGTTACCGTGGCCTTCATCATCTTTTGGGTCCGAAGCAAG
CGGAGCCGGCTGCTGCACAGCGACTACATGAACATGACCCCTAGA
CGGCCCGGACCAACCAGAAAGCACTACCAGCCTTACGCTCCTCCT
AGAGACTTCGCCGCCTACAGATCTGGCGGCGGAAGCTTCAGAACC
CCTATCCAAGAGGAACAGGCCGACGCTCACTCTACACTGGCTAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAGCCCCAGCGGAGAAAGAATCCTCAAGAGGGCCTGTATAAT
GAGCTGCAAAAGGACAAGATGGCCGAGGCCTACAGCGAGATCGGA
ATGAAGGGCGAGCGCAGAAGAGGCAAGGGACACGATGGACTGTAC
CAGGGCCTGAGCACCGCCACCAAGGATACCTATGATGCCCTGCAC
ATGCAGGCCCTGCCTCCAAGATGA
CD19 (FMC63scFV)_ICOS Transmembrane_ICOS_
Signaling_CD3Z (Other names:
FMC63scFV_ICOS_Z CAR, CD19_ICOS_Z
(SEQ ID NO: 97)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACACTGAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAGCCCCAGCGGAGAAAGAATCCTCAAGAGGGCCTGTATAAT
GAGCTGCAAAAGGACAAGATGGCCGAGGCCTACAGCGAGATCGGA
ATGAAGGGCGAGCGCAGAAGAGGCAAGGGACACGATGGACTGTAC
CAGGGCCTGAGCACCGCCACCAAGGATACCTATGATGCCCTGCAC
ATGCAGGCCCTGCCTCCAAGATGA
CD19 (FMC63scFV)_ICOS_Transmembrane_ICOS_
Signaling_4-1BB Signaling_CD3Z
(Other names: FMC63scFV_ICOS_BBwt_Z CAR,
CD19_ICOS_BBwt_Z) (SEQ ID NO: 98)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTGAAG
CGGGGCAGAAAGAAGCTGCTGTATATCTTCAAGCAGCCCTTCATG
CGGCCCGTGCAGACCACACAAGAGGAAGATGGCTGCTCCTGTCGG
TTCCCCGAGGAAGAAGAAGGCGGTTGCGAACTGAGAGTGAAGTTC
AGCAGATCCGCCGACGCTCCTGCCTATCAGCAGGGCCAAAACCAG
CTGTACAACGAGCTGAACCTGGGGAGAAGAGAAGAGTACGACGTG
CTGGACAAGCGGAGAGGCAGAGATCCTGAAATGGGCGGCAAGCCC
CAGCGGAGAAAGAATCCTCAAGAGGGCCTGTATAATGAGCTGCAA
AAGGACAAGATGGCCGAGGCCTACAGCGAGATCGGAATGAAGGGC
GAGCGCAGAAGAGGCAAGGGACACGATGGACTGTACCAGGGCCTG
AGCACCGCCACCAAGGATACCTATGATGCCCTGCACATGCAGGCC
CTGCCTCCAAGATGA
CD19 (FMC_63_scFV)_ICOS_Transmembrane_ICOS_
Signaling_4-1BB Truncated Signaling_CD3Z
(Other names: FMC63scFV_ICOS_BBt_Z
CAR, CD19_ICOS_BBt_Z (SEQ ID NO: 99)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGTTGCGAACTTAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAGCCCCAGCGGAGAAAGAATCCTCAAGAGGGCCTGTATAAT
GAGCTGCAAAAGGACAAGATGGCCGAGGCCTACAGCGAGATCGGA
ATGAAGGGCGAGCGCAGAAGAGGCAAGGGACACGATGGACTGTAC
CAGGGCCTGAGCACCGCCACCAAGGATACCTATGATGCCCTGCAC
ATGCAGGCCCTGCCTCCAAGATGA
CD19 (FMC63 scFV)_ICOS Transmembrane_ICOS_
Signaling_OX-40 Truncated
Signaling_CD3Z (Other names: FMC63scFV_
ICOS_OX40t_Z CAR, CD19_ICOS_OX40t_Z
(SEQ ID NO: 100)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACACTCGGC
GGAGGCAGCTTTAGAACCCCTATCCAAGAGGAACAGGCCGACGCT
CACTCTACACTGGCTAGAGTGAAGTTCAGCAGATCCGCCGACGCT
CCTGCCTATCAGCAGGGCCAAAACCAGCTGTACAACGAGCTGAAC
CTGGGGAGAAGAGAAGAGTACGACGTGCTGGACAAGCGGAGAGGC
AGAGATCCTGAAATGGGCGGCAAGCCCCAGCGGAGAAAGAATCCT
CAAGAGGGCCTGTATAATGAGCTGCAAAAGGACAAGATGGCCGAG
GCCTACAGCGAGATCGGAATGAAGGGCGAGCGCAGAAGAGGCAAG
GGACACGATGGACTGTACCAGGGCCTGAGCACCGCCACCAAGGAT
ACCTATGATGCCCTGCACATGCAGGCCCTGCCTCCAAGATGA
CD19 (FMC63scFV)_ICOS_Transmembrane_ICOS
Signaling_(mini CD28 Signaling)_CD3Z
(Other names: FMC63scFV_ICOS(28)_Z CAR,
CD19_ICOS(28)_Z (SEQ ID NO: 101)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGACCCCT
AGACGGCCCGGACCTACCAGAAAGCACTACCAGCCTTACGCTCCT
CCTCGGGCCGTGAACACAGCCAAGAAAAGCAGACTGACCGACGTG
ACCCTGAGAGTGAAGTTCAGCAGATCCGCCGACGCTCCTGCCTAT
CAGCAGGGCCAAAACCAGCTGTACAACGAGCTGAACCTGGGGAGA
AGAGAAGAGTACGACGTGCTGGACAAGCGGAGAGGCAGAGATCCT
GAAATGGGCGGCAAGCCCCAGCGGAGAAAGAATCCTCAAGAGGGC
CTGTATAATGAGCTGCAAAAGGACAAGATGGCCGAGGCCTACAGC
GAGATCGGAATGAAGGGCGAGCGCAGAAGAGGCAAGGGACACGAT
GGACTGTACCAGGGCCTGAGCACCGCCACCAAGGATACCTATGAT
GCCCTGCACATGCAGGCCCTGCCTCCAAGATGA
CD19 (FMC63scFV)_ICOS_Transmembrane_ICOS_
Signaling (mini CD28 Signaling)_4-1BB
Truncated Signaling_CD3Z (Other names:
FMC63scFV_ICOS(28)_BBt_Z CAR,
CD19_ICOS(28)_BBt_Z) (SEQ ID NO: 102)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGACCCCT
AGACGGCCCGGACCTACCAGAAAGCACTACCAGCCTTACGCTCCT
CCTCGGGCCGTGAACACAGCCAAGAAAAGCAGACTGACCGACGTG
ACCCTCCAGCCTTTCATGAGGCCTGTGCAGACCACACAAGAAGAG
GACGGCTGCTCCTGTCGGTTCCCCGAGGAAGAGGAAGGCGGTTGC
GAACTTAGAGTGAAGTTCAGCAGATCCGCCGACGCTCCTGCCTAT
CAGCAGGGCCAAAACCAGCTGTACAACGAGCTGAACCTGGGGAGA
AGAGAAGAGTACGACGTGCTGGACAAGCGGAGAGGCAGAGATCCT
GAAATGGGCGGCAAGCCCCAGCGGAGAAAGAATCCTCAAGAGGGC
CTGTATAATGAGCTGCAAAAGGACAAGATGGCCGAGGCCTACAGC
GAGATCGGAATGAAGGGCGAGCGCAGAAGAGGCAAGGGACACGAT
GGACTGTACCAGGGCCTGAGCACCGCCACCAAGGATACCTATGAT
GCCCTGCACATGCAGGCCCTGCCTCCAAGATGA
CD19 (FMC63 scFV)_ICOS_Transmembrane_ICOS
Signaling_(mini CD28 Signaling)_OX-40
Truncated Signaling_CD3Z (Other names:
FMC63scFV_ICOS(28)_40t_Z CAR,
CD19_ICOS(28)_40t_Z) (SEQ ID NO: 103)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGCCAGCTGAAG
TTCTGGCTGCCTATTGGCTGCGCCGCCTTCGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGACCCCT
AGACGGCCCGGACCTACCAGAAAGCACTACCAGCCTTACGCTCCT
CCTCGGGCCGTGAACACAGCCAAGAAAAGCAGACTGACCGACGTG
ACACTCGGCGGAGGCAGCTTTAGAACCCCTATCCAAGAGGAACAG
GCCGACGCTCACTCTACACTGGCTAGAGTGAAGTTCAGCAGATCC
GCCGACGCTCCTGCCTATCAGCAGGGCCAAAACCAGCTGTACAAC
GAGCTGAACCTGGGGAGAAGAGAAGAGTACGACGTGCTGGACAAG
CGGAGAGGCAGAGATCCTGAAATGGGCGGCAAGCCCCAGCGGAGA
AAGAATCCTCAAGAGGGCCTGTATAATGAGCTGCAAAAGGACAAG
ATGGCCGAGGCCTACAGCGAGATCGGAATGAAGGGCGAGCGCAGA
AGAGGCAAGGGACACGATGGACTGTACCAGGGCCTGAGCACCGCC
ACCAAGGATACCTATGATGCCCTGCACATGCAGGCCCTGCCTCCA
AGATGA
CD19_ICOSBBt_Zt CAR (CD19 binding
extracellular domain-ICOS DS-truncated CD137
intracellular domain-CD3Ztruncated domain)
(Other names: FMC63scFV_ICOS_BBt_Zt
CAR, CD19_ICOS_BBt_Zt (SEQ ID NO: 104)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGTTGCGAGCTGAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAATGA
CD19_ICOSBBt_ZE CAR (CD19 binding extracellular
domain-ICOS DS-truncated CD137
intracellular domain-CD3ZE domain)
(Other names: FMC63scFV_ICOS_BBt_ZE CAR,
CD19_ICOS_BBt_ZE (SEQ ID NO: 105)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGTTGCGAGCTGAGA
GTGAAGTTCAGCAGATCCGCCGACGCTCCTGCCTATCAGCAGGGC
CAAAACCAGCTGTACAACGAGCTGAACCTGGGGAGAAGAGAAGAG
TACGACGTGCTGGACAAGCGGAGAGGCAGAGATCCTGAAATGGGC
GGCAAGCCTGTGACTAGAGGTGCTGGTGCTGGCGGCAGACAGAGA
GGCCAGAACAAAGAAAGACCTCCTCCTGTGCCTAATCCTGACTAC
GAGCCCATCCGGAAGGGCCAGAGAGATCTGTACAGCGGCCTGAAC
CAGCGGAGAATCTGA
CD19_ICOSBBt:CD2t_Z CAR (CD19 binding
extracellular domain-ICOS DS-truncated
CD137 intracellular domain-CD2 truncated
Signaling Domain-CD3 domain) (Other names:
FMC63scFV_ICOS_BBt_CD2tZ CAR, CD19_ICOS_
BBt_CD2tZ (SEQ ID NO: 106)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGTTGCGAACTCCAG
AATCCTGCCACCTCTCAGCACCCTCCACCTCCACCTGGACACAGA
TCTCAGGCTCCTAGCCACAGACCTCCACCACCTGGTCATAGAGTG
CAGCACCAGCCTCAGAAGAGGCCTCCTGCTCCTTCTGGAACACAG
GTGCACCAGCAAAAGGGCCCTCCACTGCCTAGACCTAGGGTGCAG
CCTAAACCTCCTCATGGCGCCGCTGAGAACTCTCTGAGCCCCAGC
AGCAACAGAGTGAAGTTCAGCAGATCCGCCGACGCTCCTGCCTAT
CAGCAGGGCCAAAACCAGCTGTACAACGAGCTGAACCTGGGGAGA
AGAGAAGAGTACGACGTGCTGGACAAGCGGAGAGGCAGAGATCCT
GAAATGGGCGGCAAGCCCCAGCGGAGAAAGAATCCTCAAGAGGGC
CTGTATAATGAGCTGCAAAAGGACAAGATGGCCGAGGCCTACAGC
GAGATCGGAATGAAGGGCGAGCGCAGAAGAGGAAAGGGACACGAC
GGACTGTACCAGGGCCTGAGCACCGCCACAAAGGATACCTATGAC
GCCCTGCACATGCAGGCCCTGCCTCCAAGATGA
CD19_ICOSBBt:CD2t_ZE CAR (CD19 binding
extracellular domain-ICOS DS-truncated
CD137 intracellular domain-CD2 truncated
Signaling Domain-CD3ZE domain) (Other
names: FMC63scFV_ICOS_BBt_CD2tZE CAR,
CD19_ICOS_BBt_CD2tZE) (SEQ ID NO: 107)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGTTGCGAACTCCAG
AATCCTGCCACCTCTCAGCACCCTCCACCTCCACCTGGACACAGA
TCTCAGGCTCCTAGCCACAGACCTCCACCACCTGGTCATAGAGTG
CAGCACCAGCCTCAGAAGAGGCCTCCTGCTCCTTCTGGAACACAG
GTGCACCAGCAAAAGGGCCCTCCACTGCCTAGACCTAGGGTGCAG
CCTAAACCTCCTCATGGCGCCGCTGAGAACTCTCTGAGCCCCAGC
AGCAACAGAGTGAAGTTCAGCAGATCCGCCGACGCTCCTGCCTAT
CAGCAGGGCCAAAACCAGCTGTACAACGAGCTGAACCTGGGGAGA
AGAGAAGAGTACGACGTGCTGGACAAGCGGAGAGGCAGAGATCCT
GAAATGGGCGGCAAGCCTGTGACTAGAGGTGCTGGCGCTGGTGGA
AGGCAGAGAGGCCAGAACAAAGAACGGCCTCCTCCTGTGCCTAAT
CCTGACTACGAGCCCATCCGGAAGGGCCAGAGAGATCTGTACAGC
GGCCTGAACTACAGACACCAGCGGAGAATCTGA
CD19_ICOSBBt:IL2RB(YLRQ)_ZCAR (CD19 binding
extracellular domain-ICOS DS-truncated CD137
intracellular domain-IL2RB(YLRQ) domain-CD3Z
domain) (Other names: FMC63scFV_ICOS_BBt_IL2RB
(YLRQ)Z CAR, CD19_ICOS_BBt_IL2RB(YLRQ)Z
(SEQ ID NO: 108)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGCTGCGAACTGAAT
TGCAGAAACACAGGCCCCTGGCTGAAGAAAGTGCTGAAGTGCAAC
ACCCCTGATCCGAGCAAGTTTTTCAGCCAGCTGAGCAGCGAGCAT
GGCGGCGACGTTCAGAAATGGCTGTCTAGCCCATTTCCTAGCTCC
AGCTTCAGCCCTGGTGGACTGGCCCCTGAGATTAGCCCTCTGGAA
GTGCTGGAACGGGACAAAGTGACCCAGCTGCTGCCCCTGAATACC
GACGCCTACCTGAGCCTGCAAGAGCTGCAAGGCCAGGATCCTACA
CACCTCGTGCGCGTGAAGTTCAGCAGATCCGCTGATGCCCCTGCC
TATCAGCAGGGCCAAAACCAGCTGTACAACGAGCTGAACCTGGGG
AGAAGAGAAGAGTACGACGTGCTGGACAAGCGGAGAGGCAGAGAT
CCTGAAATGGGCGGCAAGCCCCAGCGGAGAAAGAATCCTCAAGAG
GGCCTGTATAATGAGCTGCAAAAGGACAAGATGGCCGAGGCCTAC
AGCGAGATCGGAATGAAGGGCGAGCGCAGAAGAGGCAAGGGACAC
GATGGACTGTACCAGGGCCTGAGCACCGCCACCAAGGATACCTAC
GATGCCTACAGACACCAGGCTCTGCCACCTAGATGA
CD19_ICOSBBt:IL2RB(YLRQ)_ZE_CAR (CD19 binding
extracellular domain-ICOS DS-truncated CD137
intracellular domain-IL2RB(YLRQ) domain-CD3ZE
domain (Other names: FMC63scFV_ICOS_BBt_IL2RB
(YLRQ)_ZE CAR) (SEQ ID NO: 110)
ATGGCTCTGCCTGTGACAGCTCTGCTGCTGCCTCTGGCTCTGCTT
CTGCATGCCGCCAGACCTGACATCCAGATGACCCAGACAACCAGC
AGCCTGTCTGCCAGCCTGGGCGATAGAGTGACCATCAGCTGTAGA
GCCAGCCAGGACATCAGCAAGTACCTGAACTGGTATCAGCAGAAA
CCCGACGGCACCGTGAAGCTGCTGATCTACCACACCAGCAGACTG
CACAGCGGCGTGCCAAGCAGATTTTCTGGCAGCGGCTCTGGCACC
GACTACAGCCTGACCATCTCCAACCTGGAACAAGAGGATATCGCT
ACCTACTTCTGCCAGCAAGGCAACACCCTGCCTTACACCTTTGGC
GGAGGCACCAAGCTGGAAATCACAGGCGGCGGAGGAAGCGGAGGC
GGAGGATCTGGTGGTGGTGGATCTGAAGTGAAACTGCAAGAGTCT
GGCCCTGGCCTGGTGGCCCCATCTCAATCTCTGAGCGTGACCTGT
ACCGTCAGCGGAGTGTCCCTGCCTGATTATGGCGTGTCCTGGATC
CGGCAGCCTCCTAGAAAAGGCCTGGAATGGCTGGGCGTGATCTGG
GGCAGCGAGACAACCTACTACAACAGCGCCCTGAAGTCCCGGCTG
ACCATCATCAAGGACAACTCCAAGAGCCAGGTGTTCCTGAAGATG
AACAGCCTCCAGACCGACGACACCGCCATCTACTATTGCGCCAAG
CACTACTACTACGGCGGCAGCTACGCCATGGATTATTGGGGCCAG
GGCACCAGCGTGACCGTGTCTAGTACAACAACCCCTGCTCCTCGG
CCTCCTACACCAGCTCCTACAATTGCCAGCCAGCCACTGTCTCTG
AGGCCCGAAGCTTGTAGACCTGCTGCTGGCGGAGCCGTGCATACA
AGAGGACTGGATTTCGCCTGCAGCCAGCTGTGCTGTCAGCTGAAG
TTCTGGCTGCCTATCGGCTGCGCCGCCTTTGTGGTTGTGTGTATC
CTGGGCTGCATCCTGATCTGCTGGCTGACCAAGAAAAAGTACAGC
AGCAGCGTGCACGACCCCAACGGCGAGTACATGTTCATGAGAGCC
GTGAACACCGCCAAGAAGTCCAGACTGACCGACGTGACCCTCCAG
CCTTTCATGAGGCCTGTGCAGACCACACAAGAAGAGGACGGCTGC
TCCTGTCGGTTCCCCGAGGAAGAGGAAGGCGGCTGCGAACTGAAT
TGCAGAAACACAGGCCCCTGGCTGAAGAAAGTGCTGAAGTGCAAC
ACCCCTGATCCGAGCAAGTTTTTCAGCCAGCTGAGCAGCGAGCAT
GGCGGCGACGTTCAGAAATGGCTGTCTAGCCCATTTCCTAGCTCC
AGCTTCAGCCCTGGTGGACTGGCCCCTGAGATTAGCCCTCTGGAA
GTGCTGGAACGGGACAAAGTGACCCAGCTGCTGCCCCTGAATACC
GACGCCTACCTGAGCCTGCAAGAGCTGCAAGGCCAGGATCCTACA
CACCTCGTGCGCGTGAAGTTCAGCAGATCCGCTGATGCCCCTGCC
TATCAGCAGGGCCAAAACCAGCTGTACAACGAGCTGAACCTGGGG
AGAAGAGAAGAGTACGACGTGCTGGACAAGCGGAGAGGCAGAGAT
CCTGAAATGGGCGGCAAGCCTGTGACTAGAGGTGCTGGTGCTGGC
GGCAGACAGAGAGGCCAGAACAAAGAAAGACCTCCTCCTGTGCCT
AATCCTGACTACGAGCCCATCCGGAAGGGCCAGAGAGATCTGTAC
AGCGGCCTGAACTACAGACACCAGCGGAGAATCTGA
CD28 BB signaling domain: CD28 signaling
domain_4-1BB Signaling Domain
(SEQ ID NO: 151)
CTGTTCCCCGGACCTAGCAAGCCCTTTTGGGTGCTCGTTGTTGTT
GGCGGCGTGCTGGCCTGTTATAGCCTGCTGGTTACCGTGGCCTTC
ATCATCTTTTGGGTCCGAAGCAAGCGGAGCCGGCTGCTGCACAGC
GACTACATGAACATGACCCCTAGACGGCCCGGACCAACCAGAAAG
CACTACCAGCCTTACGCTCCTCCTAGAGACTTCGCCGCCTACAGA
TCCAAGCGGGGCAGAAAGAAGCTGCTGTACATCTTCAAGCAGCCC
TTCATGCGGCCCGTGCAGACCACACAAGAGGAAGATGGCTGCTCC
TGTCGGTTCCCCGAGGAAGAAGAAGGCGGTTGCGAACTG
CD28 Signaling Domain truncated 4-1BB Signaling
Domain (Other name: CD28_BBt):
(SEQ ID NO: 152)
CTGTTCCCCGGACCTAGCAAGCCCTTTTGGGTGCTCGTTGTTGTT
GGCGGCGTGCTGGCCTGTTATAGCCTGCTGGTTACCGTGGCCTTC
ATCATCTTTTGGGTCCGAAGCAAGCGGAGCCGGCTGCTGCACAGC
GACTACATGAACATGACCCCTAGACGGCCCGGACCAACCAGAAAG
CACTACCAGCCTTACGCTCCTCCTAGAGACTTCGCCGCCTACAGA
TCCCAGCCTTTCATGAGGCCTGTGCAGACCACACAAGAAGAGGAC
GGCTGCTCCTGTCGGTTCCCCGAGGAAGAGGAAGGCGGTTGCGAA
CTT
CD28 Signaling Domain_truncated OX-40 Signaling
Domain (Other name: CD28_OX40t)
(SEQ ID NO: 153)
CTGTTCCCCGGACCTAGCAAGCCCTTTTGGGTGCTCGTTGTTGTT
GGCGGCGTGCTGGCCTGTTATAGCCTGCTGGTTACCGTGGCCTTC
ATCATCTTTTGGGTCCGAAGCAAGCGGAGCCGGCTGCTGCACAGC
GACTACATGAACATGACCCCTAGACGGCCCGGACCAACCAGAAAG
CACTACCAGCCTTACGCTCCTCCTAGAGACTTCGCCGCCTACAGA
TCTGGCGGCGGAAGCTTCAGAACCCCTATCCAAGAGGAACAGGCC
GACGCTCACTCTACACTGGCT
ICOS Transmembrane_ICOS Signaling Domain
(SEQ ID NO: 154)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTT
ICOS Transmembrane_ICOS Signaling Domain 4-1BB
Signaling Domain (SEQ ID NO: 155)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACCCTGAAGCGGGGCAGAAAGAAACTGCTG
TACATCTTCAAGCAGCCCTTCATGCGGCCCGTGCAGACCACACAA
GAGGAAGATGGCTGCTCCTGCAGATTCCCCGAGGAAGAAGAAGGC
GGCTGCGAACTT
ICOS Transmembrane_ICOS Signaling Domain_
Truncated 4-1BB Signaling Domain
(SEQ ID NO: 156)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTCAGCCTTTCATGAGGCCCGTGCAG
ACCACACAAGAAGAGGACGGCTGCTCCTGCAGATTCCCCGAGGAA
GAGGAAGGCGGTTGCGAACTT
ICOS Transmembrane_ICOS Signaling Domain_
Truncated OX-40 Signaling Domain
(ICOS_OX40t) (SEQ ID NO: 157)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTGGCGGCGGAAGCTTTAGAACCCCT
ATCCAAGAGGAACAGGCCGACGCTCACTCTACACTGGCT
ICOS Transmembrane_ICOS Signaling Domain
(mini-CD28)_Truncated 4-1BB Signaling
Domain (ICOS(28)_BBt) (SEQ ID NO: 158)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGACCCCTAGAAGGCCTGGACCTACCAGA
AAGCACTACCAGCCTTACGCTCCTCCTAGAGCCGTGAACACCGCC
AAGAAGTCCAGACTGACCGACGTGACACTTCAGCCTTTCATGAGG
CCCGTGCAGACCACACAAGAAGAGGACGGCTGCTCCTGCAGATTC
CCCGAGGAAGAGGAAGGCGGTTGCGAACTT
ICOS(28)_truncated OX-40 (CD134) intracellular
domain (ICOS(28)_OX40t) (SEQ ID NO: 159)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGACCCCTAGAAGGCCTGGACCTACCAGA
AAGCACTACCAGCCTTACGCTCCTCCTAGAGCCGTGAACACCGCC
AAGAAGTCCAGACTGACCGACGTGACACTTGGCGGCGGAAGCTTT
AGAACCCCTATCCAAGAGGAACAGGCCGACGCTCACTCTACACTG
GCT
ICOS Transmembrane_ICOS Signaling Domain_
4-1BB Signaling Domain with mutated
polybasic region (ICOS_BB(xPB))
(SEQ ID NO: 160)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACCCTGGCTGCCGGCGCTGCAGCTCTGCTG
TACATCTTCAAGCAGCCCTTCATGCGGCCCGTGCAGACCACACAA
GAGGAAGATGGCTGCTCCTGCAGATTCCCCGAGGAAGAAGAAGGC
GGCTGCGAACTT
ICOS Transmembrane_ICOS Signaling Domain_
4-1BB Signaling Domain with mutated
lysines (ICOS_BB(xUb)) (SEQ ID NO: 161)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACCCTGAAGCGGGGCAGAAAGAAACTGCTG
TACATCTTCGCACAGCCCTTCATGCGGCCCGTGCAGACCACACAA
GAGGAAGATGGCTGCTCCTGCAGATTCCCCGAGGAAGAAGAAGGC
GGCTGCGAACTT
ICOS Transmembrane_ICOS Signaling Domain 4-1BB
Signaling Domain with mutated lysines and
polybasic regions (ICOS_BB(xPBxUb))
(SEQ ID NO: 162)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACCCTGGCTGCCGGCGCTGCAGCTCTGCTG
TACATCTTCGCACAGCCCTTCATGCGGCCCGTGCAGACCACACAA
GAGGAAGATGGCTGCTCCTGCAGATTCCCCGAGGAAGAAGAAGGC
GGCTGCGAACTT
ICOS Transmembrane_ICOS Signaling Domain Wild_
Type OX-40 Signaling Domain (ICOS_40)
(SEQ ID NO: 163)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTAGGAGAGACCAGCGTCTGCCACCA
GATGCACATAAGCCACCTGGCGGCGGAAGCTTTAGAACCCCTATC
CAAGAGGAACAGGCCGACGCTCACTCTACACTGGCTAAAATC
ICOS Transmembrane_ICOS Signaling Domain_OX-40
Signaling Domain with mutated
polybasic region (ICOS_40(xPB)
(SEQ ID NO: 164)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTGCCGCGGACCAGGCCCTGCCACCA
GATGCACATAAGCCACCTGGCGGCGGAAGCTTTAGAACCCCTATC
CAAGAGGAACAGGCCGACGCTCACTCTACACTGGCTAAAATC
ICOS Transmembrane_ICOS Signaling Domain_OX-40
Signaling Domain with mutated
lysine residues (ICOS_40(xUb)):
(SEQ ID NO: 165)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTAGGAGAGACCAGCGTCTGCCACCA
GATGCACATGCACCACCTGGCGGCGGAAGCTTTAGAACCCCTATC
CAAGAGGAACAGGCCGACGCTCACTCTACACTGGCTGCAATC
ICOS Transmembrane_ICOS Signaling Domain
OX-40_Signaling Domain with mutated
lysine residues and polybasic regions
(ICOS_40(xPBxUb)): (SEQ ID NO: 166)
AGCCAGCTGTGCTGCCAGCTGAAGTTTTGGCTGCCTATCGGCTGT
GCCGCCTTCGTGGTTGTGTGTATCCTGGGCTGCATCCTGATCTGC
TGGCTGACCAAGAAAAAGTACAGCAGCTCCGTGCACGACCCCAAC
GGCGAGTACATGTTCATGAGAGCCGTGAACACCGCCAAGAAGTCC
AGACTGACCGACGTGACACTTGCCGCGGACCAGGCCCTGCCACCA
GATGCACATGCACCACCTGGCGGCGGAAGCTTTAGAACCCCTATC
CAAGAGGAACAGGCCGACGCTCACTCTACACTGGCTGCAATC
PD1 Extracellular (SEQ ID NO: 167)
TTCCTGGACTCTCCTGACAGACCCTGGAATCCTCCAACATTCAGC
CCCGCTCTGCTGGTGGTTACCGAGGGCGATAATGCCACCTTCACC
TGTAGCTTCAGCAACACCAGCGAGAGCTTCGTGCTGAACTGGTAC
AGAATGAGCCCCAGCAACCAGACCGACAAGCTGGCCGCCTTTCCT
GAGGATAGATCTCAGCCCGGCCAGGACTGCCGGTTCAGAGTTACA
CAGCTGCCCAACGGCCGGGACTTCCACATGTCTGTCGTCCGGGCC
AGAAGAAACGACAGCGGCACATATCTGTGCGGCGCCATTTCTCTG
GCCCCTAAGGCTCAGATCAAAGAGAGCCTGAGAGCCGAGCTGAGA
GTGACAGAAAGACGGGCCGAAGTGCCCACAGCTCACCCTTCACCT
TCTCCAAGACCTGCC
HLA-A2 Signal Peptide_PD1 Extracellular_PD1
Transmembrane (PD1 (HLASP-Truncated;
PD1_TLs) (SEQ ID NO: 168)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCGGC
CAGTTCCAGACACTGGTCGTGGGAGTTGTTGGCGGCCTGCTGGGA
TCTCTGGTTCTGCTTGTTTGGGTGCTCGCCGTGATCTGCTCTAGA
HLA-A2 Signal Peptide_PD1 Extracellular_CD28
Transmembrane CD28 Signaling Domain
(HLASP CD28 DS or PD1_CD28 or PD_28)
(SEQ ID NO: 169)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCTCTG
TTCCCCGGACCTAGCAAGCCCTTTTGGGTGCTCGTTGTTGTTGGC
GGCGTGCTGGCCTGTTATAGCCTGCTGGTTACCGTGGCCTTCATC
ATCTTTTGGGTCCGAAGCAAGCGGAGCCGGCTGCTGCACAGCGAC
TACATGAACATGACCCCTAGACGGCCCGGACCAACCAGAAAGCAC
TACCAGCCTTACGCTCCTCCTAGAGACTTCGCCGCCTACAGATCT
HLA-A2 Signal Peptide_PD1 Extracellular_CD28
Transmembrane_CD28 Signaling Domain_
4-1BB Signaling Domain (PD1_28_BBwt)
(SEQ ID NO: 170)
ATGGCTGTGATGGCCCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCTCTG
TTCCCCGGACCTAGCAAGCCCTTTTGGGTGCTCGTTGTTGTTGGC
GGCGTGCTGGCCTGTTATAGCCTGCTGGTTACCGTGGCCTTCATC
ATCTTTTGGGTCCGAAGCAAGCGGAGCCGGCTGCTGCACAGCGAC
TACATGAACATGACCCCTAGACGGCCCGGACCAACCAGAAAGCAC
TACCAGCCTTACGCTCCTCCTAGAGACTTCGCCGCCTACAGATCC
AAGCGGGGCAGAAAGAAGCTGCTGTACATCTTCAAGCAGCCCTTC
ATGCGGCCCGTGCAGACCACACAAGAGGAAGATGGCTGCTCCTGT
CGGTTCCCCGAGGAAGAAGAAGGCGGTTGCGAACTG
HLA-A2 Signal Peptide_PD1 Extracellular_CD28
Transmembrane_CD28 Signaling
Domain_Truncated 4-1BB Signaling Domain
(PD1_28_BBt) (SEQ ID NO: 171)
ATGGCTGTGATGGCCCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCTCTG
TTCCCCGGACCTAGCAAGCCCTTTTGGGTGCTCGTTGTTGTTGGC
GGCGTGCTGGCCTGTTATAGCCTGCTGGTTACCGTGGCCTTCATC
ATCTTTTGGGTCCGAAGCAAGCGGAGCCGGCTGCTGCACAGCGAC
TACATGAACATGACCCCTAGACGGCCCGGACCAACCAGAAAGCAC
TACCAGCCTTACGCTCCTCCTAGAGACTTCGCCGCCTACAGATCC
CAGCCTTTCATGAGGCCTGTGCAGACCACACAAGAAGAGGACGGC
TGCTCCTGTCGGTTCCCCGAGGAAGAGGAAGGCGGTTGCGAACTT
HLA-A2 Signal Peptide_PD1 Extracellular_CD28
Transmembrane_CD28 Signaling Domain Truncated
OX-40 Signaling Domain (PD1_28_OX40t)
(SEQ ID NO: 172)
ATGGCTGTGATGGCCCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCTCTG
TTCCCCGGACCTAGCAAGCCCTTTTGGGTGCTCGTTGTTGTTGGC
GGCGTGCTGGCCTGTTATAGCCTGCTGGTTACCGTGGCCTTCATC
ATCTTTTGGGTCCGAAGCAAGCGGAGCCGGCTGCTGCACAGCGAC
TACATGAACATGACCCCTAGACGGCCCGGACCAACCAGAAAGCAC
TACCAGCCTTACGCTCCTCCTAGAGACTTCGCCGCCTACAGATCT
GGCGGCGGAAGCTTCAGAACCCCTATCCAAGAGGAACAGGCCGAC
GCTCACTCTACACTGGCT
HLA-A2 Signal Peptide_PD1 Extracellular_ICOS
intracellular domain (PD1_ICOS)
(SEQ ID NO: 173)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGAGAGCCGTGAACACCGCCAAGAAGTCCAGA
CTGACCGACGTGACACTT
HLA-A2 Signal Peptide_PD1 Extracellular_ICOS
DS_CD137 (PD1_ICOS_BBwt)
(SEQ ID NO: 174)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGAGAGCCGTGAACACCGCCAAGAAGTCCAGA
CTGACCGACGTGACCCTGAAGCGGGGCAGAAAGAAACTGCTGTAC
ATCTTCAAGCAGCCCTTCATGCGGCCCGTGCAGACCACACAAGAG
GAAGATGGCTGCTCCTGCAGATTCCCCGAGGAAGAAGAAGGCGGC
TGCGAACTT
HLA-A2 Signal Peptide_PD1 Extracellular_ICOS
truncated CD137 (PD1_ICOS_BBt)
(SEQ ID NO: 175)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGAGAGCCGTGAACACCGCCAAGAAGTCCAGA
CTGACCGACGTGACACTTCAGCCTTTCATGAGGCCCGTGCAGACC
ACACAAGAAGAGGACGGCTGCTCCTGCAGATTCCCCGAGGAAGAG
GAAGGCGGTTGCGAACTT
HLA-A2 Signal Peptide_PD1 Extracellular_ICOS
truncated CD134 (PD1_ICOS_OX40t)
(SEQ ID NO: 176)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGAGAGCCGTGAACACCGCCAAGAAGTCCAGA
CTGACCGACGTGACACTTGGCGGCGGAAGCTTTAGAACCCCTATC
CAAGAGGAACAGGCCGACGCTCACTCTACACTGGCT
HLA-A2 Signal Peptide_PD1 Extracellular_ICOS
Transmembrane IC OS Signaling Domain
(mini-CD28)_Truncated 4-1BB Signaling
(PD1_ICOS(28)_BBt)
(SEQ ID NO: 177)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGACCCCTAGAAGGCCTGGACCTACCAGAAAG
CACTACCAGCCTTACGCTCCTCCTAGAGCCGTGAACACCGCCAAG
AAGTCCAGACTGACCGACGTGACACTTCAGCCTTTCATGAGGCCC
GTGCAGACCACACAAGAAGAGGACGGCTGCTCCTGCAGATTCCCC
GAGGAAGAGGAAGGCGGTTGCGAACTT
HLA-A2 Signal Peptide_PD1 Extracellular_ICOS
Transmembrane_ICOS Signaling Domain
(mini-CD28)_Truncated OX-40 Signaling Domain
(PD1 (HLASP-ICOS DS-CD28(PRRP)-
mutated CD134) (PD1_ICOS_OX40t, PD1:ICOS40t)
(SEQ ID NO: 178)
ATGGCTGTGATGGCTCCTAGAACACTGGTGCTGCTGCTGTCTGGT
GCCCTGGCTCTGACTCAGACATGGGCCTTTCTGGACAGCCCCGAC
AGACCCTGGAATCCTCCTACATTCAGCCCCGCTCTGCTGGTGGTT
ACCGAGGGCGATAATGCCACCTTCACCTGTAGCTTCAGCAACACC
AGCGAGAGCTTCGTGCTGAACTGGTACAGAATGAGCCCCAGCAAC
CAGACCGACAAGCTGGCCGCCTTTCCTGAGGATAGATCTCAGCCC
GGCCAGGACTGCCGGTTCAGAGTTACACAGCTGCCCAACGGCCGG
GACTTCCACATGTCTGTCGTTCGGGCCAGAAGAAACGACAGCGGC
ACATATCTGTGCGGCGCCATTTCTCTGGCCCCTAAGGCTCAGATC
AAAGAGAGCCTGAGAGCCGAGCTGAGAGTGACAGAAAGACGGGCC
GAAGTGCCCACAGCTCACCCTTCACCTTCTCCAAGACCTGCCAGC
CAGCTGTGCTGCCAGCTGAAGTTTTGGCTGCCTATCGGCTGTGCC
GCCTTCGTGGTTGTGTGTATCCTGGGCTGCATCCTGATCTGCTGG
CTGACCAAGAAAAAGTACAGCAGCTCCGTGCACGACCCCAACGGC
GAGTACATGTTCATGACCCCTAGAAGGCCTGGACCTACCAGAAAG
CACTACCAGCCTTACGCTCCTCCTAGAGCCGTGAACACCGCCAAG
AAGTCCAGACTGACCGACGTGACACTTGGCGGCGGAAGCTTTAGA
ACCCCTATCCAAGAGGAACAGGCCGACGCTCACTCTACACTGGCT
11-D5-3 scFv (CD8a Signal Peptide_11-D5-3
scFv, mouse_CD8a Hinge_BBZ)
(SEQ ID NO: 179)
ATGGCTCTGCCTGTGACAGCTCTGCTGCTGCCTCTGGCTCTGCTT
CTGCATGCCGCCAGACCTGACATCGTGCTGACACAGTCTCCACCT
AGCCTGGCCATGAGCCTGGGAAAGAGAGCCACCATCAGCTGTAGA
GCCAGCGAGAGCGTGACAATCCTGGGCTCTCACCTGATCCACTGG
TATCAGCAGAAGCCCGGCCAGCCTCCTACACTGCTGATTCAGCTG
GCCTCCAATGTGCAGACAGGCGTGCCAGCCAGATTTTCTGGCAGC
GGCAGCAGAACCGACTTCACCCTGACAATCGACCCCGTGGAAGAG
GACGATGTGGCCGTGTACTACTGCCTCCAGAGCCGGACAATCCCC
AGAACATTTGGCGGAGGCACCAAGCTGGAAATCAAGGGCAGCACA
AGCGGCTCTGGCAAGCCTGGATCTGGCGAGGGATCTACCAAGGGA
CAGATCCAGCTGGTGCAGTCTGGCCCCGAGCTGAAGAAACCTGGC
GAGACAGTGAAGATCAGCTGCAAGGCCAGCGGCTACACCTTCACC
GACTACAGCATCAACTGGGTCAAGAGAGCCCCTGGCAAGGGCCTG
AAATGGATGGGCTGGATCAACACCGAAACCAGAGAGCCCGCCTAC
GCCTACGACTTCAGAGGCAGATTCGCCTTCAGCCTGGAAACCAGC
GCCAGCACAGCCTACCTCCAGATCAACAACCTGAAGTACGAGGAC
ACCGCCACCTACTTTTGCGCCCTGGATTACAGCTACGCCATGGAC
TATTGGGGCCAGGGCACAAGCGTGACCGTGTCCTCTACAACAACC
CCTGCTCCTCGGCCTCCAACACCAGCTCCTACAATTGCCTCTCAG
CCCCTGTCTCTGAGGCCCGAAGCTTGTAGACCTGCTGCTGGCGGA
GCCGTGCATACAAGAGGACTGGATTTCGCCTGCGACATCTACATC
TGGGCCCCTCTGGCTGGAACATGTGGCGTGCTGCTGCTGAGCCTG
GTCATCACCCTGTACTGCAAGCGGGGCAGAAAGAAGCTGCTGTAC
ATCTTCAAGCAGCCCTTCATGCGGCCCGTGCAGACCACACAAGAG
GAAGATGGCTGCTCCTGTCGGTTCCCTGAGGAAGAAGAAGGCGGC
TGCGAGCTGAGAGTGAAGTTCAGCAGATCCGCCGACGCTCCTGCC
TATCAGCAGGGACAGAATCAGCTCTACAACGAGCTGAACCTGGGG
CGCAGAGAAGAGTACGACGTGCTGGACAAGAGAAGAGGCAGGGAC
CCTGAGATGGGCGGAAAGCCCCAGAGAAGAAAGAACCCTCAAGAG
GGCCTGTACAATGAGCTGCAAAAGGACAAGATGGCCGAGGCCTAC
AGCGAGATCGGAATGAAGGGCGAACGCAGAAGAGGAAAGGGCCAC
GACGGACTGTATCAGGGCCTGAGCACAGCCACCAAGGACACCTAT
GATGCCCTGCACATGCAGGCCCTGCCTCCAAGA
FHVH33 HVV (CD8a Signal Peptide_FHVH33 HVV_
CD8a Hinge_BBZ)
(SEQ ID NO: 180)
ATGGCTCTGCCTGTGACAGCTCTGCTGCTGCCTCTGGCTCTGCTT
CTGCATGCCGCTAGACCTGAGGTGCAGCTGCTTGAATCTGGCGGA
GGACTGGTTCAGCCTGGCGGATCTCTGAGACTGTCTTGTGCCGCC
AGCGGCTTCACCTTTAGCAGCTACGCCATGAGCTGGGTCCGACAG
GCTCCTGGCAAAGGCCTTGAATGGGTGTCCAGCATCAGCGGCAGC
GGCGACTACATCTACTACGCCGATAGCGTGAAGGGCAGATTCACC
ATCAGCCGGGACATCAGCAAGAACACCCTGTACCTCCAGATGAAC
AGCCTGAGAGCCGAGGACACCGCCGTGTACTACTGTGCCAAAGAA
GGCACCGGCGCCAATAGCAGCCTGGCCGATTATAGAGGCCAGGGC
ACACTGGTCACCGTGTCCAGTTTCGTGCCTGTGTTCCTGCCTGCC
AAGCCTACCACAACACCCGCTCCTAGACCTCCAACACCAGCTCCA
ACAATCGCCAGCCAGCCTCTGTCTCTGAGGCCAGAAGCTTGTAGA
CCTGCTGCTGGCGGAGCCGTGCATACAAGAGGACTGGATTTCGCC
TGCGACATCTACATCTGGGCCCCTCTGGCTGGAACATGTGGCGTG
CTGCTGCTGAGCCTGGTCATCACACTGTACTGCAACCACCGGAAC
AAGCGGGGCAGAAAGAAGCTGCTGTACATCTTTAAGCAGCCCTTC
ATGCGGCCCGTGCAGACCACACAAGAGGAAGATGGCTGCTCCTGT
CGGTTCCCCGAGGAAGAAGAAGGCGGCTGCGAGCTGAGAGTGAAG
TTCAGCAGATCCGCAGACGCCCCTGCCTATCAGCAGGGACAGAAC
CAGCTGTACAACGAGCTGAACCTGGGGAGAAGAGAAGAGTACGAC
GTGCTGGACAAGCGGAGAGGCAGAGATCCTGAGATGGGCGGAAAG
CCCCAGCGGAGAAAGAATCCTCAAGAGGGCCTGTATAATGAGCTG
CAAAAGGACAAGATGGCCGAGGCCTACAGCGAGATCGGAATGAAG
GGCGAGCGCAGAAGAGGCAAGGGACACGATGGACTGTACCAGGGC
CTGAGCACCGCCACCAAGGATACCTATGATGCCCTGCACATGCAG
GCCCTGCCTCCAAGA
BCAR003 HVV (CD8a Signal Peptide_BCAR003 HVV_
CD8a Hinge_BBZ)
(SEQ ID NO: 181)
ATGGCTCTGCCTGTGACAGCTCTGCTGCTGCCTCTGGCTCTGCTT
CTGCATGCCGCTAGACCCCAAGTGAAGCTGGAAGAGTCTGGCGGA
GGACTGGTGCAGGCTGGCAGATCTCTGAGACTGTCTTGTGCCGCC
AGCGAGCACACCTTTAGCTCTCACGTGATGGGCTGGTTCAGACAG
GCCCCTGGCAAAGAAAGGGAAAGCGTGGCCGTTATCGGCTGGCGG
GATATCAGCACAAGCTACGCCGATAGCGTGAAGGGCAGATTCACC
ATCAGCCGGGACAACGCCAAGAAAACCCTGTACCTCCAGATGAAC
AGCCTGAAGCCTGAGGACACCGCCGTGTACTACTGCGCCGCCAGA
AGAATTGACGCCGCCGACTTTGATTCTTGGGGCCAGGGAACCCAA
GTGACCGTTTCTAGCGGAGGCGGTGGAAGTGGCGGCGGTGGATCA
GGTGGTGGTGGATCTGGTGGCGGAGGAAGCGGCGGAGGCGGATCT
GCTGTTCAGCTTGTTGAATCAGGTGGCGGCCTGGTTCAGGCCGGG
GATTCTCTTAGACTGACCTGCACAGCCAGCGGCAGAGCCTTCAGC
ACCTACTTCATGGCCTGGTTTCGGCAGGCTCCCGGAAAAGAACGG
GAATTTGTGGCCGGAATCGCTTGGAGCGGAGGCTCTACAGCCTAT
GCCGATTCCGTGAAAGGCCGGTTTACCATCAGCAGAGATAATGCC
AAAAACACGGTGTACCTGCAAATGAACTCTCTGAAGTCCGAGGAT
ACGGCCGTCTATTACTGTGCCAGCAGAGGCATCGAGGTGGAAGAG
TTTGGAGCCTGGGGACAGGGCACACAAGTCACAGTGTCTAGCACC
AGCACCACCACACCAGCTCCTAGACCTCCAACTCCTGCTCCTACA
ATCGCCAGCCAGCCTCTGTCTCTGAGGCCAGAAGCTTGTAGACCT
GCTGCTGGCGGAGCCGTGCATACAAGAGGACTGGATTTCGCCTGC
GACATCTACATCTGGGCCCCTCTGGCTGGAACATGTGGCGTGCTG
CTGCTGAGCCTGGTCATCACCCTGTACTGCAAGCGGGGCAGAAAG
AAGCTGCTGTACATCTTCAAGCAGCCCTTCATGCGGCCCGTGCAG
ACCACACAAGAGGAAGATGGCTGCTCCTGTCGGTTCCCCGAGGAA
GAAGAAGGCGGCTGCGAGCTGAGAGTGAAGTTCAGCAGAAGTGCC
GACGCTCCCGCCTATCAGCAGGGACAGAACCAGCTGTACAACGAG
CTGAACCTGGGGAGAAGAGAAGAGTACGACGTGCTGGACAAGCGG
AGAGGCAGAGATCCTGAGATGGGCGGAAAGCCCCAGCGGAGAAAG
AATCCTCAAGAGGGCCTGTATAATGAGCTGCAAAAGGACAAGATG
GCCGAGGCCTACAGCGAGATCGGAATGAAGGGCGAGCGCAGAAGA
GGCAAGGGACACGATGGACTGTATCAGGGCCTGAGCACCGCCACC
AAGGATACCTATGATGCCCTGCACATGCAGGCCCTGCCTCCAAGA
GSI5022 HVV (CD8a Signal Peptide_G515022 HVV_
CD8a Hinge_BBZ) (SEQ ID NO: 182)
ATGGCTCTGCCTGTGACAGCTCTGCTGCTGCCTCTGGCTCTGCTT
CTGCATGCCGCTAGACCCCAAGTGAAGCTGGAAGAGTCTGGCGGA
GGACTGGTGCAGGCTGGCAGATCTCTGAGACTGTCTTGTGCCGCC
AGCGAGCACACCTTTAGCTCTCACGTGATGGGCTGGTTCAGACAG
GCCCCTGGCAAAGAAAGGGAAAGCGTGGCCGTTATCGGCTGGCGG
GATATCAGCACAAGCTACGCCGATAGCGTGAAGGGCAGATTCACC
ATCAGCCGGGACAACGCCAAGAAAACCCTGTACCTCCAGATGAAC
AGCCTGAAGCCTGAGGACACCGCCGTGTACTACTGCGCCGCCAGA
AGAATTGACGCCGCCGACTTTGATTCTTGGGGCCAGGGAACCCAA
GTGACCGTTTCTAGCGGAGGCGGTGGAAGTGGCGGCGGTGGATCA
GGTGGTGGTGGATCTGAAGTGCAGCTGGTGGAATCAGGCGGCGGT
CTTGTTCAAGCCGGTGGTTCACTGAGACTGAGCTGTGCCGCTTCC
GGCAGAACCTTTACCATGGGATGGTTTAGGCAGGCTCCAGGGAAA
GAACGCGAGTTCGTGGCCGCCATTTCTCTGTCTCCAACACTGGCC
TACTACGCCGAGTCCGTGAAAGGCCGGTTCACAATCTCCAGAGAT
AATGCCAAGAACACCGTGGTGCTGCAAATGAACTCCCTGAAGCCA
GAAGATACAGCCCTGTATTACTGTGCCGCCGACCGGAAGTCCGTG
ATGAGCATCAGACCTGACTACTGGGGACAGGGCACACAAGTCACA
GTGTCCAGCACCAGCACCACAACACCCGCTCCTAGACCTCCAACA
CCAGCTCCAACAATCGCCAGCCAGCCTCTGTCTCTGAGGCCAGAA
GCTTGTAGACCTGCTGCTGGCGGAGCCGTGCATACAAGAGGACTG
GATTTCGCCTGCGACATCTACATCTGGGCCCCTCTGGCTGGAACA
TGTGGCGTGCTGCTGCTGAGCCTGGTCATCACCCTGTACTGCAAG
CGGGGCAGAAAGAAGCTGCTGTACATCTTCAAGCAGCCCTTCATG
CGGCCCGTGCAGACCACACAAGAGGAAGATGGCTGCTCCTGTCGG
TTCCCCGAGGAAGAAGAAGGCGGCTGCGAGCTGAGAGTGAAGTTC
AGCAGATCCGCCGACGCTCCTGCCTATCAGCAGGGACAGAACCAG
CTGTACAACGAGCTGAACCTGGGGAGAAGAGAAGAGTACGACGTG
CTGGATAAGCGGAGAGGCAGAGATCCTGAGATGGGCGGAAAGCCC
CAGCGGAGAAAGAATCCTCAAGAGGGCCTGTATAATGAGCTGCAA
AAGGACAAGATGGCCGAGGCCTACAGCGAGATCGGAATGAAGGGC
GAGCGCAGAAGAGGCAAGGGACACGATGGACTGTATCAGGGCCTG
AGCACCGCCACCAAGGATACCTATGATGCCCTGCACATGCAGGCC
CTGCCTCCAAGA
BCMAsdAb1 HHV (CD8a Signal Peptide_
anti-BCMAsdAb1 HHV_CD8a Hinge)
(SEQ ID NO: 183)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGC
BCMAsdAb1 HHV CD8a Transmembrane_4-1BB
Signaling_CD3Z (BCMAsdAb1 HHV_
BB_z) (SEQ ID NO: 184)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCGACATCTACATCTGGGCCCCTCTGGCTGGA
ACATGTGGCGTGCTGCTGCTGAGCCTGGTCATCACCCTGTATTGC
AAGCGGGGCAGAAAGAAACTGCTCTACATCTTCAAGCAGCCCTTC
ATGCGGCCCGTGCAGACCACACAAGAGGAAGATGGCTGCTCCTGT
CGGTTCCCCGAGGAAGAAGAAGGCGGCTGCGAGCTGAGAGTGAAG
TTCAGCAGATCCGCCGACGCTCCTGCCTATCAGCAGGGCCAAAAC
CAGCTGTACAACGAGCTGAACCTGGGGAGAAGAGAAGAGTACGAC
GTGCTGGACAAGCGGAGAGGCAGAGATCCTGAAATGGGCGGCAAG
CCCCAGCGGAGAAAGAATCCTCAAGAGGGCCTGTATAATGAGCTG
CAAAAGGACAAGATGGCCGAGGCCTACAGCGAGATCGGAATGAAG
GGCGAGCGCAGAAGAGGCAAGGGACACGATGGACTGTACCAGGGC
CTGAGCACCGCCACCAAGGATACCTATGATGCCCTGCACATGCAG
GCCCTGCCTCCAAGA
BCMAsdAb1 HHV CD28 Transmembrane CD28
Signaling_CD3Z (BCMAsdAb1 HHV_
28_Z) (SEQ ID NO: 185)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCCTGTTTCCCGGACCTAGCAAGCCTTTCTGG
GTGCTCGTTGTTGTTGGCGGCGTGCTGGCCTGTTACAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCTAGAGTGAAGTTCAGCAGATCCGCCGAC
GCTCCTGCCTATCAGCAGGGCCAAAACCAGCTGTACAACGAGCTG
AACCTGGGGAGAAGAGAAGAGTACGACGTGCTGGACAAGCGGAGA
GGCAGAGATCCTGAAATGGGCGGCAAGCCCCAGCGGAGAAAGAAT
CCTCAAGAGGGCCTGTATAATGAGCTGCAAAAGGACAAGATGGCC
GAGGCCTACAGCGAGATCGGAATGAAGGGCGAGCGCAGAAGAGGC
AAGGGACACGATGGACTGTACCAGGGCCTGAGCACCGCCACCAAG
GATACCTATGATGCCCTGCACATGCAGGCCCTGCCTCCAAGA
BCMAsdAb1 HHV_CD28 Transmembrane_CD28
Signaling_4-1BB Signaling_CD3Z
(BCMAsdAb1 HHV_28_BBwt_z) (SEQ ID NO: 186)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCCTGTTTCCCGGACCTAGCAAGCCTTTCTGG
GTGCTCGTTGTTGTTGGCGGCGTGCTGGCCTGTTACAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCCAAGCGGGGCAGAAAGAAGCTGCTGTAC
ATCTTCAAGCAGCCCTTCATGCGGCCCGTGCAGACCACACAAGAG
GAAGATGGCTGCTCCTGTCGGTTCCCCGAGGAAGAAGAAGGCGGT
TGCGAACTGAGAGTGAAGTTCAGCAGATCCGCCGACGCTCCTGCC
TATCAGCAGGGCCAAAACCAGCTGTACAACGAGCTGAACCTGGGG
AGAAGAGAAGAGTACGACGTGCTGGACAAGCGGAGAGGCAGAGAT
CCTGAAATGGGCGGCAAGCCCCAGCGGAGAAAGAATCCTCAAGAG
GGCCTGTATAATGAGCTGCAAAAGGACAAGATGGCCGAGGCCTAC
AGCGAGATCGGAATGAAGGGCGAGCGCAGAAGAGGCAAGGGACAC
GATGGACTGTACCAGGGCCTGAGCACCGCCACCAAGGATACCTAT
GATGCCCTGCACATGCAGGCCCTGCCTCCAAGA
BCMAsdAb1 HHV_CD28 Transmembrane CD28
Signaling 4-1BB truncated
Signaling_CD3Z BCMAsdAb1 HHV_28_BBt_z
(SEQ ID NO: 187)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCCTGTTTCCCGGACCTAGCAAGCCTTTCTGG
GTGCTCGTTGTTGTTGGCGGCGTGCTGGCCTGTTACAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCCCAGCCTTTCATGAGGCCTGTGCAGACC
ACACAAGAAGAGGACGGCTGCTCCTGTCGGTTCCCCGAGGAAGAG
GAAGGCGGTTGCGAACTTAGAGTGAAGTTCAGCAGATCCGCCGAC
GCTCCTGCCTATCAGCAGGGCCAAAACCAGCTGTACAACGAGCTG
AACCTGGGGAGAAGAGAAGAGTACGACGTGCTGGACAAGCGGAGA
GGCAGAGATCCTGAAATGGGCGGCAAGCCCCAGCGGAGAAAGAAT
CCTCAAGAGGGCCTGTATAATGAGCTGCAAAAGGACAAGATGGCC
GAGGCCTACAGCGAGATCGGAATGAAGGGCGAGCGCAGAAGAGGC
AAGGGACACGATGGACTGTACCAGGGCCTGAGCACCGCCACCAAG
GATACCTATGATGCCCTGCACATGCAGGCCCTGCCTCCAAGA
BCMAsdAb1 HHV CD28 Transmembrane CD28
Signaling_OX-40 Truncated
Signaling_CD3Z (BCMAsdAb1 HHV_28_OX40t_z)
(SEQ ID NO: 188)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCCTGTTTCCCGGACCTAGCAAGCCTTTCTGG
GTGCTCGTTGTTGTTGGCGGCGTGCTGGCCTGTTACAGCCTGCTG
GTTACCGTGGCCTTCATCATCTTTTGGGTCCGAAGCAAGCGGAGC
CGGCTGCTGCACAGCGACTACATGAACATGACCCCTAGACGGCCC
GGACCAACCAGAAAGCACTACCAGCCTTACGCTCCTCCTAGAGAC
TTCGCCGCCTACAGATCTGGCGGCGGAAGCTTCAGAACCCCTATC
CAAGAGGAACAGGCCGACGCTCACTCTACACTGGCTAGAGTGAAG
TTCAGCAGATCCGCCGACGCTCCTGCCTATCAGCAGGGCCAAAAC
CAGCTGTACAACGAGCTGAACCTGGGGAGAAGAGAAGAGTACGAC
GTGCTGGACAAGCGGAGAGGCAGAGATCCTGAAATGGGCGGCAAG
CCCCAGCGGAGAAAGAATCCTCAAGAGGGCCTGTATAATGAGCTG
CAAAAGGACAAGATGGCCGAGGCCTACAGCGAGATCGGAATGAAG
GGCGAGCGCAGAAGAGGCAAGGGACACGATGGACTGTACCAGGGC
CTGAGCACCGCCACCAAGGATACCTATGATGCCCTGCACATGCAG
GCCCTGCCTCCAAGA
BCMAsdAb1 HHV_ICOS Transmembrane_ICOS
Signaling_CD3Z (BCMAsdAb1 HHV_
ICOS_Z) (SEQ ID NO: 189)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCAGCCAGCTGTGCTGCCAGCTGAAGTTCTGG
CTGCCTATTGGCTGCGCCGCCTTCGTGGTTGTGTGTATCCTGGGC
TGCATCCTGATCTGCTGGCTGACCAAGAAAAAGTACAGCAGCAGC
GTGCACGACCCCAACGGCGAGTACATGTTCATGAGAGCCGTGAAC
ACCGCCAAGAAGTCCAGACTGACCGACGTGACACTGAGAGTGAAG
TTCAGCAGATCCGCCGACGCTCCTGCCTATCAGCAGGGCCAAAAC
CAGCTGTACAACGAGCTGAACCTGGGGAGAAGAGAAGAGTACGAC
GTGCTGGACAAGCGGAGAGGCAGAGATCCTGAAATGGGCGGCAAG
CCCCAGCGGAGAAAGAATCCTCAAGAGGGCCTGTATAATGAGCTG
CAAAAGGACAAGATGGCCGAGGCCTACAGCGAGATCGGAATGAAG
GGCGAGCGCAGAAGAGGCAAGGGACACGATGGACTGTACCAGGGC
CTGAGCACCGCCACCAAGGATACCTATGATGCCCTGCACATGCAG
GCCCTGCCTCCAAGA
BCMAsdAb1 HHV_ICOS Transmembrane_ICOS
Signaling_4-1BB Signaling_CD3Z
(BCMAsdAb1 HHV_ICOS_BBwt_z)
(SEQ ID NO: 190)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCAGCCAGCTGTGCTGCCAGCTGAAGTTCTGG
CTGCCTATTGGCTGCGCCGCCTTCGTGGTTGTGTGTATCCTGGGC
TGCATCCTGATCTGCTGGCTGACCAAGAAAAAGTACAGCAGCAGC
GTGCACGACCCCAACGGCGAGTACATGTTCATGAGAGCCGTGAAC
ACCGCCAAGAAGTCCAGACTGACCGACGTGACCCTGAAGCGGGGC
AGAAAGAAGCTGCTGTATATCTTCAAGCAGCCCTTCATGCGGCCC
GTGCAGACCACACAAGAGGAAGATGGCTGCTCCTGTCGGTTCCCC
GAGGAAGAAGAAGGCGGTTGCGAACTGAGAGTGAAGTTCAGCAGA
TCCGCCGACGCTCCTGCCTATCAGCAGGGCCAAAACCAGCTGTAC
AACGAGCTGAACCTGGGGAGAAGAGAAGAGTACGACGTGCTGGAC
AAGCGGAGAGGCAGAGATCCTGAAATGGGCGGCAAGCCCCAGCGG
AGAAAGAATCCTCAAGAGGGCCTGTATAATGAGCTGCAAAAGGAC
AAGATGGCCGAGGCCTACAGCGAGATCGGAATGAAGGGCGAGCGC
AGAAGAGGCAAGGGACACGATGGACTGTACCAGGGCCTGAGCACC
GCCACCAAGGATACCTATGATGCCCTGCACATGCAGGCCCTGCCT
CCAAGA
BCMAsdAb1_ICOS Transmembrane_ICOS Signaling
4-1BB Truncated Signaling_CD3Z
(BCMAsdAb1 HHV_ICOS_BBt_z) (SEQ ID NO: 191)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCAGCCAGCTGTGCTGCCAGCTGAAGTTCTGG
CTGCCTATTGGCTGCGCCGCCTTCGTGGTTGTGTGTATCCTGGGC
TGCATCCTGATCTGCTGGCTGACCAAGAAAAAGTACAGCAGCAGC
GTGCACGACCCCAACGGCGAGTACATGTTCATGAGAGCCGTGAAC
ACCGCCAAGAAGTCCAGACTGACCGACGTGACCCTCCAGCCTTTC
ATGAGGCCTGTGCAGACCACACAAGAAGAGGACGGCTGCTCCTGT
CGGTTCCCCGAGGAAGAGGAAGGCGGTTGCGAACTTAGAGTGAAG
TTCAGCAGATCCGCCGACGCTCCTGCCTATCAGCAGGGCCAAAAC
CAGCTGTACAACGAGCTGAACCTGGGGAGAAGAGAAGAGTACGAC
GTGCTGGACAAGCGGAGAGGCAGAGATCCTGAAATGGGCGGCAAG
CCCCAGCGGAGAAAGAATCCTCAAGAGGGCCTGTATAATGAGCTG
CAAAAGGACAAGATGGCCGAGGCCTACAGCGAGATCGGAATGAAG
GGCGAGCGCAGAAGAGGCAAGGGACACGATGGACTGTACCAGGGC
CTGAGCACCGCCACCAAGGATACCTATGATGCCCTGCACATGCAG
GCCCTGCCTCCAAGA
BCMAsdAb1_ICOS Transmembrane_ICOS Signaling
OX-40 Truncated Signaling_CD3Z
(BCMAsdAb1 HHV_ICOS_OX40t_z)
(SEQ ID NO: 192)
ATGGCTCTGCCTGTGACAGCTCTGCTGCTGCCTCTGGCTCTGCTT
CTGCATGCCGCTAGACCTGAAGTGCAGTTGCAGGCTTCTGGCGGA
GGACTTGCTCAACCTGGCGGAAGCCTGAGACTGTCTTGTGCCGCC
TCTGGCAGAACCTTCAGCACCTACTTCATGGCCTGGTTCAGACAG
CCTCCTGGCAAAGGCCTGGAATACGTTGGCGGAATCCGTTGGAGT
GATGGCGTGCCACACTACGCCGATAGCGTGAAGGGCAGATTCACC
ATCAGCCGGGACAACGCCAAGAACACCGTGTACCTCCAGATGAAC
AGCCTGAGAGCCGAGGATACCGCCGTGTACTTCTGTGCCAGCAGA
GGAATCGCCGACGGCAGCGATTTTGGCTCTTATGGCCAGGGCACC
CAAGTGACCGTGTCCAGCACAACAACCCCTGCTCCTAGACCTCCT
ACACCAGCTCCTACAATCGCCAGCCAGCCTCTGTCTCTGAGGCCA
GAGGCTTGTAGACCTGCTGCTGGCGGAGCCGTGCATACAAGAGGA
CTGGATTTCGCCTGCAGCCAGCTGTGCTGCCAGCTGAAGTTCTGG
CTGCCTATTGGCTGCGCCGCCTTCGTGGTTGTGTGTATCCTGGGC
TGCATCCTGATCTGCTGGCTGACCAAGAAAAAGTACAGCAGCAGC
GTGCACGACCCCAACGGCGAGTACATGTTCATGAGAGCCGTGAAC
ACCGCCAAGAAGTCCAGACTGACCGACGTGACACTCGGCGGAGGC
AGCTTTAGAACCCCTATCCAAGAGGAACAGGCCGACGCTCACTCT
ACACTGGCTAGAGTGAAGTTCAGCAGATCCGCCGACGCTCCTGCC
TATCAGCAGGGCCAAAACCAGCTGTACAACGAGCTGAACCTGGGG
AGAAGAGAAGAGTACGACGTGCTGGACAAGCGGAGAGGCAGAGAT
CCTGAAATGGGCGGCAAGCCCCAGCGGAGAAAGAATCCTCAAGAG
GGCCTGTATAATGAGCTGCAAAAGGACAAGATGGCCGAGGCCTAC
AGCGAGATCGGAATGAAGGGCGAGCGCAGAAGAGGCAAGGGACAC
GATGGACTGTACCAGGGCCTGAGCACCGCCACCAAGGATACCTAT
GATGCCCTGCACATGCAGGCCCTGCCTCCAAGA
CD28 signaling domain: CD28 transmembrane_CD28
signaling domain (SEQ ID NO: 194)
CTGTTCCCCGGACCTAGCAAGCCCTTTTGGGTGCTCGTTGTTGTT
GGCGGCGTGCTGGCCTGTTATAGCCTGCTGGTTACCGTGGCCTTC
ATCATCTTTTGGGTCCGAAGCAAGCGGAGCCGGCTGCTGCACAGC
GACTACATGAACATGACCCCTAGACGGCCCGGACCAACCAGAAAG
CACTACCAGCCTTACGCTCCTCCTAGAGACTTCGCCGCCTACAGA
TCT
Human CD137/4-1BB (SEQ ID NO: 195)
AAGCGGGGCAGAAAGAAACTGCTGTACATCTTCAAGCAGCCCTTC
ATGCGGCCCGTGCAGACCACACAAGAGGAAGATGGCTGCTCCTGC
AGATTCCCCGAGGAAGAAGAAGGCGGCTGCGAACTT
Human CD134/0X40 (SEQ ID NO: 196)
AGGAGAGACCAGCGTCTGCCACCAGATGCACATAAGCCACCTGGC
GGCGGAAGCTTTAGAACCCCTATCCAAGAGGAACAGGCCGACGCT
CACTCTACACTGGCTAAAATC
truncated/mutated CD137/4-1BB (SEQ ID NO: 197)
CAGCCTTTCATGAGGCCCGTGCAGACCACACAAGAAGAGGACGGC
TGCTCCTGCAGATTCCCCGAGGAAGAGGAAGGCGGTTGCGAACTT
Truncated/mutated CD134/0X40 (SEQ ID NO: 198)
GGCGGCGGAAGCTTTAGAACCCCTATCCAAGAGGAACAGGCCGAC
GCTCACTCTACACTGGCT

In some embodiments, the cell disclosed herein further comprises a sequence encoding an artificial antigen receptor, a therapeutic polypeptide, an immune cell modulatory protein, or a combination thereof. In some embodiments, the artificial antigen receptor comprises a chimeric antigen receptor (CAR). In some embodiments, the artificial antigen receptor comprises a recombinant T cell receptor (rTCR). In some embodiments, the artificial antigen receptor comprises an enhanced affinity TCR. In some embodiments, the artificial antigen receptor binds to a tumor associated antigen (TAA), a pathogen associated protein, or an antigen associated with the disease or disorder is a cancer, an autoimmune disease or disorder, an infectious disease, an inflammatory disease, a renal disease or disorder, a lung disease or disorder, a liver disease or disorder a neurodegenerative disorder or disorder, or a metabolic disorder or disorder.

In some embodiments, the artificial antigen receptor binds to a TAA associated with a solid tumor or a hematologic cancer. In some embodiments, artificial antigen receptor binds to a TAA associated with a cancer selected from any one of leukemia, acute leukemia, acute lymphoblastic leukemia (ALL), acute lymphocytic leukemia, B cell, T cell or FAB ALL, acute myeloid leukemia (AML), acute myelogenous leukemia, chronic myelocytic leukemia (CIVIL), chronic lymphocytic leukemia (CLL), hairy cell leukemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, and hemangioma.

In some embodiments, the artificial antigen receptor binds to a TAA selected from kallikrein 4, papillomavirus binding factor (PBF), preferentially expressed antigen of melanoma (PRAME), Wilms' tumor-I (WTI), Hydroxysteroid Dehydrogenase Like I (HSDLI), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CAI25), folate receptor a, sperm protein 17, tumor-associated differentially expressed gene-12 (TADG-12), mucin-16 (MUC-16), LI cell adhesion molecule (LICAM), mannan-MUC-1, Human endogenous retrovirus K (HERV-K-MEL), Kita-kyushu lung cancer antigen-I (KK-LC-1), human cancer/testis antigen (KM-HN-1), cancer testis antigen (LAGE-I), melanoma antigen-Al (MAGE-A1), Sperm surface zona pellucida binding protein (Spl 7), Synovial Sarcoma, X Breakpoint 4 (SSX-4), Transient axonal glycoprotein-1 (TAG-I), Transient axonal glycoprotein-2 (TAG-2), Enabled Homolog (ENAH), mammoglobin-A, NY-BR-I, Breast Cancer Antigen, (BAGE-1), B melanoma antigen, melanoma antigen-Al (MAGE-Al), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B 1, mucin I (MUC I), p62, survivin, lymphocyte common antigen (CD45), DickkopfWNT Signaling Pathway Inhibitor I (DKKI), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor I Homolog (COA-1), 0-linked β-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (MEI), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAPl), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTCl), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FNl), Gruwih Anest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDX5), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OAl), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gp100), N-acetylglucosaminyltransferase V gene (GnTVf), Lymphocyte Antigen 6 Complex Locus K (LY6K), melanoma antigen-AlO (MAGE-AlO), melanoma antigen-Al2 (MAGE-Al2), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-Al, Fibronectin Type III Domain Containing 3B (FDNC3B) promyelocytic leukemia/retinoic acid receptor alpha fusion protein (pml-RARalpha), melanoma antigen-Cl (MAGE-Cl), membrane protein alternative spliced isoform (D393-CD20), melanoma antigen-A4 (MAGE-A4), and melanoma antigen-A3 (MAGE-A3).

In some embodiments, the artificial antigen receptor binds to an antigen associated with an autoimmune condition or disorder selected from any one of Type 1 Diabetes, rheumatoid arthritis (RA), systemic lupus erythematosis (SLE), or multiple sclerosis (MS). In some embodiments, the artificial antigen receptor binds to an antigen associated with an autoimmune condition or disorder selected from any one of Carboxypeptidase H, Chromogranin A, Glutamate decarboxylase, Imogen-38, Insulin, Insulinoma antigen-2 and 2β, Islet-specific glucose-6-phosphatase catalytic subunit related protein (IGRP), Proinsulin, α-enolase, Aquaporin-4, β-arrestin, Myelin basic protein, Myelin oligodendrocytic glycoprotein, Proteolipid protein, S100-β, Citrullinated protein, Collagen II, Heat shock proteins, Human cartilage glycoprotein, Double-stranded DNA, La antigen, Nucleosomal histones and ribonucleoproteins (snRNP), Phospholipid-β-2 glycoprotein I complex, Poly(ADP-ribose) polymerase, Sm antigens of U-1 small ribonucleoprotein complex.

In some embodiments, the artificial antigen receptor binds to a pathogen associated antigen from a bacterial, a fungal or a parasitic protein or fragment thereof. In some embodiments, the artificial antigen receptor binds to an antigen associated with HIV infection, human Cytomegalovirus infection, Hepatitis B infection, Hepatitis C infection, Ebola virus infection, Dengue, Yellow fever, Listeriosis, Tuberculosis, Cholera, Malaria, Leishmaniasis, or Trypanosoma infection, or a combination thereof.

In some embodiments, the artificial antigen receptor binds to an antigen associated with a neurodegenerative disorder or condition selected from Alzheimer's disease (AD) and other dementias, Parkinson's disease (PD) and PD-related disorders, Prion disease, Motor neurone diseases (MND), Huntington's disease (HD), Spinocerebellar ataxia (SCA) or Spinal muscular atrophy (SMA). In some embodiments, the antigen associated with the neurodegenerative disorder or condition is any one of Amyloid β (Aβ), tau, alpha-synuclein (α-syn), mHTT or prion PrPsc or a combination thereof.

In some embodiments, the therapeutic polypeptide is a cytokine, a cytokine receptor, a chemokine, a chemokine receptor, an immunogenic polypeptide, or a cell surface protein that binds to a target on the surface of another cell. In some embodiments, the immune cell modulatory protein is a cytokine, a chemokine, a transcription factor, a protein kinase, a protease, a component or an adaptor protein of a cell signaling pathway.

In some embodiments, the cell disclosed herein expresses the RTCR disclosed herein. In some embodiments, the cell disclosed herein expresses the RTCR disclosed herein stably or transiently. In some embodiments, the cell disclosed herein expresses the RTCR disclosed herein stably. In some embodiments, the cell disclosed herein expresses the RTCR disclosed herein transiently.

In some embodiments, the cell disclosed herein co-expresses at least one of the endogenous co-stimulatory molecules CD28, CD2, OX-40, ICOS, CD28, CD3, CD4, CD8 and CD40L or a combination thereof.

The present disclosure also provides a modified T lymphocyte (T cell), comprising: (a) a modification of an endogenous sequence encoding a T cell Receptor (TCR), wherein the modification reduces or eliminates a level of expression or activity of the TCR or; and (b) a recombinant T cell co-stimulatory receptor (RTCR) disclosed herein. In some embodiments, the modification of an endogenous sequence encoding a T cell Receptor (TCR) is done using a nucleic acid modifying system. In some embodiments, the nucleic acid modifying system is one or more of a CRISPR/Cas protein, a Transcription Activator-Like Effector Nuclease (TALEN), a Zinc Finger Nuclease (ZFN), and an endonuclease. In some embodiments, the modification of an endogenous sequence encoding a T cell Receptor (TCR) is done by nonhomologous end joining repair. In some embodiments, the nonhomologous end joining repair is generated by zinc finger nuclease, introduced into the cell by physical means, viral vector, or non-viral vector. In some embodiments, the nonhomologous end joining repair is generated by TALE nuclease, introduced into the cell by physical means, viral vector, or non-viral vector. In some embodiments, the modification of an endogenous sequence encoding a T cell Receptor (TCR) reduces or eliminates a level of expression of the alpha chain of the TCR. In some embodiments, the modification of an endogenous sequence encoding a T cell Receptor (TCR) reduces or eliminates a level of expression of beta chain of the TCR. In some embodiments, the modification of an endogenous sequence encoding a T cell Receptor (TCR) reduces or eliminates a level of expression of both the alpha chain and the beta chain TCR alpha chain.

In some embodiments, the modified T cell disclosed herein co-expresses at least one of the endogenous co-stimulatory molecules CD28, CD2, OX-40, ICOS, CD28, CD3, CD4, CD8 and CD40L or a combination thereof.

In some embodiments, the method disclosed herein further comprises a modification of an endogenous sequence encoding a component of major histocompatibility complex (MHC) class I (MHC-I), wherein the modification reduces or eliminates a level of expression or activity of the MHC-I. In some embodiments, the modification reduces or eliminates the expression or activity of β2-macroglobulin.

The present disclosure also provides a composition comprising the RTCR disclosed herein. The present disclosure also provides a composition comprising the nucleic acid encoding the RTCR disclosed herein. The present disclosure also provides a composition comprising the vector comprising the nucleic acid disclosed herein. The present disclosure also provides a composition comprising the cell disclosed herein. The present disclosure also provides a composition comprising the modified T cell disclosed herein.

The present disclosure also provides a composition comprising a population of cells, wherein the population comprises a plurality of the cell comprising the nucleic acid encoding or a vector comprising the nucleic acid encoding the RTCR disclosed herein. The present disclosure also provides a composition comprising a population of cells, wherein the population comprises a plurality of the modified T cell disclosed herein.

The present disclosure also provides a method of producing a plurality of modified T cells, wherein the method comprises: a) providing a plurality of primary T cells disclosed herein; b) providing a composition comprising the RTCR disclosed herein, the nucleic acid encoding the RTCR disclosed herein, or the vector comprising the nucleic acid encoding the RTCR disclosed herein; and c) introducing into the plurality of primary T cells of (a) the composition of (b), to produce a plurality of modified T cells under conditions that stably express the RTCR within the plurality of modified T cells. In some embodiments, the method of producing a plurality of modified T cells disclosed herein, further comprises a step of modifying an endogenous sequence encoding an endogenous T cell Receptor (TCR), wherein the modification reduces or eliminates a level of expression or activity of the endogenous TCR. In some embodiments, the method of producing a plurality of modified T cells disclosed herein, further comprises a step of modifying an endogenous sequence, wherein the modification reduces or eliminates a level of expression or activity of a major histocompatibility complex (MHC) class I (MHC-I).

In some embodiments, the modifying an endogenous sequence encoding a T cell Receptor (TCR) is done using a nucleic acid modifying system. In some embodiments, the modifying an endogenous sequence that reduces or eliminates a level of expression or activity of is done using a nucleic acid modifying system. In some embodiments, the nucleic acid modifying system is a one or more of a CRISPR/Cas protein, a Transcription Activator-Like Effector Nuclease (TALEN), a Zinc Finger Nuclease (ZFN), and an endonuclease. In some embodiments, the modifying an endogenous sequence is done by nonhomologous end joining repair. In some embodiments, the nonhomologous end joining repair is generated by zinc finger nuclease, introduced into the cell by physical means, viral vector, or non-viral vector. In some embodiments, the nonhomologous end joining repair is generated by TALE nuclease, introduced into the cell by physical means, viral vector, or non-viral vector. In some embodiments, the modifying an endogenous sequence encoding a T cell Receptor (TCR) reduces or eliminates a level of expression of the alpha chain of the TCR. In some embodiments, the modifying an endogenous sequence encoding a T cell Receptor (TCR) reduces or eliminates a level of expression of beta chain of the TCR. In some embodiments, the modifying an endogenous sequence encoding a T cell Receptor (TCR) reduces or eliminates a level of expression of both the alpha chain and the beta chain TCR alpha chain.

In some embodiments, the modifying an endogenous sequence that reduces or eliminates a level of expression or activity of a major histocompatibility complex (MHC) class I (MHC-I), wherein the modifying of an endogenous sequence reduces or eliminates a level of expression or activity of the MHC-I. In some embodiments, the modifying of an endogenous sequence reduces or eliminates the expression or activity of β2-macroglobulin.

In some embodiments, the method of producing a plurality of modified T cells disclosed herein, further comprises: d) maintaining or expanding the plurality of modified T cells in a suitable cell culture media; and e) either: i) cyropreserving the plurality of modified T cells in a suitable cell freezing media; or ii) preparing the plurality of modified T cells for administering to a subject suffering from a disease or disorder.

The compositions comprising the cells or modified T cells of the disclosure, and the plurality of modified T cells produced by the methods of the disclosure, intended for administration to a subject may be required to meet one or more “release criteria” that indicate that the composition is safe and efficacious for formulation as a pharmaceutical product and/or administration to a subject. Release criteria may include a requirement that a composition of the disclosure (e.g., a cell or modified T cell of the disclosure) comprises a particular percentage of cells or modified T cells expressing the RTCR of the disclosure on their cell surface. The expansion process should be continued until a specific criterion has been met (e.g., achieving a certain total number of cells or modified T cells of the disclosure or a certain percentage of total number of cells or modified T cells expressing the RTCR of the disclosure).

Certain criterion signal a point at which the expansion process should end. For example, cells should be formulated, reactivated, or cryopreserved once they reach a cell size of 300fL (otherwise, cells reaching a size above this threshold may start to die). Cryopreservation immediately once a population of cells reaches an average cell size of less than 300 fL may yield better cell recovery upon thawing and culture because the cells haven't yet reached a fully quiescent state prior to cryopreservation (a fully quiescent size is approximately 180 fL). Prior to expansion, T cells of the disclosure may have a cell size of about 180 fL, but may more than quadruple their cell size to approximately 900 fL at 3 days post-expansion. Over the next 6-12 days, the population of T cells will slowly decrease cell size to full quiescence at 180 fL.

A process for preparing a cell population for formulation may include, but is not limited to the steps of, concentrating the cells of the cell population, washing the cells, and/or further selection of the cells via drug resistance or magnetic bead sorting against a particular surface-expressed marker. A process for preparing a cell population for formulation may further include a sorting step to ensure the safety and purity of the final product. For example, if a tumor cell from a patient has been used to stimulate a modified T cell of the disclosure or that have been modified in order to stimulate a modified T cell of the disclosure that is being prepared for formulation, it is critical that no tumor cells from the patient are included in the final product.

In some embodiments, the cell disclosed herein, or the modified T cell disclosed herein, expresses on the cell surface the RTCR comprising a mutant CD137 or a mutant CD134 intracellular signaling domain disclosed herein, at a level that is at least about 2×, 3×, 4×, 5×, 6×, 7×, 8×, 9×, 10× or 20×, more as compared to the level of expression of a co-stimulatory molecule comprising a wild type CD137 or a wild type CD134 intracellular domain, respectively.

### Pharmaceutical Composition or Formulation

In some embodiments, the compositions disclosed herein, and the population of modified T cells produced using the methods disclosed herein, is in the form of a pharmaceutical formulation (or composition). In some embodiments, the pharmaceutical formulation disclosed herein comprises a pharmaceutically acceptable carrier. A pharmaceutical formulation of the disclosure may be distributed into bags for infusion, cryopreservation, and/or storage.

A pharmaceutical formulation of the disclosure may be cryopreserved using a standard protocol and, optionally, an infusible cryopreservation medium. For example, a DMSO free cryopreservant (e.g. CryoSOfree™ DMSO-free Cryopreservation Medium) may be used to reduce freezing-related toxicity. A cryopreserved pharmaceutical formulation of the disclosure may be stored for infusion to a patient at a later date. An effective treatment may require multiple administrations of a pharmaceutical formulation of the disclosure and, therefore, pharmaceutical formulations may be packaged in pre-aliquoted “doses” that may be stored frozen but separated for thawing of individual doses.

A pharmaceutical formulation of the disclosure may be stored at room temperature. An effective treatment may require multiple administrations of a pharmaceutical formulation of the disclosure and, therefore, pharmaceutical formulations may be packaged in pre-aliquoted “doses” that may be stored together but separated for administration of individual doses.

A pharmaceutical formulation of the disclosure may be archived for subsequent re-expansion and/or selection for generation of additional doses to the same patient in the case of an allogenic therapy who may need an administration at a future date following, for example, a remission and relapse of a condition.

As noted above, the disclosure provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one modified cell in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the disclosure provides an article of manufacture, comprising packaging material and at least one vial comprising a solution of at least one modified cell with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater.

The articles of manufacture of the present disclosure are useful for administration over a period ranging from immediate to twenty-four hours or greater. Accordingly, the presently claimed articles of manufacture offer significant advantages to the patient. Formulations of the disclosure can optionally be safely stored at temperatures of from about 2° C. to about 40° C. and retain the biological activity of the protein for extended periods of time, thus allowing a package label indicating that the solution can be held and/or used over a period of 6, 12, 18, 24, 36, 48, 72, or 96 hours or greater.

The products of the present disclosure include packaging material. The packaging material provides, in addition to the information required by the regulatory agencies, the conditions under which the product can be used.

The present disclosure also provided a method of treating a disease or disorder, comprising administering to a subject in need thereof a therapeutically effective number of the cell comprising the nucleic acid encoding or the vector comprising the nucleic acid encoding the RTCR disclosed herein, a therapeutically effective number of any one of the modified T cell disclosed herein, a therapeutically effective amount of any one of the compositions disclosed herein, or a therapeutically effective number of the plurality of modified T cells produced by the method disclosed herein.

In some embodiments, the subject is a mammal. In some embodiments, the mammal is any one of a human, a primate, a rodent, a canine, a feline, an ungulate, an equine and a porcine. In some embodiments, the mammal is a human. In some embodiments, the disease or disorder is any one of a cancer, an autoimmune disorder, an infectious disease, an inflammatory disease or condition, a renal disease or disorder, a lung disease or disorder, a liver disease or disorder, a cardiovascular system disease or disorder, a neurodegenerative disorder or condition, or a metabolic disorder or condition. In some embodiments, the cancer is a solid tumor or a hematologic cancer. In some embodiments, the infectious disease is caused by a bacteria, a virus, a fungi, a protozoa, or a parasite. In some embodiments, the neurodegenerative disorder or condition is any one of Alzheimer's disease (AD) and other dementias, Parkinson's disease (PD) and PD-related disorders, Prion disease, Motor neurone diseases (MND), Huntington's disease (HD), Spinocerebellar ataxia (SCA) or Spinal muscular atrophy (SMA).

The present disclosure provides a chimeric co-stimulatory intracellular protein (CIP) comprising a first and at least a second signal transduction domains, wherein the first and the at least second signal transduction domains are non-identical; and wherein the at least second signal transduction domain comprises a mutant intracellular signaling domain of a tumor necrosis factor receptor (TNFR) family protein.

The present disclosure also provides a chimeric co-stimulatory intracellular protein (CIP) comprising a first and at least a second signal transduction domains, wherein the first and the at least second signal transduction domains are non-identical; and wherein the at least second signal transduction domain comprises a mutant CD137 (4-1BB) intracellular domain or a mutant CD134 (OX-40) intracellular domain.

In some embodiments of the CIP disclosed herein, the mutant intracellular signaling domain of a TNFR family protein is any one of a mutant CD137 (4-1BB) intracellular domain or a mutant CD134 (OX-40) intracellular domain. In some embodiments, the CIP further comprises a transmembrane domain. In some embodiments of the CIP disclosed herein, the mutant CD137 intracellular domain is a truncated CD137 intracellular domain.

In some embodiments of the CIP disclosed herein, the truncated CD137 intracellular domain comprises an amino acid sequence according to amino acid position 13 to amino acid position 42 of the CD137 intracellular domain, of the present disclosure. In some embodiments of the CIP disclosed herein, the truncated CD137 intracellular domain comprises a deletion of a continuous stretch of one, two, three, four, five, six, seven, eight, nine, ten or more amino acids from the N-terminus the CD137 intracellular domain, of the present disclosure. In some embodiments of the CIP disclosed herein, the truncated CD137 intracellular domain comprises a deletion of one, two, three, four, five, six, seven, eight, nine, ten or more amino acids from amino acid position 1 to amino acid position 12 of the N-terminus of the CD137 intracellular domain, of the present disclosure. In some embodiments of the CIP disclosed herein, the CD137 intracellular domain of the present disclosure comprises an amino acid sequence according to SEQ ID NO: 1.

In some embodiments of the CIP disclosed herein, the truncated CD137 intracellular domain comprises an amino acid sequence according to SEQ ID NO: 3.

In some embodiments of the CIP disclosed herein, the mutant CD137 intracellular domain comprises a deletion of one, two, three or four lysine residue(s) from amino acid position 1 to amino acid position 12 of the N-terminus of the CD137 intracellular domain, of the present disclosure. In some embodiments of the CIP disclosed herein, the mutant CD137 intracellular domain comprises one or more lysine mutation(s) from amino acid position 1 to amino acid position 12 of the N-terminus of the CD137 intracellular domain, of the present disclosure. In some embodiments of the CIP disclosed herein, the mutant CD137 intracellular domain comprises one or more lysine mutation(s) at amino acid positions selected from amino acid positions 1, 5, 6 and 12 of the N-terminus of the CD137 intracellular domain, of the present disclosure.

In some embodiments of the CIP disclosed herein, the mutant CD137 intracellular domain comprises a deletion of one or more proximal basic amino acids from amino acid position 1 to amino acid position 12 of the N-terminus of the CD137 intracellular domain, of the present disclosure. In some embodiments of the CIP disclosed herein, the mutant CD137 intracellular domain comprises one or more proximal basic amino acid mutation(s) from amino acid position 1 to amino acid position 12 of the N-terminus of the CD137 intracellular domain, of the present disclosure. In some embodiments of the CIP disclosed herein, the mutant CD137 intracellular domain comprises one or more proximal basic amino acid mutation(s) at amino acid positions selected from amino acid positions 1, 2, 3, 4, 5 and 6 of the N-terminus of the CD137 intracellular domain, of the present disclosure. In some embodiments of the CIP disclosed herein, the mutant CD137 intracellular domain further comprises a lysine mutation at amino acid position 12 of the N-terminus of the CD137 intracellular domain, of the present disclosure.

In some embodiments of the CIP disclosed herein, the mutant CD134 intracellular domain is a truncated CD134 intracellular domain. In some embodiments of the CIP disclosed herein, the truncated CD134 intracellular domain comprises an amino acid sequence according to amino acid position 15 to amino acid position 37 of the CD134 intracellular domain, of the present disclosure. In some embodiments of the CIP disclosed herein, the truncated CD134 intracellular domain comprise a deletion of a continuous stretch of one, two, three, four, five, six, seven, eight, nine, ten, eleven, twelve, thirteen or more amino acids from the N-terminus of the CD134 intracellular domain, of the present disclosure. In some embodiments of the CIP disclosed herein, the truncated CD134 intracellular domain comprises a deletion of one, two, three, four, five, six, seven, eight, nine, ten, eleven, twelve, thirteen or more amino acids from amino acid position 1 to amino acid position 14 of the N-terminus of the CD134 intracellular domain, of the present disclosure.

In some embodiments of the CIP disclosed herein, the truncated CD134 intracellular domain comprises an amino acid sequence according to SEQ ID NO: 6.

In some embodiments of the CIP disclosed herein, the mutant CD134 intracellular domain comprises a deletion of a lysine residue from amino acid position 1 to amino acid position 14 of the N-terminus of the CD134 intracellular domain, of the present disclosure. In some embodiments of the CIP disclosed herein, the mutant CD134 intracellular domain comprises a lysine mutation at amino acid position 12 of the N-terminus of the CD134 intracellular domain, of the present disclosure.

In some embodiments of the CIP disclosed herein, the mutant CD134 intracellular domain comprises a deletion of one or more proximal basic amino acids from amino acid position 1 to amino acid position 14 of the N-terminus of the CD134 intracellular domain, of the present disclosure. In some embodiments of the CIP disclosed herein, the mutant CD134 intracellular domain comprises one or more proximal basic amino acid mutation(s) from amino acid position 1 to amino acid position 14 of the N-terminus of the CD134 intracellular domain. In some embodiments of the CIP disclosed herein, the mutant CD134 intracellular domain comprises one or more proximal basic amino acid mutation(s) at amino acid positions selected from amino acid positions 1, 2, and 5 of the N-terminus of the CD134 intracellular domain. In some embodiments of the CIP disclosed herein, the mutant CD134 intracellular domain further comprises a lysine mutation at amino acid position 12 of the N-terminus of the CD134 intracellular domain

In some embodiments, the CIP disclosed herein comprises a first signal transduction domain derived from a protein of the CD28 family. In some embodiments, the CIP disclosed herein comprises a first signal transduction domain derived from any one of CD28, CD28H, ICOS or a combination thereof.

In some embodiments, the CIP disclosed herein comprises a first signal transduction domain derived from ICOS. In some embodiments, the first signal transduction domain derived from ICOS comprises an amino acid sequence according to SEQ ID NO: 9.

In some embodiments, the CIP disclosed herein comprises a first signal transduction domain comprising a portion of a CD28 intracellular domain combined with an ICOS domain according to SEQ ID NO: 9. In some embodiments of the CIP disclosed herein, the first signal transduction domain comprises an amino acid sequence according to any one of SEQ ID NOs: 12 or 109. In some embodiments, the CIP disclosed herein comprises a first signal transduction domain derived from CD28. In some embodiments of the CIP disclosed herein, the first signal transduction domain derived from CD28 comprises an amino acid sequence according to SEQ ID NO: 10. In some embodiments of the CIP disclosed herein, the first signal transduction domain derived from CD28 comprises an amino acid sequence according to any one of SEQ ID NOs: 121-122. In some embodiments, the CIP comprises an amino acid sequence according to any one of SEQ ID NOs: 14-17.

In some embodiments, the CIP disclosed herein further comprises a third signal transduction domain. In some embodiments, the CIP disclosed herein further comprises a third signal transduction domain derived from any one of a CD3 signaling domain, a CD2 signaling domain or an interleukin 2 receptor binding (IL-2RB) protein signaling domain or a combination thereof. In some embodiments, the CD3 signaling domain of the CIP disclosed herein is derived form a CD3ζ or a CD3ε domain or a combination thereof. In some embodiments, the CD3 signaling domain of the CIP disclosed herein is a CD3 domain comprising an amino acid sequence according to any one of SEQ ID NOs: 18, 45, 46, 47 and 48.

In some embodiments, the third signal transduction domain of the CIP disclosed herein is a CD3ζ domain comprising an amino acid sequence having according to SEQ ID NO: 18. In some embodiments, the third signal transduction domain of the CIP disclosed herein is a CD3 domain comprising an amino acid sequence having at least 50%, 55%, 60%, 65%, 70%, 75%, 80%, 85%, 90%, 95%, 97% or 99% identity to according to SEQ ID NO: 18. In some embodiments, the third signal transduction domain of the CIP disclosed herein is a CD3ζ domain comprising an amino acid sequence having according to SEQ ID NO: 45. In some embodiments, the third signal transduction domain of the CIP disclosed herein is a CD3ζ domain comprising an amino acid sequence having at least 50%, 55%, 60%, 65%, 70%, 75%, 80%, 85%, 90%, 95%, 97% or 99% identity to according to SEQ ID NO: 45. In some embodiments, the third signal transduction domain of the CIP disclosed herein is a truncated CD3ζ domain comprising an amino acid sequence having according to SEQ ID NO: 46. In some embodiments, the third signal transduction domain of the CIP disclosed herein, the third signal transduction domain of the CIP disclosed herein is a truncated CD3 domain comprising an amino acid sequence having at least 50%, 55%, 60%, 65%, 70%, 75%, 80%, 85%, 90%, 95%, 97% or 99% identity to according to SEQ ID NO: 46. In some embodiments, the third signal transduction domain of the CIP disclosed herein is a CD3ε domain comprising an amino acid sequence according to SEQ ID NO: 47. In some embodiments, the third signal transduction domain of the CIP disclosed herein is a CD3ε domain comprising an amino acid sequence having at least 50%, 55%, 60%, 65%, 70%, 75%, 80%, 85%, 90%, 95%, 97% or 99% identity to SEQ ID NO: 47. In some embodiments, the third signal transduction domain of the CIP disclosed herein is a combination of a CD3ε and a truncated CD3ζ domains (CD3ζε domain). In some embodiments, the third signal transduction domain of the CIP disclosed herein is a CD3ζε domain comprising an amino acid sequence according to SEQ ID NO: 48. In some embodiments, the third signal transduction domain of the CIP disclosed herein is a CD3ζε domain comprising an amino acid sequence having at least 50%, 55%, 60%, 65%, 70%, 75%, 80%, 85%, 90%, 95%, 97% or 99% identity to SEQ ID NO: 48.

In some embodiments, the third signal transduction domain of the CIP disclosed herein is a CD2 signaling domain. In some embodiments, the third signal transduction domain of the CIP disclosed herein is a mutant CD2 signaling domain. In some embodiments, the mutant CD2 signaling domain is a truncated CD2 signaling domain. In some embodiments, the third signal transduction domain of the CIP disclosed herein is a CD2 signaling domain comprising an amino acid sequence according to SEQ ID NO: 49. In some embodiments, the third signal transduction domain of the CIP disclosed herein is a CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the third signal transduction domain of the CIP disclosed herein, is an IL-2RB protein signaling domain comprising an amino acid sequence according to SEQ ID NO: 50. In some embodiments, the third signal transduction domain of the CIP disclosed herein is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

In some embodiments, the CIP disclosed herein further comprises a fourth signal transduction domain. In some embodiments, the CIP disclosed herein further comprises a fourth signal transduction domain derived from any one of a CD3 signaling domain, a CD2 signaling domain or an interleukin 2 receptor binding (IL-2RB) protein signaling domain or a combination thereof, wherein the third and the fourth signal transduction domain are not identical. In some embodiments, the fourth signal transduction domain of the CIP disclosed herein, is derived form a CD3ζ or a CD3ε domain or a combination thereof. In some embodiments, the fourth signal transduction domain of the CIP disclosed herein is a CD3 domain comprising an amino acid sequence according to any one of SEQ ID NOs: 18, 45, 46, 47 and 48.

In some embodiments, the fourth signal transduction domain of the CIP disclosed herein is a CD3ζ domain comprising an amino acid sequence having according to SEQ ID NO: 18. In some embodiments, the fourth signal transduction domain of the CIP disclosed herein is a CD3ζ domain comprising an amino acid sequence having at least 50%, 55%, 60%, 65%, 70%, 75%, 80%, 85%, 90%, 95%, 97% or 99% identity to according to SEQ ID NO: 18. In some embodiments, the fourth signal transduction domain of the CIP disclosed herein is a CD3ζ domain comprising an amino acid sequence having according to SEQ ID NO: 45. In some embodiments, the fourth signal transduction domain of the CIP disclosed herein is a CD3ζ domain comprising an amino acid sequence having at least 50%, 55%, 60%, 65%, 70%, 75%, 80%, 85%, 90%, 95%, 97% or 99% identity to according to SEQ ID NO: 45. In some embodiments, the fourth signal transduction domain of the CIP disclosed herein is a truncated CD3ζ domain comprising an amino acid sequence having according to SEQ ID NO: 46. In some embodiments, the third signal transduction domain of the CIP disclosed herein, the fourth signal transduction domain of the CIP disclosed herein is a truncated CD3ζ domain comprising an amino acid sequence having at least 50%, 55%, 60%, 65%, 70%, 75%, 80%, 85%, 90%, 95%, 97% or 99% identity to according to SEQ ID NO: 46. In some embodiments, the fourth signal transduction domain of the CIP disclosed herein is a CD3ε domain comprising an amino acid sequence according to SEQ ID NO: 47. In some embodiments, the fourth signal transduction domain of the CIP disclosed herein is a CD3ε domain comprising an amino acid sequence having at least 50%, 55%, 60%, 65%, 70%, 75%, 80%, 85%, 90%, 95%, 97% or 99% identity to SEQ ID NO: 47. In some embodiments, the fourth signal transduction domain of the CIP disclosed herein is a combination of a CD3ε and a truncated CD3ζ domains (CD3ζε domain). In some embodiments, the fourth signal transduction domain of the CIP disclosed herein is a CD3ζε domain comprising an amino acid sequence according to SEQ ID NOs: 48. In some embodiments, the fourth signal transduction domain of the CIP disclosed herein is a CD3ζε domain comprising an amino acid sequence having at least 50%, 55%, 60%, 65%, 70%, 75%, 80%, 85%, 90%, 95%, 97% or 99% identity to SEQ ID NO: 48.

In some embodiments, the fourth signal transduction domain of the CIP disclosed herein is a CD2 signaling domain. In some embodiments, the fourth signal transduction domain of the CIP disclosed herein is a mutant CD2 signaling domain. In some embodiments, the mutant CD2 signaling domain is a truncated CD2 signaling domain. In some embodiments, the fourth signal transduction domain of the CIP disclosed herein is a CD2 signaling domain comprising an amino acid sequence according to SEQ ID NO: 49. In some embodiments, the fourth signal transduction domain of the CIP disclosed herein is a CD2 signaling domain comprising an amino acid sequence having at least 50%, 55%, 60%, 65%, 70%, 75%, 80%, 85%, 90%, 95%, 97% or 99% identity to SEQ ID NO: 49.

In some embodiments, the fourth signal transduction domain of the CIP disclosed herein, is an IL-2RB protein signaling domain comprising an amino acid sequence according to SEQ ID NO: 50. In some embodiments, the fourth signal transduction domain of the CIP disclosed herein is an IL-2RB protein signaling domain comprising an amino acid sequence having at least 50%, 55%, 60%, 65%, 70%, 75%, 80%, 85%, 90%, 95%, 97% or 99% identity to SEQ ID NO: 50.

In some embodiments, the CIP disclosed herein is for expression in a T cell, wherein the T cell co-expresses at least one of the endogenous co-stimulatory molecules CD28, CD2, OX-40, ICOS, CD28, CD3, CD4, CD8 and CD40L or a combination thereof.

In some embodiments, the CIP disclosed herein, is co-expressed with a T cell receptor (TCR) in a T cell. In some embodiments, the TCR is an endogenous TCR. In some embodiments, the TCR is an artificial TCR. In some embodiments, the artificial TCR is an affinity enhanced TCR. In some embodiments, the CIP when co-expressed with a TCR in a T cell provides a second activation signal for inducing activation and proliferation of the T cell, wherein the first activation signal is provided by antigen binding by the TCR.

In some embodiments, the CIP disclosed herein, is expressed in a T cell as a component of an artificial receptor for a target. In some embodiments, the artificial receptor is a chimeric antigen receptor (CAR), a receptor for a ligand or a component thereof, an antibody or a fragment thereof. In some embodiments, the CIP disclosed herein, is expressed as a component of a CAR. In some embodiments, the CIP disclosed herein, is expressed as a component of an antibody or a fragment thereof. In some embodiments, the antibody or a fragment thereof is a Fab fragment, a F(ab)2 fragment, a diabody, a nanobody, a sdAb, a Fv, a VHH fragment, or a single chain Fv fragment. In some embodiments, the CIP expressed as a component of an artificial receptor in a T cell, as disclosed herein induces activation and proliferation of the T cell upon target binding by the artificial receptor.

The term “about” or “approximately” can be understood as within 10%, 9%, 8%, 7%, 6%, 5%, 4%, 3%, 2%, 1%, 0.5%, 0.1%, 0.05%, or 0.01% of the stated value. In some embodiments, “about” or “approximately” can be understood as within 5%, 4%, 3%, 2%, 1%, 0.5%, 0.1%, 0.05%, or 0.01% of the stated value. In some embodiments, “about” or “approximately” can be understood as within 2%, 1%, 0.5%, 0.1%, 0.05%, or 0.01% of the stated value. In some embodiments, “about” or “approximately” can be understood as within 1%, 0.5%, 0.1%, 0.05%, or 0.01% of the stated value.

The following examples are provided to better illustrate the present disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### EXAMPLES

### Materials and Methods

### Media and Cell Lines

DMEM was supplemented with Penn/Strep/Glutamine, 20 mM HEPES, 10 μg/mL Gentamycin and 10% FBS to make complete DMEM. RPMI was supplemented with Penn/Strep/Glutamine, 20 mM HEPES, 10 μg/mL Gentamycin, 10% FBS, and 50 uM 2-ME to make complete RPMI. T cell growth media was made by supplementing complete RPMI with 50 ng/ml IL2, 10 ng/ml IL7, and 10 ng/mL IL15 (Peprotech). X-Vivo15 was supplemented with 1% Human Serum, 20 mM HEPES, Penn/Strep/Glutamine, and 10 μg/mL Gentamycin to make Cytokine Media. Human PBMCs were purchased from iSpecimen and cultured in complete RPMI. 293FT were purchased from Invitrogen. K562 and A375 cells were purchased from ATCC and cultured in complete DMEM.

Plasmids and Cloning A lentiviral plasmid containing the PGK promoter driving a truncated human EGFR receptor (huEGFRt) followed by the MSCV promoter driving GFP and a subsequent WPRE sequence was ordered from vector builder. Co-stimulatory molecules followed by a P2A sequence were ordered as a single gene block (Invitrogen) and placed in frame with the huEGFRt sequence using NEB builder homology-based recombination. CAR and TCR sequences were constructed from 3 gene block fragments (Invitrogen) and cloned with NEB builder downstream of the MSCV promoter following GFP excision. PD-L1_P2A and HLA-A2 were cloned in frame with the huEGFRt and in place of GFP, respectively.

P2A amino acid sequence
(SEQ ID NO: 111)
GSGATNFSLLKQAGDVEENPGP
Human EGFRt amino acid sequence (Other name:
huEGFRt (AA112))
(SEQ ID NO: 112)
MLLLVTSLLLCELPHPAFLLIPRKVCNGIGIGEFKDSLSINATNIK
HFKNCTSISGDLHILPVAFRGDSFTHTPPLDPQELDILKTVKEITG
FLLIQAWPENRTDLHAFENLEIIRGRTKQHGQFSLAVVSLNITSLG
LRSLKEISDGDVIISGNKNLCYANTINWKKLFGTSGQKTKIISNRG
ENSCKATGQVCHALCSPEGCWGPEPRDCVSCRNVSRGRECVDKCNL
LEGEPREFVENSECIQCHPECLPQAMNITCTGRGPDNCIQCAHYID
GPHCVKTCPAGVMGENNTLVWKYADAGHVCHLCHPNCTYGCTGPGL
EGCPTNGPKIPSIATGMVGALLLLLVVALGIGLFM
HLA-A2 signal peptide
(SEQ ID NO: 113)
MAVMAPRTLVLLLSGALALTQTWA
huGMCSF Signal Peptide, amino acid sequence
(SEQ ID NO: 119)
MLLLVTSLLLCELPHPAFLLIP
P2A nucleic acid sequence
(SEQ ID NO: 114)
GGATCCGGCGCCACCAATTTCAGCCTGCTGAAACAGGCTGGCGACG
TGGAAGAGAACCCTGGACCT
Human EGFRt nucleic acid sequence
(SEQ ID NO: 115)
ATGCTGCTGCTGGTTACATCTCTGCTGCTGTGCGAGCTGCCCCATC
CTGCCTTTCTGCTGATCCCCAGAAAAGTGTGCAACGGCATCGGCAT
CGGAGAGTTCAAGGACAGCCTGAGCATCAACGCCACCAACATCAAG
CACTTCAAGAACTGCACCAGCATCAGCGGCGACCTGCACATTCTGC
CTGTGGCCTTTAGAGGCGACAGCTTCACCCACACACCTCCACTGGA
CCCTCAAGAGCTGGACATCCTGAAAACCGTGAAAGAGATCACCGGA
TTTCTGTTGATCCAGGCTTGGCCCGAGAACCGGACAGATCTGCACG
CCTTCGAGAACCTGGAAATCATCAGAGGCCGGACCAAGCAGCACGG
CCAGTTTTCTCTGGCTGTGGTGTCCCTGAACATCACCAGCCTGGGC
CTGAGAAGCCTGAAAGAAATCAGCGACGGCGACGTGATCATCTCCG
GCAACAAGAACCTGTGCTACGCCAACACCATCAACTGGAAGAAGCT
GTTCGGCACCAGCGGCCAGAAAACAAAGATCATCAGCAACCGGGGC
GAGAACAGCTGCAAGGCTACAGGCCAAGTGTGCCACGCTCTGTGTA
GCCCTGAAGGCTGTTGGGGACCCGAGCCTAGAGATTGCGTGTCCTG
TCGGAATGTGTCCCGGGGCAGAGAATGCGTGGACAAGTGCAATCTG
CTGGAAGGCGAGCCCCGCGAGTTCGTGGAAAACAGCGAGTGCATCC
AGTGTCACCCCGAGTGTCTGCCCCAGGCCATGAACATTACCTGTAC
CGGCAGAGGCCCCGACAACTGTATTCAGTGCGCCCACTACATCGAC
GGCCCTCACTGCGTGAAAACATGTCCTGCTGGCGTGATGGGAGAGA
ACAACACCCTCGTGTGGAAGTATGCCGACGCCGGACATGTGTGCCA
CCTGTGTCACCCTAATTGCACCTACGGCTGTACAGGCCCTGGCCTG
GAAGGCTGTCCAACAAACGGACCTAAGATCCCCTCTATCGCCACCG
GCATGGTTGGAGCCCTGCTGCTTCTGCTGGTGGTGGCCCTTGGAAT
CGGCCTGTTCATGTGA
HLA-A2 signal peptide nucleic acid sequence
(SEQ ID NO: 116)
ATGGCTGTGATGGCCCCTAGAACACTGGTGCTGCTGCTGTCTGGTG
CCCTGGCTCTGACTCAGACATGGGCC
CD8a signal peptide nucleic acid sequence
(SEQ ID NO: 148)
ATGGCTCTGCCTGTGACAGCTCTGCTGCTGCCTCTGGCTCTGCTTC
TGCATGCCGCTAGACCT
CD8a Hinge
(SEQ ID NO: 149)
ACCACCACCCCCGCCCCCAGACCCCCCACCCCCGCCCCCACCATCG
CCAGCCAGCCCCTGAGCCTGAGACCCGAGGCCTGCAGACCCGCCGC
CGGCGGCGCCGTGCACACCAGAGGCCTGGACTTCGCCTGC
huGMCSF Signal Peptide, nucleic acid sequence
(SEQ ID NO: 150)
ATGCTGCTGCTGGTTACATCTCTGCTGCTGTGCGAGCTGCCCCATC
CTGCCTTTCTGCTGATCCCC

### Lentiviral Production and Preparation of Retronectin Plates

VSV pseudotyped lentivirus was produced in 6 well plates. In brief, 293FT were seeded the night before or the day of at 0.9×10⁶ or 1.4×10⁶ cells/well, respectively. Once the cells had adhered and reached at least 80% confluency a mix of lentiviral plasmid, packaging vector (psPAX2) and VSV-G envelope expressing plasmid (PMD2.G) were transfected using lipofectamine 3000 (Invitrogen), according to the manufacturer's protocol. After 18 hrs, the media was replaced with 3mLs of fresh DMEM. Viral supernatants were harvested 48 hrs following changing the media and spun down at 1500RPM to remove 293FT cell/debris. Retronectin was coated on 24 well non-tissue culture treated plates at 20 m/well in PBS−/− for 2 hrs at 37° C. or overnight at 4° C. Retronectin was removed and washed once with PBS prior to addition of lentiviral SN (2mLs). The plate was then spun at 1500 G for 90 minutes at 32° C. to concentrate viral particles onto the retronectin. Lentiviral SN was removed immediately prior to transduction of primary T cells or tumor cells. Alternatively, T cells were transduced with polybrene at 8 ug/mL with a spinfection of 800 G for 2 hrs at 32° C.

### T Cell Culture, Transduction, and Isolation

Human PBMCs were activated in T cell growth media with CD3/28 microbeads (Invitrogen) in complete RPMI (100 ul beads/50×10⁶ PBMCs). 48 hrs after activation, activated PBMCs were transferred to Lentiviral-coated Retronectin plates for 48 hrs before being transferred to 6 well plates containing fresh T cell growth media. After an additional 24 hrs in culture cell transduction was determined by flow cytometry and transduced cells were enriched based on huEGFRt expression. To isolate cells based on EGFR expression, T cells cultures were collected and activation beads removed. Cells were then stained in 1:100 anti-EGFR-APC antibody in MACS buffer at 4° C. for 30 minutes. Cells were then washed and incubated with anti-APC microbeads (Miltenyi) for 15-30 minutes at 4° C. Unbound microbeads were then removed by centrifugation and huEGFRt cells were isolated by positive selection on mini-macs columns. Cells were eluted from the mini-MACS columns and put back into culture in T cell growth media and used within 2 weeks for experiments. To create stable cell lines, cells were collected and transduced as with primary T cells. EGFR selection was performed twice, two weeks apart.

### T Cell Stimulation

In the case where T cells were stimulated with plate bound antibodies, maxisorp Flat-bottom plates (Invitrogen) were coated with the indicated amount of anti-human CD3 antibody (HIT3a-Biolegend) in PBS−/− for 2 hrs at 37° C. Plates were washed twice in basal RPMI before use. For K562 stimulation K562 cells were collected and resuspended in Cytokine media and aliquoted to U-bottom plates. Similarly, A375 cells were plated 1 day prior to the addition of T cells in DMEM in 96 well flat-bottom plates. The media was exchanged prior to the addition of cognate T cells. Following EGFR+ selection, T cells were collected, counted, and resuspended at the appropriate concentration in Cytokine media and distributed to antibody or APC-bearing wells. For K562 experiments anti-CD3 (HIT3a/Biolegend) was added at the indicated dose following 1-2 hrs of K562/T cell interaction at 37° C. In the case where T cell proliferation was to be tracked, T cells were labelled with Violet Tracking Dye (CTV) according to Biolegend's protocol prior to the addition to stimulatory plates. Supernatant was collected at 18-36 hr post stimulation to assess cytokine secretion and proliferation/T cell killing was assessed following 96 hrs of stimulation.

### Cytokine Multiplex Assay

Following collection of T cell supernatants cytokines were measured with the Legendplex Multi-Analyte Flow Assay Kit foe human Th or Th1 cytokines (Biolegend). Manufactures protocol was followed with the following exceptions: 75 uL T cell SN was used to measure cytokines and 2 uL of each reagent was used/well. Secreted cytokines were measured by flow cytometry and the values were normalized to the maximal response of the control group in order to combine and analyze multiple experiments and normalize for variability between experiments and donors.

### Conjugation Assays

To assess conjugation of T cells to target cells, T cells were labelled with CFSE (Biolegend) and K562_HLA-A2 or K562_HLA-A2_PD-L1 cells were labelled with CTV according to manufacturer's protocols. T cells and APCs were mixed in a 1:2 ratio and briefly centrifuged in a 1.5 mL eppendorf tube to encourage conjugation. Cell pellets were incubated at 37° C. for 30 minutes and then cell pellets were gently resuspended by repeat pipetting (20×) with a p200 and a cut-off pipette tip and assessed immediately by flow cytometry.

### Flow Cytometry

Cells were collected and washed in MACS Buffer (PBS−/−, 1% FBS, 1 mM EDTA) before being stained in MACS buffer containing relevant antibodies. Anti-EGFR-APC, anti-mouse TCRbeta-FITC, anti-human PD1-PE, anti-CD3 APC-Cy7, anti-CD8 PE-Cy7 were all purchased from biolegend. Following addition of antibodies, cells were stained for 30-60 minutes at 4° C. For the detection of CD-19 CAR expression cells were incubated with CD-19Fc recombinant protein in MACS buffer at 1 μg/mL for 30 minutes at RT. Cells were then washed and incubated with anti-human FC antibody at 1:100 dilution. Cells were then washed 3× in MACS buffer and analyzed on an Acea NovoCyte flow cytometer. Cells were collected at constant volume, allowing for accurate cell counts to be obtained.

### Example 1: Design of Co-Stimulatory Molecules Comprising Chimeric Intracellular Signaling Domains

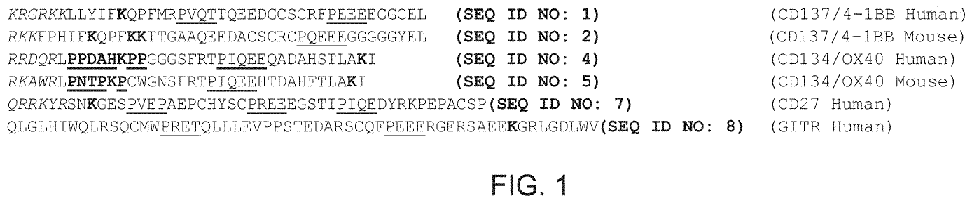

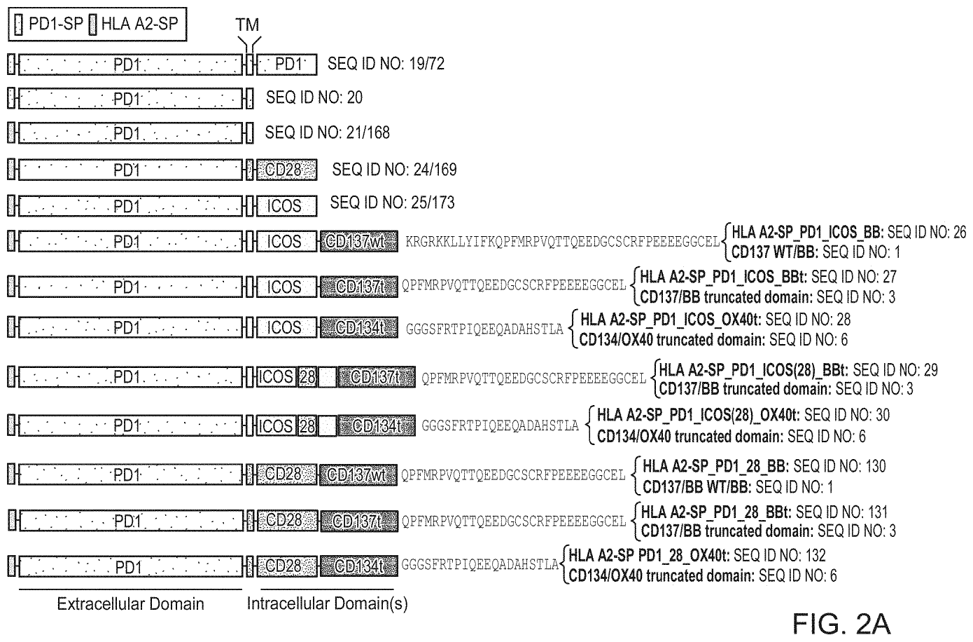

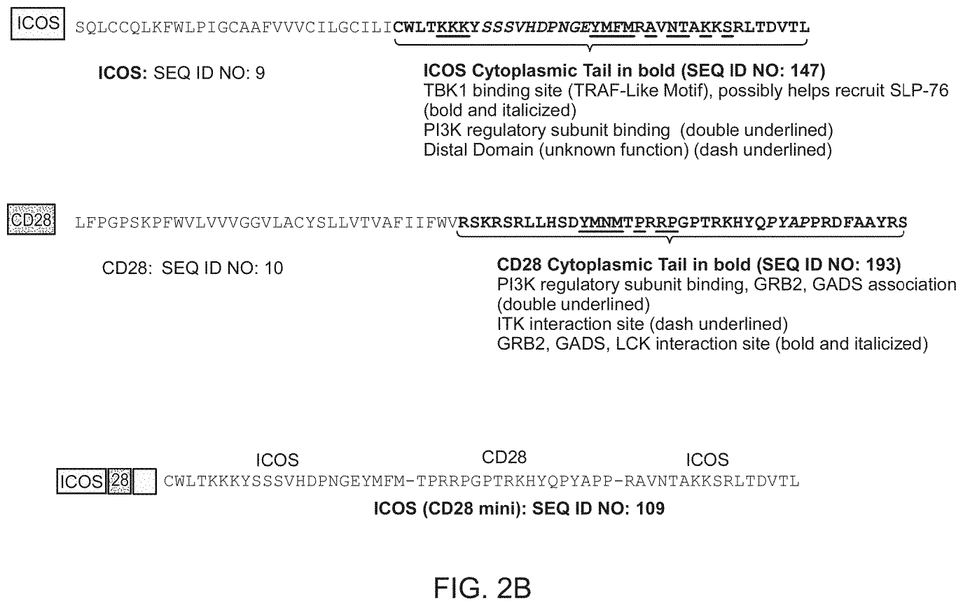

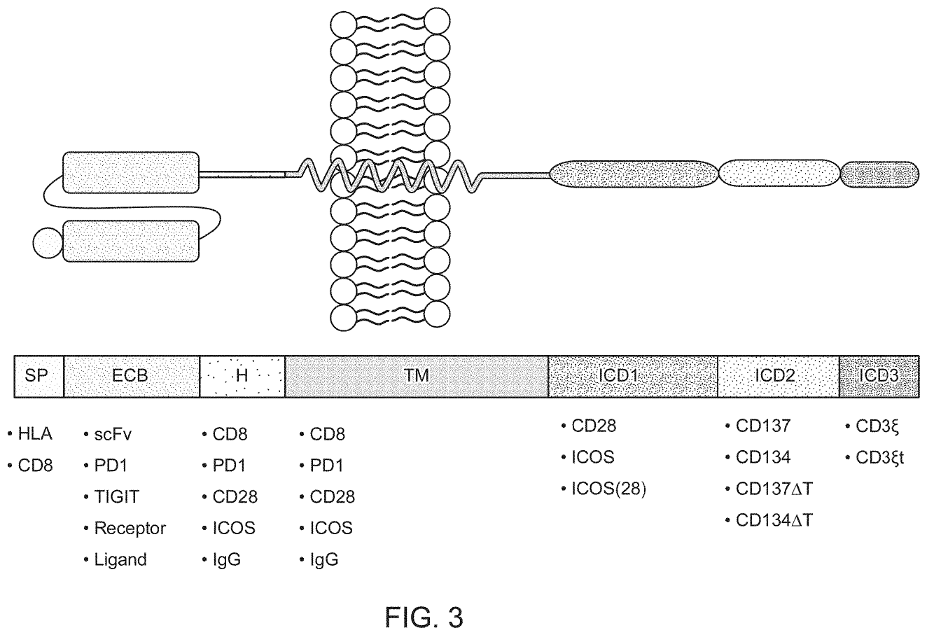

The disclosure herein provides the design of the co-stimulatory molecules comprising intracellular signaling domains comprising or derived from CD137/4-1BB or CD134/OX-40 receptors as depicted in FIG. 1. Examination of the sequence of the CD137 family of cytoplasmic tails (FIG. 1) showed a common membrane-proximal polybasic domain as well as several lysine residues that could serve as ubiquitination sites, as well as the TRAF binding domain that serves to activate the NF-κB signaling pathway following receptor ligation. Without being bound by the theories, the conserved lysine residues may function as ubiquitination sites that could control the ubiquitination and degradation CD134/CD137 and that the disrupted location of the CD137 or CD134 cytoplasmic tail in the potential CD28/ICOS-CD137 CAR or CD28/ICOS-CD134 CAR receptors, respectively, could be affecting the localization or half-life of the resulting molecule, through either the poly-basic domain or the conserved lysine residues. New fusion domains of ICOS/CD28 intracellular domain and the cytoplasmic domains of CD137 or OX-40 lacking the polybasic sequence and the conserved lysine residues, as well as their wild-type (WT) counterparts were generated (FIGS. 2A and 2B). A portion of the cytoplasmic domain of CD28 responsible for the binding of Lck and Vav3 to possible enhance stimulation was also included. The extracellular domain of PD-1 was used, creating either dominant-negative (DN) version by omitting the intracellular tail or an inhibitory-switch receptor that would change a negative regulatory signal into a positive one, thus providing a cell-intrinsic PD-1 blockade. The cytoplasmic tail, i.e., intracellular co-stimulatory domain, described herein can be expanded through the use of cytoplasmic tails of other signaling proteins of interest to create new CAR receptors or different inhibitory-switch receptors, or express other immune-modulatory extracellular domains, as detailed in FIG. 3.

### Example 2: Generation and Testing of the In-Vitro Functionality of Checkpoint Co-Stimulatory Molecules

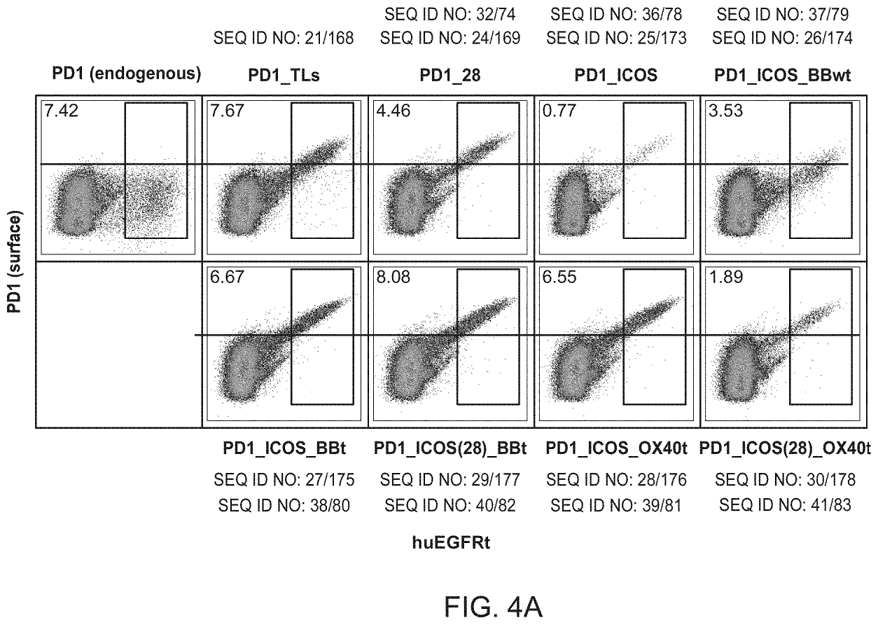

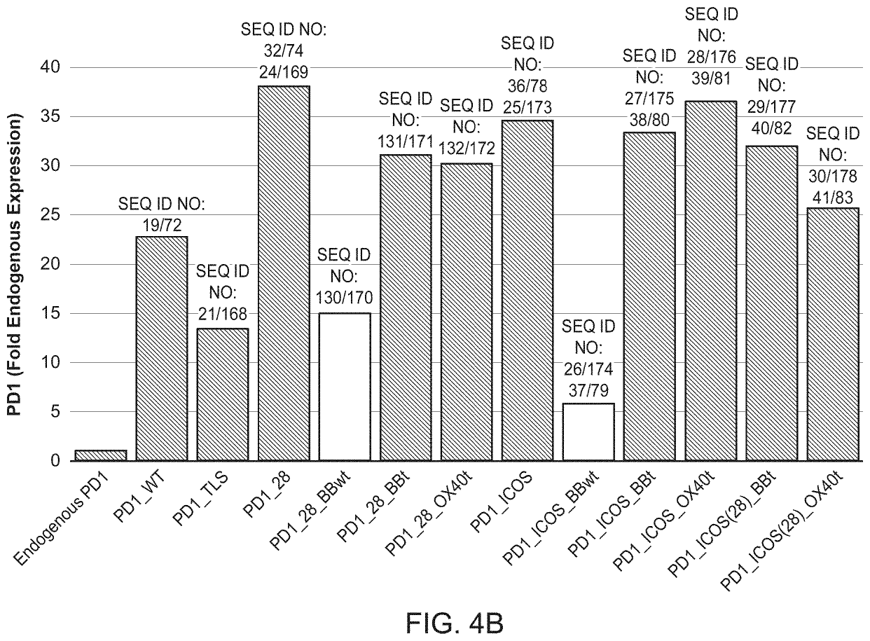

The disclosure herein provides the design of the co-stimulatory molecules and validation of their effect on function of a high affinity TCR. The co-stimulatory molecules described herein were designed as depicted in FIGS. 2A and 2B. In all recombinant receptors, the PD1 signal peptide (SP) was exchanged for the signal peptide from HLA-A2, which increases the surface expression of the receptor. As controls, the PD1-WT and a truncated PD-1 lacking the ITIM-containing intracellular tail (TLs) were included. Two second-generation receptors, containing the transmembrane and intracellular domains of CD28 or ICOS were included as 2^{nd}-generation control receptors. Additionally, fifteen 3^{rd}-generation receptors containing the transmembrane and intracellular domains derived from ICOS and/or CD28 linked to an intracellular signaling domain of a TNF-Receptor super family member were generated. The first contained the intracellular domain of wild type CD137/4-1BB (HLA A2-SP_PD1_ICOS_BB: SEQ ID NO: 26), while the second and third contained either the CD137/4-1BB domain or the CD134/OX-40 intracellular domain with key mutations incorporated to increase surface expression (HLA A2-SP_PD1_ICOS_BBt: SEQ ID NO: 27 and HLA A2-SP_PD1_ICOS_OX40t: SEQ ID NO: 28, respectively). Further 3^{rd} generation receptors described herein contain a chimeric intracellular domain comprising a portion of CD28 intracellular domain inserted within an ICOS intracellular domain that is further linked to either the mutated CD137 (ICOS4BBt) or mutated CD134/OX40 (ICOS-OX40t) domains (HLA A2-SP_PD1_ICOS(28) BBt: SEQ ID NO: 29 and HLA A2-SP_PD1_ICOS(28)_OX40t: SEQ ID NO: 30). Two more 3^{rd} generation receptors were created as described herein containing a CD28 intracellular domain linked to either mutated CD137 (28_BBt) or mutated CD134/OX40 (28_OX40t) domains (HLA A2-SP_PD1_28_BBt: SEQ ID NO: 131 and HLA A2-SP_PD_28_OX40t: SEQ ID NO: 132) (FIG. 2A). These vectors were cloned into a lentiviral vector and fused by a self-cleaving peptide to a truncated huEGFR receptor (huEGFRt) for tracking transduced cells and magnetic selection. When these receptors were expressed by lentiviral transduction into primary T cells, each receptor expresses significantly over endogenous PD-1 levels (FIG. 4A). While the 2^{nd} generation co-stimulatory molecules were well expressed, the inclusion of the CD137 (4-1BB) intracellular domain resulted in a considerable decrease in surface expression of the recombinant receptor. The disclosure herein shows that inclusion of the mutated intracellular domains, which maintain the TRAF-binding domains, rescues the surface expression of these optimized 3^{rd} generation receptors. The surface expression of huEGFRt and co-stimulatory molecules demonstrates the increased expression of the truncated CD137 (4-1BB) design (ICOS_BBt) compared to the non-truncated version (ICOS_BBwt). Similar mutations in the cytoplasmic domain of CD134 (OX-40) also resulted in high surface expression of the co-stimulatory molecules (FIG. 4B).

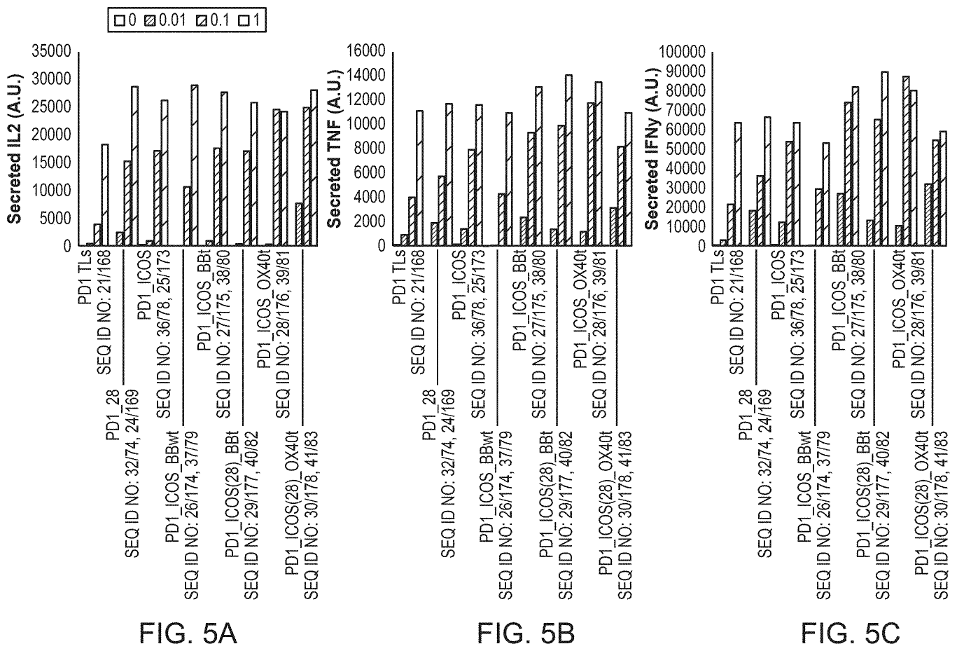

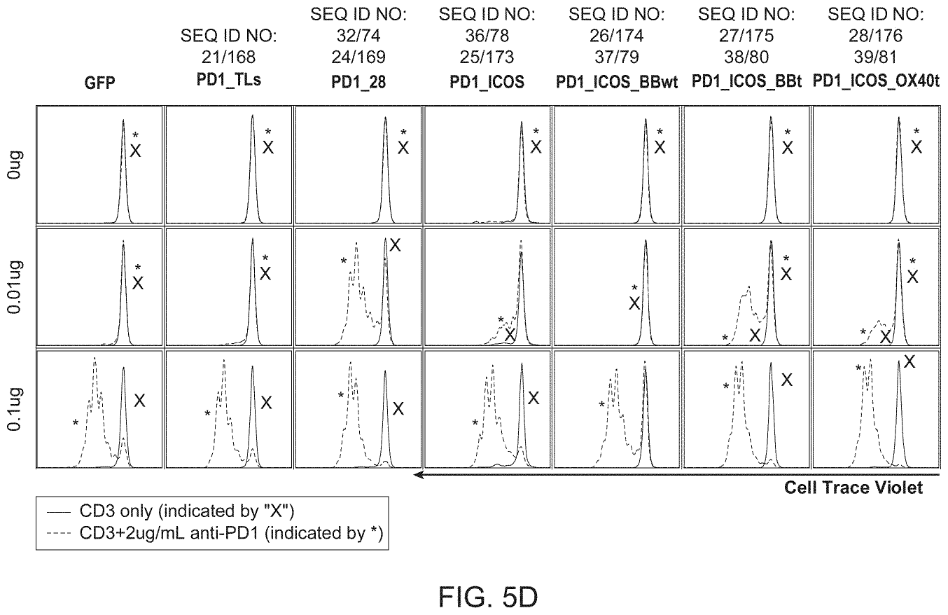

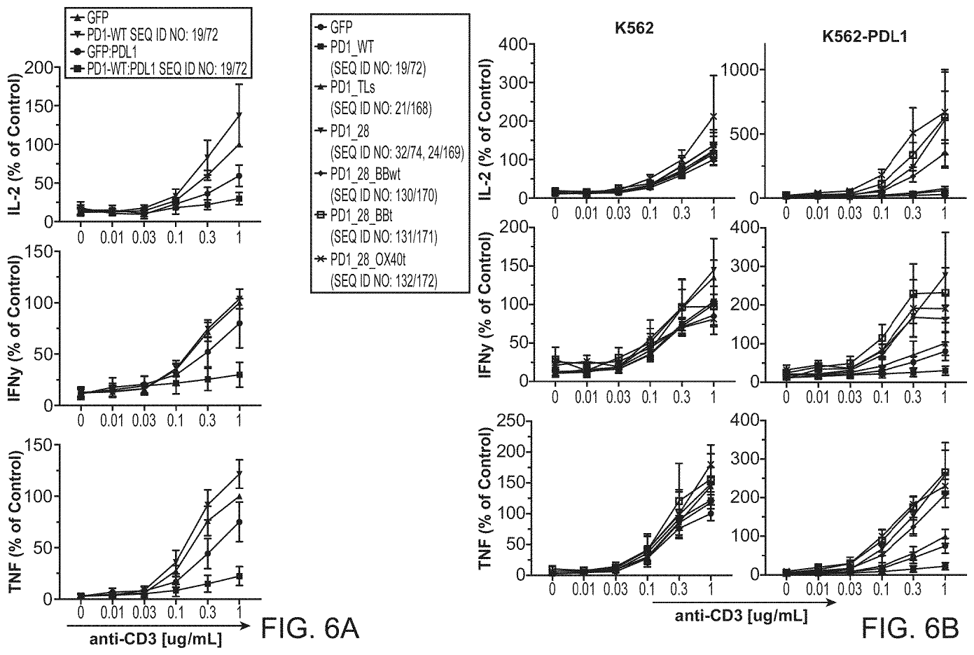

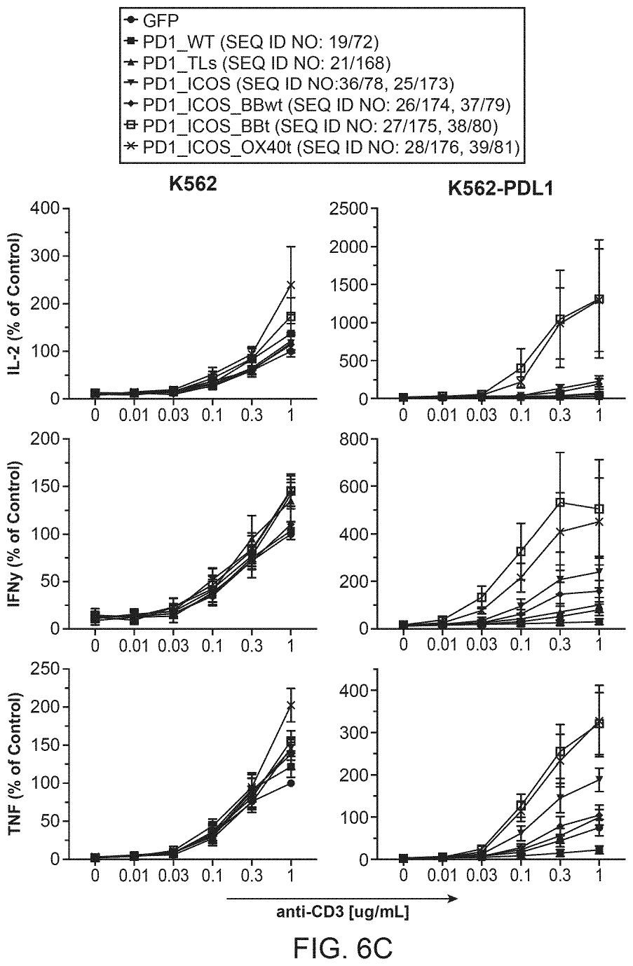

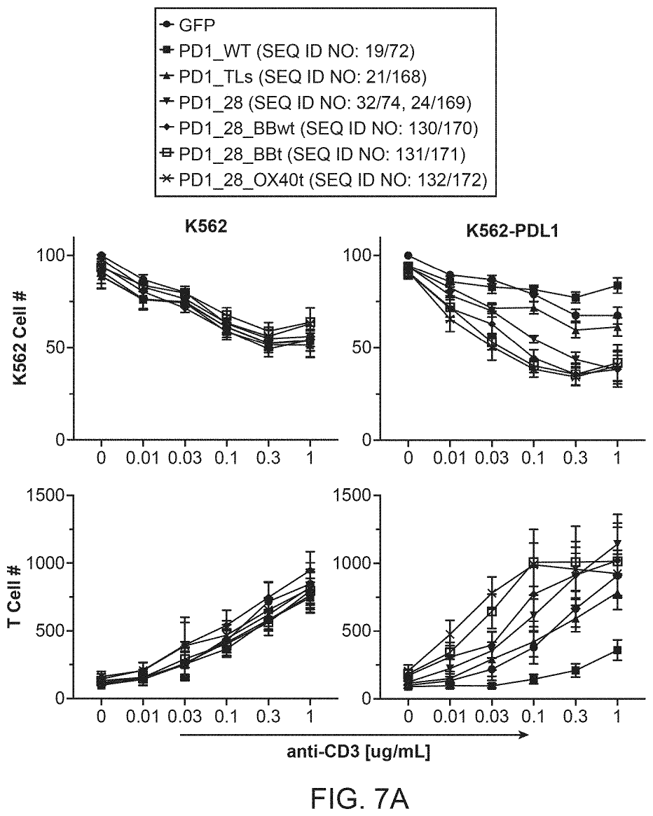

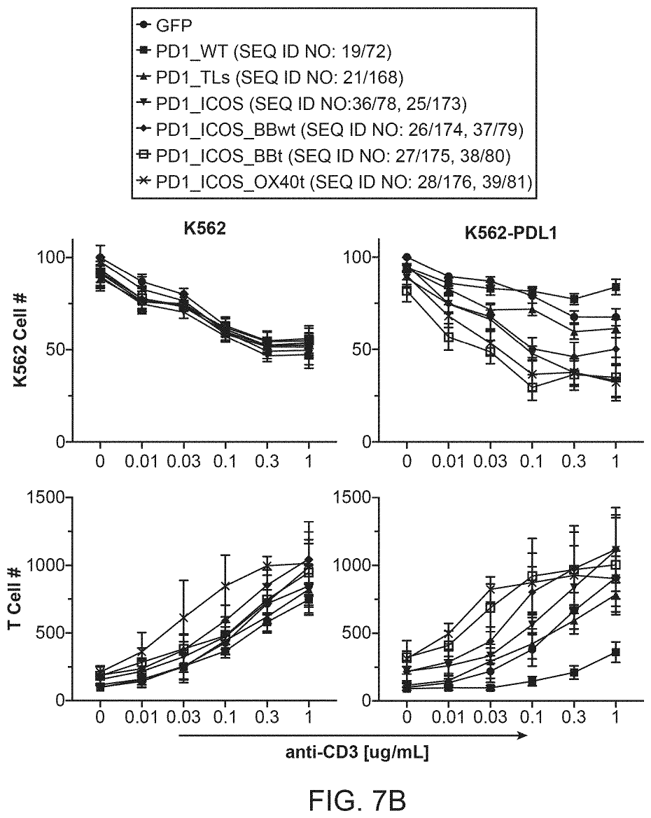

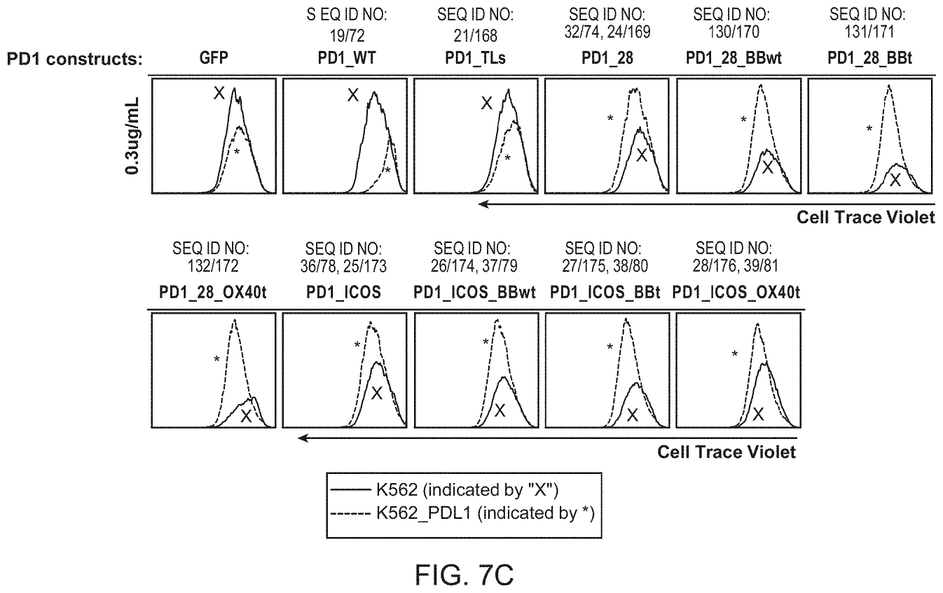

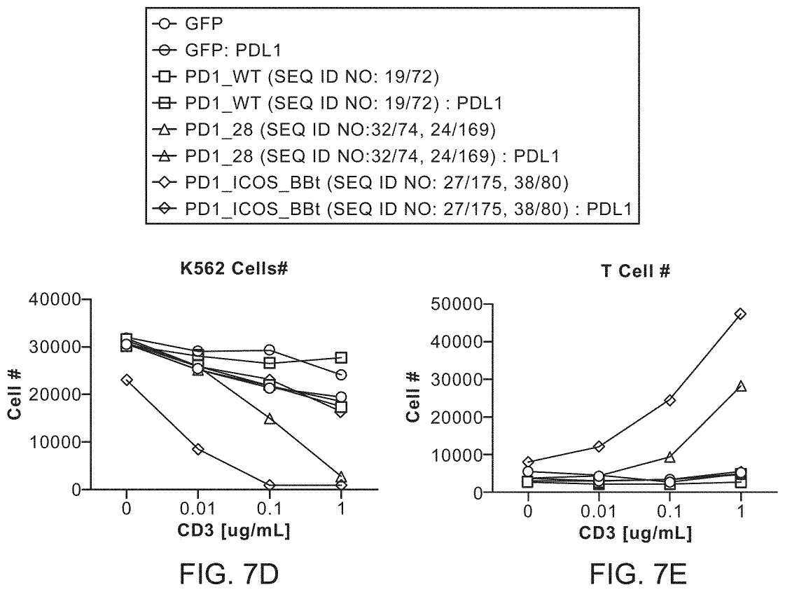

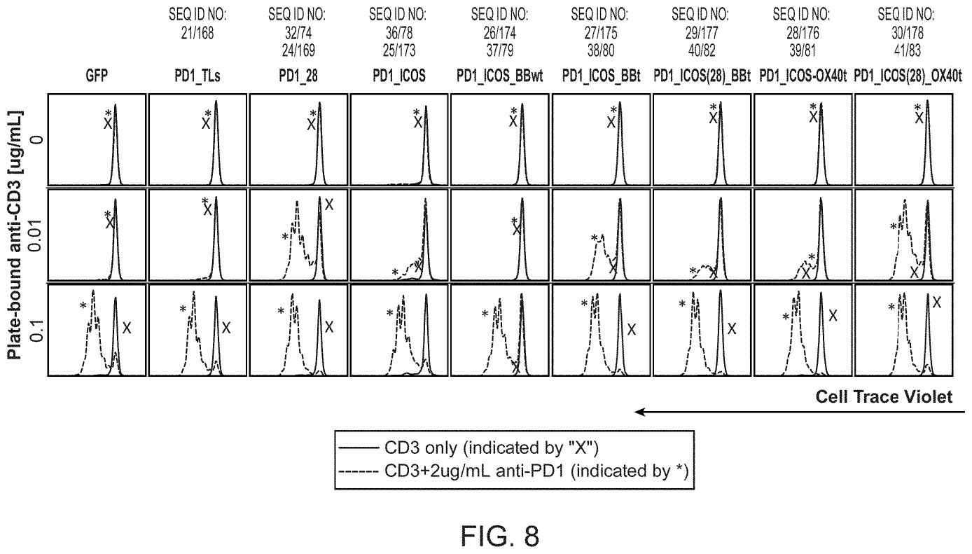

Following transduction, T cells were isolated based on the expression of huEGFRt, to >90% purity, and used in restimulation experiments. The results disclosed herein demonstrate that, in-vitro, engagement of co-stimulatory molecule enhanced T cell cytokine production and proliferation, especially at lower doses of anti-CD3 antibody (FIGS. 5A-5D). To make a more physiological system, either HLA-A2 alone or HLA-A2 alongside PD-L1 were overexpressed on K562 cells and incubated with the co-stimulatory receptor-transduced T cells and the indicated dose of anti-CD3. Incubation with K562: PD-L1 cells reduced the amount of secreted cytokine, especially with T cells overexpressing PD1_WT (FIG. 6A). While expression of either PD1_TLs or PD1_ICOS_BBwt did little to affect the secretion of IL-2, TNF, or IFNγ, the expression of PD1_28 or PD1_ICOS increased effector cytokine secretion 3-4 fold over GFP control in the presence of PD-L1 expressing K562 cells. Notably, both PD1_ICOS_BBt and PD1_ICOS_OX40t co-stimulatory molecules further improved on this effect, increasing effector cytokine secretion 2 to 3-fold over PD1_ICOS expressing cells in the presence of PD-L1 (FIG. 6C). The expression of PD1_28, PD1_28_BBt and PD1_28_OX40t resulted in comparable effector cytokine secretion (FIG. 6B). None of the constructs were constitutively active and had minimal effect on cytokine secretion in the absence of PD-L1 or anti-CD3, indicating the necessity of both PD-1 and antigen to initiate a T cell response. Fitting with the cytokine data, T cells expressing a) PD1_28, PD1_28_BBt and PD1_28_OX40t (FIG. 7A, lower panels, and FIG. 7C), and b) PD1_ICOS_BBt and PD1_ICOS_OX40t (FIG. 7B, lower panels, and FIG. 7C), proliferated best in response to K562 cells expressing PD-L1 and were best able to kill PD-L1 expressing K562 cells (FIGS. 7A-7B, upper panels). Fitting with the cytokine data, T cells expressing PD1_ICOS_BBt and PD1_ICOS_OX40t proliferated best in response to K562 cells expressing PD-L1 and were best able to kill PD-L1 expressing K562 cells (FIG. 7C-7E). Again, this response required both anti-CD3 and PD-L1 expression. The co-stimulatory molecules demonstrated co-stimulatory ability as their expression increased T cell proliferation when cells were stimulated on 96-well plates coated with anti-CD3 and anti-PD1 (FIG. 8).

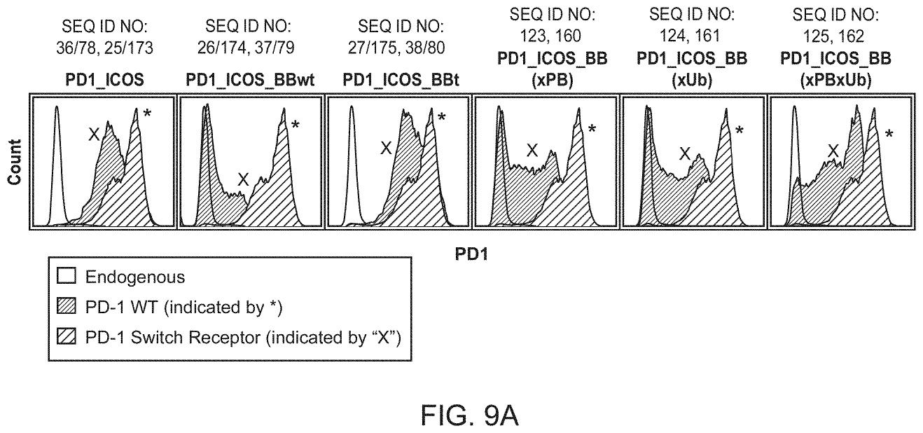

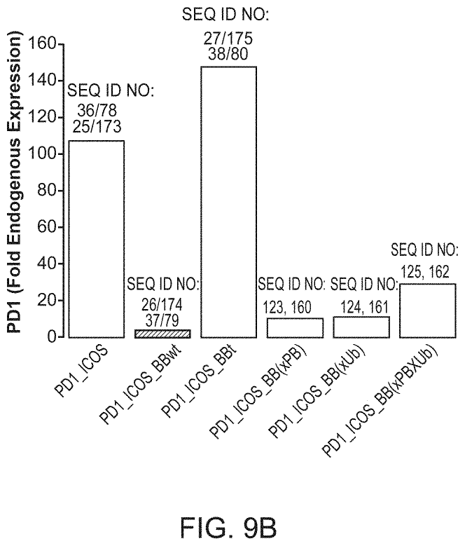

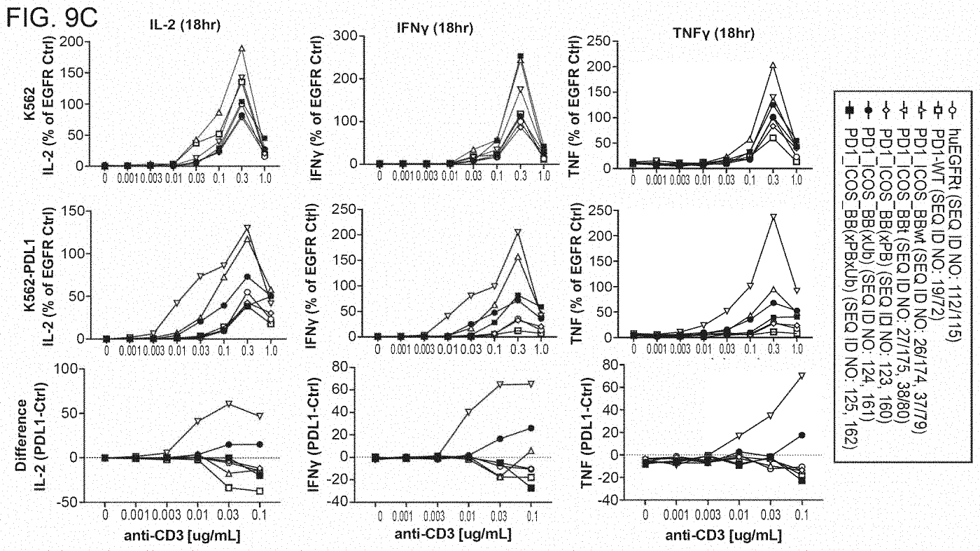

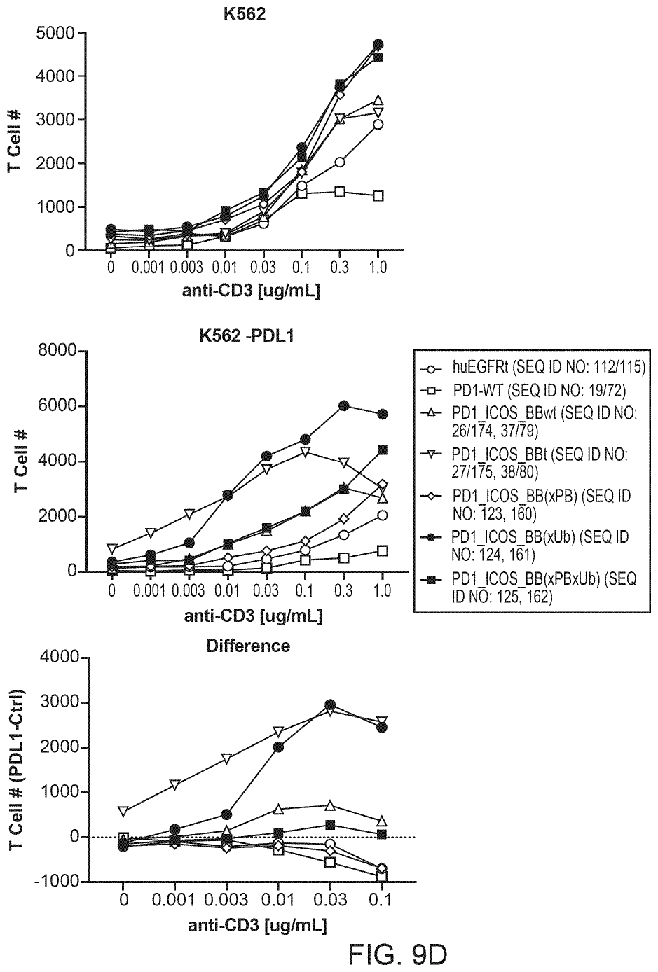

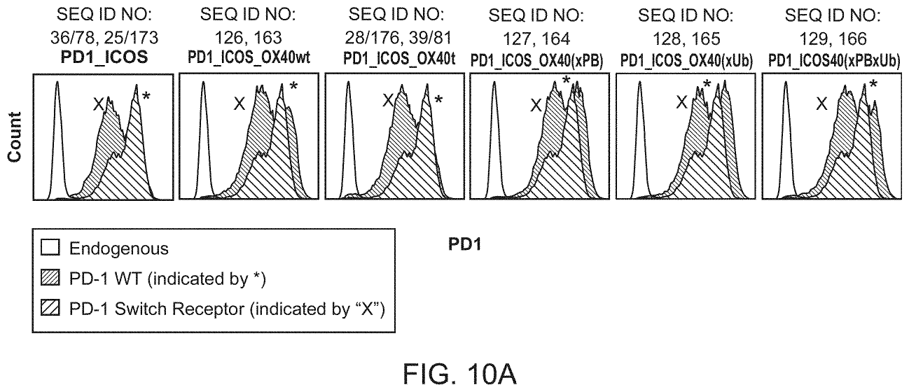

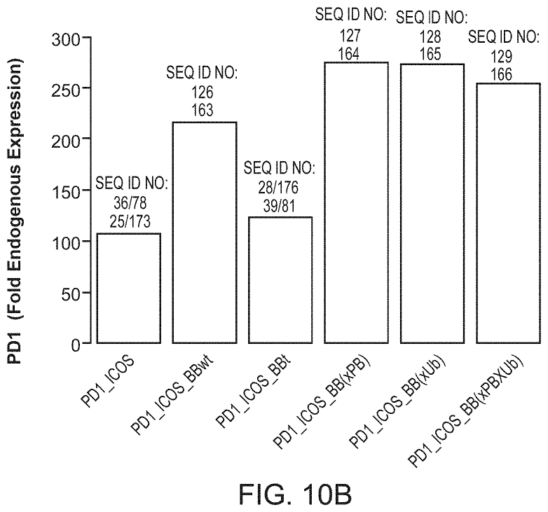

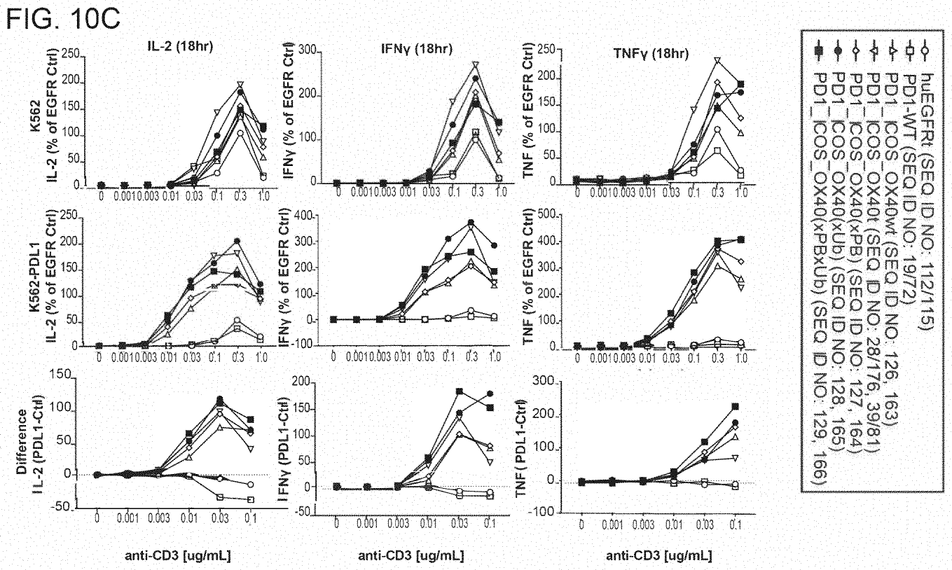

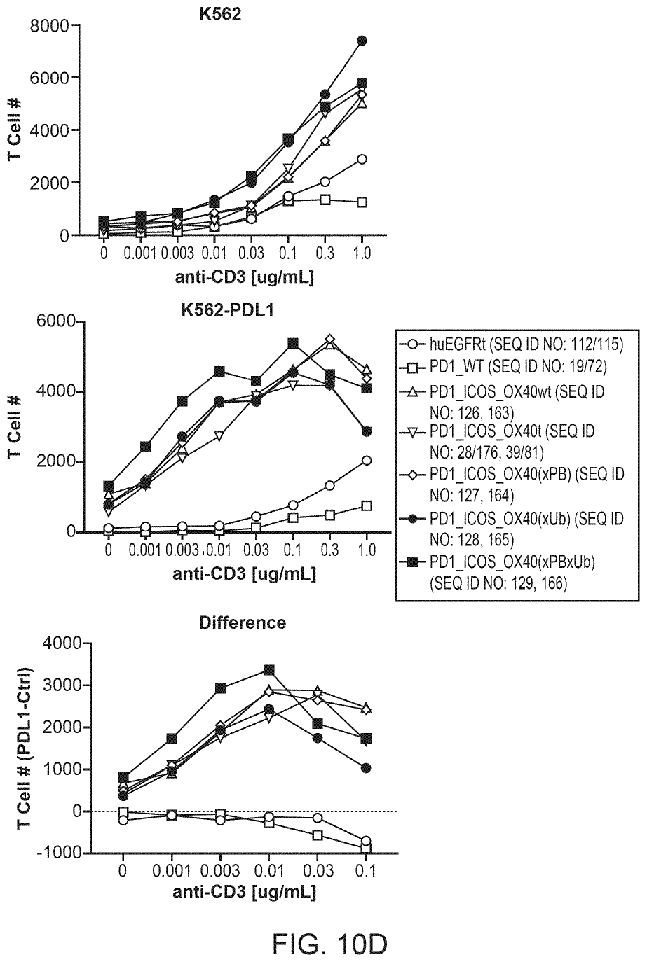

The effect of the receptors with mutation of the polybasic and lysine residues is less than the PD1_ICOS_BBt co-stimulatory molecule, in terms of both surface expression of the co-stimulatory molecule (FIGS. 9A-9B), effector cytokine production in response to stimulation with anti-CD3 antibody (FIG. 9C), and T cell proliferation in response to stimulation with target cells expressing PD-L1 (FIG. 9D). The PD1_ICOS_OX40t receptor (with truncated OX40 intracellular domain) had an effect comparable to that of the wild type PD1_ICOS_OX40wt receptor (comprising wild type OX40 intracellular domain), in terms of both surface expression of the co-stimulatory molecule (FIGS. 10A-10B), effector cytokine production in response to stimulation with anti-CD3 antibody (FIG. 10C), and T cell proliferation in response to stimulation with target cells expressing PD-L1 (FIG. 10D).

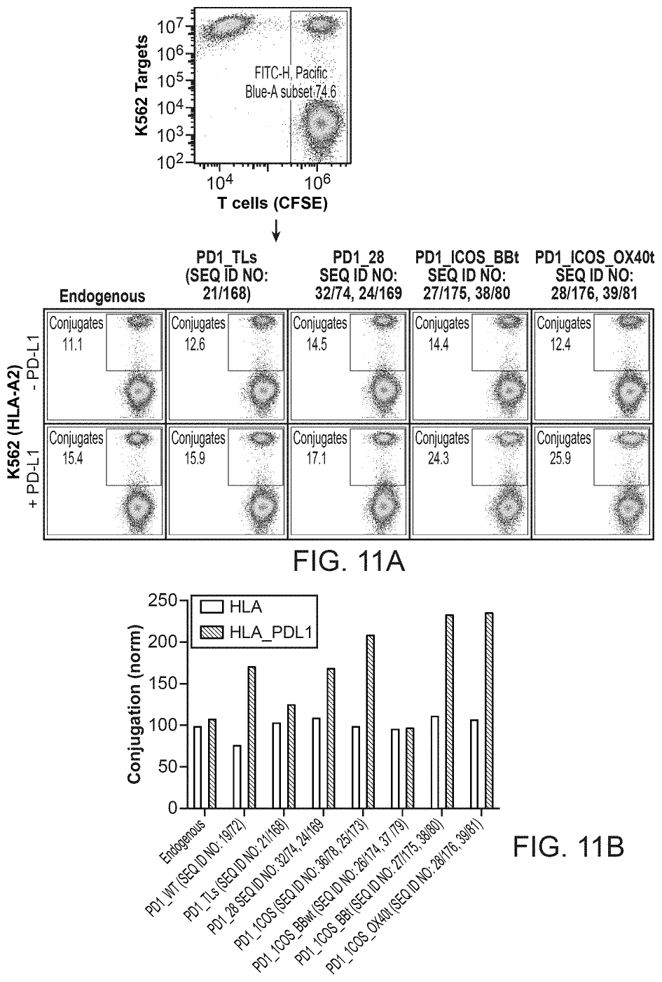

Further, the ICOS-based co-stimulatory molecules encouraged T cell: PD-L1 expressing (PD-L1+) target cell interaction in a flow-based conjugation assay, suggesting that these receptors encourage prolonged T cell—APC interactions while scanning for cognate antigen, a useful property when scanning for low-abundance antigen in the TME (FIGS. 11A-11B).

The disclosure herein shows that the co-stimulatory molecules based on the modified 3^{rd}-generation intracellular signaling domain disclosed herein are superior to currently existing PD1_28 co-stimulatory molecules in enhancing T cell effector function when responding to a PD-L1+ target cell. This includes increased T cell proliferation, cytokine secretion, and target cell killing. The 3^{rd}-generation intracellular signaling domain disclosed herein can be successfully combined with TCR-T therapy targeting TAAs.

### Example 3: In-Vitro Preclinical Studies

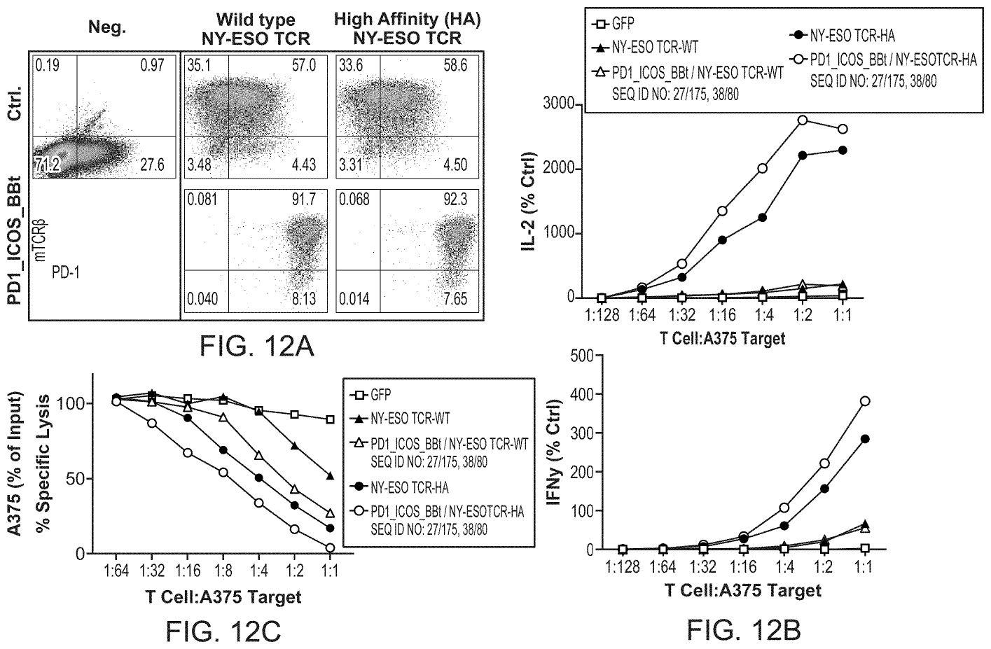

Described herein are T cells expressing specific HLA-A2/NY-ESO specific TCRs and co-stimulatory molecules comprising ICOS and mutated CD137 signaling domains, that increase expression of the co-stimulatory molecule on T cell surface (FIG. 12A), effector cytokine production (FIG. 12B), and killing of target cells expressing NY-ESO (FIG. 12C), as compared to T cells expressing the specific HLA-A2/NY-ESO specific TCRs alone.

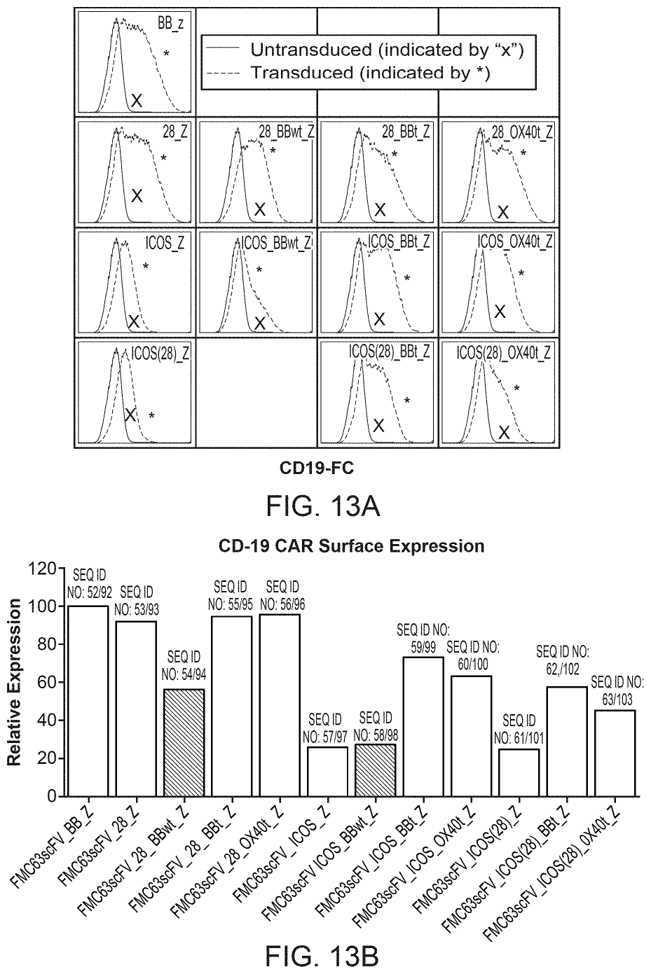

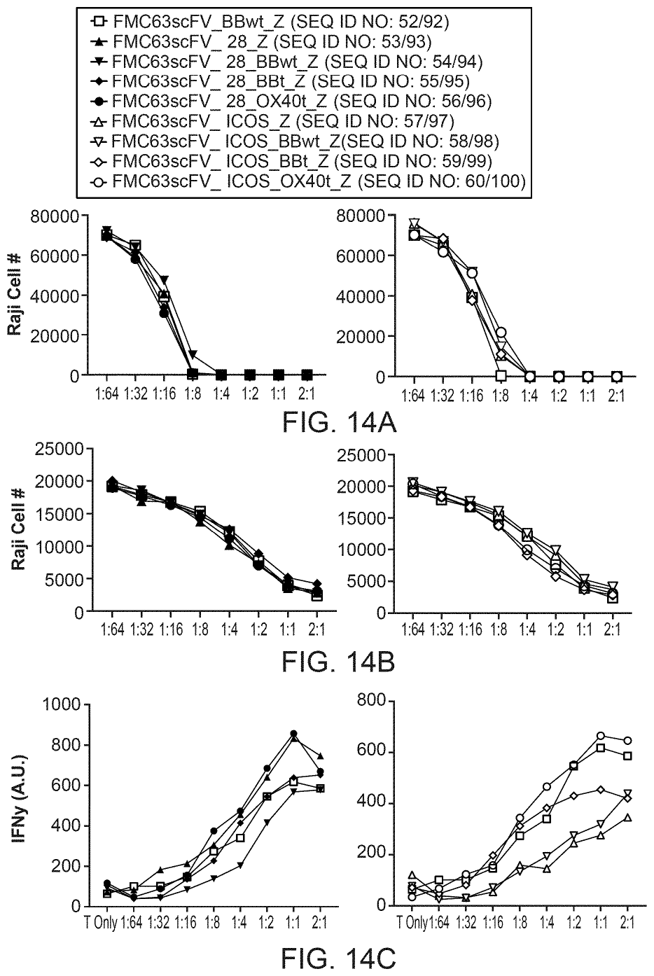

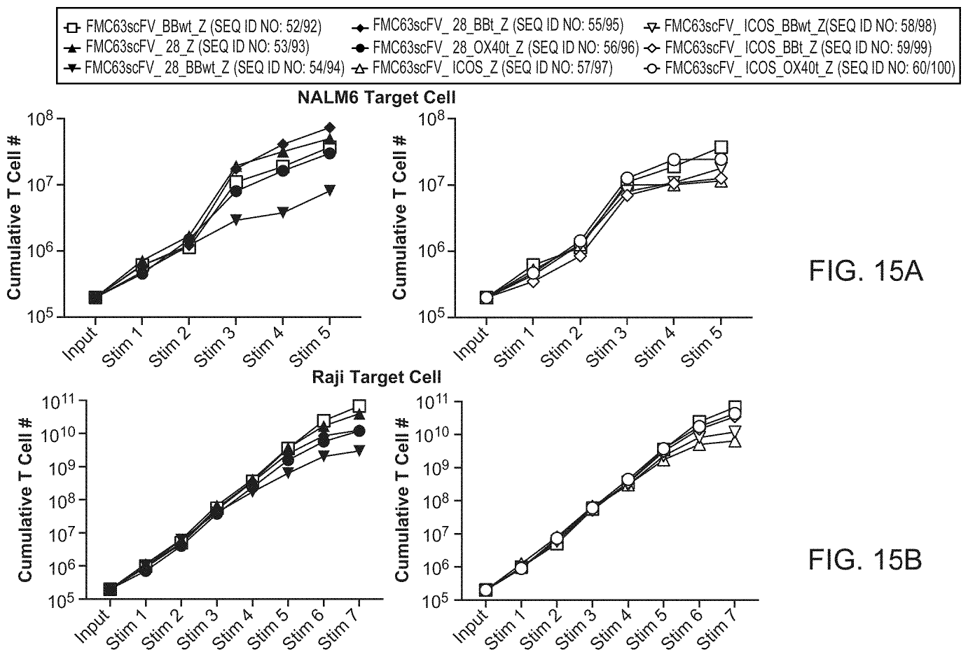

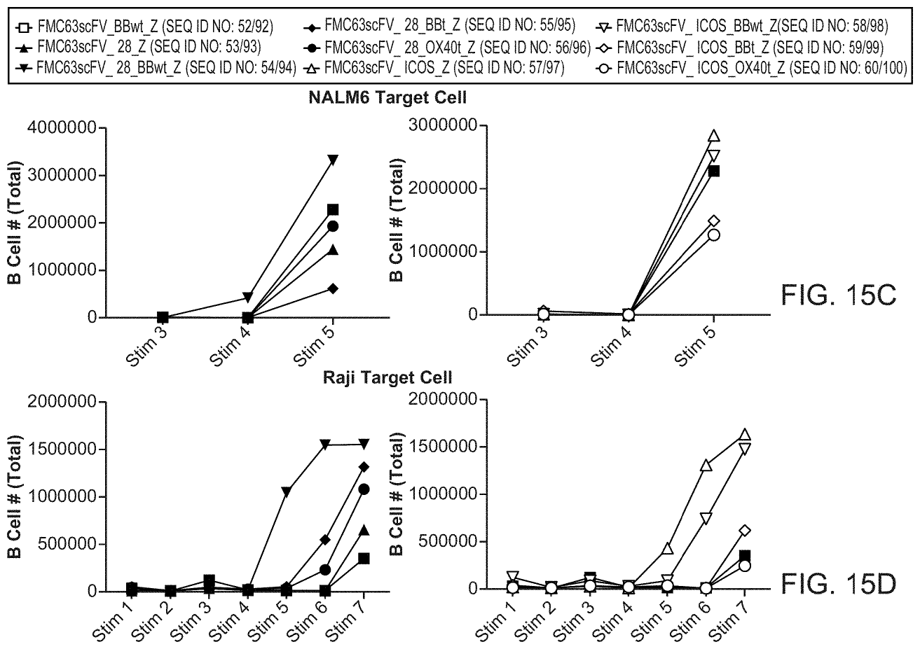

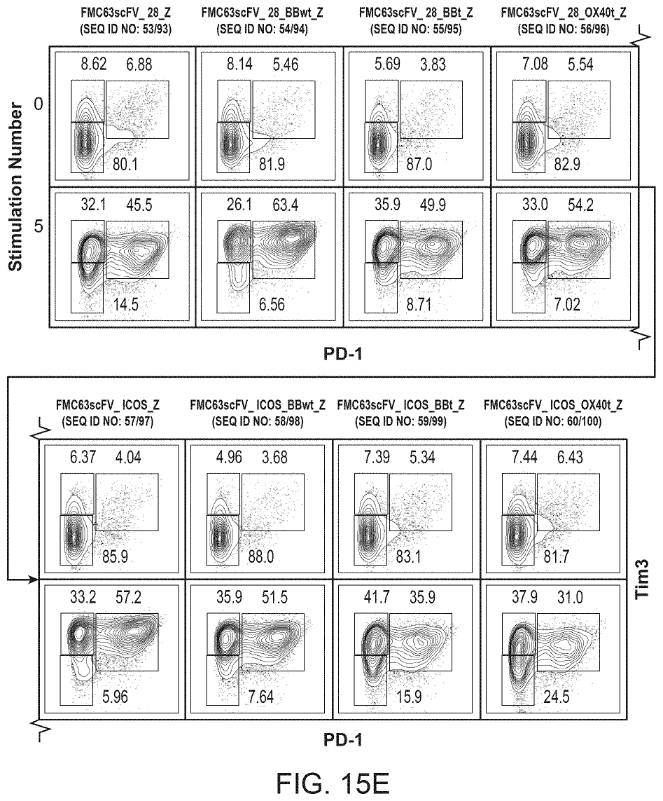

Described herein are CD-19 CAR constructs comprising the modified 3^{rd}-generation intracellular signaling domains disclosed herein. The CD-19 (FMC63scFV) CARs with 3^{rd}-generation intracellular signaling constructs described herein include constructs comprising the intracellular chimeric domains: CD28-CD137-CD3ζ (28_BBwt_z), CD28-CD137mutant-CD3ζ (28_BBt_z), CD28-CD134mutant-CD3ζ (28_OX40t_Z), ICOS-CD137-CD3ζ (ICOS_BB_z), ICOS-CD137mutant-CD3ζ (ICOS_BBt_z), and ICOS-CD134mutant-CD3ζ (ICOS_OX40t_z). Also, provided are CD-19 CARs with a 3^{rd}-generation intracellular signaling construct with a portion of CD28 inserted within the ICOS domain: ICOS(28)-CD137-CD3ζ (ICOS(28)_BBwt_z), ICOS(28)-CD137mutant-CD3ζ (ICOS(28)_BBt_z), and ICOS(28)-CD134mutant-CD3ζ(ICOS(28)_OX40t_z). Second-generation constructs comprising CD137-CD3ζ (BBwt_z), CD28-CD3ζ (28_z) and ICOS-CD3ζ (ICOS_z) are used as controls. Similar to the study described herein using the PD-1 3^{rd}-generation intracellular signaling constructs, the CD19 CAR constructs with the CD137 and CD134 mutants domains showed higher expression as compared to the corresponding constructs with wild type CD137 and CD134 domains (28_BBwt_z and ICOS_BBwt_z, respectively) (FIGS. 13A-13B). In-Vitro studies described herein show increased killing of CD19 expressing cells (CD19+) (FIGS. 14A-14B and FIGS. 15C-15D), increased effector cytokine production (FIG. 14C, right panels) and increased T cell proliferation and persistence (FIGS. 15A-15B and 15E), by primary T cells transduced with the 3^{rd} generation CD28 based and ICOS based CD19 CARs.

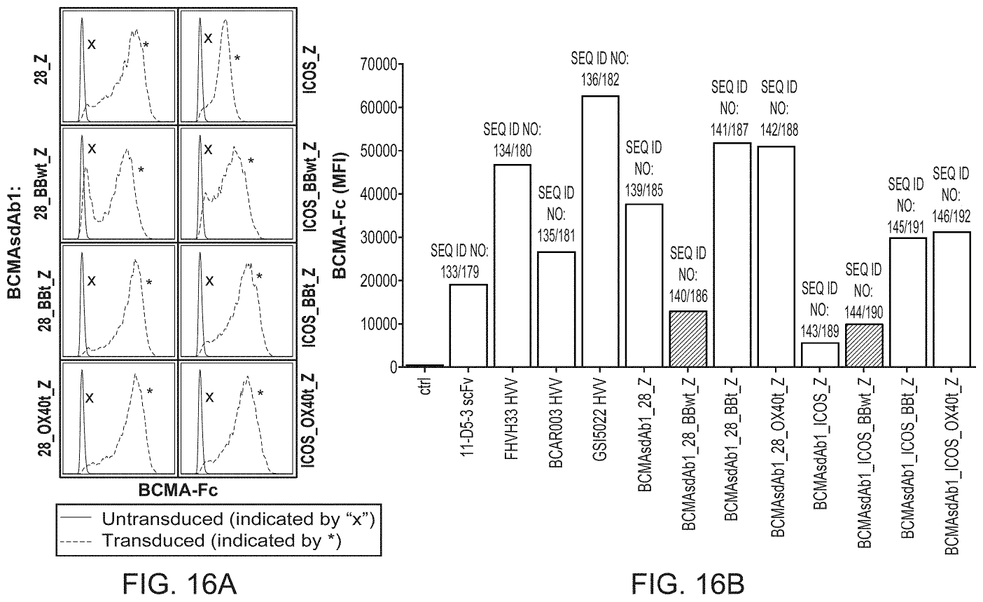

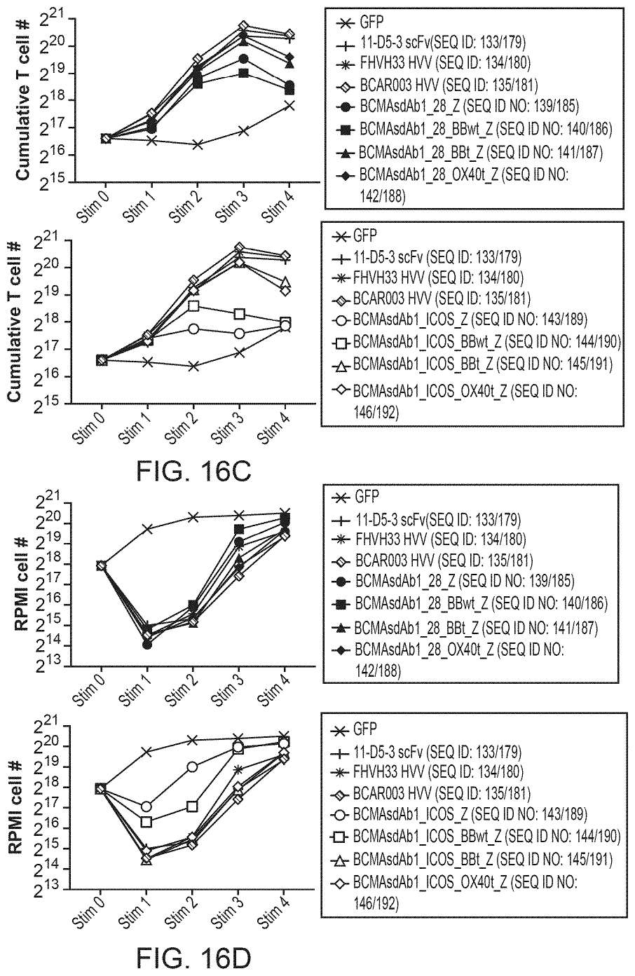

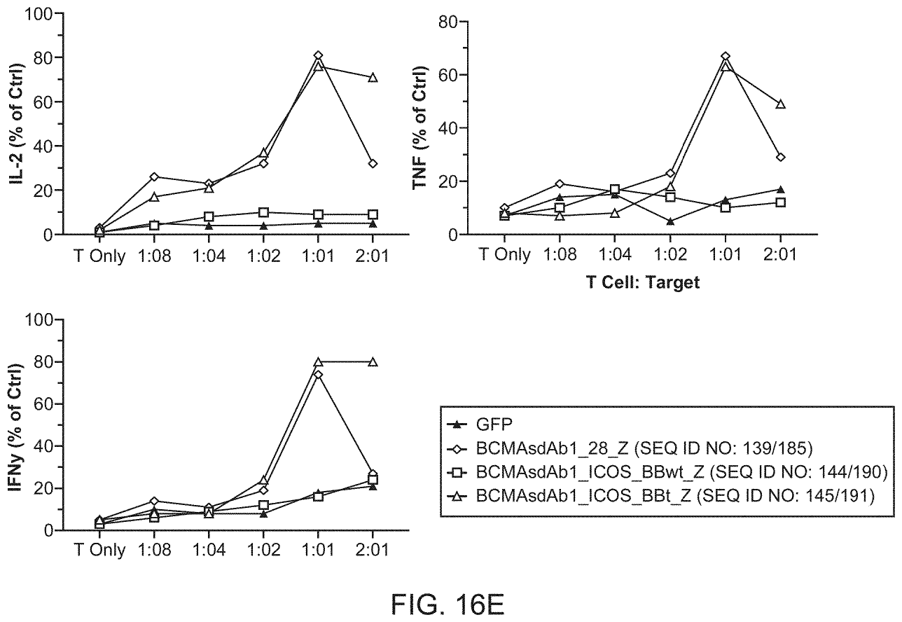

The disclosure also shows that expression of CD28 based receptors comprising a mutated CD134/CD137 signaling domains and ICOS based receptors comprising a mutated CD134/CD137 signaling domains, increased binding of BCMA specific T cells (BCMA CAR T cells) to the target antigen (BCMA-Fc) (FIGS. 16A-16B). Also, the disclosure shows that expression of the CD28 based receptors comprising a mutated CD134/CD137 signaling domains and ICOS based receptors comprising a mutated CD134/CD137 signaling domains, increased proliferation and effector cytokine production (FIGS. 16C and 16E), and target cell killing (FIG. 16D) by the BCMA specific T cells in response to myeloma cell line expressing BCMA.

1. A recombinant T cell co-stimulatory receptor (RTCR), comprising:
(a) an extracellular domain;
(b) a transmembrane domain; and
(c) a chimeric intracellular domain comprising a first and at least a second signal transduction domains,
wherein the first and the at least second signal transduction domains are non-identical; and
wherein the at least second signal transduction domain comprises a mutant CD137 (4-1BB) intracellular domain or a mutant CD134 (OX-40) intracellular domain.

2. The RTCR of claim 1, wherein the mutant CD137 intracellular domain comprises:
a) an amino acid sequence according to amino acid position 13 to amino acid position 42 of the CD137 intracellular domain;
b) a deletion of a continuous stretch of one, two, three, four, five, six, seven, eight, nine, ten or more amino acids from the N-terminus of the CD137 intracellular domain;
c) a deletion of one, two, three, four, five, six, seven, eight, nine, ten or more amino acids from amino acid position 1 to amino acid position 12 of the N-terminus of the CD137 intracellular domain;
d) a deletion of one, two, three or four lysine residue(s) from amino acid position 1 to amino acid position 12 of the N-terminus of the CD137 intracellular domain;
e) one or more lysine mutation(s) from amino acid position 1 to amino acid position 12 of the N-terminus of the CD137 intracellular domain;
f) one or more lysine mutation(s) at amino acid positions selected from amino acid positions 1, 5, 6 and 12 of the N-terminus of the CD137 intracellular domain;
g) one or more proximal basic amino acid mutation(s) or deletion(s) from amino acid position 1 to amino acid position 12 of the N-terminus of the CD137 intracellular domain; or
h) one or more proximal basic amino acid mutation(s) at amino acid positions selected from amino acid positions 1, 2, 3, 4, 5 and 6 of the N-terminus of the CD137 intracellular domain; or a combination thereof.

3.-13. (canceled)

14. The RTCR of claim 1, wherein the mutant CD134 intracellular domain comprises:
a) an amino acid sequence according to amino acid position 15 to amino acid position 37 of the CD134 intracellular domain;
b) a deletion of a continuous stretch of one, two, three, four, five, six, seven, eight, nine, ten, eleven, twelve, thirteen or more amino acids from the N-terminus of the CD134 intracellular domain;
c) a truncated CD134 intracellular domain comprising a deletion of one, two, three, four, five, six, seven, eight, nine, ten, eleven, twelve, thirteen or more amino acids from amino acid position 1 to amino acid position 14 of the N-terminus of the CD134 intracellular domain;
d) a deletion of a lysine residue from amino acid position 1 to amino acid position 14 of the N-terminus of the CD134 intracellular domain;
e) a lysine mutation at amino acid position 12 of the N-terminus of the CD134 intracellular domain;
f) one or more proximal basic amino acid mutation(s) or deletion(s) from amino acid position 1 to amino acid position 14 of the N-terminus of the CD137 intracellular domain; or
g) one or more proximal basic amino acid mutation(s) at amino acid positions selected from amino acid positions 1, 2, and 5 of the N-terminus of the CD134 intracellular domain, or a combination thereof.

15.-27. (canceled)

28. The RTCR of claim 1, wherein the RTCR comprises any one of:
a) a first signal transduction domain derived from ICOS domain comprising an amino acid sequence according to SEQ ID NO: 9;
b) a first signal transduction domain comprising a portion of a CD28 intracellular domain combined with an ICOS domain according to SEQ ID NO: 9;
c) first signal transduction domain comprising an amino acid sequence according to any one of SEQ ID NOs: 12 and 109;
d) a first signal transduction domain derived from CD28;
e) a first signal transduction domain derived from a CD28 domain comprising an amino acid sequence according to SEQ ID NO: 10;
f) a first signal transduction domain comprising an amino acid sequence according to any one of SEQ ID NOs: 121-122; or
g) an amino acid sequence according to any one of SEQ ID NOs: 14-17.

29.-34. (canceled)

35. The RTCR of claim 1, wherein the chimeric intracellular domain further comprises a third signal transduction domain that is any one of:
a) a CD3 signaling domain derived form a CD3ζ or a CD3ε domain or a combination thereof;
b) a CD2 signaling domain that is a mutant or truncated CD2 signaling domain; or
c) an interleukin 2 receptor binding (IL-2RB) protein signaling domain, or a combination thereof.

36.-37. (canceled)

38. The RTCR of claim 35, wherein the third signal transduction domain is any one of:
a) CD3 signaling domain comprising an amino acid sequence according to any one of SEQ ID NOs: 18, 45, 46, 47 and 48;
b) CD2 signaling domain comprises an amino acid sequence according to SEQ ID NO: 49; or
c) an IL-2RB protein signaling domain comprises an amino acid sequence according to SEQ ID NO: 50.

39.-42. (canceled)

43. The RTCR of claim 1, wherein the chimeric intracellular domain further comprises a fourth signal transduction domain, that is any one of:
a) a CD3 signaling domain derived form a CD3ζ or a CD3ε domain or a combination thereof;
b) a CD2 signaling domain that is a mutant or truncated CD2 signaling domain; or
c) an interleukin 2 receptor binding (IL-2RB) protein signaling domain, or a combination thereof,
wherein the third and the fourth signal transduction domain are not identical.

44.-45. (canceled)

46. The RTCR of claim 43, wherein the fourth signal transduction domain is any one of:
a) a CD3 signaling domain comprising an amino acid sequence according to any one of SEQ ID NOs: 18, 45, 46, 47 and 48;
b) CD2 signaling domain comprises an amino acid sequence according to SEQ ID NO: 49; or
c) an IL-2RB protein signaling domain comprises an amino acid sequence according to SEQ ID NO: 50.

47.-50. (canceled)

51. The RTCR of claim 1, wherein the extracellular domain comprises a protein or a portion thereof that induces activation and/or proliferation of an immune cell.

52. The RTCR of claim 51, wherein the extracellular domain comprises any one of:
a) a component of a T cell receptor (TCR) complex;
b) a component of a chimeric antigen receptor (CAR);
c) a component of a T cell co-receptor, wherein the T cell co-receptor is a T cell co-stimulatory protein or T cell inhibitory protein;
d) a ligand that binds to a cell surface receptor or a component thereof;
e) a component of a cytokine receptor;
f) a component of a chemokine receptor;
g) a component of an integrin receptor;
h) a component of an endothelial cell surface protein receptor or a fragment thereof;
i) a component of a neuronal guidance protein receptor; and
j) a component of a complement receptor.

53. The RTCR of claim 52, wherein the extracellular domain comprises any one of:
a) a component of the T cell co-receptor or a CAR that is a component of PD1, CD28, CD2, OX-40, ICOS, CTLA-4, CD28, CD3, CD4, CD8, CD40L, Lag-3, Tim-3, or TIGIT, or a combination thereof;
b) a component of the T cell co-receptor or a CAR that binds to CD19, B cell maturation Ag (BCMA), PD-L1, PD-L2, IL-10, a proliferation-inducing ligand (APRIL), BAFF, OX-40L, ICOS-L, B7-1, B7-2, CD40, CD58, CD59, nectin, CD155, or CD112, or a combination thereof;
c) a cytokine receptor that binds to IL-10, IL-27, TGF-β, IL-12, IL-1, IL-2, IL-4, IL-5, IFN-γ, or IFN-α/β, or a combination thereof;
d) a component of C3aR, C5aR, CD46/MCP, CD55, CD97, or DAF, or a combination thereof;
e) a component of epithelial growth factor receptor (EGFR), vascular-endothelial growth factor receptor (VEGFR), chemokine receptor (CCR) 4, CCR5, CCR7, CCR10, netrin-1 receptor, semaphorin receptor, lymphocyte function-associated antigen-1 (LFA-1), leukocyte-specific β2 integrin (αLβ2, αMβ2, αXβ2, or αDβ2), β7 integrin (α4β7 or αEβ7), extracellular matrix (ECM)-binding β1 integrin (α1-α6β1), L-selectin, or sialyl Lewis^{x};
f) a polypeptide, a glycoprotein, or an antibody or a fragment thereof, wherein the antibody or fragment thereof is a Fab fragment, a F(ab)2 fragment, a diabody, a nanobody, a sdAb, Fv, a VHH fragment, or a single chain Fv fragment; or
g) an extracellular domain that binds to a target selected from a tumor antigen, a pathogen associated protein, and an antigen associated with an autoimmune, an inflammatory, a metabolic, or a neurodegenerative condition or disorder.

54.-70. (canceled)

71. A nucleic acid encoding the RTCR of claim 1.

72. (canceled)

73. A cell comprising the nucleic acid of claim 71.

74. The cell of claim 73, wherein the cell is any one of: a) a modified T cell; and a modified natural killer T cell (NK-T cell),
wherein the T cell is any one of: i) an allogenic T cell; or ii) an autologous T cell.

75.-82. (canceled)

83. A modified T lymphocyte (T cell), comprising:
(a) a modification of an endogenous sequence encoding a T cell Receptor (TCR), wherein the modification reduces or eliminates a level of expression or activity of the TCR; and
(b) a recombinant T cell co-stimulatory receptor (RTCR) according to claim 1.

84.-85. (canceled)

86. A composition comprising the RTCR of any one of claim 1.

87.-91. (canceled)

92. A method of producing a plurality of modified T cells, wherein the method comprises:
a) providing a plurality of primary T cells;
b) providing a composition comprising the RTCR of claim 1; and
c) introducing into the plurality of primary T cells of (a) the composition of (b), to produce a plurality of modified T cells under conditions that stably express the RTCR within the plurality of modified T cells.

93.-95. (canceled)

96. A method of treating a disease or disorder, comprising administering to a subject in need thereof a therapeutically effective number of the cell of claim 73.

97.-106. (canceled)

107. A chimeric co-stimulatory intracellular protein (CIP) comprising a first and at least a second signal transduction domains,
wherein the CIP is the chimeric intracellular domain of the RTCR of claim 1.

108.-159. (canceled)


SEQUENCE LISTING (198 sequences)

SEQ ID NO: 1 - protein, 42 aa, Artificial Sequence
  chemically synthesized
KRGRKKLLYIFKQPFMRPVQTTQEEDGCSCRFPEEEEGGCEL

SEQ ID NO: 2 - protein, 42 aa, Artificial Sequence
  chemically synthesized
RKKFPHIFKQPFKKTTGAAQEEDACSCRCPQEEEGGGGGYEL

SEQ ID NO: 3 - protein, 30 aa, Artificial Sequence
  chemically synthesized
QPFMRPVQTTQEEDGCSCRFPEEEEGGCEL

SEQ ID NO: 4 - protein, 37 aa, Artificial Sequence
  chemically synthesized
RRDQRLPPDAHKPPGGGSFRTPIQEEQADAHSTLAKI

SEQ ID NO: 5 - protein, 36 aa, Artificial Sequence
  chemically synthesized
RKAWRLPNTPKPCWGNSFRTPIQEEHTDAHFTLAKI

SEQ ID NO: 6 - protein, 21 aa, Artificial Sequence
  chemically synthesized
GGGSFRTPIQEEQADAHSTLA

SEQ ID NO: 7 - protein, 48 aa, Artificial Sequence
  chemically synthesized
QRRKYRSNKGESPVEPAEPCHYSCPREEEGSTIPIQEDYRKPEPACSP

SEQ ID NO: 8 - protein, 58 aa, Artificial Sequence
  chemically synthesized
QLGLHIWQLRSQCMWPRETQLLLEVPPSTEDARSCQFPEEERGERSAEEKGRLGDLWV

SEQ ID NO: 9 - protein, 67 aa, Artificial Sequence
  chemically synthesized
SQLCCQLKFWLPIGCAAFVVVCILGCILICWLTKKKYSSSVHDPNGEYMFMRAVNTAKKSRLTDVTL

SEQ ID NO: 10 - protein, 76 aa, Artificial Sequence
  chemically synthesized
LFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 18 aa, Artificial Sequence
  chemically synthesized
TPRRPGPTRKHYQPYAPP

SEQ ID NO: 12 - protein, 85 aa, Artificial Sequence
  chemically synthesized
SQLCCQLKFWLPIGCAAFVVVCILGCILICWLTKKKYSSSVHDPNGEYMFMTPRRPGPTRKHYQPYAPPRAVNTAKKSRLTDVTL

SEQ ID NO: 13 - protein, 109 aa, Artificial Sequence
  chemically synthesized
SQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 14 - protein, 97 aa, Artificial Sequence
  chemically synthesized
SQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 15 - protein, 88 aa, Artificial Sequence
  chemically synthesized
SQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 16 - protein, 115 aa, Artificial Sequence
  chemically synthesized
SQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 17 - protein, 106 aa, Artificial Sequence
  chemically synthesized
SQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 18 - protein, 164 aa, Artificial Sequence
  chemically synthesized
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 288 aa, Artificial Sequence
  chemically synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 20 - protein, 194 aa, Artificial Sequence
  chemically synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSR

SEQ ID NO: 21 - protein, 195 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSR

SEQ ID NO: 22 - protein, 163 aa, Artificial Sequence
  chemically synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 23 - protein, 163 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRP

SEQ ID NO: 24 - protein, 240 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRS

SEQ ID NO: 25 - protein, 231 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTL

SEQ ID NO: 26 - protein, 273 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 27 - protein, 261 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 28 - protein, 252 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 29 - protein, 279 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 30 - protein, 270 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 31 - protein, 140 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPA

SEQ ID NO: 32 - protein, 216 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 258 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 34 - protein, 246 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 35 - protein, 237 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 36 - protein, 207 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTL

SEQ ID NO: 37 - protein, 249 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCEL

SEQ ID NO: 38 - protein, 237 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCEL

SEQ ID NO: 39 - protein, 228 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLGGGSFRTPIQEEQADAHSTLA

SEQ ID NO: 40 - protein, 255 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCEL

SEQ ID NO: 41 - protein, 246 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLA

SEQ ID NO: 42 - protein, 315 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSN

SEQ ID NO: 43 - protein, 352 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 44 - protein, 430 aa, Artificial Sequence
  chemically synthesized
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPASQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSNNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 45 - protein, 113 aa, Artificial Sequence
  chemically synthesized
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 48 aa, Artificial Sequence
  chemically synthesized
RVKFSRSADAPAYQQGQNQLYNELNLGRREEYDVLDKRRGRDPEMGGK

SEQ ID NO: 47 - protein, 47 aa, Artificial Sequence
  chemically synthesized
PVTRGAGAGGRQRGQNKERPPPVPNPDYEPIRKGQRDLYSGLNQRRI

SEQ ID NO: 48 - protein, 95 aa, Artificial Sequence
  chemically synthesized
RVKFSRSADAPAYQQGQNQLYNELNLGRREEYDVLDKRRGRDPEMGGKPVTRGAGAGGRQRGQNKERPPPVPNPDYEPIRKGQRDLYSGLNQRRI

SEQ ID NO: 49 - protein, 78 aa, Artificial Sequence
  chemically synthesized
QNPATSQHPPPPPGHRSQAPSHRPPPPGHRVQHQPQKRPPAPSGTQVHQQKGPPLPRPRVQPKPPHGAAENSLSPSSN

SEQ ID NO: 50 - protein, 115 aa, Artificial Sequence
  chemically synthesized
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVSYLRQWVVIPPPLSSPGPQAS

SEQ ID NO: 51 - protein, 307 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFAC

SEQ ID NO: 52 - protein, 487 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 53 - protein, 496 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 538 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - protein, 526 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 56 - protein, 517 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - protein, 487 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 58 - protein, 529 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 59 - protein, 517 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 60 - protein, 508 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 61 - protein, 505 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMTPRRPGPTRKHYQPYAPPRAVNTAKKSRLTDVTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - protein, 535 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMTPRRPGPTRKHYQPYAPPRAVNTAKKSRLTDVTLQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 63 - protein, 526 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMTPRRPGPTRKHYQPYAPPRAVNTAKKSRLTDVTLGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 452 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGK

SEQ ID NO: 65 - protein, 499 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGKPVTRGAGAGGRQRGQNKERPPPVPNPDYEPIRKGQRDLYSGLNQRRI

SEQ ID NO: 66 - protein, 595 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 67 - protein, 580 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPVTRGAGAGGRQRGQNKERPPPVPNPDYEPIRKGQRDLYSGLNYRHQRRI

SEQ ID NO: 68 - protein, 611 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDAYRHQALPPR

SEQ ID NO: 69 - protein, 596 aa, Artificial Sequence
  chemically synthesized
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYQQGQNQLYNELNLGRREEYDVLDKRRGRDPEMGGKPVTRGAGAGGRQRGQNKERPPPVPNPDYEPIRKGQRDLYSGLNYRHQRRI

SEQ ID NO: 70 - DNA, 234 bp, Artificial Sequence
  chemically synthesized
cagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaac

SEQ ID NO: 71 - DNA, 345 bp, Artificial Sequence
  chemically synthesized
aattgcagaaacacaggcccctggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatccctccacctctctctagtcctggacctcaggcctct

SEQ ID NO: 72 - DNA, 864 bp, Artificial Sequence
  chemically synthesized
atgcagattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggactgctgggatctctggtgctgcttgtttgggtgctcgccgtgatctgtagcagagccgccagaggaacaatcggcgccagaaggacaggccagcctctgaaagaggatccctctgctgtccccgtgttcagcgtggactatggcgagctggatttccagtggcgggaaaagacacccgagcctccagtgccttgtgtgcctgagcagacagagtacgccaccatcgtgttccctagcggcatgggcacatctagccctgccagaagaggatctgccgacggacctagatctgcccagcctctcagacctgaggatggccactgttcttggcctctt

SEQ ID NO: 73 - DNA, 420 bp, Artificial Sequence
  chemically synthesized
ttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgcc

SEQ ID NO: 74 - DNA, 648 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 75 - DNA, 774 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 76 - DNA, 738 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 77 - DNA, 711 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 78 - DNA, 621 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 79 - DNA, 747 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 80 - DNA, 711 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 81 - DNA, 684 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 82 - DNA, 765 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 83 - DNA, 738 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 84 - DNA, 945 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaac

SEQ ID NO: 85 - DNA, 1056 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggctgcgaactgaattgcagaaacacaggcccctggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatccctccacctctctctagtcctggacctcaggcctct

SEQ ID NO: 86 - DNA, 1290 bp, Artificial Sequence
  chemically synthesized
tttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggccccatctcacagacctccaccacctggtcatcgggtgcagcatcagccccagaaaagacctcctgctcctagcggaacacaggtgcaccagcaaaagggacctccactgcctagacctagagtgcagcctaagcctcctcatggcgctgccgagaatagcctgtctcctagcagcaacaactgccgcaacacaggcccctggctgaagaaagtgctgaagtgcaacacccctgatccgagcaagttctttagccagctgagcagcgagcatggcggcgacgttcagaaatggctgtctagcccatttccaagcagcagcttcagccctggtggactggcccctgagattagccctctggaagtgctggaacgggacaaagtgacccagctgctgcccctgaataccgacgcttacctgagcctgcaagagctgcaaggacaggaccctacacacctggtgtcctacctgagacagtgggtcgtgatcccacctcctttgagcagtccaggacctcaggcctct

SEQ ID NO: 87 - DNA, 339 bp, Artificial Sequence
  chemically synthesized
agagtgaagttcagcagaagcgccgacgcccccgcctaccagcagggccagaaccagctgtacaacgagctgaacctgggcagaagagaggagtacgacgtgctggacaagagaagaggcagagaccccgagatgggcggcaagccccagagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 88 - DNA, 144 bp, Artificial Sequence
  chemically synthesized
agagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaaa

SEQ ID NO: 89 - DNA, 141 bp, Artificial Sequence
  chemically synthesized
cctgtgactagaggtgctggtgctggcggcagacagagaggccagaacaaagaaagacctcctcctgtgcctaatcctgactacgagcccatccggaagggccagagagatctgtacagcggcctgaaccagcggagaatc

SEQ ID NO: 90 - DNA, 285 bp, Artificial Sequence
  chemically synthesized
agagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagcctgtgactagaggtgctggtgctggcggcagacagagaggccagaacaaagaaagacctcctcctgtgcctaatcctgactacgagcccatccggaagggccagagagatctgtacagcggcctgaaccagcggagaatc

SEQ ID NO: 91 - DNA, 921 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgc

SEQ ID NO: 92 - DNA, 1461 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 93 - DNA, 1488 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 94 - DNA, 1614 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 95 - DNA, 1578 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaacttagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 96 - DNA, 1551 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 97 - DNA, 1461 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgacactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 98 - DNA, 1587 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctgaagcggggcagaaagaagctgctgtatatcttcaagcagcccttcatgcggcccgtgcagaccacacaagaggaagatggctgctcctgtcggttccccgaggaagaagaaggcggttgcgaactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 99 - DNA, 1551 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaacttagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 100 - DNA, 1524 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgacactcggcggaggcagctttagaacccctatccaagaggaacaggccgacgctcactctacactggctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 101 - DNA, 1515 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgacccctagacggcccggacctaccagaaagcactaccagccttacgctcctcctcgggccgtgaacacagccaagaaaagcagactgaccgacgtgaccctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 102 - DNA, 1605 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgacccctagacggcccggacctaccagaaagcactaccagccttacgctcctcctcgggccgtgaacacagccaagaaaagcagactgaccgacgtgaccctccagcctttcatgaggcctgtgcagaccacacaagaagaggacggctgctcctgtcggttccccgaggaagaggaaggcggttgcgaacttagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 103 - DNA, 1578 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgacccctagacggcccggacctaccagaaagcactaccagccttacgctcctcctcgggccgtgaacacagccaagaaaagcagactgaccgacgtgacactcggcggaggcagctttagaacccctatccaagaggaacaggccgacgctcactctacactggctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 104 - DNA, 1359 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaaatga

SEQ ID NO: 105 - DNA, 1500 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagcctgtgactagaggtgctggtgctggcggcagacagagaggccagaacaaagaaagacctcctcctgtgcctaatcctgactacgagcccatccggaagggccagagagatctgtacagcggcctgaaccagcggagaatctga

SEQ ID NO: 106 - DNA, 1788 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggctcctagccacagacctccaccacctggtcatagagtgcagcaccagcctcagaagaggcctcctgctccttctggaacacaggtgcaccagcaaaagggccctccactgcctagacctagggtgcagcctaaacctcctcatggcgccgctgagaactctctgagccccagcagcaacagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggaaagggacacgacggactgtaccagggcctgagcaccgccacaaaggatacctatgacgccctgcacatgcaggccctgcctccaagatga

SEQ ID NO: 107 - DNA, 1743 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaactccagaatcctgccacctctcagcaccctccacctccacctggacacagatctcaggctcctagccacagacctccaccacctggtcatagagtgcagcaccagcctcagaagaggcctcctgctccttctggaacacaggtgcaccagcaaaagggccctccactgcctagacctagggtgcagcctaaacctcctcatggcgccgctgagaactctctgagccccagcagcaacagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagcctgtgactagaggtgctggcgctggtggaaggcagagaggccagaacaaagaacggcctcctcctgtgcctaatcctgactacgagcccatccggaagggccagagagatctgtacagcggcctgaactacagacaccagcggagaatctga

SEQ ID NO: 108 - DNA, 1836 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggctgcgaactgaattgcagaaacacaggcccctggctgaagaaagtgctgaagtgcaacacccctgatccgagcaagtttttcagccagctgagcagcgagcatggcggcgacgttcagaaatggctgtctagcccatttcctagctccagcttcagccctggtggactggcccctgagattagccctctggaagtgctggaacgggacaaagtgacccagctgctgcccctgaataccgacgcctacctgagcctgcaagagctgcaaggccaggatcctacacacctcgtgcgcgtgaagttcagcagatccgctgatgcccctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctacgatgcctacagacaccaggctctgccacctagatga

SEQ ID NO: 109 - protein, 56 aa, Artificial Sequence
  chemically synthesized
CWLTKKKYSSSVHDPNGEYMFMTPRRPGPTRKHYQPYAPPRAVNTAKKSRLTDVTL

SEQ ID NO: 110 - DNA, 1791 bp, Artificial Sequence
  chemically synthesized
atggctctgcctgtgacagctctgctgctgcctctggctctgcttctgcatgccgccagacctgacatccagatgacccagacaaccagcagcctgtctgccagcctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcacaggcggcggaggaagcggaggcggaggatctggtggtggtggatctgaagtgaaactgcaagagtctggccctggcctggtggccccatctcaatctctgagcgtgacctgtaccgtcagcggagtgtccctgcctgattatggcgtgtcctggatccggcagcctcctagaaaaggcctggaatggctgggcgtgatctggggcagcgagacaacctactacaacagcgccctgaagtcccggctgaccatcatcaaggacaactccaagagccaggtgttcctgaagatgaacagcctccagaccgacgacaccgccatctactattgcgccaagcactactactacggcggcagctacgccatggattattggggccagggcaccagcgtgaccgtgtctagtacaacaacccctgctcctcggcctcctacaccagctcctacaattgccagccagccactgtctctgaggcccgaagcttgtagacctgctgctggcggagccgtgcatacaagaggactggatttcgcctgcagccagctgtgctgtcagctgaagttctggctgcctatcggctgcgccgcctttgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggctgcgaactgaattgcagaaacacaggcccctggctgaagaaagtgctgaagtgcaacacccctgatccgagcaagtttttcagccagctgagcagcgagcatggcggcgacgttcagaaatggctgtctagcccatttcctagctccagcttcagccctggtggactggcccctgagattagccctctggaagtgctggaacgggacaaagtgacccagctgctgcccctgaataccgacgcctacctgagcctgcaagagctgcaaggccaggatcctacacacctcgtgcgcgtgaagttcagcagatccgctgatgcccctgcctatcagcagggccaaaaccagctgtacaacgagctgaacctggggagaagagaagagtacgacgtgctggacaagcggagaggcagagatcctgaaatgggcggcaagcctgtgactagaggtgctggtgctggcggcagacagagaggccagaacaaagaaagacctcctcctgtgcctaatcctgactacgagcccatccggaagggccagagagatctgtacagcggcctgaactacagacaccagcggagaatctga

SEQ ID NO: 111 - protein, 22 aa, Artificial Sequence
  chemically synthesized
GSGATNFSLLKQAGDVEENPGP

SEQ ID NO: 112 - protein, 357 aa, Artificial Sequence
  chemically synthesized
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 113 - protein, 24 aa, Artificial Sequence
  chemically synthesized
MAVMAPRTLVLLLSGALALTQTWA

SEQ ID NO: 114 - DNA, 66 bp, Artificial Sequence
  chemically synthesized
ggaagcggcgccaccaacttcagcctgcttaaacaggcaggcgacgtggaagagaaccccggacct

SEQ ID NO: 115 - DNA, 1071 bp, Artificial Sequence
  chemically synthesized
atgctgctgctggttacatctctgctgctgtgcgagctgccccatcctgcctttctgctgatccccagaaaagtgtgcaacggcatcggcatcggagagttcaaggacagcctgagcatcaacgccaccaacatcaagcacttcaagaactgcaccagcatcagcggcgacctgcacattctgcctgtggcctttagaggcgacagcttcacccacacacctccactggaccctcaagagctggacatcctgaaaaccgtgaaagagatcaccggatttctgttgatccaggcttggcccgagaaccggacagatctgcacgccttcgagaacctggaaatcatcagaggccggaccaagcagcacggccagttttctctggctgtggtgtccctgaacatcaccagcctgggcctgagaagcctgaaagaaatcagcgacggcgacgtgatcatctccggcaacaagaacctgtgctacgccaacaccatcaactggaagaagctgttcggcaccagcggccagaaaacaaagatcatcagcaaccggggcgagaacagctgcaaggctacaggccaagtgtgccacgctctgtgtagccctgaaggctgttggggacccgagcctagagattgcgtgtcctgtcggaatgtgtcccggggcagagaatgcgtggacaagtgcaatctgctggaaggcgagccccgcgagttcgtggaaaacagcgagtgcatccagtgtcaccccgagtgtctgccccaggccatgaacattacctgtaccggcagaggccccgacaactgtattcagtgcgcccactacatcgacggccctcactgcgtgaaaacatgtcctgctggcgtgatgggagagaacaacaccctcgtgtggaagtatgccgacgccggacatgtgtgccacctgtgtcaccctaattgcacctacggctgtacaggccctggcctggaaggctgtccaacaaacggacctaagatcccctctatcgccaccggcatggttggagccctgctgcttctgctggtggtggcccttggaatcggcctgttcatg

SEQ ID NO: 116 - DNA, 72 bp, Artificial Sequence
  chemically synthesized
atggctgtgatggctcctagaacactggtgctgctgctgtctggtgccctggctctgactcagacatgggcc

SEQ ID NO: 117 - protein, 21 aa, Artificial Sequence
  chemically synthesized
MALPVTALLLPLALLLHAARP

SEQ ID NO: 118 - protein, 44 aa, Artificial Sequence
  chemically synthesized
TTTPAPRPPTPAPTIASQPLSLRPEACRPAAGGAVHTRGLDFAC

SEQ ID NO: 119 - protein, 22 aa, Artificial Sequence
  chemically synthesized
MLLLVTSLLLCELPHPAFLLIP

SEQ ID NO: 120 - protein, 118 aa, Artificial Sequence
  chemically synthesized
LFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 121 - protein, 106 aa, Artificial Sequence
  chemically synthesized
LFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 122 - protein, 97 aa, Artificial Sequence
  chemically synthesized
LFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 123 - protein, 109 aa, Artificial Sequence
  chemically synthesized
SQLCCQLKFWLPIGCAAFVVVCILGCILICWLTKKKYSSSVHDPNGEYMFMRAVNTAKKSRLTDVTLAAGAAALLYIFKQPFMRPVQTTQEEDGCSCRFPEEEEGGCEL

SEQ ID NO: 124 - protein, 109 aa, Artificial Sequence
  chemically synthesized
SQLCCQLKFWLPIGCAAFVVVCILGCILICWLTKKKYSSSVHDPNGEYMFMRAVNTAKKSRLTDVTLKRGRKKLLYIFAQPFMRPVQTTQEEDGCSCRFPEEEEGGCEL

SEQ ID NO: 125 - protein, 109 aa, Artificial Sequence
  chemically synthesized
SQLCCQLKFWLPIGCAAFVVVCILGCILICWLTKKKYSSSVHDPNGEYMFMRAVNTAKKSRLTDVTLAAGAAALLYIFAQPFMRPVQTTQEEDGCSCRFPEEEEGGCEL

SEQ ID NO: 126 - protein, 104 aa, Artificial Sequence
  chemically synthesized
SQLCCQLKFWLPIGCAAFVVVCILGCILICWLTKKKYSSSVHDPNGEYMFMRAVNTAKKSRLTDVTLRRDQRLPPDAHKPPGGGSFRTPIQEEQADAHSTLAKI

SEQ ID NO: 127 - protein, 104 aa, Artificial Sequence
  chemically synthesized
SQLCCQLKFWLPIGCAAFVVVCILGCILICWLTKKKYSSSVHDPNGEYMFMRAVNTAKKSRLTDVTLAADQALPPDAHKPPGGGSFRTPIQEEQADAHSTLAKI

SEQ ID NO: 128 - protein, 104 aa, Artificial Sequence
  chemically synthesized
SQLCCQLKFWLPIGCAAFVVVCILGCILICWLTKKKYSSSVHDPNGEYMFMRAVNTAKKSRLTDVTLRRDQRLPPDAHAPPGGGSFRTPIQEEQADAHSTLAAI

SEQ ID NO: 129 - protein, 104 aa, Artificial Sequence
  chemically synthesized
SQLCCQLKFWLPIGCAAFVVVCILGCILICWLTKKKYSSSVHDPNGEYMFMRAVNTAKKSRLTDVTLAADQALPPDAHAPPGGGSFRTPIQEEQADAHSTLAAI

SEQ ID NO: 130 - protein, 282 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 131 - protein, 270 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCEL

SEQ ID NO: 132 - protein, 261 aa, Artificial Sequence
  chemically synthesized
MAVMAPRTLVLLLSGALALTQTWAFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPALFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLA

SEQ ID NO: 133 - protein, 491 aa, Artificial Sequence
  chemically synthesized
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 134 - protein, 380 aa, Artificial Sequence
  chemically synthesized
MALPVTALLLPLALLLHAARPEVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 135 - protein, 510 aa, Artificial Sequence
  chemically synthesized
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSGGGGSGGGGSAVQLVESGGGLVQAGDSLRLTCTASGRAFSTYFMAWFRQAPGKEREFVAGIAWSGGSTAYADSVKGRFTISRDNAKNTVYLQMNSLKSEDTAVYYCASRGIEVEEFGAWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 136 - protein, 499 aa, Artificial Sequence
  chemically synthesized
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSGGGGSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 137 - protein, 185 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFAC

SEQ ID NO: 138 - protein, 365 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 139 - protein, 374 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 140 - protein, 416 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 141 - protein, 404 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 142 - protein, 395 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACLFPGPSKPFWVLVVVGGVLACYSLLVTVAFIIFWVRSKRSRLLHSDYMNMTPRRPGPTRKHYQPYAPPRDFAAYRSGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 143 - protein, 365 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 144 - protein, 407 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 145 - protein, 395 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 146 - protein, 386 aa, Artificial Sequence
  chemically synthesized
MALPVTALLLPLALLLHAARPEVQLQASGGGLAQPGGSLRLSCAASGRTFSTYFMAWFRQPPGKGLEYVGGIRWSDGVPHYADSVKGRFTISRDNAKNTVYLQMNSLRAEDTAVYFCASRGIADGSDFGSYGQGTQVTVSSTTTPAPRPPTPAPTIASQPLSLRPEACRPAAGGAVHTRGLDFACSQLCCQLKFWLPIGCAAFVVVCILGCILICWLTKKKYSSSVHDPNGEYMFMRAVNTAKKSRLTDVTLGGGSFRTPIQEEQADAHSTLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 147 - protein, 38 aa, Artificial Sequence
  chemically synthesized
CWLTKKKYSSSVHDPNGEYMFMRAVNTAKKSRLTDVTL

SEQ ID NO: 148 - DNA, 63 bp, Artificial Sequence
  chemically synthesized
atggctctgcctgtgacagctctgctgctgcctctggctctgcttctgcatgccgctagacct

SEQ ID NO: 149 - DNA, 132 bp, Artificial Sequence
  chemically synthesized
accaccacccccgcccccagaccccccacccccgcccccaccatcgccagccagcccctgagcctgagacccgaggcctgcagacccgccgccggcggcgccgtgcacaccagaggcctggacttcgcctgc

SEQ ID NO: 150 - DNA, 66 bp, Artificial Sequence
  chemically synthesized
atgctgctgctggttacatctctgctgctgtgcgagctgccccatcctgcctttctgctgatcccc

SEQ ID NO: 151 - DNA, 354 bp, Artificial Sequence
  chemically synthesized
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 152 - DNA, 318 bp, Artificial Sequence
  chemically synthesized
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 153 - DNA, 291 bp, Artificial Sequence
  chemically synthesized
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 154 - DNA, 201 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 155 - DNA, 327 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 156 - DNA, 291 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 157 - DNA, 264 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 158 - DNA, 345 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 159 - DNA, 318 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 160 - DNA, 327 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctggctgccggcgctgcagctctgctgtacatcttcaagcagcccttcatgcggcccgtgcagaccacacaagaggaagatggctgctcctgcagattccccgaggaagaagaaggcggctgcgaactt

SEQ ID NO: 161 - DNA, 327 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcgcacagcccttcatgcggcccgtgcagaccacacaagaggaagatggctgctcctgcagattccccgaggaagaagaaggcggctgcgaactt

SEQ ID NO: 162 - DNA, 327 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctggctgccggcgctgcagctctgctgtacatcttcgcacagcccttcatgcggcccgtgcagaccacacaagaggaagatggctgctcctgcagattccccgaggaagaagaaggcggctgcgaactt

SEQ ID NO: 163 - DNA, 312 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttaggagagaccagcgtctgccaccagatgcacataagccacctggcggcggaagctttagaacccctatccaagaggaacaggccgacgctcactctacactggctaaaatc

SEQ ID NO: 164 - DNA, 312 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttgccgcggaccaggccctgccaccagatgcacataagccacctggcggcggaagctttagaacccctatccaagaggaacaggccgacgctcactctacactggctaaaatc

SEQ ID NO: 165 - DNA, 312 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttaggagagaccagcgtctgccaccagatgcacatgcaccacctggcggcggaagctttagaacccctatccaagaggaacaggccgacgctcactctacactggctgcaatc

SEQ ID NO: 166 - DNA, 312 bp, Artificial Sequence
  chemically synthesized
agccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttgccgcggaccaggccctgccaccagatgcacatgcaccacctggcggcggaagctttagaacccctatccaagaggaacaggccgacgctcactctacactggctgcaatc

SEQ ID NO: 167 - DNA, 420 bp, Artificial Sequence; identical to SEQ ID NO: 73
  chemically synthesized

SEQ ID NO: 168 - DNA, 585 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggcctgctgggatctctggttctgcttgtttgggtgctcgccgtgatctgctctaga

SEQ ID NO: 169 - DNA, 720 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 170 - DNA, 846 bp, Artificial Sequence
  chemically synthesized
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactg

SEQ ID NO: 171 - DNA, 810 bp, Artificial Sequence
  chemically synthesized
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaactt

SEQ ID NO: 172 - DNA, 783 bp, Artificial Sequence
  chemically synthesized
atggctgtgatggcccctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgctctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggct

SEQ ID NO: 173 - DNA, 693 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacactt

SEQ ID NO: 174 - DNA, 819 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgaccctgaagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 175 - DNA, 783 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 176 - DNA, 756 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgagagccgtgaacaccgccaagaagtccagactgaccgacgtgacacttggcggcggaagctttagaacccctatccaagaggaacaggccgacgctcactctacactggct

SEQ ID NO: 177 - DNA, 837 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttcagcctttcatgaggcccgtgcagaccacacaagaagaggacggctgctcctgcagattccccgaggaagaggaaggcggttgcgaactt

SEQ ID NO: 178 - DNA, 810 bp, Artificial Sequence
  chemically synthesized
atggctgtgatggctcctagaacactggtgctgctgctgtctggtgccctggctctgactcagacatgggcctttctggacagccccgacagaccctggaatcctcctacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgttcgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccagccagctgtgctgccagctgaagttttggctgcctatcggctgtgccgccttcgtggttgtgtgtatcctgggctgcatcctgatctgctggctgaccaagaaaaagtacagcagctccgtgcacgaccccaacggcgagtacatgttcatgacccctagaaggcctggacctaccagaaagcactaccagccttacgctcctcctagagccgtgaacaccgccaagaagtccagactgaccgacgtgacacttggcggcggaagctttagaacccctatccaagaggaacaggccgacgctcactctacactggct

SEQ ID NO: 179 - DNA, 1473 bp, Artificial Sequence
  chemically synthesized
atggctctgcctgtgacagctctgctgctgcctctggctctgcttctgcatgccgccagacctgacatcgtgctgacacagtctccacctagcctggccatgagcctgggaaagagagccaccatcagctgtagagccagcgagagcgtgacaatcctgggctctcacctgatccactggtatcagcagaagcccggccagcctcctacactgctgattcagctggcctccaatgtgcagacaggcgtgccagccagattttctggcagcggcagcagaaccgacttcaccctgacaatcgaccccgtggaagaggacgatgtggccgtgtactactgcctccagagccggacaatccccagaacatttggcggaggcaccaagctggaaatcaagggcagcacaagcggctctggcaagcctggatctggcgagggatctaccaagggacagatccagctggtgcagtctggccccgagctgaagaaacctggcgagacagtgaagatcagctgcaaggccagcggctacaccttcaccgactacagcatcaactgggtcaagagagcccctggcaagggcctgaaatggatgggctggatcaacaccgaaaccagagagcccgcctacgcctacgacttcagaggcagattcgccttcagcctggaaaccagcgccagcacagcctacctccagatcaacaacctgaagtacgaggacaccgccacctacttttgcgccctggattacagctacgccatggactattggggccagggcacaagcgtgaccgtgtcctctacaacaacccctgctcctcggcctccaacaccagctcctacaattgcctctcagcccctgtctctgaggcccgaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccctgaggaagaagaaggcggctgcgagctgagagtgaagttcagcagatccgccgacgctcctgcctatcagcagggacagaatcagctctacaacgagctgaacctggggcgcagagaagagtacgacgtgctggacaagagaagaggcagggaccctgagatgggcggaaagccccagagaagaaagaaccctcaagagggcctgtacaatgagctgcaaaaggacaagatggccgaggcctacagcgagatcggaatgaagggcgaacgcagaagaggaaagggccacgacggactgtatcagggcctgagcacagccaccaaggacacctatgatgccctgcacatgcaggccctgcctccaaga

SEQ ID NO: 180 - DNA, 1140 bp, Artificial Sequence
  chemically synthesized
atggctctgcctgtgacagctctgctgctgcctctggctctgcttctgcatgccgctagacctgaggtgcagctgcttgaatctggcggaggactggttcagcctggcggatctctgagactgtcttgtgccgccagcggcttcacctttagcagctacgccatgagctgggtccgacaggctcctggcaaaggccttgaatgggtgtccagcatcagcggcagcggcgactacatctactacgccgatagcgtgaagggcagattcaccatcagccgggacatcagcaagaacaccctgtacctccagatgaacagcctgagagccgaggacaccgccgtgtactactgtgccaaagaaggcaccggcgccaatagcagcctggccgattatagaggccagggcacactggtcaccgtgtccagtttcgtgcctgtgttcctgcctgccaagcctaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcacactgtactgcaaccaccggaacaagcggggcagaaagaagctgctgtacatctttaagcagcccttcatgcggcccgtgcagaccacacaagaggaagatggctgctcctgtcggttccccgaggaagaagaaggcggctgcgagctgagagtgaagttcagcagatccgcagacgcccctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 181 - DNA, 1530 bp, Artificial Sequence
  chemically synthesized
atggctctgcctgtgacagctctgctgctgcctctggctctgcttctgcatgccgctagaccccaagtgaagctggaagagtctggcggaggactggtgcaggctggcagatctctgagactgtcttgtgccgccagcgagcacacctttagctctcacgtgatgggctggttcagacaggcccctggcaaagaaagggaaagcgtggccgttatcggctggcgggatatcagcacaagctacgccgatagcgtgaagggcagattcaccatcagccgggacaacgccaagaaaaccctgtacctccagatgaacagcctgaagcctgaggacaccgccgtgtactactgcgccgccagaagaattgacgccgccgactttgattcttggggccagggaacccaagtgaccgtttctagcggaggcggtggaagtggcggcggtggatcaggtggtggtggatctggtggcggaggaagcggcggaggcggatctgctgttcagcttgttgaatcaggtggcggcctggttcaggccggggattctcttagactgacctgcacagccagcggcagagccttcagcacctacttcatggcctggtttcggcaggctcccggaaaagaacgggaatttgtggccggaatcgcttggagcggaggctctacagcctatgccgattccgtgaaaggccggtttaccatcagcagagataatgccaaaaacacggtgtacctgcaaatgaactctctgaagtccgaggatacggccgtctattactgtgccagcagaggcatcgaggtggaagagtttggagcctggggacagggcacacaagtcacagtgtctagcaccagcaccaccacaccagctcctagacctccaactcctgctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagaagtgccgacgctcccgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 182 - DNA, 1497 bp, Artificial Sequence
  chemically synthesized
atggctctgcctgtgacagctctgctgctgcctctggctctgcttctgcatgccgctagaccccaagtgaagctggaagagtctggcggaggactggtgcaggctggcagatctctgagactgtcttgtgccgccagcgagcacacctttagctctcacgtgatgggctggttcagacaggcccctggcaaagaaagggaaagcgtggccgttatcggctggcgggatatcagcacaagctacgccgatagcgtgaagggcagattcaccatcagccgggacaacgccaagaaaaccctgtacctccagatgaacagcctgaagcctgaggacaccgccgtgtactactgcgccgccagaagaattgacgccgccgactttgattcttggggccagggaacccaagtgaccgtttctagcggaggcggtggaagtggcggcggtggatcaggtggtggtggatctgaagtgcagctggtggaatcaggcggcggtcttgttcaagccggtggttcactgagactgagctgtgccgcttccggcagaacctttaccatgggatggtttaggcaggctccagggaaagaacgcgagttcgtggccgccatttctctgtctccaacactggcctactacgccgagtccgtgaaaggccggttcacaatctccagagataatgccaagaacaccgtggtgctgcaaatgaactccctgaagccagaagatacagccctgtattactgtgccgccgaccggaagtccgtgatgagcatcagacctgactactggggacagggcacacaagtcacagtgtccagcaccagcaccacaacacccgctcctagacctccaacaccagctccaacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgagcctggtcatcaccctgtactgcaagcggggcagaaagaagctgctgtacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggacagaaccagctgtacaacgagctgaacctggggagaagagaagagtacgacgtgctggataagcggagaggcagagatcctgagatgggcggaaagccccagcggagaaagaatcctcaagagggcctgtataatgagctgcaaaaggacaagatggccgaggcctacagcgagatcggaatgaagggcgagcgcagaagaggcaagggacacgatggactgtatcagggcctgagcaccgccaccaaggatacctatgatgccctgcacatgcaggccctgcctccaaga

SEQ ID NO: 183 - DNA, 555 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgc

SEQ ID NO: 184 - DNA, 1095 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcgacatctacatctgggcccctctggctggaacatgtggcgtgctgctgctgagcctggtcatcaccctgtattgcaagcggggcagaaagaaactgctctacatcttcaagcagcccttcatgcggcccgtgcagaccacacaagaggaagatggctgctcctgtcggttccccgaggaagaagaaggcggctgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 185 - DNA, 1122 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 186 - DNA, 1248 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgtcggttccccgaggaagaagaaggcggttgcgaactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 187 - DNA, 1212 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatcccagcctttcatgaggcctgtgcagaccacacaagaagaggacggctgctcctgtcggttccccgaggaagaggaaggcggttgcgaacttagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 188 - DNA, 1185 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcctgtttcccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatctggcggcggaagcttcagaacccctatccaagaggaacaggccgacgctcactctacactggctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 189 - DNA, 1095 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgacactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 190 - DNA, 1221 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctgaagcggggcagaaagaagctgctgtatatcttcaagcagcccttcatgcggcccgtgcagaccacacaagaggaagatggctgctcctgtcggttccccgaggaagaagaaggcggttgcgaactgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 191 - DNA, 1185 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgaccctccagcctttcatgaggcctgtgcagaccacacaagaagaggacggctgctcctgtcggttccccgaggaagaggaaggcggttgcgaacttagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 192 - DNA, 1158 bp, Artificial Sequence
  chemically synthesized
atggctctgcctgtgacagctctgctgctgcctctggctctgcttctgcatgccgctagacctgaagtgcagttgcaggcttctggcggaggacttgctcaacctggcggaagcctgagactgtcttgtgccgcctctggcagaaccttcagcacctacttcatggcctggttcagacagcctcctggcaaaggcctggaatacgttggcggaatccgttggagtgatggcgtgccacactacgccgatagcgtgaagggcagattcaccatcagccgggacaacgccaagaacaccgtgtacctccagatgaacagcctgagagccgaggataccgccgtgtacttctgtgccagcagaggaatcgccgacggcagcgattttggctcttatggccagggcacccaagtgaccgtgtccagcacaacaacccctgctcctagacctcctacaccagctcctacaatcgccagccagcctctgtctctgaggccagaggcttgtagacctgctgctggcggagccgtgcatacaagaggactggatttcgcctgcagccagctgtgctgccagctgaagttctggctgcctattggctgcgccgccttcgtggttgtgtgtatcctgggctgcatcctgatctgctggctgaccaagaaaaagtacagcagcagcgtgcacgaccccaacggcgagtacatgttcatgagagccgtgaacaccgccaagaagtccagactgaccgacgtgacactcggcggaggcagctttagaacccctatccaagaggaacaggccgacgctcactctacactggctagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagccccagcggagaaagaatcctcaagagggcctgtataatgagctgcaaaaggacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 193 - protein, 41 aa, Artificial Sequence
  chemically synthesized
RSKRSRLLHSDYMNMTPRRPGPTRKHYQPYAPPRDFAAYRS

SEQ ID NO: 194 - DNA, 228 bp, Artificial Sequence
  chemically synthesized
ctgttccccggacctagcaagcccttttgggtgctcgttgttgttggcggcgtgctggcctgttatagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagatct

SEQ ID NO: 195 - DNA, 126 bp, Artificial Sequence
  chemically synthesized
aagcggggcagaaagaaactgctgtacatcttcaagcagcccttcatgcggcccgtgcagaccacacaagaggaagatggctgctcctgcagattccccgaggaagaagaaggcggctgcgaactt

SEQ ID NO: 196 - DNA, 111 bp, Artificial Sequence
  chemically synthesized
aggagagaccagcgtctgccaccagatgcacataagccacctggcggcggaagctttagaacccctatccaagaggaacaggccgacgctcactctacactggctaaaatc

SEQ ID NO: 197 - DNA, 90 bp, Artificial Sequence
  chemically synthesized
cagcctttcatgaggcccgtgcagaccacacaagaagaggacggctgctcctgcagattccccgaggaagaggaaggcggttgcgaactt

SEQ ID NO: 198 - DNA, 63 bp, Artificial Sequence
  chemically synthesized
ggcggcggaagctttagaacccctatccaagaggaacaggccgacgctcactctacactggct